(19) United States
(12) Patent Application Publication
(10) Pub. No.: US 2023/0121433 A1
(43) Pub. Date: Apr. 20, 2023

(54) CHIMERIC STIMULATORY RECEPTORS AND METHODS OF USE IN T CELL ACTIVATION AND DIFFERENTIATION

(71) Applicant: Poseida Therapeutics, Inc., San Diego, CA (US)

(72) Inventors: Devon SHEDLOCK, San Diego, CA (US); Sumiti JAIN, San Diego, CA (US)

(21) Appl. No.: 17/906,034

(22) PCT Filed: Mar. 11, 2021
(86) PCT No.: PCT/US2021/021957

§ 371 Date: Sep. 9, 2022

Related U.S. Application Data
(60) Provisional application No. 62/988,352, filed on Mar. 11, 2020.

(51) Int. Cl. A61K 35/17; C07K 14/725; C07K 14/715

(52) U.S. Cl. CPC A61K 35/17; C07K 14/7051; C07K 14/7155; C07K 14/7151; C07K 2319/03; C07K 2319/33

(57) ABSTRACT

Disclosed are chimeric stimulatory receptors (CSRs), cell compositions comprising CSRs, methods of making and methods of using same for the treatment of a disease or disorder in a subject.

### CROSS-REFERENCE TO RELATED APPLICATIONS

This application claims priority to, and the benefit of, U.S. Provisional Application No. 62/988,352, filed Mar. 11, 2020. The contents of this application are incorporated herein by reference in their entirety.

### FIELD OF THE DISCLOSURE

The disclosure is directed to molecular biology, and more, specifically, to chimeric receptors, T cell compositions (e.g., engineered regulatory T cells, effector T cells and helper T cells), methods of making and methods of using the same.

### INCORPORATION-BY-REFERENCE OF SEQUENCE LISTING

The contents of the file named “POTH-060 001WO SequenceListing ST25.txt”, which was created on Mar. 11, 2021, and is 709 KB in size are hereby incorporated by reference in their entirety.

### BACKGROUND OF THE INVENTION

Regulatory T cells (Tregs) act as negative regulators of the cytotoxicity and proliferation of conventional T cells, are key modulators of inflammation, systemically or in tissues to maintain homeostasis, and are important for peripheral tolerance. A lack of Treg activity can result in autoimmune conditions or accelerate allograft organ or hematopoietic stem cell transplant rejection. In autoimmune disorders, the suppressive function of Tregs is impaired or insufficient and the inability to modulate inflammation contributes to the disease state. A challenge in the field has been generating sufficient ex vivo expansion of Tregs that are free from effector T cell contamination. Current strategies for improving ex vivo expansion of Tregs cause a change of Treg functional properties and cause a differentiation of Tregs to other T cell types due to overstimulus. Contamination of Treg cells with other T cell types may exacerbate inflammation if administered to a recipient. Accordingly, there exists a long-felt and unmet need in the art for improved methods of ex vivo expansion of Tregs.

Effector T cells (Teffs) describes a group of cells that includes several T cell types that actively respond to a stimulus. A challenge in the field has been rapidly generating sufficient ex vivo expansion of Teffs that are free from contamination by other T cell types. Contamination of Teffs with other T cell types may reduce the effector function of the Tcells, if administered to a recipient. Accordingly, there exists a long-felt and unmet need in the art for improved methods of ex vivo expansion of Teffs.

The disclosure provides autologous and allogeneic T cell compositions, methods of making and methods of using these compositions, which comprise non-naturally occurring structural improvements to improve ex vivo expansion of Tregs and Teffs.

### SUMMARY OF THE INVENTION

The present disclosure provides a non-naturally occurring chimeric stimulatory receptor (CSR) comprising: (a) an ectodomain comprising a activation component, wherein the activation component is isolated or derived from a first protein; (b) a transmembrane domain; and (c) at least one intracellular domain, wherein the intracellular domain is isolated or derived from a second protein; and wherein the second protein is Tumor Necrosis Factor Receptor 2 (TNFR2).

In some embodiments, the activation component comprises an Interleukin-2 Receptor subunit Beta (IL2RB) extracellular domain, an Interleukin-2 Receptor subunit Gamma (IL2RG) extracellular domain or a CD2 extracellular domain. In some embodiments, the activation component comprises a modification. In some embodiments, the modification comprises a mutation or a truncation of the amino acid sequence of the activation component of the first protein when compared to a wild type sequence of the activation component of the first protein. In some embodiments, the mutation or a truncation of the amino acid sequence of the activation component comprises a mutation or truncation of a CD2 extracellular domain or a portion thereof to which an agonist binds. In some embodiments, the CSR comprising a mutation or truncation of a CD2 extracellular domain or a portion thereof to which an agonist binds does not bind CD58. In some embodiments, CD2 extracellular domain comprising the mutation or truncation comprises an amino acid sequence of SEQ ID NO: 4.

In some embodiments, the intracellular domain further a CD3 protein. In some embodiments, the CD3ζ intracellular domain comprises the amino acid sequence of SEQ ID NO: 5.

In some embodiments, the TNFR2 intracellular domain comprises the amino acid sequence of SEQ ID NO: 6.

In some embodiments, the ectodomain further comprises a signal peptide. In some embodiments, the signal peptide is derived from a third protein. In some embodiments, the first protein and the third protein are identical.

In some embodiments, the signal peptide comprises a CD2 signal peptide, an IL2RB signal peptide, an IL2RG signal peptide or a CD8 signal peptide. In some embodiments, the CD2 signal peptide comprises an amino acid sequence of SEQ ID NO: 9. In some embodiments, the CD8 signal peptide comprises an amino acid sequence of SEQ ID NO: 12.

In some embodiments, the transmembrane domain is isolated or derived from a fourth protein. In some embodiments, the first protein and the fourth protein are identical.

In some embodiments, the transmembrane domain comprises a TNFR2 transmembrane domain, CD2 transmembrane domain, an IL2RB transmembrane domain or an IL2RG transmembrane domain.

In some embodiments, the TNFR2 transmembrane domain comprises an amino acid sequence of SEQ ID NO: 13. In some embodiments, the CD2 transmembrane domain comprises an amino acid sequence of SEQ ID NO: 14.

In some embodiments, the CSR of the disclosure comprises an amino acid sequence at least 95% identical to any one of SEQ ID NO: 17, 18, 37 or 38. In some embodiments, the CSR of the disclosure comprises an amino acid sequence at least 99% identical to any one of SEQ ID NO: 17, 18, 37 or 38. In some embodiments, the CSR of the disclosure comprises an amino acid sequence of any one of SEQ ID NO: 17, 18, 37 or 38.

The present disclosure also provides a non-naturally occurring chimeric stimulatory receptor (CSR) comprising: (a) an ectodomain comprising a signal peptide and an activation component, wherein the signal peptide comprises a CD2 signal peptide or a portion thereof or comprises a CD8 signal peptide or a portion thereof; and wherein the activation component comprises a CD2 extracellular domain or a portion thereof and wherein the CD2 extracellular domain or a portion thereof comprises a mutation or truncation when compared to a wild type sequence of CD2; (b) a transmembrane domain, wherein the transmembrane domain comprises a TNFR2 transmembrane domain or a portion thereof or comprises a CD2 transmembrane domain or portion thereof; and (c) an intracellular domain, wherein the intracellular domain comprises a TNFR2 intracellular domain or a portion thereof and a CD3ζ protein or a portion thereof.

The present disclosure also provides a nucleic acid sequence encoding the CSR. The present disclosure also provides cell comprising the CSR. The present disclosure also provides a cell comprising the nucleic encoding the CSR.

In some embodiments, the cell is an allogeneic cell. In some embodiments, the cell is an autologous cell. In some embodiments, the cell is a T-lymphocyte (T cell). In some embodiments, the T cell is a regulatory T cell. In some embodiments, the CSR is transiently expressed in the modified T cell. In some embodiments, the CSR is stably expressed in the modified T cell.

The present disclosure also provides a composition comprising a population of modified T cells, wherein a plurality of the modified T cells of the population comprise the CSR.

The present disclosure also provides a composition for use in the treatment of a disease or disorder. The present disclosure also provides a method of treating a disease or disorder comprising administering to a subject in need thereof a therapeutically-effective amount of the composition.

The present disclosure also provides a method of producing a population of modified T cells comprising introducing into a plurality of primary human T cells a composition comprising the CSR or a sequence encoding the same to produce a plurality of modified T cells under conditions that stably express the CSR within the plurality of modified T cells.

The present disclosure also provides a method of producing a population of modified T cells comprising introducing into a plurality of primary human T cells a composition comprising the CSR or a sequence encoding the same to produce a plurality of modified T cells under conditions that transiently express the CSR within the plurality of modified T cells.

The present disclosure also provides method of expanding modified Treg cells within the population of modified T cells produced by the methods of the disclosure comprising culturing the modified T cells with an activator composition under conditions to expand a plurality of activated modified Treg cells, wherein expansion of the plurality of modified Treg cells is at least 5% higher than the expansion of a plurality of Treg cells not stably expressing the CSR under the same conditions. In some embodiments, the expansion of the plurality of modified T cells is at least 10% higher than the expansion of a plurality of wild-type T cells not stably expressing the CSR under the same conditions.

The present disclosure also provides a composition comprising a population of modified T cells expanded by the methods of the disclosure. In some embodiments, the composition is for use in treatment of a disease or disorder.

The present disclosure provides a non-naturally occurring chimeric stimulatory receptor (CSR) comprising: (a) an ectodomain comprising a activation component, wherein the activation component is isolated or derived from a first protein; (b) a transmembrane domain; and (c) at least one intracellular domain, wherein the intracellular domain is isolated or derived from a second protein; wherein the first protein and the second protein are not identical.

The activation component comprises an Interleukin-2 Receptor subunit Beta (IL2RB) extracellular domain or a portion thereof, an Interleukin-2 Receptor subunit Gamma (IL2RG) extracellular domain or a portion thereof or a CD2 extracellular domain or a portion thereof. In some embodiments, the activation component can comprise a CD2 extracellular domain or a portion thereof to which an agonist binds.

In some embodiments, the IL2RB extracellular domain or a portion thereof comprises the amino acid sequence at least 80%, at least 90%, at least 95%, at least 99% or 100% identical to SEQ ID NO: 2. In a preferred embodiment, the IL2RB extracellular domain or a portion thereof comprises the amino acid sequence of SEQ ID NO: 2. In some embodiments, the IL2RG extracellular domain or a portion thereof comprises the IL2RG extracellular domain comprises the amino acid sequence at least 80%, at least 90%, at least 95%, at least 99% or 100% identical to SEQ ID NO: 3. In a preferred embodiment, the IL2RG extracellular domain or a portion thereof comprises the amino acid sequence of SEQ ID NO: 3. In some embodiments, the CD2 extracellular domain or a portion thereof comprises the amino acid sequence at least 80%, at least 90%, at least 95%, at least 99% or 100% identical to SEQ ID NO: 1. In a preferred embodiment, the CD2 extracellular domain or a portion thereof comprises the amino acid sequence of SEQ ID NO: 1.

The present disclosure also provides a non-naturally occurring chimeric stimulatory receptor (CSR) wherein the ectodomain comprises a modification. In some embodiments, the modification comprises a mutation or a truncation of the amino acid sequence of the activation component of the first protein when compared to a wild type sequence of the activation component of the first protein. In some embodiments, the mutation or a truncation of the amino acid sequence of the activation component comprises a mutation or truncation of a CD2 extracellular domain or a portion thereof to which an agonist binds. In some embodiments, the CSR comprising a mutation or truncation of a CD2 extracellular domain or a portion thereof to which an agonist binds does not bind CD58. In some embodiments, the CD2 extracellular domain comprising the mutation or truncation comprises an amino acid sequence at least 80%, at least 90%, at least 95%, at least 99% or 100% identical to SEQ ID NO: 4. In a preferred embodiment, the CD2 extracellular domain comprising the mutation or truncation comprises an amino acid sequence of SEQ ID NO: 4.

In some embodiments, the intracellular domain comprises a Tumor Necrosis Factor Receptor 2 (TNFR2) intracellular domain or a portion thereof, an IL2RB intracellular domain or a portion thereof, an IL2RG intracellular domain portion thereof, a CD3ζ protein or a portion thereof, or a combination thereof.

In some embodiments, the TNFR2 intracellular domain or portion thereof comprises an amino acid sequence at least 80%, at least 90%, at least 95%, at least 99% or 100% identical to SEQ ID NO: 6. In a preferred embodiment, the TNFR2 intracellular domain or a portion thereof comprises an amino acid sequence of SEQ ID NO: 6. In some embodiments, the IL2RB intracellular domain or portion thereof comprises an amino acid sequence at least 80%, at least 90%, at least 95%, at least 99% or 100% identical to SEQ ID NO: 2. In a preferred embodiment, the IL2RB intracellular domain or a portion thereof comprises an amino acid sequence of SEQ ID NO: 2. In some embodiments, the IL2RG intracellular domain or portion thereof comprises an amino acid sequence at least 80%, at least 90%, at least 95%, at least 99% or 100% identical to SEQ ID NO: 3. In a preferred embodiment, the IL2RG intracellular domain or a portion thereof comprises an amino acid sequence of SEQ ID NO: 3. In some embodiments, the CD3ζ intracellular domain or portion thereof comprises an amino acid sequence at least 80%, at least 90%, at least 95%, at least 99% or 100% identical to SEQ ID NO: 5. In a preferred embodiment, the CD3λ intracellular domain or a portion thereof comprises an amino acid sequence of SEQ ID NO: 5.

In some embodiments, the ectodomain further comprises a signal peptide. In some embodiments, the signal peptide is derived from a third protein. In some embodiments, the first protein and the third protein are identical. In some embodiments, the transmembrane domain is isolated or derived from a fourth protein. In some embodiments, the first protein and the fourth protein are identical.

In some embodiments, the signal peptide comprises a CD2 signal peptide or a portion thereof, an IL2RB signal peptide or a portion thereof, an IL2RG signal peptide or a portion thereof or a CD8 signal peptide or a portion thereof.

In some embodiments, the CD2 signal peptide or a portion thereof comprises an amino acid sequence at least 80%, at least 90%, at least 95%, at least 99% or 100% identical to SEQ ID NO: 9. In a preferred embodiment, the CD2 signal peptide or a portion thereof comprises and amino acid sequence of SEQ ID NO: 9. In some embodiments, the IL2RB signal peptide or a portion thereof comprises an amino acid sequence at least 80%, at least 90%, at least 95%, at least 99% or 100% identical to SEQ ID NO: 10. In a preferred embodiment, the IL2RB signal peptide or a portion thereof comprises and amino acid sequence of SEQ ID NO: 10. In some embodiments, the IL2RG signal peptide or a portion thereof comprises an amino acid sequence at least 80%, at least 90%, at least 95%, at least 99% or 100% identical to SEQ ID NO: 11. In a preferred embodiment, the IL2RG signal peptide or a portion thereof comprises and amino acid sequence of SEQ ID NO: 11. In some embodiments, the CD8 signal peptide or a portion thereof comprises an amino acid sequence at least 80%, at least 90%, at least 95%, at least 99% or 100% identical to SEQ ID NO: 12. In a preferred embodiment, the CD8 signal peptide or a portion thereof comprises and amino acid sequence of SEQ ID NO: 12.

In some embodiments, the transmembrane domain comprises a TNFR2 transmembrane domain or a portion thereof, CD2 transmembrane domain or a portion thereof, an IL2RB transmembrane domain or a portion thereof or an IL2RG transmembrane domain or a portion thereof.

In some embodiments, the TNFR2 transmembrane domain or a portion thereof comprises an amino acid sequence at least 80%, at least 90%, at least 95%, at least 99% or 100% identical to SEQ ID NO: 13. In a preferred embodiment, the TNFR2 transmembrane domain or a portion thereof comprises the amino acid sequence of SEQ ID NO ID NO: 13. In some embodiments, the CD2 transmembrane domain or a portion thereof comprises an amino acid sequence at least 80%, at least 90%, at least 95%, at least 99% or 100% identical to SEQ ID NO: 14. In a preferred embodiment, the CD2 transmembrane domain or a portion thereof comprises the amino acid sequence of SEQ ID NO ID NO: 14. In some embodiments, the IL2RB transmembrane domain or a portion thereof comprises an amino acid sequence at least 80%, at least 90%, at least 95%, at least 99% or 100% identical to SEQ ID NO: 15. In a preferred embodiment, the IL2RB transmembrane domain or a portion thereof comprises the amino acid sequence of SEQ ID NO ID NO: 15. In some embodiments, the IL2RG transmembrane domain or a portion thereof comprises an amino acid sequence at least 80%, at least 90%, at least 95%, at least 99% or 100% identical to SEQ ID NO: 16. In a preferred embodiment, the IL2RG transmembrane domain or a portion thereof comprises the amino acid sequence of SEQ ID NO ID NO: 16.

In some embodiments, the activation component does not bind a naturally-occurring molecule. In some embodiments, the activation component binds a naturally-occurring molecules but the CSR does not transduce a signal upon binding of the activation component. In some embodiments, the activation component binds to a non-naturally occurring molecule. In some embodiments, the CSR selectively transduces a signal upon binding of the activation component to a non-naturally occurring molecule.

In some embodiments, the CSR comprises an amino acid sequence at least 80%, at least 85%, at least 90%, at least 95%, at least 99% or 100% identical to SEQ ID NO: 17. In a preferred embodiment, the CSR comprises an amino acid sequence of SEQ ID NO: 17. In some embodiments, the CSR comprises an amino acid sequence at least 80%, at least 85%, at least 90%, at least 95%, at least 99% or 100% identical to SEQ ID NO: 18. In a preferred embodiment, the CSR comprises an amino acid sequence of SEQ ID NO: 18. In some embodiments, the CSR comprises an amino acid sequence at least 80%, at least 85%, at least 90%, at least 95%, at least 99% or 100% identical to SEQ ID NO: 19. In a preferred embodiment, the CSR comprises an amino acid sequence of SEQ ID NO: 19. In some embodiments, the CSR comprises an amino acid sequence at least 80%, at least 85%, at least 90%, at least 95%, at least 99% or 100% identical to SEQ ID NO: 20. In a preferred embodiment, the CSR comprises an amino acid sequence of SEQ ID NO: 20. In some embodiments, the CSR comprises an amino acid sequence at least 80%, at least 85%, at least 90%, at least 95%, at least 99% or 100% identical to SEQ ID NO: 21. In a preferred embodiment, the CSR comprises an amino acid sequence of SEQ ID NO: 21. In some embodiments, the CSR comprises an amino acid sequence at least 80%, at least 85%, at least 90%, at least 95%, at least 99% or 100% identical to SEQ ID NO: 22. In a preferred embodiment, the CSR comprises an amino acid sequence of SEQ ID NO: 22. In some embodiments, the CSR comprises an amino acid sequence at least 80%, at least 85%, at least 90%, at least 95%, at least 99% or 100% identical to SEQ ID NO: 23. In a preferred embodiment, the CSR comprises an amino acid sequence of SEQ ID NO: 23. In some embodiments, the CSR comprises an amino acid sequence at least 80%, at least 85%, at least 90%, at least 95%, at least 99% or 100% identical to SEQ ID NO: 24. In a preferred embodiment, the CSR comprises an amino acid sequence of SEQ ID NO: 24. In some embodiments, the CSR comprises an amino acid sequence at least 80%, at least 85%, at least 90%, at least 95%, at least 99% or 100% identical to SEQ ID NO: 25. In a preferred embodiment, the CSR comprises an amino acid sequence of SEQ ID NO: 25. In some embodiments, the CSR comprises an amino acid sequence at least 80%, at least 85%, at least 90%, at least 95%, at least 99% or 100% identical to SEQ ID NO: 26. In a preferred embodiment, the CSR comprises an amino acid sequence of SEQ ID NO: 26. In some embodiments, the CSR comprises an amino acid sequence at least 80%, at least 85%, at least 90%, at least 95%, at least 99% or 100% identical to SEQ ID NO: 27. In a preferred embodiment, the CSR comprises an amino acid sequence of SEQ ID NO: 27. In some embodiments, the CSR comprises an amino acid sequence at least 80%, at least 85%, at least 90%, at least 95%, at least 99% or 100% identical to SEQ ID NO: 28. In a preferred embodiment, the CSR comprises an amino acid sequence of SEQ ID NO: 28. In some embodiments, the CSR comprises an amino acid sequence at least 80%, at least 85%, at least 90%, at least 95%, at least 99% or 100% identical to SEQ ID NO: 29. In a preferred embodiment, the CSR comprises an amino acid sequence of SEQ ID NO: 29. In some embodiments, the CSR comprises an amino acid sequence at least 80%, at least 85%, at least 90%, at least 95%, at least 99% or 100% identical to SEQ ID NO: 30. In a preferred embodiment, the CSR comprises an amino acid sequence of SEQ ID NO: 30. In some embodiments, the CSR comprises an amino acid sequence at least 80%, at least 85%, at least 90%, at least 95%, at least 99% or 100% identical to SEQ ID NO: 33. In a preferred embodiment, the CSR comprises an amino acid sequence of SEQ ID NO: 33. In some embodiments, the CSR comprises an amino acid sequence at least 80%, at least 85%, at least 90%, at least 95%, at least 99% or 100% identical to SEQ ID NO: 32. In a preferred embodiment, the CSR comprises an amino acid sequence of SEQ ID NO: 32. In some embodiments, the CSR comprises an amino acid sequence at least 80%, at least 85%, at least 90%, at least 95%, at least 99% or 100% identical to SEQ ID NO: 33. In a preferred embodiment, the CSR comprises an amino acid sequence of SEQ ID NO: 33. In some embodiments, the CSR comprises an amino acid sequence at least 80%, at least 85%, at least 90%, at least 95%, at least 99% or 100% identical to SEQ ID NO: 34. In a preferred embodiment, the CSR comprises an amino acid sequence of SEQ ID NO: 34. In some embodiments, the CSR comprises an amino acid sequence at least 80%, at least 85%, at least 90%, at least 95%, at least 99% or 100% identical to SEQ ID NO: 35. In a preferred embodiment, the CSR comprises an amino acid sequence of SEQ ID NO: 35. In some embodiments, the CSR comprises an amino acid sequence at least 80%, at least 85%, at least 90%, at least 95%, at least 99% or 100% identical to SEQ ID NO: 36. In a preferred embodiment, the CSR comprises an amino acid sequence of SEQ ID NO: 36. In some embodiments, the CSR comprises an amino acid sequence at least 80%, at least 85%, at least 90%, at least 95%, at least 99% or 100% identical to SEQ ID NO: 37. In a preferred embodiment, the CSR comprises an amino acid sequence of SEQ ID NO: 37. In some embodiments, the CSR comprises an amino acid sequence at least 80%, at least 85%, at least 90%, at least 95%, at least 99% or 100% identical to SEQ ID NO: 38. In a preferred embodiment, the CSR comprises an amino acid sequence of SEQ ID NO: 38. In some embodiments, the CSR comprises an amino acid sequence at least 80%, at least 85%, at least 90%, at least 95%, at least 99% or 100% identical to SEQ ID NO: 39. In a preferred embodiment, the CSR comprises an amino acid sequence of SEQ ID NO: 39. In some embodiments, the CSR comprises an amino acid sequence at least 80%, at least 85%, at least 90%, at least 95%, at least 99% or 100% identical to SEQ ID NO: 40. In a preferred embodiment, the CSR comprises an amino acid sequence of SEQ ID NO: 40. In some embodiments, the CSR comprises an amino acid sequence at least 80%, at least 85%, at least 90%, at least 95%, at least 99% or 100% identical to SEQ ID NO: 41. In a preferred embodiment, the CSR comprises an amino acid sequence of SEQ ID NO: 41. In some embodiments, the CSR comprises an amino acid sequence at least 80%, at least 85%, at least 90%, at least 95%, at least 99% or 100% identical to SEQ ID NO: 42. In a preferred embodiment, the CSR comprises an amino acid sequence of SEQ ID NO: 42. In some embodiments, the CSR comprises an amino acid sequence at least 80%, at least 85%, at least 90%, at least 95%, at least 99% or 100% identical to SEQ ID NO: 43. In a preferred embodiment, the CSR comprises an amino acid sequence of SEQ ID NO: 43. In some embodiments, the CSR comprises an amino acid sequence at least 80%, at least 85%, at least 90%, at least 95%, at least 99% or 100% identical to SEQ ID NO: 44. In a preferred embodiment, the CSR comprises an amino acid sequence of SEQ ID NO: 44. In some embodiments, the CSR comprises an amino acid sequence at least 80%, at least 85%, at least 90%, at least 95%, at least 99% or 100% identical to SEQ ID NO: 45. In a preferred embodiment, the CSR comprises an amino acid sequence of SEQ ID NO: 45. In some embodiments, the CSR comprises an amino acid sequence at least 80%, at least 85%, at least 90%, at least 95%, at least 99% or 100% identical to SEQ ID NO: 46. In a preferred embodiment, the CSR comprises an amino acid sequence of SEQ ID NO: 46. In some embodiments, the CSR comprises an amino acid sequence at least 80%, at least 85%, at least 90%, at least 95%, at least 99% or 100% identical to SEQ ID NO: 47. In a preferred embodiment, the CSR comprises an amino acid sequence of SEQ ID NO: 47. In some embodiments, the CSR comprises an amino acid sequence at least 80%, at least 85%, at least 90%, at least 95%, at least 99% or 100% identical to SEQ ID NO: 48. In a preferred embodiment, the CSR comprises an amino acid sequence of SEQ ID NO: 48. In some embodiments, the CSR comprises an amino acid sequence at least 80%, at least 85%, at least 90%, at least 95%, at least 99% or 100% identical to SEQ ID NO: 49. In a preferred embodiment, the CSR comprises an amino acid sequence of SEQ ID NO: 49. In some embodiments, the CSR comprises an amino acid sequence at least 80%, at least 85%, at least 90%, at least 95%, at least 99% or 100% identical to SEQ ID NO: 50. In a preferred embodiment, the CSR comprises an amino acid sequence of SEQ ID NO: 50. In some embodiments, the CSR comprises an amino acid sequence at least 80%, at least 85%, at least 90%, at least 95%, at least 99% or 100% identical to SEQ ID NO: 51. In a preferred embodiment, the CSR comprises an amino acid sequence of SEQ ID NO: 51. In some embodiments, the CSR comprises an amino acid sequence at least 80%, at least 85%, at least 90%, at least 95%, at least 99% or 100% identical to SEQ ID NO: 52. In a preferred embodiment, the CSR comprises an amino acid sequence of SEQ ID NO: 52. In some embodiments, the CSR comprises an amino acid sequence at least 80%, at least 85%, at least 90%, at least 95%, at least 99% or 100% identical to SEQ ID NO: 53. In a preferred embodiment, the CSR comprises an amino acid sequence of SEQ ID NO: 53. In some embodiments, the CSR comprises an amino acid sequence at least 80%, at least 85%, at least 90%, at least 95%, at least 99% or 100% identical to SEQ ID NO: 54. In a preferred embodiment, the CSR comprises an amino acid sequence of SEQ ID NO: 54. In some embodiments, the CSR comprises an amino acid sequence at least 80%, at least 85%, at least 90%, at least 95%, at least 99% or 100% identical to SEQ ID NO: 55. In a preferred embodiment, the CSR comprises an amino acid sequence of SEQ ID NO: 55. In some embodiments, the CSR comprises an amino acid sequence at least 80%, at least 85%, at least 90%, at least 95%, at least 99% or 100% identical to SEQ ID NO: 56. In a preferred embodiment, the CSR comprises an amino acid sequence of SEQ ID NO: 56. In some embodiments, the CSR comprises an amino acid sequence at least 80%, at least 85%, at least 90%, at least 95%, at least 99% or 100% identical to SEQ ID NO: 57. In a preferred embodiment, the CSR comprises an amino acid sequence of SEQ ID NO: 57. In some embodiments, the CSR comprises an amino acid sequence at least 80%, at least 85%, at least 90%, at least 95%, at least 99% or 100% identical to SEQ ID NO: 58. In a preferred embodiment, the CSR comprises an amino acid sequence of SEQ ID NO: 58.

The present disclosure also provides a non-naturally occurring chimeric stimulatory receptor (CSR) comprising: (a) an ectodomain comprising a signal peptide and an activation component, wherein the signal peptide comprises a CD2 signal peptide or a portion thereof and wherein the activation component comprises a CD2 extracellular domain or a portion thereof and wherein the CD2 extracellular domain or a portion thereof comprises a mutation or truncation; (b) a transmembrane domain, wherein the transmembrane domain comprises a TNFR2 transmembrane domain or a portion thereof; and (c) an intracellular domain, wherein the intracellular domain comprises a TNFR2 intracellular domain or a portion thereof and a CD3ζ protein or a portion thereof.

The present disclosure also provides a non-naturally occurring chimeric stimulatory receptor (CSR) comprising: (a) an ectodomain comprising a signal peptide and an activation component, wherein the signal peptide comprises a CD2 signal peptide or a portion thereof and wherein the activation component comprises a CD2 extracellular domain or a portion thereof and wherein the CD2 extracellular domain or a portion thereof comprises a mutation or truncation; (b) a transmembrane domain, wherein the transmembrane domain comprises a CD2 transmembrane domain or a portion thereof; and (c) an intracellular domain comprising a TNFR2 intracellular domain or a portion thereof and a CD3ζ protein or a portion thereof.

The present disclosure provides a nucleic acid sequence encoding any CSR disclosed herein. The present disclosure provides a vector comprising a nucleic acid sequence encoding any CSR disclosed herein. The present disclosure provides a transposon comprising a nucleic acid sequence encoding any CSR disclosed herein.

The present disclosure provides a cell comprising any CSR disclosed herein. The present disclosure provides a cell comprising a nucleic acid sequence encoding any CSR disclosed herein. The present disclosure provides a cell comprising a vector comprising a nucleic acid sequence encoding any CSR disclosed herein. The present disclosure provides a cell comprising a transposon comprising a nucleic acid sequence encoding any CSR disclosed herein.

A modified cell disclosed herein can be an allogeneic cell or an autologous cell. In some embodiments, the modified cell is a T cell. In some embodiments, the modified cell is a regulatory T cell. In some embodiments, the modified cell is an effector T cell. In some embodiments, the modified cell is stem memory T cell (TSCM cell) or a TSCM-like cell. In some embodiments, the modified cell is a helper T cell. In some preferred aspects, the modified cell is an allogeneic cell. In some preferred aspects, the modified cell is a regulatory T cell. In some preferred aspects, the modified cell is an effector T cell.

The present disclosure provides a composition comprising any CSR disclosed herein. The present disclosure provides a composition comprising a nucleic acid sequence encoding any CSR disclosed herein. The present disclosure provides a composition comprising a vector comprising a nucleic acid sequence encoding any CSR disclosed herein. The present disclosure provides a composition comprising a transposon comprising a nucleic acid sequence encoding any CSR disclosed herein. The present disclosure provides a composition comprising a modified cell disclosed herein or a composition comprising a plurality of modified cells disclosed herein.

The present disclosure provides a modified T lymphocyte (T cell), comprising any CSR disclosed herein. In some embodiments, the modified T cell can further comprise an inducible proapoptotic polypeptide. In some embodiments, CSR is transiently expressed in the modified T cell. In some embodiments, the CSR is stably expressed in the modified T cell. In some embodiments, the inducible proapoptotic polypeptide is stably expressed in the modified T cell.

In some embodiments, the modified T cell is an allogeneic cell. In some embodiments, the modified T cell is an autologous cell. In some embodiments, the modified T cell is a regulatory T cell, an effector T cell, a stem memory T cell (T_{SCM}) or a helper T cell.

In some embodiments, the modified T cell further comprises a transgene comprising a sequence encoding an inducible promoter and a sequence encoding a transcription factor or a non-naturally occurring protein sequence. In some embodiments, the transgene comprises a sequence encoding a non-naturally occurring antigen receptor, a sequence encoding a therapeutic polypeptide, or a combination thereof. In some embodiments, the non-naturally occurring antigen receptor comprises a chimeric antigen receptor (CAR). In some preferred aspects, the modified cell is an allogeneic T cell or a modified allogeneic CAR T cell. In some embodiments, the transgene comprises a nucleic sequence encoding Forkhead Box P3 (FoxP3). In some embodiments, a transposon comprises the transgene. In some embodiments, the transposon is a piggyBac transposon, a piggy-Bac like transposon, a Sleeping Beauty transposon, a Helraiser transposon, a To12 transposon or a TcBuster transposon.

In some embodiments, the modified T cell further comprises a modification of an endogenous sequence encoding a T cell Receptor (TCR), wherein the modification reduces or eliminates a level of expression or activity of the TCR. In some embodiments, the modified T cell further comprises a modification of an endogenous sequence encoding Beta-2-Microglobulin (B2M), wherein the modification reduces or eliminates a level of expression or activity of a major histocompatibility complex (MHC) class I (MHC-I).

The present disclosure provides a composition comprising any of the modified T cell disclosed herein. In some embodiments, the composition comprises a plurality of the modified T cells of the population comprise any CSR disclosed herein. In some embodiments, the composition comprises a population of modified T cells, wherein a plurality of the modified T cells of the population comprise any of the modified T cell disclosed herein. In some embodiments, the composition disclosed herein is used for the treatment of a disease or disorder.

The present disclosure provides a method of treating a disease or disorder comprising administering to a subject in need thereof a therapeutically-effective amount of any of the compositions disclosed herein. In some embodiments, method of treating a disease or disorder further comprises at least one non-naturally occurring molecule that binds the CSR.

The present disclosure provides a method of producing a population of modified T cells comprising introducing into a plurality of primary human T cells a composition comprising any of the CSRs disclosed herein or any of the sequences encoding the same to produce a plurality of modified T cells under conditions that stably express the CSR within the plurality of modified T cells. In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprises the CSR.

The present disclosure provides a method of producing a population of modified T cells comprising introducing into a plurality of primary human T cells a composition comprising any of the CSRs disclosed herein or any of the sequences encoding the same to produce a plurality of modified T cells under conditions that transiently express the CSRs within the plurality of modified T cells. In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprises the CSR.

The present disclosure provides a method of treating a disease or disorder comprising administering to a subject in need thereof a therapeutically-effective amount of any of the compositions disclosed herein. In some embodiments, the modified T cells within the population of modified T cells administered to the subject no longer express the CSR.

The present disclosure provides a method of expanding a population of modified T cells comprising introducing into a plurality of primary human T cells a composition comprising any of the CSRs disclosed herein or any of the sequences disclosed herein encoding the same to produce a plurality of modified T cells under conditions that stably express the CSR within the plurality of modified T cells and preserve desirable stem-like properties of the plurality of modified T cells and contacting the cells with an activator composition to produce a plurality of activated modified T cells, wherein expansion of the plurality of modified T cells is at least two fold higher than the expansion of a plurality of wild-type T cells not stably expressing the CSR under the same conditions. In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprises the CSR.

The present disclosure provides a composition comprising a population of modified T cells expanded by any method disclosed herein. In some embodiments, the composition is used in the treatment of a disease or disorder.

The present disclosure provides a method of expanding a population of modified T cells comprising introducing into a plurality of primary human T cells a composition comprising any of the CSRs disclosed herein or any of the sequences disclosed herein encoding the same to produce a plurality of modified T cells under conditions that transiently express the CSR within the plurality of modified T cells and preserve desirable stem-like properties of the plurality of modified T cells and contacting the cells with an activator composition to produce a plurality of activated modified T cells, wherein expansion of the plurality of modified T cells is at least two fold higher than the expansion of a plurality of wild-type T cells not transiently expressing the CSR under the same conditions. In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprises the CSR.

The present disclosure provides a composition comprising a population of modified T cells produced by any of the methods disclosed herein. In some embodiments, the composition is used in the treatment of a disease or disorder. The present disclosure also provides a method of treating a disease or disorder comprising administering to a subject in need thereof a therapeutically-effective amount of any of the compositions disclosed herein. In some embodiments, the method further comprises administering an activator composition to the subject to activate the population of modified T cells in vivo, to induce cell division of the population of modified T cells in vivo, or a combination thereof. In some embodiments, the modified T cells within the population of modified T cells administered to the subject no longer express the CSR.

Any of the above aspects can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram depicting exemplary chimeric stimulatory receptors (CSRs) of the disclosure. A schematic of a CSR control is shown (CD2z V2). The CSRs of the disclosure contain a TNFR2 and a CD3z intracellular domain (11. 2DH.TF2z; 12. 2DH.2TF2z WT; 33. 82DH.TF2z; 34. 82DH.2TF2z WT). These CSRs contain a mutated CD2 ectodomain with either a CD2 or a CD8a signal peptide. These CSRs contain either a TNFR2 or a CD2 transmembrane domain. These CSR constructs specifically promote differentiation and expansion of Treg or Treg-like cells.

FIG. 2 is a schematic diagram depicting exemplary CSRs of the disclosure. These CSRs contain an IL2RB, IL2RG or CD3z intracellular domain, or a combination thereof. Some of these CSRs contain a mutated CD2 ectodomain with a CD2 signal peptide. Others contain an IL2RB or IL2RG ectodomain with a native signal peptide. These CSRs contain either an IL2RB, IL2RG, or CD2 transmembrane domain. These CSR constructs specifically promote differentiation, expansion, and function of Treg or Treg-like cells. These CSR constructs also specifically promote differentiation, expansion, and function of effector T cells or effector-like T cells.

FIG. 3 is a schematic diagram depicting exemplary CSRs of the disclosure. These CSRs contain an IL2RB, IL2RG or CD3z intracellular domain, or a combination thereof. Some of these CSRs contain a mutated CD2 ectodomain. Others contain an IL2RB or IL2RG ectodomain with a native signal peptide. These CSRs contain either an IL2RB, IL2RG, or CD2 transmembrane domain. These CSR constructs specifically promote differentiation, expansion, and function of Treg or Treg-like cells. These CSR constructs also specifically promote differentiation, expansion, and function of effector T cells or effector-like T cells. These CSR constructs have a CD8a signal peptide.

FIG. 4 is a schematic outlining timeline and protocol for the expansion of Tregs using a CSR booster.

FIG. 5A-5B is a series of graphs depicting the cellular expansion and viability of Treg cells that were modified to express a CSR or not (mock), during the expansion process outlined in FIG. 4. FIG. 5A shows the fold cellular expansion and viability of modified Treg cells in Donor 3. FIG. 5B shows the fold cellular expansion and viability of modified Treg cells in Donor 6.

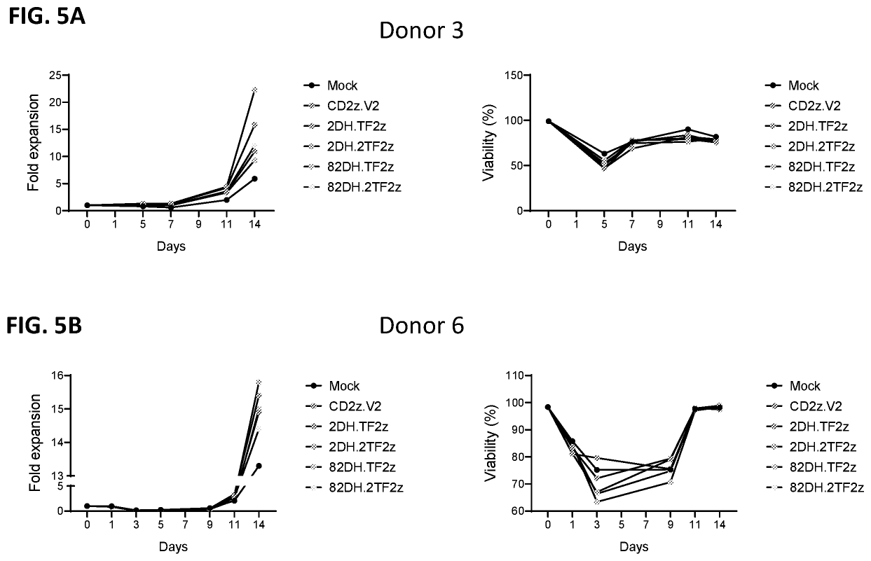

### DETAILED DESCRIPTION OF THE INVENTION

Successful immunosuppressive therapy with regulatory T (Treg) cells requires persistence and suppressive function. Phenotypic plasticity may be a necessary homeostatic adaptive function of Tregs, but in the setting of transplant, autoimmune, allergy or inflammation disease, it raises clinical safety concerns. A key challenge in the field of Treg expansion, manufacturing and production is to achieve sufficient ex vivo expansion of bona-fide Tregs that are free from effector CD4 T cell contamination and that maintain their classical Forkhead box P3 (FoxP3)+ signature.

There are several mechanisms by which Tregs maintain FoxP3+ expression and immunosuppressive function. The inventors have further recognized that tumor necrosis factor receptor-2 (TNFR2) and interleukin-2 (IL-2) are crucial for the maintenance of FoxP3 expression in patients and in murine models. TNFR2 costimulation has been shown to provide signals to Treg progenitors to specifically promote Treg differentiation and shape the Treg repertoire in vivo. Tregs also express IL-2 receptors (IL2R) and signaling through IL2R pathway is essential for the proliferation and function of Tregs. Binding of a ligand to IL2R drives STATS activation which correlates with Treg proliferative capacity, with expression of FoxP3, and with maintenance of the immunosuppressive function of Tregs. Therefore, approaches that induce TNFR2 and IL2R signaling are desirable for Treg expansion and therapy.

Due to their inherent poor expansion ex vivo, several strategies have previously been implemented to produce sufficient Treg cells for a one-time autologous dose. For example, Treg cells can be supplemented with a high dose IL-2 to enable Treg expansion. However, IL-2 promotes the expansion of both Tregs and effector T cells. Unlike Tregs which have an immunosuppressive function, effector T cells have a pro-inflammatory function which may have a detrimental effect in the recipient. Other strategies include treatment of T cells with multiple rounds of CD3 or CD28 restimulation and/or addition of supplements such as rapamycin. These strategies may cause differentiation of Tregs due to over-stimulus and may contribute to a shift in the basal properties of bona-fide Tregs.

The present disclosure provides an engineered regulatory T cell that expresses one or more chimeric stimulatory receptors (CSRs) that specifically promote ex vivo expansion of Treg without the differentiation of the Treg cells into other T cell types. Tregs that express CSRs provide an opportunity to generate antigen-specific Tregs for adoptive cell therapy. There are differences in function and signaling between Tregs and T effector cells (Teff). Accordingly, embodiments of the invention are directed to CSRs with a signaling region that maximizes the suppressive capacity and stability of Tregs for use in, but not limited to, cell therapies for treatment of autoimmune disorders (e.g., multiple sclerosis, rheumatoid arthritis, Type 1 diabetes, Crohn's disease), graft-versus-host disease, inflammatory diseases, and transplant rejection (e.g., kidney transplant or lung transplant).

The present disclosure provides an engineered regulatory T cell that express one or more chimeric stimulatory receptors (CSRs) that specifically promote ex vivo expansion of Teff without the differentiation of the Teff cells into other T cell types. Accordingly, embodiments of the invention are directed to CSRs with a signaling region that maximizes the effector function of Teffs for use in, but not limited to, treatment of cancer or infectious diseases (such as but not limited to bacterial, viral, pathogenic fungi and yeast or parasitic infections).

The present disclosure provides an engineered T cell that expresses an inducible cell surface markers of Tregs in order to direct and promote the ex vivo expansion of Treg cells without the differentiation into other cell types. Accordingly, some embodiments of the invention are directed to the constitutive expression of a FoxP3 Treg cell surface marker on T cells, for use in but not limited to cell therapies for autoimmune disorders, graft-versus-host disease, inflammatory diseases, and transplant rejection. Embodiments of the invention are directed to the expression of transcription factors that cause the expression of FoxP3 Treg cell surface marker on T cells, for use in but not limited to cell therapies for treatment of autoimmune disorders (e.g., multiple sclerosis, rheumatoid arthritis, Type 1 diabetes, Crohn's disease), graft-versus-host disease, inflammatory diseases, and transplant rejection (e.g., kidney transplant or lung transplant).

The present disclosure provides compositions and methods of optimizing the intracellular signaling region of a CSR for regulatory T cell function. This includes, but is not limited to, incorporating the signaling domains or combinations of intracellular signaling domains embodied herein, subsets of their sequences, domains derived from other species or viruses, non-immune signaling domains, synthetic domains or combinations thereof. These signaling architectures designed to maximize Treg function can be present in a CSR or any other chimeric receptor whose extracellular antigen recognition moiety includes, but is not limited to, a single chain antibody fragment (scFv), a VH, a VHH, a Centyrin, a nanobody, a single domain antibody or another type of antibody-based molecule, or a functional non-T cell receptor, or any other antigen recognition molecule.

In some embodiments the CSR includes an extracellular antigen recognition moiety that does not bind to IL2 and includes an intracellular signaling domain of TNFR2, an intracellular signaling domain of IL2 receptor beta (IL2RB) and/or an intracellular signaling domain of IL2 receptor gamma (IL2RG). Addition of a ligand that binds to the extracellular domain allows controlled signaling through the IL2RB, IL2RG and TNFR2 intracellular domains resulting in the ability to control Treg proliferation, differentiation and ultimately stability and longevity. Accordingly, the compositions of the present disclosure may bypass the need to supplement a T cell medium with a high dose IL-2 to enable Treg expansion. Without the use of IL-2 ligand that promotes the expansion of both Tregs and effector T cells, the compositions may eliminate effector T cell contamination during T cell expansion. The compositions of the present disclosure also may eliminate the requirement to use multiple rounds of CD3 or CD28 restimulation and/or addition of supplements such as rapamycin that may contribute to overstimulation and differentiation of Tregs to other T cell types.

In some embodiments, the disclosure provides compositions and methods that result in increased longevity and persistence of Treg phenotype. In some embodiments, the disclosure provides Treg cells and methods that combine persistence and longevity. In some embodiments, Treg cells are maintained in the suppressive state. Without the use of the presently disclosed compositions and methods, adoptively transferred Treg cells can differentiate to other T cell types, whether the autologous or allogeneic, and may become pro-inflammatory and thus cause exacerbation of inflammatory disease.

The present disclosure provides a non-naturally occurring chimeric stimulatory receptor (CSR) comprising, consisting essential of, or consisting of: (a) an ectodomain comprising a activation component, wherein the activation component is isolated or derived from a first protein; (b) a transmembrane domain; and (c) at least one intracellular domain, wherein the intracellular domain is isolated or derived from a second protein, wherein the first and the second protein are not identical.

The activation component can comprise, consist essentially of, or consist of: one or more of a component of a human transmembrane receptor, a human cell-surface receptor, a T cell Receptor (TCR), a component of a TCR complex, a component of a TCR co-receptor, a component of a TCR co-stimulatory protein, a component of a TCR inhibitory protein, a cytokine receptor, or a chemokine receptor. The activation component can comprise, consist essentially of, or consist of, but are not limited to: a portion of one or more of a component of a T cell Receptor (TCR), a component of a TCR complex, a component of a TCR co-receptor, a component of a TCR co-stimulatory protein, a component of a TCR inhibitory protein, a cytokine receptor, or a chemokine receptor to which an agonist of the activation component binds.

The ectodomain can comprise, consist essentially of, or consist of: a CD2 extracellular domain or a portion thereof to which an agonist binds, or the ectodomain can comprise, consist essentially of, or consist of: an IL2RB extracellular domain or a portion thereof to which an agonist binds, or the ectodomain can comprise, consist essentially of, or consist of: an IL2RG extracellular domain or a portion thereof to which an agonist binds. The activation component can comprise, consist essentially of, or consist of: a CD2 extracellular domain or a portion thereof to which an agonist binds, or the activation component can comprise, consist essentially of, or consist of: an IL2RB extracellular domain or a portion thereof to which an agonist binds or the activation component can comprise, consist essentially of, or consist of: an IL2RG extracellular domain or a portion thereof to which an agonist binds. The CD2 extracellular domain to which an agonist binds comprises, consists essentially of, or consists of the amino acid sequence at least 95% identical to the amino acid sequence of SEQ ID NO: 1. The CD2 extracellular domain to which an agonist binds comprises, consists essentially of, or consists of the amino acid sequence at least 99% identical to the amino acid sequence of SEQ ID NO: 1. The CD2 extracellular domain to which an agonist binds comprises, consists essentially of, or consists of the amino acid sequence of SEQ ID NO: 1. The IL2RB extracellular domain to which an agonist binds comprises, consists essentially of, or consists of the amino acid sequence at least 95% identical to the amino acid sequence of SEQ ID NO: 2. The IL2RB extracellular domain to which an agonist binds comprises, consists essentially of, or consists of the amino acid sequence at least 99% identical to the amino acid sequence of SEQ ID NO: 2. The IL2RB extracellular domain to which an agonist binds comprises, consists essentially of, or consists of the amino acid sequence of SEQ ID NO: 2. The IL2RG extracellular domain to which an agonist binds comprises, consists essentially of, or consists of the amino acid sequence at least 95% identical to the amino acid sequence of SEQ ID NO: 3. The IL2RG extracellular domain to which an agonist binds comprises, consists essentially of, or consists of the amino acid sequence at least 99% identical to the amino acid sequence of SEQ ID NO: 3. The IL2RG extracellular domain to which an agonist binds comprises, consists essentially of, or consists of the amino acid sequence of SEQ ID NO: 3.

In some aspects, the ectodomain of the CSR of the present disclosure can comprise a modification. The modification can comprise a mutation or a truncation in the amino acid sequence of the activation component or the first protein when compared to a wild type amino acid sequence of the activation component or the first protein. The mutation or a truncation in the amino acid sequence of the activation component or the first protein can comprise a mutation or truncation of a CD2 extracellular domain or a portion thereof to which an agonist binds. The mutation or truncation of the CD2 extracellular domain reduces or eliminates binding with naturally occurring CD58.

A reduction in binding is when at least 50%, at least 75%, at least 90%, at least 91%, at least 92%, at least 93%, at least 94%, at least 95%, at least 96%, at least 97%, at least 98%, or at least 99% of the binding ability of the mutated or truncated CD2 extracellular domain is reduced when compared to the naturally occurring wild-type counterpart. An elimination in binding is when 100% of the binding ability of the mutated or truncated CD2 extracellular domain is reduced when compared to the naturally occurring wild-type CD2 extracellular domain.

The mutated or truncated CD2 extracellular domain binds anti-CD2 activating agonists and anti-CD2 activating molecules but does not bind naturally occurring CD58. The mutated or truncated CD2 extracellular domain comprises, consists essentially of, or consists of the amino acid sequence at least 80% identical to the amino acid sequence of SEQ ID NO: 4. The mutated or truncated CD2 extracellular domain comprises, consists essentially of, or consists of the amino acid sequence at least 85% identical to the amino acid sequence of SEQ ID NO: 4. The mutated or truncated CD2 extracellular domain comprises, consists essentially of, or consists of the amino acid sequence at least 90% identical to the amino acid sequence of SEQ ID NO: 4. The mutated or truncated CD2 extracellular domain comprises, consists essentially of, or consists of the amino acid sequence at least 95% identical to the amino acid sequence of SEQ ID NO: 4. The mutated or truncated CD2 extracellular domain comprises, consists essentially of, or consists of the amino acid sequence at least 99% identical to the amino acid sequence of SEQ ID NO: 4. The mutated or truncated CD2 extracellular domain comprises, consists essentially of, or consists of the amino acid sequence of SEQ ID NO: 4.

The intracellular domain can be isolated or derived from a third protein. In some aspects, the first protein and the third protein of a CSR of the present disclosure are identical. The intracellular domain can comprise, consist essentially of, or consist of: one or more of a component of a human signal transduction domain, T cell Receptor (TCR), a component of a TCR complex, a component of a TCR co-receptor, a component of a TCR co-stimulatory protein, a component of a TCR inhibitory protein, a cytokine receptor, or a chemokine receptor. The second protein can comprise, consist essentially of, or consist of: a CD3 protein or a portion thereof. The intracellular transduction domain can comprise, consist essentially of, or consist of a CD3 protein or a portion thereof. The CD3 protein can comprise, consist essentially of, or consist of a CD3ζ protein or a portion thereof. The CD3ζ protein comprises, consists essentially of, or consists of the amino acid sequence at least 95% identical to the amino acid sequence of SEQ ID NO: 5. The CD3ζ protein comprises, consists essentially of, or consists of the amino acid sequence at least 99% identical to the amino acid sequence of SEQ ID NO: 5. The CD3 protein comprises, consists essentially of, or consists of the amino acid sequence of SEQ ID NO: 5.

The intracellular domain can comprise, consist essentially of, or consist of a TNFR2 intracellular domain or a portion thereof, or the intracellular domain can comprise, consist essentially of, or consist of an IL2RB intracellular domain or a portion thereof, or the intracellular domain can comprise, consist essentially of, or consist of an IL2RG intracellular domain or a portion thereof. The TNFR2 intracellular domain comprises, consists essentially of, or consists of the amino acid sequence at least 95% identical to the amino acid sequence of SEQ ID NO: 6. The TNFR2 intracellular domain comprises, consists essentially of, or consists of the amino acid sequence at least 99% identical to the amino acid sequence of SEQ ID NO: 6. The TNFR2 intracellular domain comprises, consists essentially of, or consists of the amino acid sequence of SEQ ID NO: 6. The IL2RB intracellular domain comprises, consists essentially of, or consists of the amino acid sequence at least 95% identical to the amino acid sequence of SEQ ID NO: 7. The IL2RB intracellular domain comprises, consists essentially of, or consists of the amino acid sequence at least 99% identical to the amino acid sequence of SEQ ID NO: 7. The IL2RB intracellular domain comprises, consists essentially of, or consists of the amino acid sequence of SEQ ID NO: 7. The IL2RG intracellular domain comprises, consists essentially of, or consists of the amino acid sequence at least 95% identical to the amino acid sequence of SEQ ID NO: 8. The IL2RG intracellular domain comprises, consists essentially of, or consists of the amino acid sequence at least 99% identical to the amino acid sequence of SEQ ID NO: 8. The IL2RG intracellular domain comprises, consists essentially of, or consists of the amino acid sequence of SEQ ID NO: 8.

The ectodomain of a CSR of the present disclosure can further comprise, consist essentially of, or consist of a signal peptide. The signal peptide can be isolated or derived from a third protein. In some aspects, the first protein and the third protein of a CSR of the present disclosure are identical. The signal peptide can comprise, consist essentially of, or consist of: a CD2 signal peptide or a portion thereof; the signal peptide can comprise, consist essentially of, or consist of: an IL2RB signal peptide or a portion thereof; the signal peptide can comprise, consist essentially of, or consist of: an IL2RG signal peptide or a portion thereof; or the signal peptide can comprise, consist essentially of, or consist of: a CD8a signal peptide or a portion thereof. A CSR comprising a CD8a signal peptide may increase the levels of CSR surface expression. The CD2 signal peptide comprises, consists essentially of, or consists of the amino acid sequence at least 95% identical to the amino acid sequence of SEQ ID NO: 9. The CD2 signal peptide comprises, consists essentially of, or consists of the amino acid sequence at least 99% identical to the amino acid sequence of SEQ ID NO: 9. The CD2 signal peptide comprises, consists essentially of, or consists of the amino acid sequence of SEQ ID NO: 9. The IL2RB signal peptide comprises, consists essentially of, or consists of the amino acid sequence at least 95% identical to the amino acid sequence of SEQ ID NO: 10. The IL2RB signal peptide comprises, consists essentially of, or consists of the amino acid sequence at least 99% identical to the amino acid sequence of SEQ ID NO: 10. The IL2RB signal peptide comprises, consists essentially of, or consists of the amino acid sequence of SEQ ID NO: 10. The IL2RG signal peptide comprises, consists essentially of, or consists of the amino acid sequence at least 95% identical to the amino acid sequence of SEQ ID NO: 11. The IL2RG signal peptide comprises, consists essentially of, or consists of the amino acid sequence at least 99% identical to the amino acid sequence of SEQ ID NO: 11. The IL2RG signal peptide comprises, consists essentially of, or consists of the amino acid sequence of SEQ ID NO: 11. The CD8a signal peptide comprises, consists essentially of, or consists of the amino acid sequence at least 95% identical to the amino acid sequence of SEQ ID NO: 12. The CD8a signal peptide comprises, consists essentially of, or consists of the amino acid sequence at least 99% identical to the amino acid sequence of SEQ ID NO: 12. The CD8a signal peptide comprises, consists essentially of, or consists of the amino acid sequence of SEQ ID NO: 12.

The transmembrane domain of a CSR of the present disclosure can be isolated or derived from a fourth protein. In some aspects, the first protein and the fourth protein of a CSR of the present disclosure are identical. The transmembrane domain can comprise, consist essentially of, or consist of: a TNFR2 transmembrane domain or a portion thereof; the transmembrane domain can comprise, consist essentially of or consist of: a CD2 transmembrane domain or a portion thereof; the transmembrane domain can comprise, consist essentially of or consist of: an IL2RB transmembrane domain or a portion thereof; or the transmembrane domain can comprise, consist essentially of, or consist of: an IL2RG transmembrane domain or a portion thereof. The TNFR2 transmembrane domain comprises, consists essentially of, or consists of the amino acid sequence at least 95% identical to the amino acid sequence of SEQ ID NO: 13. The TNFR2 transmembrane domain comprises, consists essentially of, or consists of the amino acid sequence at least 99% identical to the amino acid sequence of SEQ ID NO: 13. The TNFR2 transmembrane domain comprises, consists essentially of, or consists of the amino acid sequence of SEQ ID NO: 13. The CD2 transmembrane domain comprises, consists essentially of, or consists of the amino acid sequence at least 95% identical to the amino acid sequence of SEQ ID NO: 14. The CD2 transmembrane domain comprises, consists essentially of, or consists of the amino acid sequence at least 99% identical to the amino acid sequence of SEQ ID NO: 14. The CD2 transmembrane domain comprises, consists essentially of, or consists of the amino acid sequence of SEQ ID NO: 14. The IL2RB transmembrane domain comprises, consists essentially of, or consists of the amino acid sequence at least 95% identical to the amino acid sequence of SEQ ID NO: 15. The IL2RB transmembrane domain comprises, consists essentially of, or consists of the amino acid sequence at least 99% identical to the amino acid sequence of SEQ ID NO: 15. The IL2RB transmembrane domain comprises, consists essentially of, or consists of the amino acid sequence of SEQ ID NO: 15. The IL2RG transmembrane domain comprises, consists essentially of, or consists of the amino acid sequence at least 95% identical to the amino acid sequence of SEQ ID NO: 16. The IL2RG transmembrane domain comprises, consists essentially of, or consists of the amino acid sequence at least 99% identical to the amino acid sequence of SEQ ID NO: 16. The IL2RG transmembrane domain comprises, consists essentially of, or consists of the amino acid sequence of SEQ ID NO: 16.

The CSR of the disclosure comprising a CD2 signal peptide, a mutated or truncated CD2 extracellular domain, a TNFR2 transmembrane domain, a TNFR2 intracellular domain and a CD3z intracellular domain comprises, consists essentially of, or consists of the amino acid sequence at least 90% identical to the amino acid sequence of SEQ ID NO: 17. The CSR of the disclosure comprising a CD2 signal peptide, a mutated or truncated CD2 extracellular domain, a TNFR2 transmembrane domain, a TNFR2 intracellular domain and a CD3z intracellular domain comprises, consists essentially of, or consists of the amino acid sequence at least 95% identical to the amino acid sequence of SEQ ID NO: 141. The CSR of the disclosure comprising a CD2 signal peptide, a mutated or truncated CD2 extracellular domain, a TNFR2 transmembrane domain, a TNFR2 intracellular domain and a CD3z intracellular domain comprises, consists essentially of, or consists of the amino acid sequence at least 99% identical to the amino acid sequence of SEQ ID NO: 141. The CSR of the disclosure comprising a CD2 signal peptide, a mutated or truncated CD2 extracellular domain, a TNFR2 transmembrane domain, a TNFR2 intracellular domain and a CD3z intracellular domain comprises, consists essentially of, or consists of the amino acid sequence of SEQ ID NO: 141.

The CSR of the disclosure comprising a CD2 signal peptide, a mutated or truncated CD2 extracellular domain, a CD2 transmembrane domain, a TNFR2 intracellular domain and a CD3z intracellular domain comprises, consists essentially of, or consists of the amino acid sequence at least 90% identical to the amino acid sequence of SEQ ID NO: 18. The CSR of the disclosure comprising a CD2 signal peptide, a mutated or truncated CD2 extracellular domain, a CD2 transmembrane domain, a TNFR2 intracellular domain and a CD3z intracellular domain comprises, consists essentially of, or consists of the amino acid sequence at least 95% identical to the amino acid sequence of SEQ ID NO: 18. The CSR of the disclosure comprising a CD2 signal peptide, a mutated or truncated CD2 extracellular domain, a CD2 transmembrane domain, a TNFR2 intracellular domain and a CD3z intracellular domain comprises, consists essentially of, or consists of the amino acid sequence at least 99% identical to the amino acid sequence of SEQ ID NO: 18. The CSR of the disclosure comprising a CD2 signal peptide, a mutated or truncated CD2 extracellular domain, a CD2 transmembrane domain, a TNFR2 intracellular domain and a CD3z intracellular domain comprises, consists essentially of, or consists of the amino acid sequence of SEQ ID NO: 18.

The CSR of the disclosure comprising an IL2RB signal peptide, an IL2RB extracellular domain, an IL2RB transmembrane domain, an IL2RB intracellular domain and a CD3z intracellular domain comprises, consists essentially of, or consists of the amino acid sequence at least 90% identical to the amino acid sequence of SEQ ID NO: 19. The CSR of the disclosure comprising an IL2RB signal peptide, an IL2RB extracellular domain, an IL2RB transmembrane domain, an IL2RB intracellular domain and a CD3z intracellular domain comprises, consists essentially of, or consists of the amino acid sequence at least 95% identical to the amino acid sequence of SEQ ID NO: 19. The CSR of the disclosure comprising an IL2RB signal peptide, an IL2RB extracellular domain, an IL2RB transmembrane domain, an IL2RB intracellular domain and a CD3z intracellular domain comprises, consists essentially of, or consists of the amino acid sequence at least 99% identical to the amino acid sequence of SEQ ID NO: 19. The CSR of the disclosure comprising an IL2RB signal peptide, an IL2RB extracellular domain, an IL2RB transmembrane domain, an IL2RB intracellular domain and a CD3z intracellular domain comprises, consists essentially of, or consists of the amino acid sequence of SEQ ID NO: 19.

The CSR of the disclosure comprising a CD2 signal peptide, a mutated or truncated CD2 extracellular domain, an IL2RB transmembrane domain and an IL2RB intracellular domain comprises, consists essentially of, or consists of the amino acid sequence at least 90% identical to the amino acid sequence of SEQ ID NO: 20. The CSR of the disclosure comprising a CD2 signal peptide, a mutated or truncated CD2 extracellular domain, an IL2RB transmembrane domain and an IL2RB intracellular domain comprises, consists essentially of, or consists of the amino acid sequence at least 95% identical to the amino acid sequence of SEQ ID NO: 20. The CSR of the disclosure comprising a CD2 signal peptide, a mutated or truncated CD2 extracellular domain, an IL2RB transmembrane domain and an IL2RB intracellular domain comprises, consists essentially of, or consists of the amino acid sequence at least 99% identical to the amino acid sequence of SEQ ID NO: 20. The CSR of the disclosure comprising a CD2 signal peptide, a mutated or truncated CD2 extracellular domain, an IL2RB transmembrane domain and an IL2RB intracellular domain comprises, consists essentially of, or consists of the amino acid sequence of SEQ ID NO: 20.

The CSR of the disclosure comprising a CD2 signal peptide, a mutated or truncated CD2 extracellular domain, an IL2RB transmembrane domain, an IL2RB intracellular domain and a CD3z intracellular domain comprises, consists essentially of, or consists of the amino acid sequence at least 90% identical to the amino acid sequence of SEQ ID NO: 21. The CSR of the disclosure comprising a CD2 signal peptide, a mutated or truncated CD2 extracellular domain, an IL2RB transmembrane domain, IL2RB intracellular domain and a CD3z intracellular domain comprises, consists essentially of, or consists of the amino acid sequence at least 95% identical to the amino acid sequence of SEQ ID NO: 21. The CSR of the disclosure comprising a CD2 signal peptide, a mutated or truncated CD2 extracellular domain, an IL2RB transmembrane domain, IL2RB intracellular domain and a CD3z intracellular domain comprises, consists essentially of, or consists of the amino acid sequence at least 99% identical to the amino acid sequence of SEQ ID NO: 21. The CSR of the disclosure comprising a CD2 signal peptide, a mutated or truncated CD2 extracellular domain, an IL2RB transmembrane domain, an IL2RB intracellular domain and a CD3z intracellular domain comprises, consists essentially of, or consists of the amino acid sequence of SEQ ID NO: 21.

The CSR of the disclosure comprising a CD2 signal peptide, a mutated or truncated CD2 extracellular domain, a CD2 transmembrane domain and an IL2RB intracellular domain comprises, consists essentially of, or consists of the amino acid sequence at least 90% identical to the amino acid sequence of SEQ ID NO: 22. The CSR of the disclosure comprising a CD2 signal peptide, a mutated or truncated CD2 extracellular domain, a CD2 transmembrane domain and an IL2RB intracellular domain comprises, consists essentially of, or consists of the amino acid sequence at least 95% identical to the amino acid sequence of SEQ ID NO: 22. The CSR of the disclosure comprising a CD2 signal peptide, a mutated or truncated CD2 extracellular domain, a CD2 transmembrane domain and an IL2RB intracellular domain comprises, consists essentially of, or consists of the amino acid sequence at least 99% identical to the amino acid sequence of SEQ ID NO: 22. The CSR of the disclosure comprising a CD2 signal peptide, a mutated or truncated CD2 extracellular domain, a CD2 transmembrane domain and an IL2RB intracellular domain comprises, consists essentially of, or consists of the amino acid sequence of SEQ ID NO: 22.

The CSR of the disclosure comprising a CD2 signal peptide, a mutated or truncated CD2 extracellular domain, a CD2 transmembrane domain, an IL2RB intracellular domain and a CD3z intracellular domain comprises, consists essentially of, or consists of the amino acid sequence at least 90% identical to the amino acid sequence of SEQ ID NO: 23. The CSR of the disclosure comprising a CD2 signal peptide, a mutated or truncated CD2 extracellular domain, a CD2 transmembrane domain, IL2RB intracellular domain and a CD3z intracellular domain comprises, consists essentially of, or consists of the amino acid sequence at least 95% identical to the amino acid sequence of SEQ ID NO: 23. The CSR of the disclosure comprising a CD2 signal peptide, a mutated or truncated CD2 extracellular domain, a CD2 transmembrane domain, IL2RB intracellular domain and a CD3z intracellular domain comprises, consists essentially of, or consists of the amino acid sequence at least 99% identical to the amino acid sequence of SEQ ID NO: 23. The CSR of the disclosure comprising a CD2 signal peptide, a mutated or truncated CD2 extracellular domain, a CD2 transmembrane domain, an IL2RB intracellular domain and a CD3z intracellular domain comprises, consists essentially of, or consists of the amino acid sequence of SEQ ID NO: 23.

The CSR of the disclosure comprising an IL2RG signal peptide, an IL2RG extracellular domain, an IL2RG transmembrane domain, an IL2RG intracellular domain and a CD3z intracellular domain comprises, consists essentially of, or consists of the amino acid sequence at least 90% identical to the amino acid sequence of SEQ ID NO: 24. The CSR of the disclosure comprising an IL2RG signal peptide, an IL2RG extracellular domain, an IL2RG transmembrane domain, an IL2RG intracellular domain and a CD3z intracellular domain comprises, consists essentially of, or consists of the amino acid sequence at least 95% identical to the amino acid sequence of SEQ ID NO: 24. The CSR of the disclosure comprising an IL2RG signal peptide, an IL2RG extracellular domain, an IL2RG transmembrane domain, an IL2RG intracellular domain and a CD3z intracellular domain comprises, consists essentially of, or consists of the amino acid sequence at least 99% identical to the amino acid sequence of SEQ ID NO: 24. The CSR of the disclosure comprising an IL2RG signal peptide, an IL2RG extracellular domain, an IL2RG transmembrane domain, an IL2RG intracellular domain and a CD3z intracellular domain comprises, consists essentially of, or consists of the amino acid sequence of SEQ ID NO: 24.

The CSR of the disclosure comprising a CD2 signal peptide, a mutated or truncated CD2 extracellular domain, an IL2RG transmembrane domain and an IL2RG intracellular domain comprises, consists essentially of, or consists of the amino acid sequence at least 90% identical to the amino acid sequence of SEQ ID NO: 25. The CSR of the disclosure comprising a CD2 signal peptide, a mutated or truncated CD2 extracellular domain, an IL2RG transmembrane domain and an IL2RG intracellular domain comprises, consists essentially of, or consists of the amino acid sequence at least 95% identical to the amino acid sequence of SEQ ID NO: 25. The CSR of the disclosure comprising a CD2 signal peptide, a mutated or truncated CD2 extracellular domain, an IL2RG transmembrane domain and an IL2RG intracellular domain comprises, consists essentially of, or consists of the amino acid sequence at least 99% identical to the amino acid sequence of SEQ ID NO: 25. The CSR of the disclosure comprising a CD2 signal peptide, a mutated or truncated CD2 extracellular domain, an IL2RG transmembrane domain and an IL2RG intracellular domain comprises, consists essentially of, or consists of the amino acid sequence of SEQ ID NO: 25.

The CSR of the disclosure comprising a CD2 signal peptide, a mutated or truncated CD2 extracellular domain, an IL2RG transmembrane domain, an IL2RG intracellular domain and a CD3z intracellular domain comprises, consists essentially of, or consists of the amino acid sequence at least 90% identical to the amino acid sequence of SEQ ID NO: 26. The CSR of the disclosure comprising a CD2 signal peptide, a mutated or truncated CD2 extracellular domain, an IL2RG transmembrane domain, IL2RG intracellular domain and a CD3z intracellular domain comprises, consists essentially of, or consists of the amino acid sequence at least 95% identical to the amino acid sequence of SEQ ID NO: 26. The CSR of the disclosure comprising a CD2 signal peptide, a mutated or truncated CD2 extracellular domain, an IL2RG transmembrane domain, IL2RG intracellular domain and a CD3z intracellular domain comprises, consists essentially of, or consists of the amino acid sequence at least 99% identical to the amino acid sequence of SEQ ID NO: 26. The CSR of the disclosure comprising a CD2 signal peptide, a mutated or truncated CD2 extracellular domain, an IL2RG transmembrane domain, an IL2RG intracellular domain and a CD3z intracellular domain comprises, consists essentially of, or consists of the amino acid sequence of SEQ ID NO: 26.

The CSR of the disclosure comprising a CD2 signal peptide, a mutated or truncated CD2 extracellular domain, a CD2 transmembrane domain and an IL2RG intracellular domain comprises, consists essentially of, or consists of the amino acid sequence at least 90% identical to the amino acid sequence of SEQ ID NO: 27. The CSR of the disclosure comprising a CD2 signal peptide, a mutated or truncated CD2 extracellular domain, a CD2 transmembrane domain and an IL2RG intracellular domain comprises, consists essentially of, or consists of the amino acid sequence at least 95% identical to the amino acid sequence of SEQ ID NO: 27. The CSR of the disclosure comprising a CD2 signal peptide, a mutated or truncated CD2 extracellular domain, a CD2 transmembrane domain and an IL2RG intracellular domain comprises, consists essentially of, or consists of the amino acid sequence at least 99% identical to the amino acid sequence of SEQ ID NO: 27. The CSR of the disclosure comprising a CD2 signal peptide, a mutated or truncated CD2 extracellular domain, a CD2 transmembrane domain and an IL2RG intracellular domain comprises, consists essentially of, or consists of the amino acid sequence of SEQ ID NO: 27.

The CSR of the disclosure comprising a CD2 signal peptide, a mutated or truncated CD2 extracellular domain, a CD2 transmembrane domain, an IL2RG intracellular domain and a CD3z intracellular domain comprises, consists essentially of, or consists of the amino acid sequence at least 90% identical to the amino acid sequence of SEQ ID NO: 28. The CSR of the disclosure comprising a CD2 signal peptide, a mutated or truncated CD2 extracellular domain, a CD2 transmembrane domain, IL2RG intracellular domain and a CD3z intracellular domain comprises, consists essentially of, or consists of the amino acid sequence at least 95% identical to the amino acid sequence of SEQ ID NO: 28. The CSR of the disclosure comprising a CD2 signal peptide, a mutated or truncated CD2 extracellular domain, a CD2 transmembrane domain, IL2RG intracellular domain and a CD3z intracellular domain comprises, consists essentially of, or consists of the amino acid sequence at least 99% identical to the amino acid sequence of SEQ ID NO: 28. The CSR of the disclosure comprising a CD2 signal peptide, a mutated or truncated CD2 extracellular domain, a CD2 transmembrane domain, an IL2RG intracellular domain and a CD3z intracellular domain comprises, consists essentially of, or consists of the amino acid sequence of SEQ ID NO: 28.

The CSR of the disclosure comprising a CD2 signal peptide, a mutated or truncated CD2 extracellular domain, an IL2RB transmembrane domain, an IL2RB intracellular domain and an IL2RG intracellular domain comprises, consists essentially of, or consists of the amino acid sequence at least 90% identical to the amino acid sequence of SEQ ID NO: 29. The CSR of the disclosure comprising a CD2 signal peptide, a mutated or truncated CD2 extracellular domain, an IL2RB transmembrane domain, an IL2RB intracellular domain and an IL2RG intracellular domain comprises, consists essentially of, or consists of the amino acid sequence at least 95% identical to the amino acid sequence of SEQ ID NO: 29. The CSR of the disclosure comprising a CD2 signal peptide, a mutated or truncated CD2 extracellular domain, an IL2RB transmembrane domain, an IL2RB intracellular domain and an IL2RG intracellular domain comprises, consists essentially of, or consists of the amino acid sequence at least 99% identical to the amino acid sequence of SEQ ID NO: 29. The CSR of the disclosure comprising a CD2 signal peptide, a mutated or truncated CD2 extracellular domain, an IL2RB transmembrane domain, an IL2RB intracellular domain and an IL2RG intracellular domain comprises, consists essentially of, or consists of the amino acid sequence of SEQ ID NO: 29.

The CSR of the disclosure comprising a CD2 signal peptide, a mutated or truncated CD2 extracellular domain, an IL2RB transmembrane domain, an IL2RB intracellular domain, an IL2RG intracellular domain and a CD3z intracellular domain comprises, consists essentially of, or consists of the amino acid sequence at least 90% identical to the amino acid sequence of SEQ ID NO: 30. The CSR of the disclosure comprising a CD2 signal peptide, a mutated or truncated CD2 extracellular domain, an IL2RB transmembrane domain, an IL2RB intracellular domain, an IL2RG intracellular domain and a CD3z intracellular domain comprises, consists essentially of, or consists of the amino acid sequence at least 95% identical to the amino acid sequence of SEQ ID NO: 30. The CSR of the disclosure comprising a CD2 signal peptide, a mutated or truncated CD2 extracellular domain, an IL2RB transmembrane domain, an IL2RB intracellular domain, an IL2RG intracellular domain and a CD3z intracellular domain comprises, consists essentially of, or consists of the amino acid sequence at least 99% identical to the amino acid sequence of SEQ ID NO: 30. The CSR of the disclosure comprising a CD2 signal peptide, a mutated or truncated CD2 extracellular domain, an IL2RB transmembrane domain, an IL2RB intracellular domain, an IL2RG intracellular domain and a CD3z intracellular domain comprises, consists essentially of, or consists of the amino acid sequence of SEQ ID NO: 30.

The CSR of the disclosure comprising a CD2 signal peptide, a mutated or truncated CD2 extracellular domain, a CD2 transmembrane domain, an IL2RB intracellular domain and an IL2RG intracellular domain comprises, consists essentially of, or consists of the amino acid sequence at least 90% identical to the amino acid sequence of SEQ ID NO: 31. The CSR of the disclosure comprising a CD2 signal peptide, a mutated or truncated CD2 extracellular domain, a CD2 transmembrane domain, an IL2RB intracellular domain and an IL2RG intracellular domain comprises, consists essentially of, or consists of the amino acid sequence at least 95% identical to the amino acid sequence of SEQ ID NO: 31. The CSR of the disclosure comprising a CD2 signal peptide, a mutated or truncated CD2 extracellular domain, a CD2 transmembrane domain, an IL2RB intracellular domain and an IL2RG intracellular domain comprises, consists essentially of, or consists of the amino acid sequence at least 99% identical to the amino acid sequence of SEQ ID NO: 31. The CSR of the disclosure comprising a CD2 signal peptide, a mutated or truncated CD2 extracellular domain, a CD2 transmembrane domain, an IL2RB intracellular domain and an IL2RG intracellular domain comprises, consists essentially of, or consists of the amino acid sequence of SEQ ID NO: 31.

The CSR of the disclosure comprising a CD2 signal peptide, a mutated or truncated CD2 extracellular domain, a CD2 transmembrane domain, an IL2RB intracellular domain, an IL2RG intracellular domain and a CD3z intracellular domain comprises, consists essentially of, or consists of the amino acid sequence at least 90% identical to the amino acid sequence of SEQ ID NO: 32. The CSR of the disclosure comprising a CD2 signal peptide, a mutated or truncated CD2 extracellular domain, a CD2 transmembrane domain, an IL2RB intracellular domain, an IL2RG intracellular domain and a CD3z intracellular domain comprises, consists essentially of, or consists of the amino acid sequence at least 95% identical to the amino acid sequence of SEQ ID NO: 32. The CSR of the disclosure comprising a CD2 signal peptide, a mutated or truncated CD2 extracellular domain, a CD2 transmembrane domain, an IL2RB intracellular domain, an IL2RG intracellular domain and a CD3z intracellular domain comprises, consists essentially of, or consists of the amino acid sequence at least 99% identical to the amino acid sequence of SEQ ID NO: 32. The CSR of the disclosure comprising a CD2 signal peptide, a mutated or truncated CD2 extracellular domain, a CD2 transmembrane domain, an IL2RB intracellular domain, an IL2RG intracellular domain and a CD3z intracellular domain comprises, consists essentially of, or consists of the amino acid sequence of SEQ ID NO: 32.

The CSR of the disclosure comprising a CD2 signal peptide, a mutated or truncated CD2 extracellular domain, an IL2RG transmembrane domain, an IL2RG intracellular domain and an IL2RB intracellular domain comprises, consists essentially of, or consists of the amino acid sequence at least 90% identical to the amino acid sequence of SEQ ID NO: 33. The CSR of the disclosure comprising a CD2 signal peptide, a mutated or truncated CD2 extracellular domain, an IL2RG transmembrane domain, an IL2RG intracellular domain and an IL2RB intracellular domain comprises, consists essentially of, or consists of the amino acid sequence at least 95% identical to the amino acid sequence of SEQ ID NO: 33. The CSR of the disclosure comprising a CD2 signal peptide, a mutated or truncated CD2 extracellular domain, an IL2RG transmembrane domain, an IL2RG intracellular domain and an IL2RB intracellular domain comprises, consists essentially of, or consists of the amino acid sequence at least 99% identical to the amino acid sequence of SEQ ID NO: 33. The CSR of the disclosure comprising a CD2 signal peptide, a mutated or truncated CD2 extracellular domain, an IL2RG transmembrane domain, an IL2RG intracellular domain and an IL2RB intracellular domain comprises, consists essentially of, or consists of the amino acid sequence of SEQ ID NO: 33.

The CSR of the disclosure comprising a CD2 signal peptide, a mutated or truncated CD2 extracellular domain, an IL2RG transmembrane domain, an IL2RG intracellular domain, an IL2RB intracellular domain and a CD3z intracellular domain comprises, consists essentially of, or consists of the amino acid sequence at least 90% identical to the amino acid sequence of SEQ ID NO: 34. The CSR of the disclosure comprising a CD2 signal peptide, a mutated or truncated CD2 extracellular domain, an IL2RG transmembrane domain, an IL2RG intracellular domain, an IL2RB intracellular domain and a CD3z intracellular domain comprises, consists essentially of, or consists of the amino acid sequence at least 95% identical to the amino acid sequence of SEQ ID NO: 34. The CSR of the disclosure comprising a CD2 signal peptide, a mutated or truncated CD2 extracellular domain, an IL2RG transmembrane domain, an IL2RG intracellular domain, an IL2RB intracellular domain and a CD3z intracellular domain comprises, consists essentially of, or consists of the amino acid sequence at least 99% identical to the amino acid sequence of SEQ ID NO: 34. The CSR of the disclosure comprising a CD2 signal peptide, a mutated or truncated CD2 extracellular domain, an IL2RG transmembrane domain, an IL2RG intracellular domain, an IL2RB intracellular domain and a CD3z intracellular domain comprises, consists essentially of, or consists of the amino acid sequence of SEQ ID NO: 34.

The CSR of the disclosure comprising a CD2 signal peptide, a mutated or truncated CD2 extracellular domain, a CD2 transmembrane domain, an IL2RG intracellular domain and an IL2RB intracellular domain comprises, consists essentially of, or consists of the amino acid sequence at least 90% identical to the amino acid sequence of SEQ ID NO: 35. The CSR of the disclosure comprising a CD2 signal peptide, a mutated or truncated CD2 extracellular domain, a CD2 transmembrane domain, an IL2RG intracellular domain and an IL2RB intracellular domain comprises, consists essentially of, or consists of the amino acid sequence at least 95% identical to the amino acid sequence of SEQ ID NO: 35. The CSR of the disclosure comprising a CD2 signal peptide, a mutated or truncated CD2 extracellular domain, a CD2 transmembrane domain, an IL2RG intracellular domain and an IL2RB intracellular domain comprises, consists essentially of, or consists of the amino acid sequence at least 99% identical to the amino acid sequence of SEQ ID NO: 35. The CSR of the disclosure comprising a CD2 signal peptide, a mutated or truncated CD2 extracellular domain, a CD2 transmembrane domain, an IL2RG intracellular domain and an IL2RB intracellular domain comprises, consists essentially of, or consists of the amino acid sequence of SEQ ID NO: 35.

The CSR of the disclosure comprising a CD2 signal peptide, a mutated or truncated CD2 extracellular domain, a CD2 transmembrane domain, an IL2RG intracellular domain, an IL2RB intracellular domain and a CD3z intracellular domain comprises, consists essentially of, or consists of the amino acid sequence at least 90% identical to the amino acid sequence of SEQ ID NO: 36. The CSR of the disclosure comprising a CD2 signal peptide, a mutated or truncated CD2 extracellular domain, a CD2 transmembrane domain, an IL2RG intracellular domain, an IL2RB intracellular domain and a CD3z intracellular domain comprises, consists essentially of, or consists of the amino acid sequence at least 95% identical to the amino acid sequence of SEQ ID NO: 36. The CSR of the disclosure comprising a CD2 signal peptide, a mutated or truncated CD2 extracellular domain, a CD2 transmembrane domain, an IL2RG intracellular domain, an IL2RB intracellular domain and a CD3z intracellular domain comprises, consists essentially of, or consists of the amino acid sequence at least 99% identical to the amino acid sequence of SEQ ID NO: 36. The CSR of the disclosure comprising a CD2 signal peptide, a mutated or truncated CD2 extracellular domain, a CD2 transmembrane domain, an IL2RG intracellular domain, an IL2RB intracellular domain and a CD3z intracellular domain comprises, consists essentially of, or consists of the amino acid sequence of SEQ ID NO: 36.

The CSR of the disclosure comprising a CD8 signal peptide, a mutated or truncated CD2 extracellular domain, a TNFR2 transmembrane domain, a TNFR2 intracellular domain and a CD3z intracellular domain comprises, consists essentially of, or consists of the amino acid sequence at least 90% identical to the amino acid sequence of SEQ ID NO: 37. The CSR of the disclosure comprising a CD8 signal peptide, a mutated or truncated CD2 extracellular domain, a TNFR2 transmembrane domain, a TNFR2 intracellular domain and a CD3z intracellular domain comprises, consists essentially of, or consists of the amino acid sequence at least 95% identical to the amino acid sequence of SEQ ID NO: 37. The CSR of the disclosure comprising a CD8 signal peptide, a mutated or truncated CD2 extracellular domain, a TNFR2 transmembrane domain, a TNFR2 intracellular domain and a CD3z intracellular domain comprises, consists essentially of, or consists of the amino acid sequence at least 99% identical to the amino acid sequence of SEQ ID NO: 37. The CSR of the disclosure comprising a CD8 signal peptide, a mutated or truncated CD2 extracellular domain, a TNFR2 transmembrane domain, a TNFR2 intracellular domain and a CD3z intracellular domain comprises, consists essentially of, or consists of the amino acid sequence of SEQ ID NO: 37.

The CSR of the disclosure comprising a CD8 signal peptide, a mutated or truncated CD2 extracellular domain, a CD2 transmembrane domain, a TNFR2 intracellular domain and a CD3z intracellular domain comprises, consists essentially of, or consists of the amino acid sequence at least 90% identical to the amino acid sequence of SEQ ID NO: 38. The CSR of the disclosure comprising a CD8 signal peptide, a mutated or truncated CD2 extracellular domain, a CD2 transmembrane domain, a TNFR2 intracellular domain and a CD3z intracellular domain comprises, consists essentially of, or consists of the amino acid sequence at least 95% identical to the amino acid sequence of SEQ ID NO: 38. The CSR of the disclosure comprising a CD8 signal peptide, a mutated or truncated CD2 extracellular domain, a CD2 transmembrane domain, a TNFR2 intracellular domain and a CD3z intracellular domain comprises, consists essentially of, or consists of the amino acid sequence at least 99% identical to the amino acid sequence of SEQ ID NO: 38. The CSR of the disclosure comprising a CD8 signal peptide, a mutated or truncated CD2 extracellular domain, a CD2 transmembrane domain, a TNFR2 intracellular domain and a CD3z intracellular domain comprises, consists essentially of, or consists of the amino acid sequence of SEQ ID NO: 38.

The CSR of the disclosure comprising a CD8a signal peptide, an IL2RB extracellular domain, an IL2RB transmembrane domain and an IL2RB intracellular domain comprises, consists essentially of, or consists of the amino acid sequence at least 90% identical to the amino acid sequence of SEQ ID NO: 39. The CSR of the disclosure comprising a CD8a signal peptide, an IL2RB extracellular domain, an IL2RB transmembrane domain and an IL2RB intracellular domain comprises, consists essentially of, or consists of the amino acid sequence at least 95% identical to the amino acid sequence of SEQ ID NO: 39. The CSR of the disclosure comprising a CD8a signal peptide, an IL2RB extracellular domain, an IL2RB transmembrane domain and an IL2RB intracellular domain comprises, consists essentially of, or consists of the amino acid sequence at least 99% identical to the amino acid sequence of SEQ ID NO: 39. The CSR of the disclosure comprising a CD8a signal peptide, an IL2RB extracellular domain, an IL2RB transmembrane domain and an IL2RB intracellular domain comprises, consists essentially of, or consists of the amino acid sequence of SEQ ID NO: 39.

The CSR of the disclosure comprising a CD8a signal peptide, an IL2RB extracellular domain, an IL2RB transmembrane domain, an IL2RB intracellular domain and a CD3z intracellular domain comprises, consists essentially of, or consists of the amino acid sequence at least 90% identical to the amino acid sequence of SEQ ID NO: 40. The CSR of the disclosure comprising a CD8a signal peptide, an IL2RB extracellular domain, an IL2RB transmembrane domain, an IL2RB intracellular domain and a CD3z intracellular domain comprises, consists essentially of, or consists of the amino acid sequence at least 95% identical to the amino acid sequence of SEQ ID NO: 40. The CSR of the disclosure comprising a CD8a signal peptide, an IL2RB extracellular domain, an IL2RB transmembrane domain, an IL2RB intracellular domain and a CD3z intracellular domain comprises, consists essentially of, or consists of the amino acid sequence at least 99% identical to the amino acid sequence of SEQ ID NO: 40. The CSR of the disclosure comprising a CD8a signal peptide, an IL2RB extracellular domain, an IL2RB transmembrane domain, an IL2RB intracellular domain and a CD3z intracellular domain comprises, consists essentially of, or consists of the amino acid sequence of SEQ ID NO: 40.

The CSR of the disclosure comprising a CD8a signal peptide, a mutated or truncated CD2 extracellular domain, an IL2RB transmembrane domain and an IL2RB intracellular domain comprises, consists essentially of, or consists of the amino acid sequence at least 90% identical to the amino acid sequence of SEQ ID NO: 41. The CSR of the disclosure comprising a CD8a signal peptide, a mutated or truncated CD2 extracellular domain, an IL2RB transmembrane domain and an IL2RB intracellular domain comprises, consists essentially of, or consists of the amino acid sequence at least 95% identical to the amino acid sequence of SEQ ID NO: 41. The CSR of the disclosure comprising a CD8a signal peptide, a mutated or truncated CD2 extracellular domain, an IL2RB transmembrane domain and an IL2RB intracellular domain comprises, consists essentially of, or consists of the amino acid sequence at least 99% identical to the amino acid sequence of SEQ ID NO: 41. The CSR of the disclosure comprising a CD8a signal peptide, a mutated or truncated CD2 extracellular domain, an IL2RB transmembrane domain and an IL2RB intracellular domain comprises, consists essentially of, or consists of the amino acid sequence of SEQ ID NO: 41.

The CSR of the disclosure comprising a CD8a signal peptide, a mutated or truncated CD2 extracellular domain, an IL2RB transmembrane domain, an IL2RB intracellular domain and a CD3z intracellular domain comprises, consists essentially of, or consists of the amino acid sequence at least 90% identical to the amino acid sequence of SEQ ID NO: 42. The CSR of the disclosure comprising a CD8a signal peptide, a mutated or truncated CD2 extracellular domain, an IL2RB transmembrane domain, an IL2RB intracellular domain and a CD3z intracellular domain comprises, consists essentially of, or consists of the amino acid sequence at least 95% identical to the amino acid sequence of SEQ ID NO: 42. The CSR of the disclosure comprising a CD8a signal peptide, a mutated or truncated CD2 extracellular domain, an IL2RB transmembrane domain, an IL2RB intracellular domain and a CD3z intracellular domain comprises, consists essentially of, or consists of the amino acid sequence at least 99% identical to the amino acid sequence of SEQ ID NO: 42. The CSR of the disclosure comprising a CD8a signal peptide, a mutated or truncated CD2 extracellular domain, an IL2RB transmembrane domain, an IL2RB intracellular domain and a CD3z intracellular domain comprises, consists essentially of, or consists of the amino acid sequence of SEQ ID NO: 42.

The CSR of the disclosure comprising a CD8a signal peptide, a mutated or truncated CD2 extracellular domain, a CD2 transmembrane domain and an IL2RB intracellular domain comprises, consists essentially of, or consists of the amino acid sequence at least 90% identical to the amino acid sequence of SEQ ID NO: 43. The CSR of the disclosure comprising a CD8a signal peptide, a mutated or truncated CD2 extracellular domain, a CD2 transmembrane domain and an IL2RB intracellular domain comprises, consists essentially of, or consists of the amino acid sequence at least 95% identical to the amino acid sequence of SEQ ID NO: 43. The CSR of the disclosure comprising a CD8a signal peptide, a mutated or truncated CD2 extracellular domain, a CD2 transmembrane domain and an IL2RB intracellular domain comprises, consists essentially of, or consists of the amino acid sequence at least 99% identical to the amino acid sequence of SEQ ID NO: 43. The CSR of the disclosure comprising a CD8a signal peptide, a mutated or truncated CD2 extracellular domain, a CD2 transmembrane domain and an IL2RB intracellular domain comprises, consists essentially of, or consists of the amino acid sequence of SEQ ID NO: 43.

The CSR of the disclosure comprising a CD8a signal peptide, a mutated or truncated CD2 extracellular domain, a CD2 transmembrane domain, an IL2RB intracellular domain and a CD3z intracellular domain comprises, consists essentially of, or consists of the amino acid sequence at least 90% identical to the amino acid sequence of SEQ ID NO: 44. The CSR of the disclosure comprising a CD8a signal peptide, a mutated or truncated CD2 extracellular domain, a CD2 transmembrane domain, an IL2RB intracellular domain and a CD3z intracellular domain comprises, consists essentially of, or consists of the amino acid sequence at least 95% identical to the amino acid sequence of SEQ ID NO: 44. The CSR of the disclosure comprising a CD8a signal peptide, a mutated or truncated CD2 extracellular domain, a CD2 transmembrane domain, an IL2RB intracellular domain and a CD3z intracellular domain comprises, consists essentially of, or consists of the amino acid sequence at least 99% identical to the amino acid sequence of SEQ ID NO: 44. The CSR of the disclosure comprising a CD8a signal peptide, a mutated or truncated CD2 extracellular domain, a CD2 transmembrane domain, an IL2RB intracellular domain and a CD3z intracellular domain comprises, consists essentially of, or consists of the amino acid sequence of SEQ ID NO: 44.

The CSR of the disclosure comprising a CD8a signal peptide, an IL2RG extracellular domain, an IL2RG transmembrane domain and an IL2RG intracellular domain comprises, consists essentially of, or consists of the amino acid sequence at least 90% identical to the amino acid sequence of SEQ ID NO: 45. The CSR of the disclosure comprising a CD8a signal peptide, an IL2RG extracellular domain, an IL2RG transmembrane domain and an IL2RG intracellular domain comprises, consists essentially of, or consists of the amino acid sequence at least 95% identical to the amino acid sequence of SEQ ID NO: 45. The CSR of the disclosure comprising a CD8a signal peptide, an IL2RG extracellular domain, an IL2RG transmembrane domain and an IL2RG intracellular domain comprises, consists essentially of, or consists of the amino acid sequence at least 99% identical to the amino acid sequence of SEQ ID NO: 45. The CSR of the disclosure comprising a CD8a signal peptide, an IL2RG extracellular domain, an IL2RG transmembrane domain and an IL2RG intracellular domain comprises, consists essentially of, or consists of the amino acid sequence of SEQ ID NO: 45.

The CSR of the disclosure comprising a CD8a signal peptide, an IL2RG extracellular domain, an IL2RG transmembrane domain, an IL2RG intracellular domain and a CD3z intracellular domain comprises, consists essentially of, or consists of the amino acid sequence at least 90% identical to the amino acid sequence of SEQ ID NO: 46. The CSR of the disclosure comprising a CD8a signal peptide, an IL2RG extracellular domain, an IL2RG transmembrane domain, an IL2RG intracellular domain and a CD3z intracellular domain comprises, consists essentially of, or consists of the amino acid sequence at least 95% identical to the amino acid sequence of SEQ ID NO: 46. The CSR of the disclosure comprising a CD8a signal peptide, an IL2RG extracellular domain, an IL2RG transmembrane domain, an IL2RG intracellular domain and a CD3z intracellular domain comprises, consists essentially of, or consists of the amino acid sequence at least 99% identical to the amino acid sequence of SEQ ID NO: 46. The CSR of the disclosure comprising a CD8a signal peptide, an IL2RG extracellular domain, an IL2RG transmembrane domain, an IL2RG intracellular domain and a CD3z intracellular domain comprises, consists essentially of, or consists of the amino acid sequence of SEQ ID NO: 46.

The CSR of the disclosure comprising a CD8a signal peptide, a mutated or truncated CD2 extracellular domain, an IL2RG transmembrane domain and an IL2RG intracellular domain comprises, consists essentially of, or consists of the amino acid sequence at least 90% identical to the amino acid sequence of SEQ ID NO: 47. The CSR of the disclosure comprising a CD8a signal peptide, a mutated or truncated CD2 extracellular domain, an IL2RG transmembrane domain and an IL2RG intracellular domain comprises, consists essentially of, or consists of the amino acid sequence at least 95% identical to the amino acid sequence of SEQ ID NO: 47. The CSR of the disclosure comprising a CD8a signal peptide, a mutated or truncated CD2 extracellular domain, an IL2RG transmembrane domain and an IL2RG intracellular domain comprises, consists essentially of, or consists of the amino acid sequence at least 99% identical to the amino acid sequence of SEQ ID NO: 47. The CSR of the disclosure comprising a CD8a signal peptide, a mutated or truncated CD2 extracellular domain, an IL2RG transmembrane domain and an IL2RG intracellular domain comprises, consists essentially of, or consists of the amino acid sequence of SEQ ID NO: 47.

The CSR of the disclosure comprising a CD8a signal peptide, a mutated or truncated CD2 extracellular domain, an IL2RG transmembrane domain, an IL2RG intracellular domain and a CD3z intracellular domain comprises, consists essentially of, or consists of the amino acid sequence at least 90% identical to the amino acid sequence of SEQ ID NO: 48. The CSR of the disclosure comprising a CD8a signal peptide, a mutated or truncated CD2 extracellular domain, an IL2RG transmembrane domain, an IL2RG intracellular domain and a CD3z intracellular domain comprises, consists essentially of, or consists of the amino acid sequence at least 95% identical to the amino acid sequence of SEQ ID NO: 48. The CSR of the disclosure comprising a CD8a signal peptide, a mutated or truncated CD2 extracellular domain, an IL2RG transmembrane domain, an IL2RG intracellular domain and a CD3z intracellular domain comprises, consists essentially of, or consists of the amino acid sequence at least 99% identical to the amino acid sequence of SEQ ID NO: 48. The CSR of the disclosure comprising a CD8a signal peptide, a mutated or truncated CD2 extracellular domain, an IL2RG transmembrane domain, an IL2RG intracellular domain and a CD3z intracellular domain comprises, consists essentially of, or consists of the amino acid sequence of SEQ ID NO: 48.

The CSR of the disclosure comprising a CD8a signal peptide, a mutated or truncated CD2 extracellular domain, a CD2 transmembrane domain and an IL2RG intracellular domain comprises, consists essentially of, or consists of the amino acid sequence at least 90% identical to the amino acid sequence of SEQ ID NO: 49. The CSR of the disclosure comprising a CD8a signal peptide, a mutated or truncated CD2 extracellular domain, a CD2 transmembrane domain and an IL2RG intracellular domain comprises, consists essentially of, or consists of the amino acid sequence at least 95% identical to the amino acid sequence of SEQ ID NO: 49. The CSR of the disclosure comprising a CD8a signal peptide, a mutated or truncated CD2 extracellular domain, a CD2 transmembrane domain and an IL2RG intracellular domain comprises, consists essentially of, or consists of the amino acid sequence at least 99% identical to the amino acid sequence of SEQ ID NO: 49. The CSR of the disclosure comprising a CD8a signal peptide, a mutated or truncated CD2 extracellular domain, a CD2 transmembrane domain and an IL2RG intracellular domain comprises, consists essentially of, or consists of the amino acid sequence of SEQ ID NO: 49.

The CSR of the disclosure comprising a CD8a signal peptide, a mutated or truncated CD2 extracellular domain, a CD2 transmembrane domain, an IL2RG intracellular domain and a CD3z intracellular domain comprises, consists essentially of, or consists of the amino acid sequence at least 90% identical to the amino acid sequence of SEQ ID NO: 50. The CSR of the disclosure comprising a CD8a signal peptide, a mutated or truncated CD2 extracellular domain, a CD2 transmembrane domain, an IL2RG intracellular domain and a CD3z intracellular domain comprises, consists essentially of, or consists of the amino acid sequence at least 95% identical to the amino acid sequence of SEQ ID NO: 50. The CSR of the disclosure comprising a CD8a signal peptide, a mutated or truncated CD2 extracellular domain, a CD2 transmembrane domain, an IL2RG intracellular domain and a CD3z intracellular domain comprises, consists essentially of, or consists of the amino acid sequence at least 99% identical to the amino acid sequence of SEQ ID NO: 50. The CSR of the disclosure comprising a CD8a signal peptide, a mutated or truncated CD2 extracellular domain, a CD2 transmembrane domain, an IL2RG intracellular domain and a CD3z intracellular domain comprises, consists essentially of, or consists of the amino acid sequence of SEQ ID NO: 50.

The CSR of the disclosure comprising a CD8a signal peptide, a mutated or truncated CD2 extracellular domain, an IL2RB transmembrane domain, an IL2RB intracellular domain and an IL2RG intracellular domain comprises, consists essentially of, or consists of the amino acid sequence at least 90% identical to the amino acid sequence of SEQ ID NO: 51. The CSR of the disclosure comprising a CD8a signal peptide, a mutated or truncated CD2 extracellular domain, an IL2RB transmembrane domain, an IL2RB intracellular domain and an IL2RG intracellular domain comprises, consists essentially of, or consists of the amino acid sequence at least 95% identical to the amino acid sequence of SEQ ID NO: 51. The CSR of the disclosure comprising a CD8a signal peptide, a mutated or truncated CD2 extracellular domain, an IL2RB transmembrane domain, an IL2RB intracellular domain and an IL2RG intracellular domain comprises, consists essentially of, or consists of the amino acid sequence at least 99% identical to the amino acid sequence of SEQ ID NO: 51. The CSR of the disclosure comprising a CD8a signal peptide, a mutated or truncated CD2 extracellular domain, an IL2RB transmembrane domain, an IL2RB intracellular domain and an IL2RG intracellular domain comprises, consists essentially of, or consists of the amino acid sequence of SEQ ID NO: 51.

The CSR of the disclosure comprising a CD8a signal peptide, a mutated or truncated CD2 extracellular domain, an IL2RB transmembrane domain, an IL2RB intracellular domain, an IL2RG intracellular domain and a CD3z intracellular domain comprises, consists essentially of, or consists of the amino acid sequence at least 90% identical to the amino acid sequence of SEQ ID NO: 52. The CSR of the disclosure comprising a CD8a signal peptide, a mutated or truncated CD2 extracellular domain, an IL2RB transmembrane domain, an IL2RB intracellular domain, an IL2RG intracellular domain and a CD3z intracellular domain comprises, consists essentially of, or consists of the amino acid sequence at least 95% identical to the amino acid sequence of SEQ ID NO: 52. The CSR of the disclosure comprising a CD8a signal peptide, a mutated or truncated CD2 extracellular domain, an IL2RB transmembrane domain, an IL2RB intracellular domain, an IL2RG intracellular domain and a CD3z intracellular domain comprises, consists essentially of, or consists of the amino acid sequence at least 99% identical to the amino acid sequence of SEQ ID NO: 52. The CSR of the disclosure comprising a CD8a signal peptide, a mutated or truncated CD2 extracellular domain, an IL2RB transmembrane domain, an IL2RB intracellular domain, an IL2RG intracellular domain and a CD3z intracellular domain comprises, consists essentially of, or consists of the amino acid sequence of SEQ ID NO: 52.

The CSR of the disclosure comprising a CD8a signal peptide, a mutated or truncated CD2 extracellular domain, a CD2 transmembrane domain, an IL2RB intracellular domain and an IL2RG intracellular domain comprises, consists essentially of, or consists of the amino acid sequence at least 90% identical to the amino acid sequence of SEQ ID NO: 53. The CSR of the disclosure comprising a CD8a signal peptide, a mutated or truncated CD2 extracellular domain, a CD2 transmembrane domain, an IL2RB intracellular domain and an IL2RG intracellular domain comprises, consists essentially of, or consists of the amino acid sequence at least 95% identical to the amino acid sequence of SEQ ID NO: 53. The CSR of the disclosure comprising a CD8a signal peptide, a mutated or truncated CD2 extracellular domain, a CD2 transmembrane domain, an IL2RB intracellular domain and an IL2RG intracellular domain comprises, consists essentially of, or consists of the amino acid sequence at least 99% identical to the amino acid sequence of SEQ ID NO: 53. The CSR of the disclosure comprising a CD8a signal peptide, a mutated or truncated CD2 extracellular domain, a CD2 transmembrane domain, an IL2RB intracellular domain and an IL2RG intracellular domain comprises, consists essentially of, or consists of the amino acid sequence of SEQ ID NO: 53.

The CSR of the disclosure comprising a CD8a signal peptide, a mutated or truncated CD2 extracellular domain, a CD2 transmembrane domain, an IL2RB intracellular domain, an IL2RG intracellular domain and a CD3z intracellular domain comprises, consists essentially of, or consists of the amino acid sequence at least 90% identical to the amino acid sequence of SEQ ID NO: 54. The CSR of the disclosure comprising a CD8a signal peptide, a mutated or truncated CD2 extracellular domain, a CD2 transmembrane domain, an IL2RB intracellular domain, an IL2RG intracellular domain and a CD3z intracellular domain comprises, consists essentially of, or consists of the amino acid sequence at least 95% identical to the amino acid sequence of SEQ ID NO: 54. The CSR of the disclosure comprising a CD8a signal peptide, a mutated or truncated CD2 extracellular domain, a CD2 transmembrane domain, an IL2RB intracellular domain, an IL2RG intracellular domain and a CD3z intracellular domain comprises, consists essentially of, or consists of the amino acid sequence at least 99% identical to the amino acid sequence of SEQ ID NO: 54. The CSR of the disclosure comprising a CD8a signal peptide, a mutated or truncated CD2 extracellular domain, a CD2 transmembrane domain, an IL2RB intracellular domain, an IL2RG intracellular domain and a CD3z intracellular domain comprises, consists essentially of, or consists of the amino acid sequence of SEQ ID NO: 54.

The CSR of the disclosure comprising a CD8a signal peptide, a mutated or truncated CD2 extracellular domain, an IL2RG transmembrane domain, an IL2RG intracellular domain and an IL2RB intracellular domain comprises, consists essentially of, or consists of the amino acid sequence at least 90% identical to the amino acid sequence of SEQ ID NO: 55. The CSR of the disclosure comprising a CD8a signal peptide, a mutated or truncated CD2 extracellular domain, an IL2RG transmembrane domain, an IL2RG intracellular domain and an IL2RB intracellular domain comprises, consists essentially of, or consists of the amino acid sequence at least 95% identical to the amino acid sequence of SEQ ID NO: 55. The CSR of the disclosure comprising a CD8a signal peptide, a mutated or truncated CD2 extracellular domain, an IL2RG transmembrane domain, an IL2RG intracellular domain and an IL2RB intracellular domain comprises, consists essentially of, or consists of the amino acid sequence at least 99% identical to the amino acid sequence of SEQ ID NO: 55. The CSR of the disclosure comprising a CD8a signal peptide, a mutated or truncated CD2 extracellular domain, an IL2RG transmembrane domain, an IL2RG intracellular domain and an IL2RB intracellular domain comprises, consists essentially of, or consists of the amino acid sequence of SEQ ID NO: 55.

The CSR of the disclosure comprising a CD8a signal peptide, a mutated or truncated CD2 extracellular domain, an IL2RG transmembrane domain, an IL2RG intracellular domain, an IL2RB intracellular domain and a CD3z intracellular domain comprises, consists essentially of, or consists of the amino acid sequence at least 90% identical to the amino acid sequence of SEQ ID NO: 56. The CSR of the disclosure comprising a CD8a signal peptide, a mutated or truncated CD2 extracellular domain, an IL2RG transmembrane domain, an IL2RG intracellular domain, an IL2RB intracellular domain and a CD3z intracellular domain comprises, consists essentially of, or consists of the amino acid sequence at least 95% identical to the amino acid sequence of SEQ ID NO: 56. The CSR of the disclosure comprising a CD8a signal peptide, a mutated or truncated CD2 extracellular domain, an IL2RG transmembrane domain, an IL2RG intracellular domain, an IL2RB intracellular domain and a CD3z intracellular domain comprises, consists essentially of, or consists of the amino acid sequence at least 99% identical to the amino acid sequence of SEQ ID NO: 56. The CSR of the disclosure comprising a CD8a signal peptide, a mutated or truncated CD2 extracellular domain, an IL2RG transmembrane domain, an IL2RG intracellular domain, an IL2RB intracellular domain and a CD3z intracellular domain comprises, consists essentially of, or consists of the amino acid sequence of SEQ ID NO: 56.

The CSR of the disclosure comprising a CD8a signal peptide, a mutated or truncated CD2 extracellular domain, a CD2 transmembrane domain, an IL2RG intracellular domain and an IL2RB intracellular domain comprises, consists essentially of, or consists of the amino acid sequence at least 90% identical to the amino acid sequence of SEQ ID NO: 57. The CSR of the disclosure comprising a CD8a signal peptide, a mutated or truncated CD2 extracellular domain, a CD2 transmembrane domain, an IL2RG intracellular domain and an IL2RB intracellular domain comprises, consists essentially of, or consists of the amino acid sequence at least 95% identical to the amino acid sequence of SEQ ID NO: 57. The CSR of the disclosure comprising a CD8a signal peptide, a mutated or truncated CD2 extracellular domain, a CD2 transmembrane domain, an IL2RG intracellular domain and an IL2RB intracellular domain comprises, consists essentially of, or consists of the amino acid sequence at least 99% identical to the amino acid sequence of SEQ ID NO: 57. The CSR of the disclosure comprising a CD8a signal peptide, a mutated or truncated CD2 extracellular domain, a CD2 transmembrane domain, an IL2RG intracellular domain and an IL2RB intracellular domain comprises, consists essentially of, or consists of the amino acid sequence of SEQ ID NO: 57.

The CSR of the disclosure comprising a CD8a signal peptide, a mutated or truncated CD2 extracellular domain, a CD2 transmembrane domain, an IL2RG intracellular domain, an IL2RB intracellular domain and a CD3z intracellular domain comprises, consists essentially of, or consists of the amino acid sequence at least 90% identical to the amino acid sequence of SEQ ID NO: 58. The CSR of the disclosure comprising a CD8a signal peptide, a mutated or truncated CD2 extracellular domain, a CD2 transmembrane domain,an IL2RG intracellular domain, an IL2RB intracellular domain and a CD3z intracellular domain comprises, consists essentially of, or consists of the amino acid sequence at least 95% identical to the amino acid sequence of SEQ ID NO: 58. The CSR of the disclosure comprising a CD8a signal peptide, a mutated or truncated CD2 extracellular domain, a CD2 transmembrane domain, an IL2RG intracellular domain, an IL2RB intracellular domain and a CD3z intracellular domain comprises, consists essentially of, or consists of the amino acid sequence at least 99% identical to the amino acid sequence of SEQ ID NO: 58. The CSR of the disclosure comprising a CD8a signal peptide, a mutated or truncated CD2 extracellular domain, a CD2 transmembrane domain, an IL2RG intracellular domain, an IL2RB intracellular domain and a CD3z intracellular domain comprises, consists essentially of, or consists of the amino acid sequence of SEQ ID NO: 58.

In some aspects, the activation component of the CSR of the present disclosure does not bind or is incapable of binding a naturally-occurring molecule. In some aspects, the activation component of the CSR of the present disclosure binds or is capable of binding a naturally-occurring molecule and the CSR transduces a signal upon binding of the activation component to the naturally-occurring molecule. In other aspects, the activation component of the CSR of the present disclosure can bind a naturally-occurring molecule but the CSR does not transduce a signal upon binding of the activation component to a naturally-occurring molecule. In preferred aspects, the activation component of the CSR of the present disclosure binds or is capable of binding to a non-naturally occurring molecule. The activation component of the CSR of the present disclosure selectively transduces a signal upon binding of a non-naturally occurring molecule to the activation component. In one aspect, the naturally occurring molecule is a naturally occurring agonist/activating agent for the activation component of the CSR. The naturally occurring agonist/activating agent that can bind a CSR activation component can be any naturally occurring antibody, antibody fragment or any other binding peptide that is specific to the CSR activation component. The naturally occurring antibody or antibody fragment can be a naturally occurring anti-CD3 antibody or fragment thereof, an anti-CD2 antibody or fragment thereof, an anti-CD28 antibody or fragment thereof, or any combination thereof. In some aspects, the naturally occurring agonist/activating agent that can bind a CSR activation component can be one or more of an anti-human CD3 monospecific tetrameric antibody complex, an anti-human CD2 monospecific tetrameric antibody complex, an anti-human CD28 monospecific tetrameric antibody complex, or a combination thereof. In one aspect, the non-naturally occurring molecule is a non-naturally occurring agonist/activating agent for the activation component of the CSR. The non-naturally occurring agonist/activating agent that can bind a CSR activation component can be any non-naturally occurring antibody or antibody fragment. The non-naturally occurring antibody or antibody fragment can be a non-naturally occurring anti-CD3 antibody or fragment thereof, an anti-CD2 antibody or fragment thereof, an anti-CD28 antibody or fragment thereof, or any combination thereof. In some aspects, the non-naturally occurring agonist/activating agent that can bind a CSR activation component can be one or more of an anti-human CD3 monospecific tetrameric antibody complex, an anti-human CD2 monospecific tetrameric antibody complex, an anti-human CD28 monospecific tetrameric antibody complex, or a combination thereof. In some aspects, the non-naturally occurring agonist/activating agent that can bind a CSR activation component can be selected from the group consisting of anti-CD2 monoclonal antibody, BTI-322 (Przepiorka et al., Blood 92(11):4066-4071, 1998) and humanized anti-CD2 monoclonal antibody clone AFC-TAB-104 (Siplizumab)(Bissonnette et al. Arch. Dermatol. Res. 301(6):429-442, 2009). Other antibody clones include but are not limited to TS1/8, RPA-2.10, 127, 1E7E8.G4, 3.22, 3A10B2, 3F3, 3H667, 4E4, 5F8, 5H3, 6B2D3F12, 6G4, 7H196, 9-1, 9.6, 9D1, 13H78, AB75, AFBD-3, AHC0031, B-E2, B4-D1, BH1, CB.219, DFA-7, E17-P, G11, G531.1, hCD2, H22, IHC531, LT2, LFA2/600, MEM-65, MT110, MT910, MU3, OKT11, OKT12, OTI2C3, OTI3E11, OX-34, REA972, S5.5, T6.3, TP1/31, TS2/9, TS2/18, UMCD2, UMCD2/1E7E8, YTH655.

The present disclosure also provides a non-naturally occurring chimeric stimulatory receptor (CSR) comprising, consisting essential of, or consisting of: (a) an ectodomain comprising a activation component, wherein the activation component is isolated or derived from a first protein and wherein the activation component binds to a non-naturally occurring molecule but does not bind a naturally-occurring molecule; (b) a transmembrane domain; and (c) at least one intracellular domain, wherein the at least one intracellular signaling domain is isolated or derived from a second protein; wherein the first protein and the second protein are not identical.

The present disclosure also provides a non-naturally occurring chimeric stimulatory receptor (CSR) comprising, consisting essential of, or consisting of: (a) an ectodomain comprising a activation component, wherein the activation component is isolated or derived from a first protein; (b) a transmembrane domain; and (c) at least one intracellular domain, wherein the at least one intracellular signaling domain is isolated or derived from a second protein; wherein the first protein and the second protein are not identical and wherein the CSR does not transduce a signal upon binding of a naturally-occurring molecule to the activation component.

The present disclosure also provides a non-naturally occurring chimeric stimulatory receptor (CSR) comprising, consisting essential of, or consisting of: (a) an ectodomain comprising a activation component, wherein the activation component is isolated or derived from a first protein; (b) a transmembrane domain; and (c) at least one intracellular domain, wherein the at least one intracellular signaling domain is isolated or derived from a second protein; wherein the first protein and the second protein are not identical and wherein the CSR transduces a signal upon binding of a non-naturally-occurring molecule to the activation component.

The present disclosure further provides a non-naturally occurring chimeric stimulatory receptor (CSR) comprising, consisting essential of, or consisting of: (a) an ectodomain comprising a signal peptide and an activation component, wherein the signal peptide comprises a CD2 signal peptide or a portion thereof and wherein the activation component comprises a mutation or truncation of a wild-type CD2 extracellular domain or a portion thereof to which an agonist binds; (b) a transmembrane domain, wherein the transmembrane domain comprises a TNFR2 transmembrane domain or a portion thereof; and (c) an intracellular domain, wherein the intracellular domain comprises a TNFR2 intracellular domain or a portion thereof and a CD3ζ protein or a portion thereof. In one aspect, the mutation or truncation of the CD2 extracellular domain reduces or eliminates binding with naturally occurring CD58. In another aspect, the mutated or truncated CD2 extracellular domain binds anti-CD2 activating agonists and anti-CD2 activating molecules but does not bind naturally occurring CD58.

The present disclosure further provides a non-naturally occurring chimeric stimulatory receptor (CSR) comprising, consisting essentially of, or consisting of: (a) an ectodomain comprising a signal peptide comprising the amino acid sequence of SEQ ID NO: 9 and a activation component comprising the amino acid sequence of SEQ ID NO: 4; (b) a transmembrane domain of SEQ ID NO: 13; and (c) an intracellular domain comprising the amino acid sequence of SEQ ID NO: 6 and comprising the amino acid sequence of SEQ ID NO: 5. The non-naturally occurring chimeric stimulatory receptor (CSR) can comprise, consist essentially of, or consist of an amino acid sequence at least 80% identical to SEQ ID NO: 17. The non-naturally occurring chimeric stimulatory receptor (CSR) can comprise, consist essentially of, or consist of an amino acid sequence at least 85% identical to SEQ ID NO: 17. The non-naturally occurring chimeric stimulatory receptor (CSR) can comprise, consist essentially of, or consist of an amino acid sequence at least 90% identical to SEQ ID NO: 17. The non-naturally occurring chimeric stimulatory receptor (CSR) can comprise, consist essentially of, or consist of an acid sequence at least 95% identical to SEQ ID NO: 17. The non-naturally occurring chimeric stimulatory receptor (CSR) can comprise, consist essentially of, or consist of an acid sequence at least 99% identical to SEQ ID NO: 17. The non-naturally occurring chimeric stimulatory receptor (CSR) can comprise, consist essentially of, or consist of an amino acid sequence of SEQ ID NO: 17.

The present disclosure further provides a non-naturally occurring chimeric stimulatory receptor (CSR) comprising, consisting essential of, or consisting of: (a) an ectodomain comprising a signal peptide and an activation component, wherein the signal peptide comprises a CD2 signal peptide or a portion thereof and wherein the activation component comprises a mutation or truncation of a wild-type CD2 extracellular domain or a portion thereof to which an agonist binds; (b) a transmembrane domain, wherein the transmembrane domain comprises a CD2 transmembrane domain or a portion thereof; and (c) an intracellular domain, wherein the intracellular domain comprises a TNFR2 intracellular domain or a portion thereof and a CD3ζ protein or a portion thereof. In one aspect, the mutation or truncation of the CD2 extracellular domain reduces or eliminates binding with naturally occurring CD58. In another aspect, the mutated or truncated CD2 extracellular domain binds anti-CD2 activating agonists and anti-CD2 activating molecules but does not bind naturally occurring CD58.

The present disclosure further provides a non-naturally occurring chimeric stimulatory receptor (CSR) comprising, consisting essentially of, or consisting of: (a) an ectodomain comprising a signal peptide comprising the amino acid sequence of SEQ ID NO: 9 and a activation component comprising the amino acid sequence of SEQ ID NO: 4; (b) a transmembrane domain of SEQ ID NO: 14; and (c) an intracellular domain comprising the amino acid sequence of SEQ ID NO: 6 and comprising the amino acid sequence of SEQ ID NO: 5. The non-naturally occurring chimeric stimulatory receptor (CSR) can comprise, consist essentially of, or consist of an amino acid sequence at least 80% identical to SEQ ID NO: 18. The non-naturally occurring chimeric stimulatory receptor (CSR) can comprise, consist essentially of, or consist of an amino acid sequence at least 85% identical to SEQ ID NO: 18. The non-naturally occurring chimeric stimulatory receptor (CSR) can comprise, consist essentially of, or consist of an amino acid sequence at least 90% identical to SEQ ID NO: 18. The non-naturally occurring chimeric stimulatory receptor (CSR) can comprise, consist essentially of, or consist of an amino acid sequence at least 95% identical to SEQ ID NO: 18. The non-naturally occurring chimeric stimulatory receptor (CSR) can comprise, consist essentially of, or consist of an amino acid sequence at least 99% identical to SEQ ID NO: 18. The non-naturally occurring chimeric stimulatory receptor (CSR) can comprise, consist essentially of, or consist of an amino acid sequence of SEQ ID NO: 18.

The present disclosure also provides a nucleic acid sequence encoding an amino acid sequence of any chimeric stimulatory receptor (CSR) disclosed herein. The present disclosure also provides transposon, a vector, a donor sequence or a donor plasmid comprising, consisting essential of or consisting of a nucleic acid sequence encoding the amino acid sequence of any chimeric stimulatory receptor (CSR) disclosed herein. In one aspect, the vector can be a viral vector. In one aspect, a viral vector can be an adenoviral vector, adeno-associated viral (AAV) vector, retroviral vector, lentiviral vector or a chimeric viral vector.

The present disclosure also provides a cell comprising, consisting essential of or consisting of any chimeric stimulatory receptor (CSR) disclosed herein. The present disclosure also provides a cell comprising, consisting essential of or consisting of a nucleic acid sequence encoding an amino acid sequence of any chimeric stimulatory receptor (CSR) disclosed herein. The present disclosure also provides a cell comprising, consisting essential of or consisting of a transposon, a vector, a donor sequence or a donor plasmid comprising, consisting essential of or consisting of a nucleic acid sequence encoding the amino acid sequence of any chimeric stimulatory receptor (CSR) disclosed herein. In one aspect, the vector can be a viral vector. In one aspect, a viral vector can be an adenoviral vector, adeno-associated viral (AAV) vector, retroviral vector, lentiviral vector or a chimeric viral vector. A cell of the present disclosure comprising, consisting essential of or consisting of any chimeric stimulatory receptor (CSR) disclosed herein can be an allogeneic cell or an autologous cell. In some preferred embodiments, the cell is an allogeneic cell.

The present disclosure also provides a composition comprising, consisting essential of or consisting of any chimeric stimulatory receptor (CSR) disclosed herein. The present disclosure also provides a composition comprising, consisting essential of or consisting of a nucleic acid sequence encoding an amino acid sequence of any chimeric stimulatory receptor (CSR) disclosed herein. The present disclosure also provides a composition comprising, consisting essential of or consisting of a transposon, a vector, a donor sequence or a donor plasmid comprising, consisting essential of or consisting of a nucleic acid sequence encoding the amino acid sequence of any chimeric stimulatory receptor (CSR) disclosed herein. In one aspect, the vector can be a viral vector. In one aspect, a viral vector can be an an adenoviral vector, adeno-associated viral (AAV) vector, retroviral vector, lentiviral vector or a chimeric viral vector. The present disclosure also provides a composition comprising, consisting essential of or consisting of a cell or a plurality of cells comprising, consisting essential of or consisting of any chimeric stimulatory receptor (CSR) disclosed herein.

The present disclosure provides a modified cell comprising, consisting essential of, or consisting of a chimeric stimulatory receptor (CSR) comprising, consisting essential of, or consisting of: (i) an ectodomain comprising an activation component, wherein the activation component is isolated or derived from a first protein; (ii) a transmembrane domain; and (iii) an endodomain comprising at least one signal transduction domain, wherein the at least one signal transduction domain is isolated or derived from a second protein; wherein the first protein and the second protein are not identical.

The present disclosure also provides a modified cell comprising, consisting essential of, or consisting of (a) a chimeric stimulatory receptor (CSR) comprising: (i) an ectodomain comprising an activation component, wherein the activation component is isolated or derived from a first protein; (ii) a transmembrane domain; and (iii) an endodomain comprising at least one signal transduction domain, wherein the at least one signal transduction domain is isolated or derived from a second protein; wherein the first protein and the second protein are not identical; and (b) an inducible proapoptotic polypeptide.

The present disclosure also provides a modified cell comprising, consisting essential of, or consisting of: (a) a chimeric stimulatory receptor (CSR) comprising: (i) an ectodomain comprising an activation component, wherein the activation component is isolated or derived from a first protein; (ii) a transmembrane domain; and (iii) an endodomain comprising at least one signal transduction domain, wherein the at least one signal transduction domain is isolated or derived from a second protein; wherein the first protein and the second protein are not identical; (b) a sequence encoding an inducible proapoptotic polypeptide; and wherein the cell is a T cell, (c) a modification of an endogenous sequence encoding a T cell Receptor (TCR), wherein the modification reduces or eliminates a level of expression or activity of the TCR.

The present disclosure provides a modified cell comprising, consisting essential of, or consisting of: (a) a modification of an endogenous sequence encoding Beta-2-Microglobulin (B2M), wherein the modification reduces or eliminates a level of expression or activity of a major histocompatibility complex (MEW) class I (MHC-I); and (b) a non-naturally occurring sequence comprising an HLA class I histocompatibility antigen, alpha chain E (HLA-E) polypeptide.

The present disclosure provides a modified T lymphocyte (T cell) comprising, consisting essential of, or consisting of: (a) a modification of an endogenous sequence encoding a T cell Receptor (TCR), wherein the modification reduces or eliminates a level of expression or activity of the TCR; and (b) chimeric stimulatory receptor (CSR) comprising: (i) an ectodomain comprising an activation component, wherein the activation component is isolated or derived from a first protein; (ii) a transmembrane domain; and (iii) an endodomain comprising at least one signal transduction domain, wherein the at least one signal transduction domain is isolated or derived from a second protein; wherein the first protein and the second protein are not identical.

The present disclosure provides a modified T lymphocyte (T cell) comprising, consisting essential of, or consisting of: (a) a modification of an endogenous sequence encoding a T cell Receptor (TCR), wherein the modification reduces or eliminates a level of expression or activity of the TCR; (b) chimeric stimulatory receptor (CSR) comprising: (i) an ectodomain comprising an activation component, wherein the activation component is isolated or derived from a first protein; (ii) a transmembrane domain; and (iii) an endodomain comprising at least one signal transduction domain, wherein the at least one signal transduction domain is isolated or derived from a second protein; wherein the first protein and the second protein are not identical; and (c) a non-naturally occurring chimeric antigen receptor.

The present disclosure provides a modified T lymphocyte (T cell) comprising, consisting essential of, or consisting of: (a) a modification of an endogenous sequence encoding a T cell Receptor (TCR), wherein the modification reduces or eliminates a level of expression or activity of the TCR; (b) a modification of an endogenous sequence encoding Beta-2-Microglobulin (B2M), wherein the modification reduces or eliminates a level of expression or activity of a major histocompatibility complex (MHC) class I (MHC-I); and (c) a chimeric stimulatory receptor (CSR) comprising: (i) an ectodomain comprising an activation component, wherein the activation component is isolated or derived from a first protein; (ii) a transmembrane domain; and (iii) an endodomain comprising at least one signal transduction domain, wherein the at least one signal transduction domain is isolated or derived from a second protein; wherein the first protein and the second protein are not identical.

The present disclosure provides a modified T lymphocyte (T cell) comprising, consisting essential of, or consisting of: (a) a modification of an endogenous sequence encoding a T cell Receptor (TCR), wherein the modification reduces or eliminates a level of expression or activity of the TCR; (b) a modification of an endogenous sequence encoding Beta-2-Microglobulin (B2M), wherein the modification reduces or eliminates a level of expression or activity of a major histocompatibility complex (MHC) class I (MHC-I); (c) a chimeric stimulatory receptor (CSR) comprising: (i) an ectodomain comprising an activation component, wherein the activation component is isolated or derived from a first protein; (ii) a transmembrane domain; and (iii) an endodomain comprising at least one signal transduction domain, wherein the at least one signal transduction domain is isolated or derived from a second protein; wherein the first protein and the second protein are not identical; and (d) a non-naturally occurring chimeric antigen receptor.

The present disclosure also provides a modified T lymphocyte (T cell) comprising, consisting essential of, or consisting of: (a) a modification of an endogenous sequence encoding a T cell Receptor (TCR), wherein the modification reduces or eliminates a level of expression or activity of the TCR; (b) a modification of an endogenous sequence encoding Beta-2-Microglobulin (B2M), wherein the modification reduces or eliminates a level of expression or activity of a major histocompatibility complex (MHC) class I (MHC-I); (c) a non-naturally occurring sequence comprising an HLA class I histocompatibility antigen, alpha chain E (HLA-E); and (d) a chimeric stimulatory receptor (CSR) comprising: (i) an ectodomain comprising an activation component, wherein the activation component is isolated or derived from a first protein; (ii) a transmembrane domain; and (iii) an endodomain comprising at least one signal transduction domain, wherein the at least one signal transduction domain is isolated or derived from a second protein; wherein the first protein and the second protein are not identical.

The present disclosure also provides a modified T lymphocyte (T cell) comprising, consisting essential of, or consisting of: (a) a modification of an endogenous sequence encoding a T cell Receptor (TCR), wherein the modification reduces or eliminates a level of expression or activity of the TCR; (b) a modification of an endogenous sequence encoding Beta-2-Microglobulin (B2M), wherein the modification reduces or eliminates a level of expression or activity of a major histocompatibility complex (MHC) class I (MHC-I); (c) a non-naturally occurring sequence comprising an HLA class I histocompatibility antigen, alpha chain E (HLA-E); (d) a chimeric stimulatory receptor (CSR) comprising: (i) an ectodomain comprising an activation component, wherein the activation component is isolated or derived from a first protein; (ii) a transmembrane domain; and (iii) an endodomain comprising at least one signal transduction domain, wherein the at least one signal transduction domain is isolated or derived from a second protein; wherein the first protein and the second protein are not identical; and (e) a non-naturally occurring chimeric antigen receptor.

The present disclosure also provides a modified T lymphocyte (T cell), consisting essential of, or consisting of: (a) a modification of an endogenous sequence encoding a T cell Receptor (TCR), wherein the modification reduces or eliminates a level of expression or activity of the TCR; (b) a modification that reduces or eliminates a level of expression or activity of a HLA class I histocompatibility antigen, alpha chain A (HLA-A), HLA class I histocompatibility antigen, alpha chain B (HLA-B), HLA class I histocompatibility antigen, alpha chain C (HLA-C), or a combination thereof; and (c) a chimeric stimulatory receptor (CSR) comprising: (i) an ectodomain comprising an activation component, wherein the activation component is isolated or derived from a first protein; (ii) a transmembrane domain; and (iii) an endodomain comprising at least one signal transduction domain, wherein the at least one signal transduction domain is isolated or derived from a second protein; wherein the first protein and the second protein are not identical.

The present disclosure also provides a modified T lymphocyte (T cell), consisting essential of, or consisting of: (a) a modification of an endogenous sequence encoding a T cell Receptor (TCR), wherein the modification reduces or eliminates a level of expression or activity of the TCR; (b) a modification that reduces or eliminates a level of expression or activity of a HLA class I histocompatibility antigen, alpha chain A (HLA-A), HLA class I histocompatibility antigen, alpha chain B (HLA-B), HLA class I histocompatibility antigen, alpha chain C (HLA-C), or a combination thereof; (c) a non-naturally occurring sequence comprising an HLA class I histocompatibility antigen, alpha chain E (HLA-E); and (d) a chimeric stimulatory receptor (CSR) comprising: (i) an ectodomain comprising an activation component, wherein the activation component is isolated or derived from a first protein; (ii) a transmembrane domain; and (iii) an endodomain comprising at least one signal transduction domain, wherein the at least one signal transduction domain is isolated or derived from a second protein; wherein the first protein and the second protein are not identical.

A modified cell of the present disclosure (preferably a modified T cell of the present disclosure) can further comprise, consist essentially of, or consist of an inducible proapoptotic polypeptide. The inducible proapoptotic polypeptide comprises, consists essentially of, or consists of the amino acid sequence at least 95% identical to the amino acid sequence of SEQ ID NO: 62. The inducible proapoptotic polypeptide comprises, consists essentially of, or consists of the amino acid sequence at least 99% identical to the amino acid sequence of SEQ ID NO: 62. The inducible proapoptotic polypeptide comprises, consists essentially of, or consists of the amino acid sequence of SEQ ID NO: 62.

A modified cell of the present disclosure (preferably a modified T cell of the present disclosure) can further comprise, consist essentially of, or consist of a modification of an endogenous sequence encoding Beta-2-Microglobulin (B2M), wherein the modification reduces or eliminates a level of expression or activity of a major histocompatibility complex (MHC) class I (MHC-I). A reduction of a level of expression or activity is when at least 50%, at least 75%, at least 90%, at least 91%, at least 92%, at least 93%, at least 94%, at least 95%, at least 96%, at least 97%, at least 98%, or at least 99% of the expression of the MHC-I in a cell or the functional activity of the MHC-I in a cell is reduced when compared to the naturally occurring wild-type counterpart of the cell. A reduction of a level of expression or activity is when at least 50%, at least 75%, at least 90%, at least 91%, at least 92%, at least 93%, at least 94%, at least 95%, at least 96%, at least 97%, at least 98%, or at least 99% of the expression of the MHC-I in a T cell or the functional activity of the MHC-I in a T cell is reduced when compared to a naturally occurring wild-type T cell. An elimination a level of expression or activity is when 100% of the expression of the MHC-I in a cell or the functional activity of the MHC-I in a cell is reduced when compared to the naturally occurring wild-type counterpart of the cell. An elimination a level of expression or activity is when 100% of the expression of the MHC-I in a T cell or the functional activity of the MEW-I in a T cell is reduced when compared to the naturally occurring wild-type T cell.

A modified cell of the present disclosure (preferably a modified T cell of the present disclosure) can further comprise, consist essentially of, or consist of a non-naturally occurring polypeptide comprising an HLA class I histocompatibility antigen, alpha chain E (HLA-E). The HLA-E polypeptide comprises, consists essentially of, or consists of the amino acid sequence at least 95% identical to the amino acid sequence of SEQ ID NO: 63. The HLA-E polypeptide comprises, consists essentially of, or consists of the amino acid sequence at least 99% identical to the amino acid sequence of SEQ ID NO: 63. The HLA-E polypeptide comprises, consists essentially of, or consists of the amino acid sequence of SEQ ID NO: 63.

The non-naturally occurring polypeptide comprising a HLA-E can further comprise, consist essentially of, or consist of a B2M signal peptide. The B2M signal peptide comprises, consists essentially of, or consists of the amino acid sequence at least 95% identical to the amino acid sequence of SEQ ID NO: 64. The B2M signal peptide comprises, consists essentially of, or consists of the amino acid sequence at least 99% identical to the amino acid sequence of SEQ ID NO: 64. The B2M signal peptide comprises, consists essentially of, or consists of the amino acid sequence of SEQ ID NO: 64.

The non-naturally occurring polypeptide comprising a HLA-E can further comprise, consist essentially of, or consist of a B2M polypeptide. The B2M polypeptide comprises, consists essentially of, or consists of the amino acid sequence at least 95% identical to the amino acid sequence of SEQ ID NO: 65. The B2M polypeptide comprises, consists essentially of, or consists of the amino acid sequence at least 99% identical to the amino acid sequence of SEQ ID NO: 65. The B2M polypeptide comprises, consists essentially of, or consists of the amino acid sequence of SEQ ID NO: 65.

The non-naturally occurring polypeptide comprising a HLA-E can further comprise, consist essentially of, or consist of a linker molecule (referred to herein as a linker). The non-naturally occurring polypeptide comprising a HLA-E can further comprise, consist essentially of, or consist of a linker, wherein the linker is positioned between the B2M polypeptide and the HLA-E polypeptide. The linker comprises, consists essentially of, or consists of the amino acid sequence at least 95% identical to the amino acid sequence of SEQ ID NO: 66. The linker comprises, consists essentially of, or consists of the amino acid sequence at least 99% identical to the amino acid sequence of SEQ ID NO: 66. The linker comprises, consists essentially of, or consists of the amino acid sequence of SEQ ID NO: 66.

The non-naturally occurring polypeptide comprising a HLA-E can further comprise, consist essentially of, or consist of a peptide and a B2M polypeptide. The peptide comprises, consists essentially of, or consists of the amino acid sequence at least 95% identical to the amino acid sequence of SEQ ID NO: 67. The peptide comprises, consists essentially of, or consists of the amino acid sequence at least 99% identical to the amino acid sequence of SEQ ID NO: 67. The peptide comprises, consists essentially of, or consists of the amino acid sequence of SEQ ID NO: 67.

The non-naturally occurring polypeptide comprising a HLA-E can further comprise, consist essentially of, or consist of a first linker positioned between the B2M signal peptide and the peptide, and a second linker positioned between the B2M polypeptide and the HLA-E polypeptide. The first linker comprises, consists essentially of, or consists of the amino acid sequence at least 95% identical to the amino acid sequence of SEQ ID NO: 68. The first linker comprises, consists essentially of, or consists of the amino acid sequence at least 99% identical to the amino acid sequence of SEQ ID NO: 68. The first linker comprises, consists essentially of, or consists of the amino acid sequence of SEQ ID NO: 68. The second linker comprises, consists essentially of, or consists of the amino acid sequence at least 95% identical to the amino acid sequence of SEQ ID NO: 66. The second linker comprises, consists essentially of, or consists of the amino acid sequence at least 99% identical to the amino acid sequence of SEQ ID NO: 66. The second linker comprises, consists essentially of, or consists of the amino acid sequence of SEQ ID NO: 66.

In one aspect, the non-naturally occurring polypeptide comprising an HLA-E comprises, consists essentially of, or consists of a B2M signal peptide, a peptide, a first linker, a B2M polypeptide, a second linker and an HLA-E polypeptide. The peptide can be positioned between the B2M signal peptide and the first linker, the B2M polypeptide can be positioned between the first linker and the second linker and the second linker can be positioned between the B2M polypeptide and the HLA-E polypeptide. The non-naturally occurring polypeptide comprising an HLA-E comprises, consists essentially of, or consists of the amino acid sequence at least 95% identical to the amino acid sequence of SEQ ID NO: 69. The non-naturally occurring polypeptide comprising an HLA-E comprises, consists essentially of, or consists of the amino acid sequence at least 99% identical to the amino acid sequence of SEQ ID NO: 69. The non-naturally occurring polypeptide comprising an HLA-E comprises, consists essentially of, or consists of the amino acid sequence of SEQ ID NO: 69. The non-naturally occurring polypeptide comprising an HLA-E can be encoded by the nucleic acid have the sequence of SEQ ID NO: 70.

In one aspect, the non-naturally occurring polypeptide comprising an HLA-E comprises, consists essentially of, or consists of a B2M signal peptide, a B2M polypeptide, a linker and an HLA-E polypeptide. The B2M polypeptide can be positioned between the B2M signal peptide and the linker, the linker can be positioned between the B2M polypeptide and the HLA-E polypeptide. The non-naturally occurring polypeptide comprising an HLA-E comprises, consists essentially of, or consists of the amino acid sequence at least 95% identical to the amino acid sequence of SEQ ID NO: 71. The non-naturally occurring polypeptide comprising an HLA-E comprises, consists essentially of, or consists of the amino acid sequence at least 99% identical to the amino acid sequence of SEQ ID NO: 71. The non-naturally occurring polypeptide comprising an HLA-E comprises, consists essentially of, or consists of the amino acid sequence of SEQ ID NO: 71. The non-naturally occurring polypeptide comprising an HLA-E can be encoded by the nucleic acid have the sequence of SEQ ID NO: 72.

In one aspect, the non-naturally occurring polypeptide comprising an HLA-E comprises, consists essentially of, or consists of a B2M signal peptide and an HLA-E polypeptide. The B2M signal peptide can be positioned before (e.g., 5′ in the context of a nucleic acid sequence or amino terminus in the context of an amino acid sequence) HLA-E polypeptide. The non-naturally occurring polypeptide comprising an HLA-E comprises, consists essentially of, or consists of the amino acid sequence at least 95% identical to the amino acid sequence of SEQ ID NO: 73. The non-naturally occurring polypeptide comprising an HLA-E comprises, consists essentially of, or consists of the amino acid sequence at least 99% identical to the amino acid sequence of SEQ ID NO: 73. The non-naturally occurring polypeptide comprising an HLA-E comprises, consists essentially of, or consists of the amino acid sequence of SEQ ID NO: 73. The non-naturally occurring polypeptide comprising an HLA-E can be encoded by the nucleic acid have the sequence of SEQ ID NO: 74.

A modified cell of the present disclosure (preferably a modified T cell of the present disclosure) can further comprise, consist essentially of, or consist of a non-naturally occurring antigen receptor, a sequence encoding a therapeutic polypeptide, or a combination thereof. In a preferred aspect, the non-naturally occurring antigen receptor comprises, consists essentially of or consists of a chimeric antigen receptor (CAR). The CAR comprise, consist essentially of, or consist of (a) an ectodomain comprising an antigen recognition region, (b) a transmembrane domain, and (c) an endodomain comprising at least one costimulatory domain. The ectodomain of the CAR can further comprise, consist essentially of, or consist of a signal peptide. The ectodomain of the CAR can further comprise, consist essentially of, or consist of a hinge between the antigen recognition region and the transmembrane domain. The endodomain of the CAR can further comprise, consist essentially of, or consist of a human CD3 endodomain. The at least one costimulatory domain of the CAR can further comprise, consist essentially of, or consist of a human 4-1BB, CD28, CD40, ICOS, MyD88, OX-40 intracellular segment, or any combination thereof. In a preferred aspect, at least one costimulatory domain comprises a human CD28 and/or a 4-1BB costimulatory domain.

A modified cell of the present disclosure can be an immune cell or an immune cell precursor. The immune cell can be a regulatory T cell (Treg), a lymphoid progenitor cell, a natural killer (NK) cell, a cytokine induced killer (CIK) cell, a T lymphocyte (T cell), including effector T-cells, a B lymphocyte (B-cell) or an antigen presenting cell (APC). In preferred aspects, the immune cell is a T cell, an early memory T cell, a stem cell-like T cell, a stem memory T cell (T_{SCM}), a central memory T cell (T_{CM}) or a stem cell-like T cell. The immune cell precursor can a hematopoietic stem cell (HSC). The modified cell can be a stem cell, a differentiated cell, a somatic cell or an antigen presenting cell (APC). The modified cell can be an autologous cell or an allogeneic cell. In one aspect, the cell is a modified allogeneic T cell. In another aspect, the cell is modified allogeneic T cell expressing a chimeric antigen receptor (CAR), a CAR T cell.

A modified cell of the present disclosure (preferably a modified T cell of the present disclosure) can express a CSR of the present disclosure transiently or stably. In one aspect, a CSR of the present disclosure is transiently expressed in a modified cell of the present disclosure (preferably a modified T cell of the present disclosure). In one aspect, a CSR of the present disclosure is stably expressed in a modified cell of the present disclosure (preferably a modified T cell of the present disclosure).

A modified cell of the present disclosure (preferably a modified T cell of the present disclosure) can express a non-naturally occurring polypeptide comprising the HLA-E of the present disclosure transiently or stably. In one aspect, a non-naturally occurring polypeptide comprising the HLA-E of the present disclosure is transiently expressed in a modified cell of the present disclosure (preferably a modified T cell of the present disclosure). In one aspect, a non-naturally occurring polypeptide comprising the HLA-E of the present disclosure is stably expressed in a modified cell of the present disclosure (preferably a modified T cell of the present disclosure).

A modified cell of the present disclosure (preferably a modified T cell of the present disclosure) can express an inducible proapoptotic polypeptide of the present disclosure transiently or stably. In one aspect, an inducible proapoptotic polypeptide of the present disclosure is transiently expressed in a modified cell of the present disclosure (preferably a modified T cell of the present disclosure). In a preferred aspect, an inducible proapoptotic polypeptide of the present disclosure is stably expressed in a modified cell of the present disclosure (preferably a modified T cell of the present disclosure).

A modified cell of the present disclosure (preferably a modified T cell of the present disclosure) can express a non-naturally occurring antigen receptor or a sequence encoding a therapeutic protein of the present disclosure transiently or stably. In one aspect, a non-naturally occurring antigen receptor or a sequence encoding a therapeutic protein of the present disclosure is transiently expressed in a modified cell of the present disclosure (preferably a modified T cell of the present disclosure). In a preferred aspect, a non-naturally occurring antigen receptor or a sequence encoding a therapeutic protein of the present disclosure is stably expressed in a modified cell of the present disclosure (preferably a modified T cell of the present disclosure).

In one aspect, a CSR of the present disclosure is stably expressed, the inducible proapoptotic polypeptide of the present disclosure is stably expressed and a non-naturally occurring antigen receptor or a sequence encoding a therapeutic protein is stably expressed in a modified cell of the present disclosure (preferably a modified T cell of the present disclosure).

In one aspect, a CSR of the present disclosure is stably expressed, a non-naturally occurring polypeptide comprising the HLA-E of the present disclosure is stably expressed, the inducible proapoptotic polypeptide of the present disclosure is stably expressed and a non-naturally occurring antigen receptor or a sequence encoding a therapeutic protein is stably expressed in a modified cell of the present disclosure (preferably a modified T cell of the present disclosure).

In one aspect, a CSR of the present disclosure is stably expressed, a non-naturally occurring polypeptide comprising the HLA-E of the present disclosure is transiently expressed, the inducible proapoptotic polypeptide of the present disclosure is stably expressed and a non-naturally occurring antigen receptor or a sequence encoding a therapeutic protein is stably expressed in a modified cell of the present disclosure (preferably a modified T cell of the present disclosure).

In one aspect, a CSR of the present disclosure is transiently expressed, the inducible proapoptotic polypeptide of the present disclosure is stably expressed and the non-naturally occurring antigen receptor or a sequence encoding a therapeutic protein is stably expressed in a modified cell of the present disclosure (preferably a modified T cell of the present disclosure).

In one aspect, a CSR of the present disclosure is transiently expressed, a non-naturally occurring polypeptide comprising the HLA-E of the present disclosure is transiently expressed, the inducible proapoptotic polypeptide of the present disclosure is stably expressed and a non-naturally occurring antigen receptor or a sequence encoding a therapeutic protein is stably expressed in a modified cell of the present disclosure (preferably a modified T cell of the present disclosure).

In one aspect, a CSR of the present disclosure is transiently expressed, a non-naturally occurring polypeptide comprising the HLA-E of the present disclosure is stably expressed, the inducible proapoptotic polypeptide of the present disclosure is stably expressed and a non-naturally occurring antigen receptor or a sequence encoding a therapeutic protein is stably expressed in a modified cell of the present disclosure (preferably a modified T cell of the present disclosure).

The present disclosure provides a modified cell (preferably a modified T cell comprising, consisting essential of, or consisting of (a) a modification of an endogenous sequence encoding a T cell Receptor (TCR), wherein the modification reduces or eliminates a level of expression or activity of the TCR; and (b) a sequence encoding a chimeric stimulatory receptor (CSR) comprising: (i) an ectodomain comprising an activation component, wherein the activation component is isolated or derived from a first protein; (ii) a transmembrane domain; and (iii) an endodomain comprising at least one signal transduction domain, wherein the at least one signal transduction domain is isolated or derived from a second protein; wherein the first protein and the second protein are not identical.

The modified cell further can further comprise, consist essentially of or consist of a sequence encoding an inducible proapoptotic polypeptide. The modified cell can further comprise, consist essentially of or consist of a sequence encoding a non-naturally occurring antigen receptor, a sequence encoding a therapeutic polypeptide, or a combination thereof. The non-naturally occurring antigen receptor can comprise, consist essentially of or consist of a chimeric antigen receptor (CAR).

A transposon, a vector, a donor sequence or a donor plasmid can comprise, consist essentially of or consist of the sequence encoding the CSR, the sequence encoding the inducible proapoptotic polypeptide, or a combination thereof. The transposon, the vector, the donor sequence or the donor plasmid can further comprise, consist essentially of or consist of a sequence encoding a non-naturally occurring antigen receptor or a sequence encoding a therapeutic protein. The transposon, the vector, the donor sequence, or the donor plasmid can further comprise, consist essentially of or consist of a sequence encoding a selection marker. The transposon can be a piggyBac® transposon, a piggyBac® like transposon, a Sleeping Beauty transposon, a Helraiser transposon, a To12 transposon or a TcBuster transposon. The sequence encoding the CSR can be transiently expressed in the cell. The sequence encoding the CSR can be stably expressed in the cell. The sequence encoding the inducible proapoptotic polypeptide can be stably expressed in the cell. The sequence encoding a non-naturally occurring antigen receptor or a sequence encoding a therapeutic protein is stably expressed in the cell. In some aspects, the sequence encoding the CSR can be transiently expressed in the cell and the sequence encoding the inducible proapoptotic polypeptide can be stably expressed in the cell. In some aspects, the sequence encoding the CSR can be stably expressed in the cell and the sequence encoding the inducible proapoptotic polypeptide can be stably expressed in the cell. In some aspects, the sequence encoding the CSR can be transiently expressed in the cell, the sequence encoding the inducible proapoptotic polypeptide can be stably expressed in the cell and sequence encoding a non-naturally occurring antigen receptor or a sequence encoding a therapeutic protein is stably expressed in the cell. In some aspects, the sequence encoding the CSR can be stably expressed in the cell, the sequence encoding the inducible proapoptotic polypeptide can be stably expressed in the cell and sequence encoding a non-naturally occurring antigen receptor or a sequence encoding a therapeutic protein is stably expressed in the cell. In one aspect, the vector can be a viral vector. In one aspect, a viral vector can be an an adenoviral vector, adeno-associated viral (AAV) vector, retroviral vector, lentiviral vector or a chimeric viral vector.

A first transposon, a first vector, a first donor sequence, or a first donor plasmid can comprise, consist essentially of or consist of the sequence encoding the CSR. The first transposon, the first vector, the first donor sequence, or the first donor plasmid can further comprise, consist essentially of or consist of a sequence encoding a first selection marker.

A second transposon, a second vector, a second donor sequence, or a second donor plasmid can comprise, consist essentially of or consist of one or more of the sequence encoding the inducible proapoptotic polypeptide, the sequence encoding a non-naturally occurring antigen receptor, and the sequence encoding a therapeutic protein. The second transposon, the second vector, the second donor sequence, or the second donor plasmid can further comprise, consist essentially of or consist of a sequence encoding a second selection marker. The first selection marker and the second selection marker are identical. The first selection marker and the second selection marker are not identical. The selection marker can comprise, consist essentially of or consist of a cell surface marker. The selection marker can comprise, consist essentially of or consist of a protein that is active in dividing cells and not active in non-dividing cells. The selection marker can comprise, consist essentially of or consist of a metabolic marker.

In one aspect, the selection marker can comprise, consist essentially of or consist of a dihydrofolate reductase (DHFR) mutein enzyme. The DHFR mutein enzyme can comprise, consist essentially of or consist of the amino acid sequence of SEQ ID NO: 75.

The DHFR mutein enzyme of SEQ ID NO: 75 can further comprise, consist essentially of or consist of a mutation at one or more of positions 80, 113, or 153. The amino acid sequence of the DHFR mutein enzyme of SEQ ID NO: 75 can further comprise, consist essentially of or consist of one or more of a substitution of a Phenylalanine (F) or a Leucine (L) at position 80; a substitution of a Leucine (L) or a Valine (V) at position 113, and a substitution of a Valine (V) or an Aspartic Acid (D) at position 153.

A modified cell of the present disclosure (preferably a modified T cell of the present disclosure) can further comprise, consist essentially of or consist of a gene editing composition. The gene editing composition can comprise, consist essentially of or consist of a sequence encoding a DNA binding domain and a sequence encoding a nuclease protein or a nuclease domain thereof. The gene editing composition can be expressed transiently by the modified cell. The gene editing composition can be expressed stably by the modified cell.

The gene editing composition can comprise, consist essentially of or consist of a sequence encoding a nuclease protein or a sequence encoding a nuclease domain thereof. The sequence encoding a nuclease protein or the sequence encoding a nuclease domain thereof can comprise, consist essentially of or consist of a DNA sequence, an RNA sequence, or a combination thereof. The nuclease or the nuclease domain thereof can comprise, consist essentially of or consist of one or more of a CRISPR/Cas protein, a Transcription Activator-Like Effector Nuclease (TALEN), a Zinc Finger Nuclease (ZFN), and an endonuclease. The CRISPR/Cas protein can comprise, consist essentially of or consist of a nuclease-inactivated Cas (dCas) protein. The nuclease or the nuclease domain thereof can comprise, consist essentially of or consist of a nuclease-inactivated Cas (dCas) protein and an endonuclease. The endonuclease can comprise, consist essentially of or consist of a Clo051 nuclease or a nuclease domain thereof. The gene editing composition can comprise, consist essentially of or consist of a fusion protein. The fusion protein can comprise, consist essentially of or consist of a nuclease-inactivated Cas9 (dCas9) protein and a Clo051 nuclease or a Clo051 nuclease domain. The fusion protein can comprise, consist essentially of or consist of the amino acid sequence of SEQ ID NO: 76. The fusion protein is encoded by a nucleic acid comprising, consisting essentially of or consisting of the sequence of SEQ ID NO: 77. The fusion protein can comprise, consist essentially of or consist of the amino acid sequence of SEQ ID NO: 78. The fusion protein is encoded by a nucleic acid comprising, consisting essential of or consisting of the sequence of SEQ ID NO: 79.

The gene editing composition can further comprise, consist essentially of or consist of a guide sequence. The guide sequence can comprise, consist essentially of or consist of an RNA sequence. In aspects when the modified cell is a T cell, e.g., a Treg or an effector T-cell, the guide RNA can comprise, consist essentially of or consist of a sequence complementary to a target sequence encoding an endogenous TCR. The guide RNA can comprise, consist essentially of or consist of a sequence complementary to a target sequence encoding a B2M polypeptide. The guide RNA can comprise, consist essentially of or consist of a sequence complementary to a target sequence within a safe harbor site of a genomic DNA sequence.

The transposon, the vector, the donor sequence or the donor plasmid can further comprise, consist essentially of or consist of a gene editing composition comprising a guide sequence and a sequence encoding a fusion protein comprising a sequence encoding an inactivated Cas9 (dCas9) and a sequence encoding a Clo051 nuclease or a nuclease domain thereof.

The first transposon, the first vector, the first donor sequence or the first donor plasmid can further comprise, consist essentially of or consist of a gene editing composition comprising a guide sequence and a sequence encoding a fusion protein comprising a sequence encoding an inactivated Cas9 (dCas9) and a sequence encoding a Clo051 nuclease or a nuclease domain thereof.

The second transposon, the second vector, the second donor sequence or the second donor plasmid can further comprise, consist essentially of or consist of a gene editing composition comprising a guide sequence and a sequence encoding a fusion protein comprising a sequence encoding an inactivated Cas9 (dCas9) and a sequence encoding a Clo051 nuclease or a nuclease domain thereof.

A third transposon, a third vector, a third donor sequence or a third donor plasmid can comprise, consist essentially of or consist of a gene editing composition comprising a guide sequence and a sequence encoding a fusion protein comprising a sequence encoding an inactivated Cas9 (dCas9) and a sequence encoding a Clo051 nuclease or a nuclease domain thereof.

The Clo051 nuclease or a nuclease domain thereof can induce a single or double strand break in a target sequence. The donor sequence or a donor plasmid can integrate at a position of single or double strand break or at a position of cellular repair within a target sequence, or a combination thereof.

The present disclosure provides a composition comprising, consisting essential of, or consisting of a modified cell of the present disclosure (preferably a modified T cell of the present disclosure).

The present disclosure provides a plurality of modified cells comprising any non-naturally occurring chimeric stimulatory receptor (CSR) disclosed herein and provides a plurality of modified cells comprising any modified cell disclosed herein. The plurality of modified cells can comprise, consist essentially of, or consist of immune cells or an immune cell precursors. The plurality of immune cells can comprise, consist essentially of, or consist of lymphoid progenitor cells, natural killer (NK) cells, cytokine induced killer (CIK) cells, T lymphocytes (T cells), e.g., a Treg or an effector T cell, B lymphocytes (B-cells) or antigen presenting cells (APCs).

The present disclosure provides a composition comprising a population of modified cells, wherein a plurality of the modified cells of the population comprise any non-naturally occurring chimeric stimulatory receptor (CSR) disclosed herein and provides a composition comprising a population of modified cells, wherein a plurality of the modified cells of the population comprise any modified cell disclosed herein. The population of modified cells can comprise, consist essentially of, or consist of immune cells or an immune cell precursors. The population of immune cells can comprise, consist essentially of, or consist of lymphoid progenitor cells, natural killer (NK) cells, cytokine induced killer (CIK) cells, T lymphocytes (T cells), e.g., a Treg or an effector T cell, B lymphocytes (B-cells) or antigen presenting cells (APCs). The composition can comprise a pharmaceutically-acceptable carrier.

The present disclosure provides a composition comprising a population of modified T lymphocytes (T cells), wherein a plurality of the modified T cells of the population comprise any non-naturally occurring chimeric stimulatory receptor (CSR) disclosed herein and provides a composition comprising a population of T lymphocytes (T cells), e.g., a Treg or an effector T cell, wherein a plurality of the T cells of the population comprise any modified T cell disclosed herein. The composition can comprise a pharmaceutically-acceptable carrier.

Preferably, the present disclosure provides a composition comprising a population of T lymphocytes (T cells), e.g., a Treg or an effector T cell, wherein a plurality of the T cells of the population comprise a non-naturally occurring chimeric stimulatory receptor (CSR) comprising, consisting essential of, or consisting of: (a) an ectodomain comprising a activation component, wherein the activation component is isolated or derived from a first protein; (b) a transmembrane domain; and (c) an endodomain comprising at least one signal transduction domain, wherein the at least one signal transduction domain is isolated or derived from a second protein, wherein the first protein and the second protein are not identical. The composition can comprise a pharmaceutically-acceptable carrier. In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprise the CSR.

The plurality of the T cells of the population can further comprise an inducible proapoptotic polypeptide. In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprise the inducible proapoptotic polypeptide.

The plurality of the T cells of the population can further comprise a modification of an endogenous sequence encoding a T cell Receptor (TCR), wherein the modification reduces or eliminates a level of expression or activity of the TCR. In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprise the modification of the endogenous sequence encoding the TCR, wherein the modification reduces or eliminates a level of expression or activity of the TCR.

The plurality of the T cells of the population can further comprise a modification of an endogenous sequence encoding Beta-2-Microglobulin (B2M), wherein the modification reduces or eliminates a level of expression or activity of a major histocompatibility complex (MHC) class I (MHC-I). In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprise the modification of the endogenous sequence encoding B2M, wherein the modification reduces or eliminates a level of expression or activity of MHC-I.

The plurality of the T cells of the population can further comprise a modification of an endogenous sequence encoding a T cell Receptor (TCR), wherein the modification reduces or eliminates a level of expression or activity of the TCR and a modification of an endogenous sequence encoding Beta-2-Microglobulin (B2M), wherein the modification reduces or eliminates a level of expression or activity of a major histocompatibility complex (MHC) class I (MHC-I).

In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprise both modification of the endogenous sequence encoding the TCR, wherein the modification reduces or eliminates a level of expression or activity of the TCR and the modification of the endogenous sequence encoding B2M, wherein the modification reduces or eliminates a level of expression or activity of MHC-I.

The plurality of the T cells of the population can further comprise a non-naturally occurring sequence comprising an HLA class I histocompatibility antigen, alpha chain E (HLA-E) polypeptide. In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprise the non-naturally occurring sequence comprising the HLA-E polypeptide.

The plurality of the T cells of the population can further comprise a non-naturally occurring antigen receptor, a sequence encoding a therapeutic polypeptide, or a combination thereof. In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprise the non-naturally occurring antigen receptor, the sequence encoding a therapeutic polypeptide, or a combination thereof. In preferred aspects, the non-naturally occurring antigen receptor is a chimeric antigen receptor (CAR).

The plurality of the T cells of the population can comprise an early memory T cell, a stem cell-like T cell, a stem memory T cell (T_{SCM}), a central memory T cell (T_{CM}) or a stem cell-like T cell. In some aspects, one or more of a stem cell-like T cell, a stem cell memory T cell (T_{SCM}) and a central memory T cell (T_{CM}) comprise at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population of modified T cells.

In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprising the CSR expresses one or more cell-surface marker(s) of a stem memory T cell (T_{SCM}) or a T_{SCM}-like cell; and wherein the one or more cell-surface marker(s) comprise CD45RA and CD62L.

In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population expresses one or more cell-surface marker(s) of a central memory T cell (T_{CM}) or a T_{CM}-like cell; and wherein the one or more cell-surface marker(s) comprise CD45RO and CD62L.

In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population expresses one or more of CD127, CD45RO, CD95 and IL-2Rβ cell-surface marker(s).

The present disclosure provides compositions for use in the treatment of a disease or disorder disclosed herein or the use of a composition for the treatment of any disease or disorder disclosed herein. The present disclosure also provides methods of treating a disease or disorder comprising, consisting essential of, or consisting of administering to a subject in need thereof a therapeutically-effective amount of a composition disclosed herein. The compositions can comprise, consist essentially of or consist of any of the modified cells or populations of modified cells disclosed herein. Preferably, any of the modified T cells or CAR T cells disclosed herein.

The present disclosure provides a method of producing a modified T cell comprising, consisting essential of, or consisting of, introducing into a primary human T cell a composition comprising a Chimeric Stimulator Receptor (CSR) of the present disclosure or a sequence encoding the same to produce a modified T cell under conditions that stably express the CSR within the modified T cell and preserve desirable stem-like properties of the modified T cell. The primary human T cell can be a resting primary human T cell. The present disclosure provides a modified T cell produced by the disclosed method. The present disclosure provides a method of administering the modified T cell comprising the stably expressed CSR produced by the disclosed method. The present disclosure provides the method of administering the modified T cell comprising the stably expressed CSR produced by the disclosed method to treat a disease or disorder.

The present disclosure provides a method of producing a population of modified T cells comprising, consisting essential of, or consisting of, introducing into a plurality of primary human T cells a composition comprising a Chimeric Stimulator Receptor (CSR) of the present disclosure or a sequence encoding the same to produce a plurality of modified T cells under conditions that stably express the CSR within the plurality of modified T cells and preserve desirable stem-like properties of the plurality of modified T cells. The primary human T cells can comprise resting primary human T cells. The present disclosure provides a population of modified T cells produced by the disclosed method. The present disclosure provides a method of administering the population of modified T cells comprising the stably expressed CSR produced by the disclosed method. The present disclosure provides a method of administering the population of modified T cells comprising the stably expressed CSR produced by the disclosed method to treat a disease or disorder.

The present disclosure provides a method of producing a modified T cell comprising, consisting essential of, or consisting of, introducing into a primary human T cell a composition comprising a Chimeric Stimulator Receptor (CSR) of the present disclosure or a sequence encoding the same to produce a modified T cell under conditions that transiently express the CSR within the modified T cell and preserve desirable stem-like properties of the modified T cell. The primary human T cell can be a resting primary human T cell. The present disclosure provides a modified T cell produced by the disclosed method. The present disclosure provides a method of administering the modified T cell comprising the transiently expressed CSR produced by the disclosed method. In one aspect, the present disclosure provides a method of administering the modified T cell produced by the disclosed method after the modified T cell no longer expresses the CSR. The present disclosure provides a method of administering a modified T cell comprising the transiently expressed CSR produced by the disclosed method to treat a disease or disorder. In one aspect, the present disclosure provides a method of administering the modified T cell produced by the disclosed method after the modified T cell no longer expresses the CSR to treat a disease or disorder.

The present disclosure provides a method of producing a population of modified T cells comprising, consisting essential of, or consisting of, introducing into a plurality of primary human T cells a composition comprising a Chimeric Stimulator Receptor (CSR) of the present disclosure or a sequence encoding the same to produce a plurality of modified T cells under conditions that transiently express the CSR within the plurality of modified T cells and preserve desirable stem-like properties of the plurality of modified T cells. The primary human T cells can comprise resting primary human T cells. The present disclosure provides a population of modified T cell produced by the disclosed method. The present disclosure provides a method of administering the population of modified T cells comprising the transiently expressed CSR produced by the disclosed method. In one aspect, the present disclosure provides a method of administering the population of modified T cells produced by the disclosed method after the plurality of T cells no longer express the CSR. The present disclosure provides a method of administering the population of modified T cells comprising the transiently expressed CSR produced by the disclosed method to treat a disease or disorder. In one aspect, the present disclosure provides a method of administering the population of modified T cells produced by the disclosed method after the plurality of modified T cells no longer express the CSR to treat a disease or disorder.

The method of producing a modified T cell or producing a population of modified T cells can further comprise introducing a modification of an endogenous sequence encoding a T cell Receptor (TCR), wherein the modification reduces or eliminates a level of expression or activity of the TCR. The method of producing a modified T cell or producing a population of modified T cells can further comprise introducing a modification of an endogenous sequence encoding Beta-2-Microglobulin (B2M), wherein the modification reduces or eliminates a level of expression or activity of a major histocompatibility complex (MHC) class I (MHC-1). In some aspects, the method of producing a modified T cell or producing a population of modified T cells can further comprising introducing both a modification of an endogenous sequence encoding TCR, wherein the modification reduces or eliminates a level of expression or activity of the TCR and introducing a modification of an endogenous sequence encoding B2M, wherein the modification reduces or eliminates a level of expression or activity of MHC-1.

The method of producing a modified T cell or producing a population of modified T cells can further comprise introducing into the primary human T cell or plurality of primary human T cells a composition comprising an antigen receptor, a therapeutic protein or a sequence encoding the same. In one aspect, the antigen receptor is a non-naturally occurring antigen receptor. In a preferred aspect, the method of producing a modified T cell or producing a population of modified T cells can further comprise introducing into the primary human T cell or plurality of primary human T cells a composition comprising a Chimeric Antigen Receptor (CAR) or a sequence encoding the same. The method can further comprise introducing into the primary human T cell or plurality of primary human T cells a composition comprising an inducible proapoptotic polypeptide or a sequence encoding the same. The method of producing a modified T cell or producing a population of modified T cells can further comprise introducing into the primary human T cell or plurality of primary human T cells a composition comprising an antigen receptor, a therapeutic protein or a sequence encoding the same and a composition comprising an inducible proapoptotic polypeptide or a sequence encoding the same.

The method of producing a modified T cell or producing a population of modified T cells can further comprise contacting the modified T cell or population of modified T cells with an activator composition. The activator composition can comprise, consist essentially of, or consist of one or more agonists or activating agents that can bind a CSR activation component of the modified T cell or plurality of modified T cells. The agonist/activating agent can be naturally occurring or non-naturally occurring. In preferred aspects, the agonist/activating agent is an antibody or antibody fragment. The agonist/activating agent can be one or more of an anti-CD3 antibody or fragment thereof, an anti-CD2 antibody or fragment thereof, an anti-CD28 antibody or fragment thereof, or any combination thereof. In some aspects, the agonist/activating agent that can be one or more of an anti-human CD3 monospecific tetrameric antibody complex, an anti-human CD2 monospecific tetrameric antibody complex, an anti-human CD28 monospecific tetrameric antibody complex, or a combination thereof. The agonist/activating can contact the modified T cell or population of modified T cells in vitro, ex vivo or in vivo. In a preferred aspect, the agonist/activating activates the modified T cell or population of modified T cells, induces cell division in the modified T cell or population of modified T cells, increases cell division (e.g., cell doubling time) in the modified T cell or population of modified T cells, increases fold expansion in the modified T cell or population of modified T cells, or any combination thereof.

The present disclosure provides a method of expanding modified Treg cells within a population of modified T cells comprising, consisting essential of, or consisting of, introducing into a plurality of primary human T cells a composition comprising a Chimeric Stimulator Receptor (CSR) of the present disclosure or a sequence encoding the same to produce a plurality of modified T cells under conditions that stably express the CSR within the plurality of modified T cells and preserve desirable Treg properties of the plurality of modified T cells and culturing the modified T cells in the presence of an activator composition to produce a plurality of activated modified Treg cells, wherein expansion of the plurality of modified Treg cells is higher than the expansion of a plurality of Treg cells not stably expressing a CSR of the present disclosure under the same conditions.

In some aspects, a plurality is wherein at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% or any percentage in between of the modified T cells are modified Treg cells.

Expansion of CSR expressing Treg cells can be wherein the total population of CSR expressing Treg cells increases by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% or any percentage in between in comparison to Treg cells not expressing the CSR under the same conditions. The expansion of CSR expressing Treg cells can be wherein the total population of CSR expressing Treg cells increase by 5% to 10%, 10% to 15%, 15% to 20%, 20% to 25%, 25% to 30%, 30% to 35%, 35% to 40%, 40% to 45%, 45% to 50%, 50% to 55%, 55% to 60%, 60% to 65%, 65% to 70%, 70% to 75%, 75% to 80%, 80% to 85%, 85% to 90%, 90% to 95% or 95% to 100% in comparison to Treg cells not expressing the CSR under the same conditions.

Expansion of CSR expressing Treg cells can be wherein the total population of CSR expressing Treg cells increases by at least 1.0 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 21 fold, at least 22 fold, at least 23 fold, at least 24 fold, at least 25 fold, at least 26 fold, at least 27 fold, at least 28 fold, at least 29 fold or at least 30 fold in comparison to Treg cells not expressing the CSR under the same conditions.

The present disclosure provides a method of expanding modified Treg cells within a population of modified T cells comprising, consisting essential of, or consisting of, introducing into a plurality of primary human T cells a composition comprising a Chimeric Stimulator Receptor (CSR) of the present disclosure or a sequence encoding the same to produce a plurality of modified T cells under conditions that transiently express the CSR within the plurality of modified T cells and preserve desirable Treg properties of the plurality of modified T cells, and culturing the modified T cells in the presence of an activator composition to produce a plurality of activated modified Treg cells, wherein expansion of the plurality of modified Treg cells is higher than the expansion of a plurality of Treg cells not transiently expressing a CSR of the present disclosure under the same conditions.

In some aspects, a plurality is wherein at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% or any percentage in between of the modified T cells are modified Treg cells.

Expansion of CSR expressing Treg cells can be wherein the total population of CSR expressing Treg cells increases by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% or any percentage in between in comparison to Treg cells not expressing the CSR under the same conditions. The expansion of CSR expressing Treg cells can be wherein the total population of CSR expressing Treg cells increase by 5% to 10%, 10% to 15%, 15% to 20%, 20% to 25%, 25% to 30%, 30% to 35%, 35% to 40%, 40% to 45%, 45% to 50%, 50% to 55%, 55% to 60%, 60% to 65%, 65% to 70%, 70% to 75%, 75% to 80%, 80% to 85%, 85% to 90%, 90% to 95% or 95% to 100% or any percentage in between in comparison to Treg cells not expressing the CSR under the same conditions.

Expansion of CSR expressing Treg cells can be wherein the total population of CSR expressing Treg cells increases by at least 1.0 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 21 fold, at least 22 fold, at least 23 fold, at least 24 fold, at least 25 fold, at least 26 fold, at least 27 fold, at least 28 fold, at least 29 fold or at least 30 fold in comparison to Treg cells not expressing the CSR under the same conditions.

The activator composition of the methods of expanding a population of can comprise, consist essentially of, or consist of one or more agonists or activating agents that can bind a CSR activation component of the modified T cell or plurality of modified T cells. The agonist/activating agent can be naturally occurring or non-naturally occurring. Exemplary naturally occurring agonist/activating agents include but are not limited to CD2 ligands such as CD48, CD58 or CD59, or any combination thereof. CD2 ligands can be full length, partial insoluble or non-soluble form, monomeric or other, homogenous or heterologous. In preferred aspects, the agonist/activating agent is an antibody or antibody fragment. The agonist/activating agent can be one or more of an anti-CD3 antibody or fragment thereof, an anti-CD2 antibody or fragment thereof, an anti-CD28 antibody or fragment thereof, or any combination thereof. In some aspects, the agonist/activating agent that can be one or more of an anti-human CD3 monospecific tetrameric antibody complex, an anti-human CD2 monospecific tetrameric antibody complex, an anti-human CD28 monospecific tetrameric antibody complex, or a combination thereof.

The conditions can comprise culturing the modified T cell or plurality of modified T cells in a media comprising a sterol; an alkane; phosphorus and one or more of an octanoic acid, a palmitic acid, a linoleic acid, and an oleic acid. The culturing can be in vivo or ex vivo. The modified T cell can be an allogeneic T cell or the plurality of modified T cells can be allogeneic T cells. The modified T cell can be an autologous T cell or the plurality of modified T cells can be autologous T cells.

In some aspects, the media can comprise one or more of octanoic acid at a concentration of between 0.9 mg/kg to 90 mg/kg, inclusive of the endpoints; palmitic acid at a concentration of between 0.2 mg/kg to 20 mg/kg, inclusive of the endpoints; linoleic acid at a concentration of between 0.2 mg/kg to 20 mg/kg, inclusive of the endpoints; oleic acid at a concentration of 0.2 mg/kg to 20 mg/kg, inclusive of the endpoints; and a sterol at a concentration of about 0.1 mg/kg to 10 mg/kg, inclusive of the endpoints.

In some aspects, the media can comprise one or more of octanoic acid at a concentration of about 9 mg/kg, palmitic acid at a concentration of about 2 mg/kg, linoleic acid at a concentration of about 2 mg/kg, oleic acid at a concentration of about 2 mg/kg and a sterol at a concentration of about 1 mg/kg.

In some aspects, the media can comprise one or more of octanoic acid at a concentration of between 6.4 μmol/kg and 640 μmol/kg, inclusive of the endpoints; palmitic acid at a concentration of between 0.7 μmol/kg and 70 μmol/kg, inclusive of the endpoints; linoleic acid at a concentration of between 0.75 μmol/kg and 75 μmol/kg, inclusive of the endpoints; oleic acid at a concentration of between 0.75 μmol/kg and 75 μmol/kg, inclusive of the endpoints; and a sterol at a concentration of between 0.25 μmol/kg and 25 μmol/kg, inclusive of the endpoints.

In some aspects, the media can comprise one or more of octanoic acid at a concentration of about 64 μmol/kg, palmitic acid at a concentration of about 7 μmol/kg, linoleic acid at a concentration of about 7.5 μmol/kg, oleic acid at a concentration of about 7.5 μmol/kg and a sterol at a concentration of about 2.5 μmol/kg.

The present disclosure provides compositions comprising any modified T cell produced by a method disclosed herein. The present disclosure provides compositions comprising any population of modified T cell produced by a method disclosed herein. The present disclosure provides compositions comprising any modified T cell expanded by a method disclosed herein. The present disclosure provides compositions comprising any population of modified T cell expanded by a method disclosed herein.

The present disclosure provides compositions for use in the treatment of a disease or disorder disclosed herein or the use of a composition for the treatment of any disease or disorder disclosed herein. The present disclosure also provides methods of treating a disease or disorder comprising, consisting essential of, or consisting of administering to a subject in need thereof a therapeutically-effective amount of a composition disclosed herein and at least one non-naturally occurring molecule which binds to the activation component of a CSR disclosed herein. The compositions can comprise, consist essentially of or consist of any of the modified cells or populations of modified cells disclosed herein. Preferably, any of the modified T cells or CSR T cells disclosed herein. Any non-naturally occurring molecule capable of binding to the activation component of the CSR of the present disclosure and selectively transducing a signal upon binding can be administered. Preferably, the non-naturally occurring molecule is an non-naturally CSR agonist/activating agent for the activation component. The non-naturally occurring agonist/activating agent that can bind a CSR activation component can be any non-naturally occurring antibody or antibody fragment. The non-naturally occurring antibody or antibody fragment can be a non-naturally occurring anti-CD3 antibody or fragment thereof, an anti-CD2 antibody or fragment thereof, an anti-CD28 antibody or fragment thereof, or any combination thereof. In some aspects, the non-naturally occurring agonist/activating agent that can bind a CSR activation component can be one or more of an anti-human CD3 monospecific tetrameric antibody complex, an anti-human CD2 monospecific tetrameric antibody complex, an anti-human CD28 monospecific tetrameric antibody complex, or a combination thereof. In some aspects, the non-naturally occurring agonist/activating agent that can bind an activation component can be selected from the group consisting of anti-CD2 monoclonal antibody, BTI-322 (Przepiorka et al., Blood 92(11):4066-4071, 1998) and humanized anti-CD2 monoclonal antibody clone AFC-TAB-104 (Siplizumab)(Bissonnette et al. Arch. Dermatol. Res. 301(6):429-442, 2009). In some aspects, administration of non-naturally occurring molecule capable of binding to the activation component of the CSR stimulates cell division of the modified cells in vivo. Thus, the present disclosure provides a method of stimulating cell division of a modified cell of the present disclosure in vivo by administering a non-naturally CSR agonist/activating agent for the activation component to a subject harboring the modified cell of the present disclosure.

In some aspects, the disease or disorder is a cell proliferation disease or disorder. In some aspects, the cell proliferation disease or disorder is cancer. The cancer can be a solid tumor cancer or a hematologic cancer. In some aspects, the solid tumor is prostate cancer or breast cancer. In preferred aspects, the prostate cancer is castrate-resistant prostate cancer. In some aspects, the hematologic cancer is multiple myeloma.

The modified cells or population of modified cells comprised within the disclosed compositions can be cultured in vitro or ex vivo prior to administration to a subject in need thereof. The modified cells can be allogenic modified cells or autologous modified cells. In some aspects, the cells are allogeneic modified T cells or autologous modified T cells. In some aspects, the cells are allogeneic modified CAR T cells or autologous modified CAR T cells. In some aspects, the cells are allogeneic modified CAR T cells comprising a CSR of the present disclosure or autologous modified CAR T cells comprising a CSR of the present disclosure.

The modified cell compositions or the compositions comprising populations of modified cells can be administered to the patient by any means known in the art. In some aspects, the composition is administered by systemic administration. In some aspects, the composition is administered by intravenous administration. The intravenous administration can be in an intravenous injection or an intravenous infusion. In some aspects, the composition is administered by local administration. In some aspects, the composition is administered by an intraspinal, intracerebroventricular, intraocular or intraosseous injection or infusion.

The therapeutically effective amount can be a single dose or multiple doses of modified cell compositions or the compositions comprising populations of modified cells. In some aspects, the therapeutically effective dose is a single dose and wherein the allogeneic cells of the composition engraft and/or persist for a sufficient time to treat the disease or disorder. In some aspects, the single dose is one of at least 2, 5, 10, 15, 20, 25, 30, 35, 40, 45, 50, 55, 60, 65, 70, 75, 80, 85, 90, 95, 100 or any number of doses in between that are manufactured simultaneously

In some aspects, the uses and methods for the treatment of a disease or disorder further provide that subjects do not develop graft v host (GvH) disease, host v graft (HvG) disease, or a combination thereof, following administration of modified cell compositions disclosed herein or the compositions comprising populations of modified cells disclosed herein.

Allogeneic cells of the disclosure are engineered to prevent adverse reactions to engraftment following administration to a subject. Allogeneic cells may be any type of cell.

In some embodiments of the composition and methods of the disclosure, allogeneic cells are stem cells. In some embodiments, allogeneic cells are derived from stem cells. Exemplary stem cells include, but are not limited to, embryonic stem cells, adult stem cells, induced pluripotent stem cells (iPSCs), multipotent stem cells, pluripotent stem cells, and hematopoetic stem cells (HSCs).

In some embodiments of the composition and methods of the disclosure, allogeneic cells are differentiated somatic cells.

In some embodiments of the composition and methods of the disclosure, allogeneic cells are immune cells. In some embodiments, allogeneic cells are T lymphocytes (T cells). In some embodiments, allogeneic cells are T cells that do not express one or more components of a naturally-occurring T cell Receptor (TCR). In some embodiments, allogeneic cells are T cells that express a non-naturally occurring antigen receptor. Alternatively, or in addition, in some embodiments, allogeneic cells are T cells that express a non-naturally occurring Chimeric Stimulatory Receptor (CSR). In some embodiments, the non-naturally occurring CSR comprises or consists of a switch receptor. In some embodiments, the switch receptor comprises an extracellular domain, a transmembrane domain, and an intracellular domain. In some embodiments, the extracellular domain of the switch receptor binds to a TCR co-stimulatory molecule and transduces a signal to the intracellular space of the allogeneic cell that recapitulates TCR signaling or TCR co-stimulatory signaling.

Forkhead box P3 (FoxP3)

The disclosure provides compositions, methods of making and methods of using Tregs comprising a CSR with a persistent phenotype both ex vivo and when transplanted in a patient. In some embodiments, this persistent phenotype is due to the expression of forkhead box P3 (FoxP3) in a CD4+ T cell, Treg cell, CD4+ CAR-T cell, or CAR-Treg cell. In some embodiments, CD4+ T cells that are not Tregs can be genetically engineered into Tregs through the forced expression of FoxP3 using the methods and compositions of the disclosure. In some embodiments, FoxP3 can be encoded in a transgene with an inducible promoter, such that FoxP3 expression can be induced to create engineered Tregs or “forced Tregs”.

In some embodiments of the compositions and methods of the disclosure, the FoxP3 is wild-type (WT) FoxP3. Exemplary wild type human Foxp3 sequences are described in NP 054728.2, the contents of which are incorporated herein by reference. In some embodiments, the wild type FoxP3 comprises an amino acid sequence of having at least 80% identity, at least 90% identity, at least 95% identity, at least 99% identity or is identical to a sequence of

(SEQ ID NO: 80)

| | |
|---|---|
| 1 | MPNPRPGKPS APSLALGPSP GASPSWRAAP KASDLLGARG |
| | PGGTFQGRDL RGGAHASSSS |
| 61 | LNPMPPSQLQ LPTLPLVMVA PSGARLGPLP HLQALLQDRP |
| | HFMHQLSTVD AHARTPVLQV |
| 121 | HPLESPAMIS LTPPTTATGV FSLKARPGLP PGINVASLEW |
| | VSREPALLCT FPNPSAPRKD |
| 181 | STLSAVPQSS YPLLANGVCK WPGCEKVFEE PEDFLKHCQA |
| | DHLLDEKGRA QCLLQREMVQ |
| 241 | SLEQQLVLEK EKLSAMQAHL AGKMALTKAS SVASSDKGSC |
| | CIVAAGSQGP WPAWSGPRE |
| 301 | APDSLFAVRR HLWGSHGNST FPEFLHNMDY FKFHNMRPPF |
| | TYATLIRWAI LEAPEKQRTL |
| 361 | NEIYHWFTRM FAFFRNHPAT WKNAIRHNLS LHKCFVRVES |
| | EKGAVWTVDE LEFRKKRSQR |
| 421 | PSRCSNPTPG P. |

In some embodiments, the wild type FoxP3 comprises or consists essentially of SEQ ID NO: 80.

In some embodiments, the wild type FoxP3 is encoded by a nucleotide sequence having at least 80% identity, at least 90% identity, at least 95% identity, at least 99% identity or is identical to a sequence of

(SEQ ID NO: 81)

| | |
|---|---|
| 1 | atgcccaacc ccaggcctgg caagccctcg gccccttcct |
| | tggcccttgg cccatcccca |
| 61 | ggagcctcgc ccagctggag ggctgcaccc aaagcctcag |
| | acctgctggg ggcccggggc |
| 121 | ccagggggaa ccttccaggg ccgagatctt cgaggcgggg |
| | cccatgcctc ctcttcttcc |
| 181 | ttgaacccca tgccaccatc gcagctgcag ctgcccacac |
| | tgcccctagt catggtggca |
| 241 | ccctccgggg cacggctggg ccccttgccc cacttacagg |
| | cactcctcca ggacaggcca |
| 301 | catttcatgc accagctctc aacggtggat gcccacgccc |
| | ggacccctgt gctgcaggtg |
| 361 | caccccctgg agagcccagc catgatcagc ctcacaccac |
| | ccaccaccgc cactggggtc |
| 421 | ttctccctca aggcccggcc tggcctccca cctgggatca |
| | acgtggccag cctggaatgg |
| 481 | gtgtccaggg agccggcact gctctgcacc ttcccaaatc |
| | ccagtgcacc caggaaggac |
| 541 | agcacccttt cggctgtgcc ccagagctcc tacccactgc |
| | tggcaaatgg tgtctgcaag |
| 601 | tggcccggat gtgagaaggt cttcgaagag ccagaggact |
| | tcctcaagca ctgccaggcg |
| 661 | gaccatcttc tggatgagaa gggcagggca caatgtctcc |
| | tccagagaga gatggtacag |
| 721 | tctctggagc agcagctggt gctggagaag gagaagctga |
| | gtgccatgca ggcccacctg |
| 781 | gctgggaaaa tggcactgac caaggcttca tctgtggcat |
| | catccgacaa gggctcctgc |
| 841 | tgcatcgtag ctgctggcag ccaaggccct gtcgtcccag |
| | cctggtctgg cccccgggag |
| 901 | gcccctgaca gcctgtttgc tgtccggagg cacctgtggg |
| | gtagccatgg aaacagcaca |
| 961 | ttcccagagt tcctccacaa catggactac ttcaagttcc |
| | acaacatgcg accccctttc |
| 1021 | acctacgcca cgctcatccg ctgggccatc ctggaggctc |
| | cagagaagca gcggacactc |
| 1081 | aatgagatct accactggtt cacacgcatg tttgccttct |
| | tcagaaacca tcctgccacc |
| 1141 | tggaagaacg ccatccgcca caacctgagt ctgcacaagt |
| | gctttgtgcg ggtggagagc |
| 1201 | gagaaggggg ctgtgtggac cgtggatgag ctggagttcc |
| | gcaagaaacg gagccagagg |
| 1261 | cccagcaggt gttccaaccc tacacctggc ccc. |

### Chimeric Stimulatory Receptors (CSRs)

Adoptive cell compositions that are “universally” safe for administration to any patient requires a significant reduction or elimination of alloreactivity.

Towards this end, allogeneic cells of the disclosure are modified to interrupt expression or function of a T cell Receptor (TCR) and/or a class of Major Histocompatibility Complex (MHC). The TCR mediates graft vs host (GvH) reactions whereas the MHC mediates host vs graft (HvG) reactions. In preferred embodiments, any expression and/or function of the TCR is eliminated in allogeneic cells of the disclosure to prevent T cell mediated GvH that could cause death to the subject. Thus, in particularly preferred embodiments, the disclosure provides a pure TCR-negative allogeneic T cell composition (e.g., each cell of the composition expresses at a level so low as to either be undetectable or non-existent).

In preferred embodiments, expression and/or function of MEW class I (MHC-I, specifically, HLA-A, HLA-B, and HLA-C) is reduced or eliminated in allogeneic cells of the disclosure to prevent HvG and, consequently, to improve engraftment of allogeneic cells of the disclosure in a subject. Improved engraftment of the allogeneic cells of the disclosure results in longer persistence of the cells, and, therefore, a larger therapeutic window for the subject. Specifically, in the allogeneic cells of the disclosure, expression and/or function of a structural element of MHC-I, Beta-2-Microglobulin (B2M), is reduced or eliminated in allogeneic cells of the disclosure.

The above strategies for generating an allogeneic cell of the disclosure induce further challenges. T Cell Receptor (TCR) knockout (KO) in T cells results in loss of expression of CD3-zeta (CD3z or CD3ζ), which is part of the TCR complex. The loss of CD3λ in TCR-KO T cells dramatically reduces the ability of optimally activating and expanding these cells using standard stimulation/activation reagents, including, but not limited to, agonist anti-CD3 mAb. When the expression or function of any one component of the TCR complex is interrupted, all components of the complex are lost, including TCR-alpha (TCRα), TCR-beta (TCRβ), CD3-gamma (CD3γ), CD3-epsilon (CD3ε), CD3-delta (CD3 δ), and CD3-zeta (CD3ζ). Both CD3ε and CD3ζ are required for T cell activation and expansion. Agonist anti-CD3 mAbs typically recognize CD3ε and possibly another protein within the complex which, in turn, signals to CD3ζ. CD3ζ provides the primary stimulus for T cell activation (along with a secondary co-stimulatory signal) for optimal activation and expansion. Under normal conditions, full T cell activation depends on the engagement of the TCR in conjunction with a second signal mediated by one or more co-stimulatory receptors (e.g., CD28, CD2, 4-1BBL, etc. . . . ) that boost the immune response. However, when the TCR is not present, T cell expansion is severely reduced when stimulated using standard activation/stimulation reagents, including agonist anti-CD3 mAb. In fact, T cell expansion is reduced to only 20-40% of the normal level of expansion when stimulated using standard activation/stimulation reagents, including agonist anti-CD3 mAb.

The disclosure provides a Chimeric Stimulatory Receptor (CSR) to deliver CD3z primary stimulation to allogeneic T cells in the absence of an endogenous TCR (and, consequently, an endogenous CD3) when stimulated using standard activation/stimulation reagents, including agonist anti-CD3 mAb.

In the absence of an endogenous TCR, Chimeric Stimulatory Receptors (CSRs) of the disclosure provide a CD3 stimulus to enhance activation and expansion of allogeneic T cells. In other words, in the absence of an endogenous TCR, Chimeric Stimulatory Receptors (CSRs) of the disclosure rescue the allogeneic cell from an activation-based disadvantage when compared to non-allogeneic T cells that express an endogenous TCR. In some embodiments, CSRs of the disclosure comprise an agonist mAb epitope extracellularly and a CD3ζ stimulatory domain intracellularly and, functionally, convert an anti-CD28 or anti-CD2 binding event on the surface into a CD3z signaling event in an allogeneic T cell modified to express the CSR. In some embodiments, a CSR comprises a wild type CD28 or CD2 protein and a CD3z intracellular stimulation domain, to produce CD28z CSR and CD2z CSR, respectively. In preferred embodiments, CD28z CSR and/or CD2z CSR further express a non-naturally occurring antigen receptor and/or a therapeutic protein. In preferred embodiments, the non-naturally occurring antigen receptor comprises a Chimeric Antigen Receptor.

The data provided herein demonstrate that modified allogeneic T cells of the disclosure comprising/expressing a CSR of the disclosure improve or rescue, the expansion of allogeneic T cells that no longer express endogenous TCR when compared to those cells that do not comprise/express a CSR of the disclosure.

A wildtype/natural human CD28 protein (NCBI: CD28 HUMAN; UniProt/Swiss-Prot: P10747.1) comprises or consists of the amino acid sequence of:

(SEQ ID NO: 82)
MLRLLLALNLEPSIQVTGNKILVKQSPMLVAYDNAVNLSCKYSYNLFSRE
FRASLHKGLDSAVEVCVVYGNYSQQLQVYSKTGFNCDGKLGNESVTFYLQ
NLYVNQTDIYFCKIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRS

A nucleotide sequence encoding wildtype/natural CD28 protein (NCBI: CCDS2361.1) comprises or consists of the nucleotide sequence of:

(SEQ ID NO: 83)
ATGCTCAGGCTGCTCTTGGCTCTCAACTTATTCCCTTCAATTCAAGTAAC
AGGAAACAAGATTTTGGTGAAGCAGTCGCCCATGCTTGTAGCGTACGACA
ATGCGGTCAACCTTAGCTGCAAGTATTCCTACAATCTCTTCTCAAGGGAG
TTCCGGGCATCCCTTCACAAAGGACTGGATAGTGCTGTGGAAGTCTGTGT
TGTATATGGGAATTACTCCCAGCAGCTTCAGGTTTACTCAAAAACGGGGT
TCAACTGTGATGGGAAATTGGGCAATGAATCAGTGACATTCTACCTCCAG
AATTTGTATGTTAACCAAACAGATATTTACTTCTGCAAAATTGAAGTTAT
GTATCCTCCTCCTTACCTAGACAATGAGAAGAGCAATGGAACCATTATCC
ATGTGAAAGGGAAACACCTTTGTCCAAGTCCCCTATTTCCCGGACCTTCT
AAGCCCTTTTGGGTGCTGGTGGTGGTTGGTGGAGTCCTGGCTTGCTATAG
CTTGCTAGTAACAGTGGCCTTTATTATTTTCTGGGTGAGGAGTAAGAGGA
GCAGGCTCCTGCACAGTGACTACATGAACATGACTCCCCGCCGCCCCGGG
CCCACCCGCAAGCATTACCAGCCCTATGCCCCACCACGCGACTTCGCAGC
CTATCGCTCCTGA

An exemplary CSR CD28z protein of the disclosure comprises or consists of the amino acid sequence of (CD28 Signal peptide, CD28 Extracellular Domain, CD28 Transmembrane domain, CD28 Cytoplasmic Domain, CD3z Intracellular Domain):

(SEQ ID NO: 84)
MLRLLLALNLFPSIQVTGNKILVKQSPMLVAYDNAVNLSCKYSYNLFSREFRASLHKGLDSA
VEVCVVYGNYSQQLQVYSKTGFNCDGKLGNESVTFYLQNLYVNQTDIYFCKIEVMYPPPYLD
NEKSNGTIIHVKGKHLCPSPLFPGPSKPFWVLVVVGGYEACYSLLVTVAFIIFWV [symbol] [symbol] [symbol]
RVKFSRSADAPAYKQGQNQLYNELNLGR
REEYDVLDKRRGRDPEMGGKPRRKNPQEGLYNELQKDKMAEAYSEIGMKGERRRGKGHDGLY
QGLSTATKDTYDALHMQALPPR
CD28 Signal peptide:
(SEQ ID NO: 85)
MLRLLLALNLFPSIQVTG
CD28 Extracellular Domain:
(SEQ ID NO: 86)
NKILVKQSPMLVAYDNAVNLSCKYSYNLFSREFRASLHKGLDSAVEVCVVYGNYSQQLQVYS
KTGFNCDGKLGNESVTFYLQNLYVNQTDIYFCKIEVMYPPPYLDNEKSNGTIIHVKGKHLCP
SPLFPGPSKP
CD28 Transmembrane domain:
(SEQ ID NO: 87)
FWVLVVVGGVLACYSLLVTVAFIIFWV
CD28 Cytoplasmic Domain.
(SEQ ID NO: 88)
RSKRSRLLHSDYMNMTPRRPGPTRKHYQPYAPPRDFAAYRS
CD3z Intracellular Domain:
(SEQ ID NO: 5)
RVKFSRSADAPAYKQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR

An exemplary nucleotide sequence encoding a CSR CD28z protein of the disclosure comprises or consists of the nucleotide sequence of (CD28 Signal peptide, CD28 Extracellular Domain, CD28 Transmembrane domain, CD28 Cytoplasmic Domain, CD3z Intracellular Domain):

(SEQ ID NO: 89)
ATGCTGAGACTGCTGCTGGCCCTGAATCTGTTCCCCAGCATCCAAGTGACCGGCAACAAGAT
CCTGGTCAAGCAGAGCCCTATGCTGGTGGCCTACGACAACGCCGTGAACCTGAGCTGCAAGT
ACAGCTACAACCTGTTCAGCAGAGAGTTCCGGGCCAGCCTGCACAAAGGACTGGATTCTGCT
GTGGAAGTGTGCGTGGTGTACGGCAACTACAGCCAGCAGCTGCAGGTCTACAGCAAGACCGG
CTTCAACTGCGACGGCAAGCTGGGCAATGAGAGCGTGACCTTCTACCTGCAAAACCTGTACG
TGAACCAGACCGACATCTATTTCTGCAAGATCGAAGTGATGTACCCGCCTCCTTACCTGGAC
AACGAGAAGTCCAACGGCACCATCATCCACGTGAAGGGCAAGCACCTGTGTCCTTCTCCACT
GTTCCCCGGACCTAGCAAGCCTTTCTGGGTGCTCGTTGTTGTTGGCGGCGTGCTGGCCTGTT
ATAGCCTGCTGGTTACAGTGGCCTTCATCATCTTTTGGGTC

[symbol]

[symbol]

[symbol]

[symbol]

[symbol]

[symbol]

AGAGTGAAGTTCTCCAGATCCG
CCGATGCTCCCGCCTATAAGCAGGGCCAGAACCAGCTGTACAACGAGCTGAACCTGGGGAGA
AGAGAAGAGTACGATGTGCTGGACAAGCGGAGAGGCAGAGATCCTGAGATGGGCGGCAAGCC
CAGACGGAAGAATCCTCAAGAGGGCCTGTACAATGAACTGCAGAAAGACAAGATGGCCGAGG
CCTACAGCGAGATCGGAATGAAGGGCGAGCGCAGAAGAGGCAAGGGACACGATGGACTGTAC
CAGGGCCTGAGCACCGCCACCAAGGATACCTATGATGCCCTGCACATGCAGGCCCTGCCTCC
AAGA
CD28 Signal peptide:
(SEQ ID NO: 90)
ATGCTGAGACTGCTGCTGGCCCTGAATCTGTTCCCCAGCATCCAAGTGACCGGC
CD28 Extracellular Domain:
(SEQ ID NO: 91)
AACAAGATCCTGGTCAAGCAGAGCCCTATGCTGGTGGCCTACGACAACGCCGTGAACCTGAG
CTGCAAGTACAGCTACAACCTGTTCAGCAGAGAGTTCCGGGCCAGCCTGCACAAAGGACTGG
ATTCTGCTGTGGAAGTGTGCGTGGTGTACGGCAACTACAGCCAGCAGCTGCAGGTCTACAGC
AAGACCGGCTTCAACTGCGACGGCAAGCTGGGCAATGAGAGCGTGACCTTCTACCTGCAAAA
CCTGTACGTGAACCAGACCGACATCTATTTCTGCAAGATCGAAGTGATGTACCCGCCTCCTT
ACCTGGACAACGAGAAGTCCAACGGCACCATCATCCACGTGAAGGGCAAGCACCTGTGTCCT
TCTCCACTGTTCCCCGGACCTAGCAAGCCT
CD28 Transmembrane domain:
(SEQ ID NO: 92)
TTCTGGGTGCTCGTTGTTGTTGGCGGCGTGCTGGCCTGTTATAGCCTGCTGGTTACAGTGGC
CTTCATCATCTTTTGGGTC
CD28 Cytoplasmic Domain:
(SEQ ID NO: 93)
CGAAGCAAGCGGAGCCGGCTGCTGCACAGCGACTACATGAACATGACCCCTAGACGGCCCGG
ACCAACCAGAAAGCACTACCAGCCTTACGCTCCTCCTAGAGACTTCGCCGCCTACCGGTCC
CD3z Intracellular Domain:
(SEQ ID NO: 94)
AGAGTGAAGTTCTCCAGATCCGCCGATGCTCCCGCCTATAAGCAGGGCCAGAACCAGCTGTA
CAACGAGCTGAACCTGGGGAGAAGAGAAGAGTACGATGTGCTGGACAAGCGGAGAGGCAGAG
ATCCTGAGATGGGCGGCAAGCCCAGACGGAAGAATCCTCAAGAGGGCCTGTACAATGAACTG
CAGAAAGACAAGATGGCCGAGGCCTACAGCGAGATCGGAATGAAGGGCGAGCGCAGAAGAGG
CAAGGGACACGATGGACTGTACCAGGGCCTGAGCACCGCCACCAAGGATACCTATGATGCCC
TGCACATGCAGGCCCTGCCTCCAAGA

A wildtype/natural human CD2 protein (NCBI: CD2 HUMAN; UniProt/Swiss-Prot: P06729.2) comprises or consists of the amino acid sequence of:

(SEQ ID NO: 95)
MSFPCKFVASFLLIFNVSSKGAVSKEITNALETWGALGQDINLDIPSFQM
SDDIDDIKWEKTSDKKKIAQFRKEKETFKEKDTYKLFKNGTLKIKHLKTD
DQDIYKVSIYDTKGKNVLEKIFDLKIQERVSKPKISWTCINTTLTCEVMN
GTDPELNLYQDGKHLKLSQRVITHKWTTSLSAKFKCTAGNKVSKESSVEP
VSCPEKGLDIYLIIGICGGGSLLMVFVALLVFYITKRKKQRSRRNDEELE
TRAHRVATEERGRKPHQIPASTPQNPATSQHPPPPPGHRSQAPSHRPPPP
GHRVQHQPQKRPPAPSGTQVHQQKGPPLPRPRVQPKPPHGAAENSLSPSS
N

A nucleotide sequence encoding wildtype/natural CD2 protein (NCBI: CCDS889.1) comprises or consists of the nucleotide sequence of:

(SEQ ID NO: 96)
ATGAGCTTTCCATGTAAATTTGTAGCCAGCTTCCTTCTGATTTTCAATGT
TTCTTCCAAAGGTGCAGTCTCCAAAGAGATTACGAATGCCTTGGAAACCT
GGGGTGCCTTGGGTCAGGACATCAACTTGGAGATTCCTAGTTTTCAAATG
AGTGATGATATTGAGGATATAAAATGGGAAAAAACTTCAGACAAGAAAAA
GATTGCACAATTCAGAAAAGAGAAAGAGACTTTCAAGGAAAAAGATACAT
ATAAGCTATTTAAAAATGGAACTCTGAAAATTAAGCATCTGAAGACCGAT
GATCAGGATATCTACAAGGTATCAATATATGATACAAAAGGAAAAAATGT
GTTGGAAAAAATATTTGATTTGAAGATTCAAGAGAGGGTCTCAAAACCAA
AGATCTCCTGGAGTTGTATCAACACAACCCTGAGCTGTGAGGTAATGAAT
GGAACTGACCCCGAATTAAACCTGTATCAAGATGGGAAACATCTAAAACT
TTCTCAGAGGGTCATCACACACAAGTGGACCACCAGCCTGAGTGCAAAAT
TCAAGTGCACAGCAGGGAACAAAGTCAGCAAGGAATCCAGTGTCGAGCCT
GTCAGCTGTCCAGAGAAAGGTCTGGACATCTATCTCATCATTGGCATATG
TGGAGGAGGCAGCCTCTTGATGGTCTTTGTGGCACTGCTCGTTTTCTATA
TCACCAAAAGGAAAAAACAGAGGAGTCGGAGAAATGATGAGGAGCTGGAG
ACAAGAGCCCACAGAGTAGCTACTGAAGAAAGGGGCCGGAAGCCCCACCA
AATTCCAGCTTCAACCCCTCAGAATCCAGCAACTTCCCAACATCCTCCTC
CACCACCTGGTCATCGTTCCCAGGCACCTAGTCATCGTCCCCCGCCTCCT
GGACACCGTGTTCAGCACCAGCCTCAGAAGAGGCCTCCTGCTCCGTCGGG
CACACAAGTTCACCAGCAGAAAGGCCCGCCCCTCCCCAGACCTCGAGTTC
AGCCAAAACCTCCCCATGGGGCAGCAGAAAACTCATTGTCCCCTTCCTCT
AATTAA

An exemplary CSR CD2z protein of the disclosure comprises or consists of the amino acid sequence of (CD2 Signal peptide, CD2 Extracellular Domain, CD2 Transmembrane domain, CD2 Cytoplasmic Domain, CD3z Intracellular Domain):

(SEQ ID NO: 97)
MSFPCKFVASFLLIFNVSSKGAVSKEITNALETWGALGQDINLDIPSFQMSDDIDDIKWEKT
SDKKKIAQFRKEKETFKEKDTYKLFKNGTLKIKHLKTDDQDIYKVSIYDTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYLIIGICGGGSLLMVFVALLVFYIT

[symbol]

[symbol]

[symbol]

[symbol]

[symbol]

[symbol]

RVKFSRSADAPAYKQGQNQLY
NELNLGRREEYDVLDKRRGRDPEMGGKPRRKNPQEGLYNELQKDKMAEAYSEIGMKGERRRG
KGHDGLYQGLSTATKDTYDALHMQALPPR
CD2 Signal peptide:
(SEQ ID NO: 9)
MSFPCKFVASFLLIFNVSSKGAVS
CD2 Extracellular Domain:
(SEQ ID NO: 1)
KEITNALETWGALGQDINLDIPSFQMSDDIDDIKWEKTSDKKKIAQFRKEKETFKEKDTYKL
FKNGTLKIKHLKTDDQDIYKVSIYDTKGKNVLEKIFDLKIQERVSKPKISWTCINTTLTCEV
MNGTDPELNLYQDGKHLKLSQRVITHKWTTSLSAKFKCTAGNKVSKESSVEPVSCPEKGLD
CD2 Transmembrane domain:
(SEQ ID NO: 14)
IYLIIGICGGGSLLMVFVALLVFYIT
CD2 Cytoplasmic Domain:
(SEQ ID NO: 98)
KRKKQRSRRNDEELETRAHRVATEERGRKPHQIPASTPQNPATSQHPPPPPGHRSQAPSHRP
PPPGHRVQHQPQKRPPAPSGTQVHQQKGPPLPRPRVQPKPPHGAAENSLSPSSN
CD3z Intracellular Domain:
(SEQ ID NO: 5)
RVKFSRSADAPAYKQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR

The present disclosure provides a non-naturally occurring CSR CD2 protein comprising, consisting essential of, or consisting of an amino acid sequence at least 80%, at least 85%, at least 90%, at least 91%, at least 92%, at least 93%, at least 94%, at least 95%, at least 96%, at least 97%, at least 98%, at least 99% or 100% identical to SEQ ID NO:97. The present disclosure provides a CD2 signal peptide comprising, consisting essential of, or consisting of an amino acid sequence at least 80%, at least 85%, at least 90%, at least 91%, at least 92%, at least 93%, at least 94%, at least 95%, at least 96%, at least 97%, at least 98%, at least 99% or 100% identical to SEQ ID NO:9. The present disclosure provides a CD2 extracellular domain comprising, consisting essential of, or consisting of an amino acid sequence at least 80%, at least 85%, at least 90%, at least 91%, at least 92%, at least 93%, at least 94%, at least 95%, at least 96%, at least 97%, at least 98%, at least 99% or 100% identical to SEQ ID NO: 1. The present disclosure provides a CD2 transmembrande domain comprising, consisting essential of, or consisting of an amino acid sequence at least 80%, at least 85%, at least 90%, at least 91%, at least 92%, at least 93%, at least 94%, at least 95%, at least 96%, at least 97%, at least 98%, at least 99% or 100% identical to SEQ ID NO:14. The present disclosure provides a CD2 cytoplasmic domain comprising, consisting essential of, or consisting of an amino acid sequence at least 80%, at least 85%, at least 90%, at least 91%, at least 92%, at least 93%, at least 94%, at least 95%, at least 96%, at least 97%, at least 98%, at least 99% or 100% identical to SEQ ID NO:98. The present disclosure provides a CD3z intracellular domain comprising, consisting essential of, or consisting of an amino acid sequence at least 80%, at least 85%, at least 90%, at least 91%, at least 92%, at least 93%, at least 94%, at least 95%, at least 96%, at least 97%, at least 98%, at least 99% or 100% identical to SEQ ID NO:5.

An exemplary nucleotide sequence encoding a CSR CD2z protein of the disclosure comprises or consists of the amino acid sequence of (CD2 Signal peptide, CD2 Extracellular Domain, CD2 Transmembrane domain, CD2 Cytoplasmic Domain, CD3z Intracellular Domain):

(SEQ ID NO: 99)
ATGAGCTTCCCTTGCAAGTTCGTGGCCAGCTTCCTGCTGATCTTCAACGTGTCCTCTAAGGG
CGCCGTGTCCAAAGAGATCACAAACGCCCTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACGACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGAAGCCTGCTGATGGTGTTTGTGG
CTCTGCTGGTGTTCTAGATCACC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

GAGTGAAGTTCAGCCGCAGCGCCGATGCTCCTGCCTATAAGCAGGGACAGAACCAGCTGTAC
AACGAGCTGAATCTGGGGCGCAGAGAAGAGTACGATGTGCTGGACAAGCGGAGAGGCAGAGA
TCCTGAGATGGGCGGCAAGCCCAGACGGAAGAATCCTCAAGAGGGCCTGTATAATGAGCTGC
AGAAAGACAAGATGGCCGAGGCCTACAGCGAGATCGGAATGAAGGGCGAGCGCAGAAGAGGC
AAGGGACACGATGGACTGTATCAGGGCCTGAGCACCGCCACCAAGGATACCTATGATGCCCT
GCACATGCAGGCCCTGCCTCCAAGA
CD2 Signal peptide:
(SEQ ID NO: 100)
ATGAGCTTCCCTTGCAAGTTCGTGGCCAGCTTCCTGCTGATCTTCAACGTGTCCTCTAAGGG
CGCCGTGTCC
CD2 Extracellular Domain:
(SEQ ID NO: 101)
AAAGAGATCACAAACGCCCTGGAAACCTGGGGAGCCCTCGGCCAGGATATTAACCTGGACAT
CCCCAGCTTCCAGATGAGCGACGACATCGATGACATCAAGTGGGAGAAAACCAGCGACAAGA
AGAAGATCGCCGAGTTCCGGAAAGAGAAAGAGACATTCAAAGAGAAGGAGACCTACAAGCTG
TTCAAGAACGGCACCCTGAAGATCAAGCACCTGAAAACCGACGACCAGGACATCTATAAGGT
GTCCATCTACGACACCAAGGGCAAGAACGTGCTGGAAAAGATCTTCGACCTCAAGATCCAAG
AGCGGGTGTCCAAGCCTAAGATCAGCTGGACCTGCATCAACACCACACTGACCTGCGAAGTG
ATGAACGGCACAGACCCCGAGCTGAACCTGTACCAGGATGGCAAACACCTGAAGCTGAGCCA
GCGCGTGATCACCCACAAGTGGACAACAAGCCTGAGCGCCAAGTTCAAGTGCACCGCCGGAA
ACAAAGTGTCTAAAGAGTCCAGCGTCGAGCCCGTGTCTTGCCCTGAAAAAGGACTGGAC
CD2 Transmembrane domain:
(SEQ ID NO: 102)
ATCTACCTGATCATCGGCATCTGTGGCGGCGGAAGCCTGCTGATGGTGTTTGTGGCTCTGCT
GGTGTTCTACATCACC
CD2 Cytoplasmic Domain:
(SEQ ID NO: 103)
AAGCGGAAGAAGCAGCGGAGCAGACGGAACGACGAGGAACTGGAAACACGGGCCCATAGAGT
GGCCACCGAGGAAAGAGGCAGAAAGCCCCACCAGATTCCAGCCAGCACACCCCAGAATCCTG
CCACCTCTCAACACCCTCCACCTCCACCTGGACACAGATCTCAGGCCCCATCTCACAGACCT
CCACCACCTGGTCATCGGGTGCAGCACCAGCCTCAGAAAAGACCTCCTGCTCCTAGCGGCAC
ACAGGTGCACCAGCAAAAAGGACCTCCACTGCCTCGGCCTAGAGTGCAGCCTAAACCTCCTC
ATGGCGCCGCTGAGAACAGCCTGTCTCCAAGCAGCAAC
CD3z Intracellular Domain:
(SEQ ID NO: 94)
AGAGTGAAGTTCAGCCGCAGCGCCGATGCTCCTGCCTATAAGCAGGGACAGAACCAGCTGTA
CAACGAGCTGAATCTGGGGCGCAGAGAAGAGTACGATGTGCTGGACAAGCGGAGAGGCAGAG
ATCCTGAGATGGGCGGCAAGCCCAGACGGAAGAATCCTCAAGAGGGCCTGTATAATGAGCTG
CAGAAAGACAAGATGGCCGAGGCCTACAGCGAGATCGGAATGAAGGGCGAGCGCAGAAGAGG
CAAGGGACACGATGGACTGTATCAGGGCCTGAGCACCGCCACCAAGGATACCTATGATGCCC
TGCACATGCAGGCCCTGCCTCCAAGA

An exemplary mutant CSR CD2z-D111H protein of the disclosure comprises or consists of the amino acid sequence of (CD2 Signal peptide, CD2 Extracellular domain with D111H mutation within the CD2 Extracellular domain, CD2 Transmembrane domain, CD2 Cytoplasmic domain, CD3z Intracellular domain):

(SEQ ID NO: 104)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYLIIGICGGGSLLMVFVALLVFYIT

[symbol]

[symbol]

[symbol]

[symbol]

[symbol]

[symbol]

RVKFSRSADAPAYKQGQNQLY
NELNLGRREEYDVLDKRRGRDPEMGGKPRRKNPQEGLYNELQKDKMAEAYSEIGMKGERRRG
KGHDGLYQGLSTATKDTYDALHMQALPPR
CD2 Signal peptide:
(SEQ ID NO: 9)
MSFPCKFVASELLIFNVSSKGAVS
CD2 Extracellular domain with D111H mutation within the CD2 Extracellular domain:
(SEQ ID NO: 4)
KEITNALETWGALGQDINLDIPSFQMSDDIDDIKWEKTSDKKKIAQFRKEKETFKEKDTYKL
FKNGTLKIKHLKTDDQDIYKVSIYHTKGKNVLEKIFDLKIQERVSKPKISWTCINTTLTCEV
MNGTDPELNLYQDGKHLKLSQRVITHKWTTSLSAKFKCTAGNKVSKESSVEPVSCPEKGL
CD2 Transmembrane domain:
(SEQ ID NO: 14)
IYLIIGICGGGSLLMVFVALLVFYIT
CD2 Cytoplasmic domain:
(SEQ ID NO: 98)
KRKKQRSRRNDEELETRAHRVATEERGRKPHQIPASTPQNPATSQHPPPPPGHRSQAPSHRP
PPPGHRVQHQPQKRPPAPSGTQVHQQKGPPLPRPRVQPKPPHGAAENSLSPSSN
CD3z Intracellular domain:
(SEQ ID NO: 5)
RVKFSRSADAPAYKQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR

The present disclosure provides a non-naturally occurring CSR CD2 protein comprising, consisting essential of, or consisting of an amino acid sequence at least 80%, at least 85%, at least 90%, at least 91%, at least 92%, at least 93%, at least 94%, at least 95%, at least 96%, at least 97%, at least 98%, at least 99% or 100% identical to SEQ ID NO:104. The present disclosure provides a CD2 extracellular domain comprising, consisting essential of, or consisting of an amino acid sequence at least 80%, at least 85%, at least 90%, at least 91%, at least 92%, at least 93%, at least 94%, at least 95%, at least 96%, at least 97%, at least 98%, at least 99% or 100% identical to SEQ ID NO:4.

An exemplary nucleotide sequence encoding a mutant CSR CD2z-D111H protein of the disclosure comprises or consists of the amino acid sequence of (CD2 Signal peptide, CD2 Extracellular domain with D111H mutation within the CD2 Extracellular domain, CD2 Transmembrane domain, CD2 Cytoplasmic domain, CD3z Intracellular domain):

(SEQ ID NO: 105)
ATGAGCTTCCCTTGCAAGTTCGTGGCCAGCTTCCTGCTGATCTTCAACGTGTCCTCTAAGGG
CGCCGTGTCCAAAGAGATCACAAACGCCCTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGAAGCCTGCTGATGGTGTTTGTGG
CTCTGCTGGTGTTCTAGATCACC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

GAGTGAAGTTCAGCCGCAGCGCCGATGCTCCTGCCTATAAGCAGGGACAGAACCAGCTGTAC
AACGAGCTGAATCTGGGGCGCAGAGAAGAGTACGATGTGCTGGACAAGCGGAGAGGCAGAGA
TCCTGAGATGGGCGGCAAGCCCAGACGGAAGAATCCTCAAGAGGGCCTGTATAATGAGCTGC
AGAAAGACAAGATGGCCGAGGCCTACAGCGAGATCGGAATGAAGGGCGAGCGCAGAAGAGGC
AAGGGACACGATGGACTGTATCAGGGCCTGAGCACCGCCACCAAGGATACCTATGATGCCCT
GCACATGCAGGCCCTGCCTCCAAGA
CD2 Signal peptide:
(SEQ ID NO: 100)
ATGAGCTTCCCTTGCAAGTTCGTGGCCAGCTTCCTGCTGATCTTCAACGTGTCCTCTAAGGG
CGCCGTGTCC
CD2 Extracellular domain with D111H mutation within the CD2 Extracellular domain:
(SEQ ID NO: 106)
AAAGAGATCACAAACGCCCTGGAAACCTGGGGAGCCCTCGGCCAGGATATTAACCTGGACAT
CCCCAGCTTCCAGATGAGCGACGACATCGATGACATCAAGTGGGAGAAAACCAGCGACAAGA
AGAAGATCGCCGAGTTCCGGAAAGAGAAAGAGACATTCAAAGAGAAGGAGACCTACAAGCTG
TTCAAGAACGGCACCCTGAAGATCAAGCACCTGAAAACCGACGACCAGGACATCTATAAGGT
GTCCATCTACCACACCAAGGGCAAGAACGTGCTGGAAAAGATCTTCGACCTCAAGATCCAAG
AGCGGGTGTCCAAGCCTAAGATCAGCTGGACCTGCATCAACACCACACTGACCTGCGAAGTG
ATGAACGGCACAGACCCCGAGCTGAACCTGTACCAGGATGGCAAACACCTGAAGCTGAGCCA
GCGCGTGATCACCCACAAGTGGACAACAAGCCTGAGCGCCAAGTTCAAGTGCACCGCCGGAA
ACAAAGTGTCTAAAGAGTCCAGCGTCGAGCCCGTGTCTTGCCCTGAAAAAGGACTGGAC
CD2 Transmembrane domain:
(SEQ ID NO: 102)
ATCTACCTGATCATCGGCATCTGTGGCGGCGGAAGCCTGCTGATGGTGTTTGTGGCTCTGCT
GGTGTTCTACATCACC
CD2 Cytoplasmic domain:
(SEQ ID NO: 103)
AAGCGGAAGAAGCAGCGGAGCAGACGGAACGACGAGGAACTGGAAACACGGGCCCATAGAGT
GGCCACCGAGGAAAGAGGCAGAAAGCCCCACCAGATTCCAGCCAGCACACCCCAGAATCCTG
CCACCTCTCAACACCCTCCACCTCCACCTGGACACAGATCTCAGGCCCCATCTCACAGACCT
CCACCACCTGGTCATCGGGTGCAGCACCAGCCTCAGAAAAGACCTCCTGCTCCTAGCGGCAC
ACAGGTGCACCAGCAAAAAGGACCTCCACTGCCTCGGCCTAGAGTGCAGCCTAAACCTCCTC
ATGGCGCCGCTGAGAACAGCCTGTCTCCAAGCAGCAAC
CD3z Intracellular domain:
(SEQ ID NO: 94)
AGAGTGAAGTTCAGCCGCAGCGCCGATGCTCCTGCCTATAAGCAGGGACAGAACCAGCTGTA
CAACGAGCTGAATCTGGGGCGCAGAGAAGAGTACGATGTGCTGGACAAGCGGAGAGGCAGAG
ATCCTGAGATGGGCGGCAAGCCCAGACGGAAGAATCCTCAAGAGGGCCTGTATAATGAGCTG
CAGAAAGACAAGATGGCCGAGGCCTACAGCGAGATCGGAATGAAGGGCGAGCGCAGAAGAGG
CAAGGGACACGATGGACTGTATCAGGGCCTGAGCACCGCCACCAAGGATACCTATGATGCCC
TGCACATGCAGGCCCTGCCTCCAAGA

An exemplary mutant “CSR 01 CD2.DH.z” or “CD2z.V2” protein of the disclosure comprises or consists of the amino acid sequence of (CD2 Signal peptide, CD2 Extracellular domain with D111H mutation within the CD2 Extracellular domain, CD2 Transmembrane domain, CD2 Intracellular domain, CD3z Intracellular domain):

(SEQ ID NO: 107)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYLIIGICGGGSLLMVFVALLVFYIT

[symbol]

[symbol]

[symbol]

[symbol]

[symbol]

[symbol]

RVKFSRSADAPAYKQGQNQLY
NELNLGRREEYDVLDKRRGRDPEMGGKPRRKNPQEGLYNELQKDKMAEAYSEIGMKGERRRG
KGHDGLYQGLSTATKDTYDALHMQALPPR
CD2 Signal peptide:
(SEQ ID NO: 9)
CD2 Extracellular domain with D111H mutation within the CD2 Extracellular domain:
(SEQ ID NO: 4)
CD2 Transmembrane domain:
(SEQ ID NO: 14)
CD2 Intracellular domain:
(SEQ ID NO: 98)
CD3z Intracellular domain:
(SEQ ID NO: 5)

An exemplary nucleotide sequence encoding a mutant “CSR 01 CD2.DH.z” or “CD2z.V2” protein of the disclosure comprises or consists of the nucleotide sequence of (CD2 Signal peptide, CD2 Extracellular domain with D111H mutation within the CD2 Extracellular domain, CD2 Transmembrane domain, CD2 Intracellular domain, CD3z Intracellular domain):

(SEQ ID NO: 108)
ATGAGCTTCCCTTGCAAGTTCGTGGCCAGCTTCCTGCTGATCTTCAACGTGTCCTCTAAGGG
CGCCGTGTCCAAAGAAATCACGAATGCATTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGATCCCTGCTGATGGTGTTTGTGG
CTCTGCTGGTGTTCTACATCACC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

GAGTGAAATTCAGCCGCAGCGCCGATGCTCCTGCCTATAAGCAGGGACAGAACCAGCTGTAC
AACGAGCTGAATCTGGGGCGCAGAGAAGAGTACGATGTGCTGGACAAGCGGAGAGGCAGAGA
TCCTGAGATGGGCGGCAAGCCCAGACGGAAGAATCCTCAAGAGGGCCTGTATAATGAGCTGC
AGAAAGACAAGATGGCCGAGGCCTACAGCGAGATCGGAATGAAGGGCGAGCGCAGAAGAGGC
AAGGGACACGATGGACTGTATCAGGGCCTGAGCACCGCCACCAAGGATACCTATGATGCCCT
GCACATGCAGGCCCTGCCTCCAAGA
CD2 Signal peptide:
(SEQ ID NO: 100)
CD2 Extracellular domain with D111H mutation within the CD2 Extracellular domain:
(SEQ ID NO: 106)
CD2 Transmembrane domain:
(SEQ ID NO: 102)
CD2 Intracellular domain:
(SEQ ID NO: 103)
CD3z Intracellular domain:
(SEQ ID NO: 94)

An exemplary mutant CSR 01b CD2.8.DH.z protein of the disclosure comprises or consists of the amino acid sequence of (CD8a Signal peptide, CD2 Extracellular domain with D111H mutation within the CD2 Extracellular domain, CD2 Transmembrane domain, CD2 Intracellular domain, CD3z Intracellular domain):

(SEQ ID NO: 109)
MALPVTALLLPLALLLHAARPKEITNALETWGALGQDINLDIPSFQMSDDIDDIKWEKTSDK
KKIAQFRKEKETFKEKDTYKLFKNGTLKIKHLKTDDQDIYKVSIYHTKGKNVLEKIFDLKIQ
ERVSKPKISWTCINTTLTCEVMNGTDPELNLYQDGKHLKLSQRVITHKWTTSLSAKFKCTAG
NKVSKESSVEPVSCPEKGLDIYLIIGICGGGSLLMVFVALLVFYIT

[symbol]

[symbol]

[symbol]

[symbol]

[symbol]

[symbol]

RVKFSRSADAPAYKQGQNQLYNEL
NLGRREEYDVLDKRRGRDPEMGGKPRRKNPQEGLYNELQKDKMAEAYSEIGMKGERRRGKGH
DGLYQGLSTATKDTYDALHMQALPPR
CD8a Signal peptide:
(SEQ ID NO: 12)
CD2 Extracellular domain with D111H mutation within the CD2 Extracellular domain:
(SEQ ID NO: 4)
CD2 Transmembrane domain:
(SEQ ID NO: 14)
CD2 Intracellular domain:
(SEQ ID NO: 98)
CD3z Intracellular domain:
(SEQ ID NO: 5)

An exemplary nucleotide sequence encoding a mutant CSR 01b CD2.8.DH.z protein of the disclosure comprises or consists of the nucleotide sequence of (CD8a Signal peptide, CD2 Extracellular domain with D111H mutation within the CD2 Extracellular domain, CD2 Transmembrane domain, CD2 Intracellular domain, CD3z Intracellular domain):

(SEQ ID NO: 110)
ATGGCTCTGCCTGTGACAGCTCTGCTGCTGCCTCTGGCTCTGCTTCTGCATGCCGCCAGACC
TAAAGAAATCACGAATGCATTGGAAACCTGGGGAGCCCTCGGCCAGGATATTAACCTGGACA
TCCCCAGCTTCCAGATGAGCGACGACATCGATGACATCAAGTGGGAGAAAACCAGCGACAAG
AAGAAGATCGCCCAGTTCCGGAAAGAGAAAGAGACATTCAAAGAGAAGGACACCTACAAGCT
GTTCAAGAACGGCACCCTGAAGATCAAGCACCTGAAAACCGACGACCAGGACATCTATAAGG
TGTCCATCTACCACACCAAGGGCAAGAACGTGCTGGAAAAGATCTTCGACCTCAAGATCCAA
GAGCGGGTGTCCAAGCCTAAGATCAGCTGGACCTGCATCAACACCACACTGACCTGCGAAGT
GATGAACGGCACAGACCCCGAGCTGAACCTGTACCAGGATGGCAAACACCTGAAGCTGAGCC
AGCGCGTGATCACCCACAAGTGGACAACAAGCCTGAGCGCCAAGTTCAAGTGCACCGCCGGA
AACAAAGTGTCTAAAGAGTCCAGCGTCGAGCCCGTGTCTTGCCCTGAAAAAGGACTGGACAT
CTACCTGATCATCGGCATCTGTGGCGGCGGATCCCTGCTGATGGTGTTTGTGGCTCTGCTGG
TGTTCTACATCACC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

AGAGTGAAAT
TCAGCCGCAGCGCCGATGCTCCTGCCTATAAGCAGGGACAGAACCAGCTGTACAACGAGCTG
AATCTGGGGCGCAGAGAAGAGTACGATGTGCTGGACAAGCGGAGAGGCAGAGATCCTGAGAT
GGGCGGCAAGCCCAGACGGAAGAATCCTCAAGAGGGCCTGTATAATGAGCTGCAGAAAGACA
AGATGGCCGAGGCCTACAGCGAGATCGGAATGAAGGGCGAGCGCAGAAGAGGCAAGGGACAC
GATGGACTGTATCAGGGCCTGAGCACCGCCACCAAGGATACCTATGATGCCCTGCACATGCA
GGCCCTGCCTCCAAGA
CD8a Signal peptide:
(SEQ ID NO: 111)
ATGGCTCTGCCTGTGACAGCTCTGCTGCTGCCTCTGGCTCTGCTTCTGCATGCCGCCAGACC
T
CD2 Extracellular domain with D111H mutation within the CD2 Extracellular domain:
(SEQ ID NO: 106)
CD2 Transmembrane domain:
(SEQ ID NO: 102)
CD2 Intracellular domain:
(SEQ ID NO: 103)
CD3z Intracellular domain:
(SEQ ID NO: 94)

An exemplary mutant CSR 02 CD2.DH.28z protein of the disclosure comprises or consists of the amino acid sequence of (CD2 Signal peptide, CD2 Extracellular domain with D111H mutation within the CD2 Extracellular domain, CD2 Transmembrane domain, CD28 Intracellular domain, CD3z Intracellular domain):

(SEQ ID NO: 112)
MSFPCKFVASFLLIFNVSSKGAVSKEITNALETWGALGQDINLDTPSFQMSDDIDDIKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYLIIGICGGGSLLMVFVALLVFYIT [symbol] [symbol] [symbol]
RVKFSRSADAPAYKQGQNQLYNELNLGRREEYDV
LDKRRGRDPEMGGKPRRKNPQEGLYNELQKDKMAEAYSEIGMKGERRRGKGHDGLYQGLSTA
TKDTYDALHMQALPPR
CD2 Signal peptide:
(SEQ ID NO: 9)
CD2 Extracellular domain with D111H mutation within the CD2 Extracellular domain:
(SEQ ID NO: 4)
CD2 Transmembrane domain:
(SEQ ID NO: 14)
CD28 Intracellular domain:
(SEQ ID NO: 113)

[symbol]

[symbol]

CD3z Intracellular domain:
(SEQ ID NO: 5)

An exemplary nucleotide sequence encoding a mutant CSR 02 CD2.DH.28z protein of the disclosure comprises or consists of the nucleotide sequence of (CD2 Signal peptide, CD2 Extracellular domain with D111H mutation within the CD2 Extracellular domain, CD2 Transmembrane domain, CD28 Intracellular domain, CD3z Intracellular domain):

(SEQ ID NO: 114)
ATGAGCTTCCCTTGCAAGTTCGTGGCCAGCTTCCTGCTGATCTTCAACGTGTCCTCTAAGGG
CGCCGTGTCCAAAGAAATCACGAATGCATTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGATCCCTGCTGATGGTGTTTGTGG
CTCTGCTGGTGTTCTACATCACC

[symbol]

[symbol]

[symbol]

[symbol]

[symbol]

[symbol]

AGAGTGAAATTCAGCCGCAGCGCCGATGCTCCTGCCTATA
AGCAGGGACAGAACCAGCTGTACAACGAGCTGAATCTGGGGCGCAGAGAAGAGTACGATGTG
CTGGACAAGCGGAGAGGCAGAGATCCTGAGATGGGCGGCAAGCCCAGACGGAAGAATCCTCA
AGAGGGCCTGTATAATGAGCTGCAGAAAGACAAGATGGCCGAGGCCTACAGCGAGATCGGAA
TGAAGGGCGAGCGCAGAAGAGGCAAGGGACACGATGGACTGTATCAGGGCCTGAGCACCGCC
ACCAAGGATACCTATGATGCCCTGCACATGCAGGCCCTGCCTCCAAGA
CD2 Signal peptide:
(SEQ ID NO: 100)
CD2 Extracellular domain with D111H mutation within the CD2 Extracellular domain:
(SEQ ID NO: 106)
CD2 Transmembrane domain:
(SEQ ID NO: 102)
CD28 Intracellular domain:
(SEQ ID NO: 115)

[symbol]

[symbol]

[symbol]

[symbol]

[symbol]

[symbol]

CD3z Intracellular domain:
(SEQ ID NO: 94)

An exemplary mutant CSR 02b CD2.8.DH.28z protein of the disclosure comprises or consists of the amino acid sequence of (CD8a Signal peptide, CD2 Extracellular domain with D111H mutation within the CD2 Extracellular domain, CD2 Transmembrane domain, CD28 Intracellular domain, CD3z Intracellular domain):

(SEQ ID NO: 116)
MALPVTALLLPLALLLHAARPKEITNALETWGALGQDINLDIPSFQMSDDIDDIKWEKTSDK
KKIAQFRKEKETFKEKDTYKLFKNGTLKIKHLKTDDQDIYKVSIYHTKGKNVLEKIFDLKIQ
ERVSKPKISWTCINTTLTCEVMNGTDPELNLYQDGKHLKLSQRVITHKWTTSLSAKFKCTAG
NKVSKESSVEPVSCPEKGLDIYLIIGICGGGSLLMVFVALLVFYIT [symbol] [symbol]
RVKFSRSADAPAYKQGQNQLYNELNLGRREEYDVLDK
RRGRDPEMGGKPRRKNPQEGLYNELQKDKMAEAYSEIGMKGERRRGKGHDGLYQGLSTATKD
TYDALHMQALPPR
CD8a Signal peptide:
(SEQ ID NO: 12)
CD2 Extracellular domain with D111H mutation within the CD2 Extracellular domain:
(SEQ ID NO: 4)
CD2 Transmembrane domain:
(SEQ ID NO: 14)
CD28 Intracellular domain:
(SEQ ID NO: 113)
CD3z Intracellular domain:
(SEQ ID NO: 5)

An exemplary nucleotide sequence encoding a mutant CSR 02b CD2.8.DH.28z protein of the disclosure comprises or consists of the nucleotide sequence of (CD8a Signal peptide, CD2 Extracellular domain with D111H mutation within the CD2 Extracellular domain, CD2 Transmembrane domain, CD28 Intracellular domain, CD3z Intracellular domain):

(SEQ ID NO: 117)
ATGGCTCTGCCTGTGACAGCTCTGCTGCTGCCTCTGGCTCTGCTTCTGCATGCCGCCAGACC
TAAAGAAATCACGAATGCATTGGAAACCTGGGGAGCCCTCGGCCAGGATATTAACCTGGACA
TCCCCAGCTTCCAGATGAGCGACGACATCGATGACATCAAGTGGGAGAAAACCAGCGACAAG
AAGAAGATCGCCCAGTTCCGGAAAGAGAAAGAGACATTCAAAGAGAAGGACACCTACAAGCT
GTTCAAGAACGGCACCCTGAAGATCAAGCACCTGAAAACCGACGACCAGGACATCTATAAGG
TGTCCATCTACCACACCAAGGGCAAGAACGTGCTGGAAAAGATCTTCGACCTCAAGATCCAA
GAGCGGGTGTCCAAGCCTAAGATCAGCTGGACCTGCATCAACACCACACTGACCTGCGAAGT
GATGAACGGCACAGACCCCGAGCTGAACCTGTACCAGGATGGCAAACACCTGAAGCTGAGCC
AGCGCGTGATCACCCACAAGTGGACAACAAGCCTGAGCGCCAAGTTCAAGTGCACCGCCGGA
AACAAAGTGTCTAAAGAGTCCAGCGTCGAGCCCGTGTCTTGCCCTGAAAAAGGACTGGACAT
CTACCTGATCATCGGCATCTGTGGCGGCGGATCCCTGCTGATGGTGTTTGTGGCTCTGCTGG
TGTTCTACATCACC

[symbol]

[symbol]

[symbol]

[symbol]

[symbol]

[symbol]

[symbol]

AGAGTGAAATTCAGCCGCAGCGCCGATGCTCCTGCCTATAAGCAGGGAC
AGAACCAGCTGTACAACGAGCTGAATCTGGGGCGCAGAGAAGAGTACGATGTGCTGGACAAG
CGGAGAGGCAGAGATCCTGAGATGGGCGGCAAGCCCAGACGGAAGAATCCTCAAGAGGGCCT
GTATAATGAGCTGCAGAAAGACAAGATGGCCGAGGCCTACAGCGAGATCGGAATGAAGGGCG
AGCGCAGAAGAGGCAAGGGACACGATGGACTGTATCAGGGCCTGAGCACCGCCACCAAGGAT
ACCTATGATGCCCTGCACATGCAGGCCCTGCCTCCAAGA
CD8a Signal peptide:
(SEQ ID NO: 111)
ATGGCTCTGCCTGTGACAGCTCTGCTGCTGCCTCTGGCTCTGCTTCTGCATGCCGCCAGACC
T
CD2 Extracellular domain with D111H mutation within the
CD2 Extracellular domain:
(SEQ ID NO: 106)
CD2 Transmembrane domain:
(SEQ ID NO: 102)
CD28 Intracellular domain:
(SEQ ID NO: 115)

[symbol]

[symbol]

[symbol]

[symbol]

[symbol]

[symbol]

CD3z Intracellular domain:
(SEQ ID NO: 94)

An exemplary mutant CSR 03 CD2.DH.BBz protein of the disclosure comprises or consists of the amino acid sequence of (CD2 Signal peptide, CD2 Extracellular domain with D111H mutation within the CD2 Extracellular domain, CD2 Transmembrane domain, 4-1BB Intracellular domain, CD3z Intracellular domain):

(SEQ ID NO: 118)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYLIIGICGGGSLLMVFVALLVFYIT [symbol] [symbol] [symbol]
RVKFSRSADAPAYKQGQNQLYNELNLGRREEYD
VLDKRRGRDPEMGGKPRRKNPQEGLYNELQKDKMAEAYSEIGMKGERRRGKGHDGLYQGLST
ATKDTYDALHMQALPPR
CD2 Signal peptide:
(SEQ ID NO: 9)
CD2 Extracellular domain with D111H mutation within
the CD2 Extracellular domain:
(SEQ ID NO: 4)
CD2 Transmembrane domain:
(SEQ ID NO: 14)
4-1BB Intracellular domai:
(SEQ ID NO: 119)

[symbol]

[symbol]

CD3z Intracellular domain:
(SEQ ID NO: 5)

An exemplary nucleotide sequence encoding a mutant CSR 03 CD2.DH.BBz protein of the disclosure comprises or consists of the nucleotide sequence of (CD2 Signal peptide, CD2 Extracellular domain with D111H mutation within the CD2 Extracellular domain, CD2 Transmembrane domain, 4-1BB Intracellular domain, CD3z Intracellular domain):

(SEQ ID NO: 120)
ATGAGCTTCCCTTGCAAGTTCGTGGCCAGCTTCCTGCTGATCTTCAACGTGTCCTCTAAGGG
CGCCGTGTCCAAAGAAATCACGAATGCATTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGATCCCTGCTGATGGTGTTTGTGG
CTCTGCTGGTGTTCTAGATGAGC

[symbol]

[symbol]

[symbol]

[symbol]

[symbol]

[symbol]

[symbol]

AGAGTGAAATTCAGCCGCAGCGCCGATGCTCCTGCCT
ATAAGCAGGGACAGAACCAGCTGTACAACGAGCTGAATCTGGGGCGCAGAGAAGAGTACGAT
GTGCTGGACAAGCGGAGAGGCAGAGATCCTGAGATGGGCGGCAAGCCCAGACGGAAGAATCC
TCAAGAGGGCCTGTATAATGAGCTGCAGAAAGACAAGATGGCCGAGGCCTACAGCGAGATCG
GAATGAAGGGCGAGCGCAGAAGAGGCAAGGGACACGATGGACTGTATCAGGGCCTGAGCACC
GCCACCAAGGATACCTATGATGCCCTGCACATGCAGGCCCTGCCTCCAAGA
CD2 Signal peptide:
(SEQ ID NO: 100)
CD2 Extracellular domain with D111H mutation
within the CD2 Extracellular domain:
(SEQ ID NO: 106)
CD2 Transmembrane domain:
(SEQ ID NO: 102)
4-1BB Intracellular domain:
(SEQ ID NO: 121)

[symbol]

[symbol]

[symbol]

[symbol]

[symbol]

[symbol]

TG
CD3z Intracellular domain:
(SEQ ID NO: 94)

An exemplary mutant CSR 03b CD2.8.DH.BBz protein of the disclosure comprises or consists of the amino acid sequence of (CD8a Signal peptide, CD2 Extracellular domain with D111H mutation within the CD2 Extracellular domain, CD2 Transmembrane domain, 4-1BB Intracellular domain, CD3z Intracellular domain):

(SEQ ID NO: 122)
MALPVTALLLPLALLLHAARPKEITNALETWGALGQDINLDIPSFQMSDDIDDIKWEKTSDK
KKIAQFRKEKETFKEKDTYKLFKNGTLKIKHLKTDDQDIYKVSIYHTKGKNVLEKIFDLKIQ
ERVSKPKISWTCINTTLTCEVMNGTDPELNLYQDGKHLKLSQRVITHKWTTSLSAKFKCTAG
NKVSKESSVEPVSCPEKGLDIYLIIGICGGGSLLMVFVALLVFYIT [symbol] [symbol]
RVKFSRSADAPAYKQGQNQLYNELNLGRREEYDVLD
KRRGRDPEMGGKPRRKNPQEGLYNELQKDKMAEAYSEIGMKGERRRGKGHDGLYQGLSTATK
DTYDALHMQALPPR
CD8a Signal peptide:
(SEQ ID NO: 12)
CD2 Extracellular domain with D111H mutation within
the CD2 Extracellular domain
(SEQ ID NO: 4)
CD2 Transmembrane domain:
(SEQ ID NO: 14)
4-1BB Intracellular domain:
(SEQ ID NO: 119)
CD3z Intracellular domain:
(SEQ ID NO: 5)

An exemplary nucleotide sequence encoding a mutant CSR 03b CD2.8.DH.BBz protein of the disclosure comprises or consists of the nucleotide sequence of (CD8a Signal peptide, CD2 Extracellular domain with D111H mutation within the CD2 Extracellular domain, CD2 Transmembrane domain, 4-1BB Intracellular domain, CD3z Intracellular domain):

(SEQ ID NO: 123)
ATGGCTCTGCCTGTGACAGCTCTGCTGCTGCCTCTGGCTCTGCTTCTGCATGCCGCCAGACC
TAAAGAAATCACGAATGCATTGGAAACCTGGGGAGCCCTCGGCCAGGATATTAACCTGGACA
TCCCCAGCTTCCAGATGAGCGACGACATCGATGACATCAAGTGGGAGAAAACCAGCGACAAG
AAGAAGATCGCCCAGTTCCGGAAAGAGAAAGAGACATTCAAAGAGAAGGACACCTACAAGCT
GTTCAAGAACGGCACCCTGAAGATCAAGCACCTGAAAACCGACGACCAGGACATCTATAAGG
TGTCCATCTACCACACCAAGGGCAAGAACGTGCTGGAAAAGATCTTCGACCTCAAGATCCAA
GAGCGGGTGTCCAAGCCTAAGATCAGCTGGACCTGCATCAACACCACACTGACCTGCGAAGT
GATGAACGGCACAGACCCCGAGCTGAACCTGTACCAGGATGGCAAACACCTGAAGCTGAGCC
AGCGCGTGATCACCCACAAGTGGACAACAAGCCTGAGCGCCAAGTTCAAGTGCACCGCCGGA
AACAAAGTGTCTAAAGAGTCCAGCGTCGAGCCCGTGTCTTGCCCTGAAAAAGGACTGGACAT
CTACCTGATCATCGGCATCTGTGGCGGCGGATCCCTGCTGATGGTGTTTGTGGCTCTGCTGG
TGTTCTACATCACC

[symbol]

[symbol]

[symbol]

[symbol]

[symbol]

[symbol]

[symbol]

AGAGTGAAATTCAGCCGCAGCGCCGATGCTCCTGCCTATAAGCAGG
GACAGAACCAGCTGTACAACGAGCTGAATCTGGGGCGCAGAGAAGAGTACGATGTGCTGGAC
AAGCGGAGAGGCAGAGATCCTGAGATGGGCGGCAAGCCCAGACGGAAGAATCCTCAAGAGGG
CCTGTATAATGAGCTGCAGAAAGACAAGATGGCCGAGGCCTACAGCGAGATCGGAATGAAGG
GCGAGCGCAGAAGAGGCAAGGGACACGATGGACTGTATCAGGGCCTGAGCACCGCCACCAAG
GATACCTATGATGCCCTGCACATGCAGGCCCTGCCTCCAAGA
CD8a Signal peptide:
(SEQ ID NO: 111)
ATGGCTCTGCCTGTGACAGCTCTGCTGCTGCCTCTGGCTCTGCTTCTGCATGCCGCCAGACC
T
CD2 Extracellular domain with D111H mutation within
the CD2 Extracellular domain:
(SEQ ID NO: 106)
CD2 Transmembrane domain:
(SEQ ID NO: 102)
4-1BB Intracellular domain:
(SEQ ID NO: 121)

[symbol]

[symbol]

[symbol]

[symbol]

[symbol]

[symbol]

[symbol]

CD3z Intracellular domain:
(SEQ ID NO: 94)

An exemplary mutant CSR 04 CD2.DH.7z protein of the disclosure comprises or consists of the amino acid sequence of (CD2 Signal peptide, CD2 Extracellular domain with D111H mutation within the CD2 Extracellular domain, CD2 Transmembrane domain, IL7RA Intracellular domain, CD3z Intracellular domain):

(SEQ ID NO: 124)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYLIIGICGGGSLLMVFVALLVFYIT

[symbol]

[symbol]

[symbol]

[symbol]

[symbol]

[symbol]

[symbol]

[symbol]

[symbol]

[symbol]

RYRF
SRSADAPAYKQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
CD2 Signal peptide:
(SEQ ID NO: 9)
CD2 Extracellular domain with D111H mutation within
the CD2 Extracellular domain:
(SEQ ID NO: 4)
CD2 Transmembrane domain:
(SEQ ID NO: 14)
IL7RA Intracellular domain:
(SEQ ID NO: 125)

[symbol]

[symbol]

[symbol]

[symbol]

[symbol]

[symbol]

[symbol]

[symbol]

[symbol]

[symbol]

CD3z Intracellular domain:
(SEQ ID NO: 5)

An exemplary nucleotide sequence encoding a mutant CSR 04 CD2.DH.7z protein of the disclosure comprises or consists of the nucleotide sequence of (CD2 Signal peptide, CD2 Extracellular domain with D111H mutation within the CD2 Extracellular domain, CD2 Transmembrane domain, IL7RA Intracellular domain, CD3z Intracellular domain):

(SEQ ID NO: 126)
ATGAGCTTCCCTTGCAAGTTCGTGGCCAGCTTCCTGCTGATCTTCAACGTGTCCTCTAAGGG
CGCCGTGTCCAAAGAAATCACGAATGCATTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGATCCCTGCTGATGGTGTTTGTGG
CTCTGCTGGTGTTCTACATCACC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

AGAGAAATTC
AGCCGCAGCGCCGATGCTCCTGCCTATAAGCAGGGACAGAACCAGCTGTACAACGAGCTGAA
TCTGGGGCGCAGAGAAGAGTACGATGTGCTGGACAAGCGGAGAGGCAGAGATCCTGAGATGG
GCGGCAAGCCCAGACGGAAGAATCCTCAAGAGGGCCTGTATAATGAGCTGCAGAAAGACAAG
ATGGCCGAGGCCTACAGCGAGATCGGAATGAAGGGCGAGCGCAGAAGAGGCAAGGGACACGA
TGGACTGTATCAGGGCCTGAGCACCGCCACCAAGGATACCTATGATGCCCTGCACATGCAGG
CCCTGCCTCCAAGA
CD2 Signal peptide:
(SEQ ID NO: 100)
CD2 Extracellular domain with D111H mutation within the CD2
Extracellular domain:
(SEQ ID NO: 106)
CD2 Transmembrane domain:
(SEQ ID NO: 102)
IL7RA Intracellular domain:
(SEQ ID NO: 127)

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

CD3z Intracellular domain:
(SEQ ID NO: 94)

An exemplary mutant CSR 04b CD2.8.DH.7z protein of the disclosure comprises or consists of the amino acid sequence of (CD8a Signal peptide, CD2 Extracellular domain with D111H mutation within the CD2 Extracellular domain, CD2 Transmembrane domain, IL7RA Intracellular domain, CD3z Intracellular domain):

(SEQ ID NO: 128)
MALPVTALLLPLALLLHAARPKEITTNALETWGALGQDINLDIPSFQMSDDIDDIKWEKTSDK
KKIAQFRKEKETFKEKDTYKLFKNGTLKIKHLKTDDQDIYKVSIYHTKGKNVLEKIFDLKIQ
ERVSKPKISWTCINTTLTCEVMNGTDPELNLYQDGKHLKLSQRVITHKWTTSLSAKFKCTAG
NKVSKESSVEPVSCPEKGLDIYLIIGICGGGSLLMVFVALLVFYIT

[symbol]

[symbol]

[symbol]

[symbol]

[symbol]

[symbol]

[symbol]

[symbol]

[symbol]

[symbol]

RVKFSRS
ADAPAYKQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR
CD8a Signal peptide:
(SEQ ID NO: 12)
CD2 Extracellular domain with D111H mutation
within the CD2 Extracellular domain:
(SEQ ID NO: 4)
CD2 Transmembrane domain:
(SEQ ID NO: 14)
IL7RA Intracellular domain:
(SEQ ID NO: 125)
CD3z Intracellular domain:
(SEQ ID NO: 5)

An exemplary nucleotide sequence encoding a mutant CSR 04b CD2.8.DH.7z protein of the disclosure comprises or consists of the nucleotide sequence of (CD8a Signal peptide, CD2 Extracellular domain with D111H mutation within the CD2 Extracellular domain, CD2 Transmembrane domain, IL7RA Intracellular domain, CD3z Intracellular domain):

(SEQ ID NO: 129)
ATGGCTCTGCCTGTGACAGCTCTGCTGCTGCCTCTGGCTCTGCTTCTGCATGCCGCCAGACC
TAAAGAAATCACGAATGCATTGGAAACCTGGGGAGCCCTCGGCCAGGATATTAACCTGGACA
TCCCCAGCTTCCAGATGAGCGACGACATCGATGACATCAAGTGGGAGAAAACCAGCGACAAG
AAGAAGATCGCCCAGTTCCGGAAAGAGAAAGAGACATTCAAAGAGAAGGACACCTACAAGCT
GTTCAAGAACGGCACCCTGAAGATCAAGCACCTGAAAACCGACGACCAGGACATCTATAAGG
TGTCCATCTACCACACCAAGGGCAAGAACGTGCTGGAAAAGATCTTCGACCTCAAGATCCAA
GAGCGGGTGTCCAAGCCTAAGATCAGCTGGACCTGCATCAACACCACACTGACCTGCGAAGT
GATGAACGGCACAGACCCCGAGCTGAACCTGTACCAGGATGGCAAACACCTGAAGCTGAGCC
AGCGCGTGATCACCCACAAGTGGACAACAAGCCTGAGCGCCAAGTTCAAGTGCACCGCCGGA
AACAAAGTGTCTAAAGAGTCCAGCGTCGAGCCCGTGTCTTGCCCTGAAAAAGGACTGGACAT
CTACCTGATCATCGGCATCTGTGGCGGCGGATCCCTGCTGATGGTGTTTGTGGCTCTGCTGG
TGTTCTACATCACC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

AGAGTGAAATCAGCCGCAGC
GCCGATGCTCCTGCCTATAAGCAGGGACAGAACCAGCTGTACAACGAGCTGAATCTGGGGCG
CAGAGAAGAGTACGATGTGCTGGACAAGCGGAGAGGCAGAGATCCTGAGATGGGCGGCAAGC
CCAGACGGAAGAATCCTCAAGAGGGCCTGTATAATGAGCTGCAGAAAGACAAGATGGCCGAG
GCCTACAGCGAGATCGGAATGAAGGGCGAGCGCAGAAGAGGCAAGGGACACGATGGACTGTA
TCAGGGCCTGAGCACCGCCACCAAGGATACCTATGATGCCCTGCACATGCAGGCCCTGCCTC
CAAGA
CD8a Signal peptide:
(SEQ ID NO: 111)
ATGGCTCTGCCTGTGACAGCTCTGCTGCTGCCTCTGGCTCTGCTTCTGCATGCCGCCAGACC
T
CD2 Extracellular domain with D111H mutation within the CD2
Extracellular domain:
(SEQ ID NO: 106)
CD2 Transmembrane domain:
(SEQ ID NO: 102)
IL7RA Intracellular domain:
(SEQ ID NO: 127)
CD3z Intracellular domain:
(SEQ ID NO: 94)

An exemplary mutant CSR 05 CD2.DH.15z protein of the disclosure comprises or consists of the amino acid sequence of (CD2 Signal peptide, CD2 Extracellular domain with D111H mutation within the CD2 Extracellular domain, CD2 Transmembrane domain, IL15RA Intracellular domain, CD3z Intracellular domain):

(SEQ ID NO: 130)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYLIIGICGGGSLLMVFVALLVFYIT [symbol] [symbol]
RVKFSRSADAPAYKQGQNQLYNELNLGRREEYDVLD
KRRGRDPEMGGKPRRKNPQEGLYNELQKDKMAEAYSEIGMKGERRRGKGHDGLYQGLSTATK
DTYDALHMQALPPR
CD2 Signal peptide:
(SEQ ID NO: 9)
CD2 Extracellular domain with D111H mutation within
the CD2 Extracellular domain:
(SEQ ID NO: 4)
CD2 Transmembrane domain:
(SEQ ID NO: 14)
IL15 RA Intracellular domain:
(SEQ ID NO: 131)

[symbol]

[symbol]

CD3z Intracellular domain:
(SEQ ID NO: 5)

An exemplary nucleotide sequence encoding a mutant CSR 05 CD2.DH.15z protein of the disclosure comprises or consists of the nucleotide sequence of (CD2 Signal peptide, CD2 Extracellular domain with D111H mutation within the CD2 Extracellular domain, CD2 Transmembrane domain, IL15RA Intracellular domain, CD3z Intracellular domain):

(SEQ ID NO: 132)
ATGAGCTTCCCTTGCAAGTTCGTGGCCAGCTTCCTGCTGATCTTCAACGTGTCCTCTAAGGG
CGCCGTGTCCAAAGAAATCACGAATGCATTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGATCCCTGCTGATGGTGTTTGTGG
CTCTGCTGGTGTTCTACATCACC

[symbol]

[symbol]

[symbol]

[symbol]

[symbol]

[symbol]

AGAGTGAAATTCAGCCGCAGCGCCGATGCTCCTGCCTATAAGCAGG
GACAGAACCAGCTGTACAACGAGCTGAATCTGGGGCGCAGAGAAGAGTACGATGTGCTGGAC
AAGCGGAGAGGCAGAGATCCTGAGATGGGCGGCAAGCCCAGACGGAAGAATCCTCAAGAGGG
CCTGTATAATGAGCTGCAGAAAGACAAGATGGCCGAGGCCTACAGCGAGATCGGAATGAAGG
GCGAGCGCAGAAGAGGCAAGGGACACGATGGACTGTATCAGGGCCTGAGCACCGCCACCAAG
GATACCTATGATGCCCTGCACATGCAGGCCCTGCCTCCAAGA
CD2 Signal peptide:
(SEQ ID NO: 100)
CD2 Extracellular domain with D111H mutation
within the CD2 Extracellular domain:
(SEQ ID NO: 106)
CD2 Transmembrane domain:
(SEQ ID NO: 102)
IL15RA Intracellular domain:
(SEQ ID NO: 133)

[symbol]

[symbol]

[symbol]

[symbol]

[symbol]

[symbol]

CD3z Intracellular domain:
(SEQ ID NO: 94)

An exemplary mutant CSR 05b CD2.8.DH.15z protein of the disclosure comprises or consists of the amino acid sequence of (CD8a Signal peptide, CD2 Extracellular domain with D111H mutation within the CD2 Extracellular domain, CD2 Transmembrane domain, IL15RA Intracellular domain, CD3z Intracellular domain):

(SEQ ID NO: 134)
MALPVTALLLPLALLLHAARPKEITTNALETWGALGQDINLDIPSFQMSDDIDDIKWEKTSDK
KKIAQFRKEKETFKEKDTYKLFKNGTLKIKHLKTDDQDIYKVSIYHTKGKNVLEKIFDLKIQ
ERVSKPKISWTCINTTLTCEVMNGTDPELNLYQDGKHLKLSQRVITHKWTTSLSAKFKCTAG
TAGNKVSKESSVEPVSCPEKGL DIYLIIGICGGGSLLMVFVALLVFYIT [symbol] [symbol]
RVKFSRSADAPAYKQGQNQLYNELNLGRREEYDVLDKRR
GRDPEMGGKPRRKNPQEGLYNELQKDKMAEAYSEIGMKGERRRGKGHDGLYQGLSTATKDTY
DALHMQALPPR
CD8a Signal peptide:
(SEQ ID NO: 12)
CD2 Extracellular domain with D111H mutation within
the CD2 Extracellular domain:
(SEQ ID NO: 4)
CD2 Transmembrane domain:
(SEQ ID NO: 14)
IL15RA Intracellular domain″.
(SEQ ID NO: 131)
CD3z Intracellular domain:
(SEQ ID NO: 5)

An exemplary nucleotide sequence encoding a mutant CSR 05b CD2.8.DH.15z protein of the disclosure comprises or consists of the nucleotide sequence of (CD8a Signal peptide, CD2 Extracellular domain with D111H mutation within the CD2 Extracellular domain, CD2 Transmembrane domain, IL15RA Intracellular domain, CD3z Intracellular domain):

(SEQ ID NO: 135)
ATGGCTCTGCCTGTGACAGCTCTGCTGCTGCCTCTGGCTCTGCTTCTGCATGCCGCCAGACC
TAAAGAAATCACGAATGCATTGGAAACCTGGGGAGCCCTCGGCCAGGATATTAACCTGGACA
TCCCCAGCTTCCAGATGAGCGACGACATCGATGACATCAAGTGGGAGAAAACCAGCGACAAG
AAGAAGATCGCCCAGTTCCGGAAAGAGAAAGAGACATTCAAAGAGAAGGACACCTACAAGCT
GTTCAAGAACGGCACCCTGAAGATCAAGCACCTGAAAACCGACGACCAGGACATCTATAAGG
TGTCCATCTACCACACCAAGGGCAAGAACGTGCTGGAAAAGATCTTCGACCTCAAGATCCAA
GAGCGGGTGTCCAAGCCTAAGATCAGCTGGACCTGCATCAACACCACACTGACCTGCGAAGT
GATGAACGGCACAGACCCCGAGCTGAACCTGTACCAGGATGGCAAACACCTGAAGCTGAGCC
AGCGCGTGATCACCCACAAGTGGACAACAAGCCTGAGCGCCAAGTTCAAGTGCACCGCCGGA
AACAAAGTGTCTAAAGAGTCCAGCGTCGAGCCCGTGTCTTGCCCTGAAAAAGGACTGGACAT
CTACCTGATCATCGGCATCTGTGGCGGCGGATCCCTGCTGATGGTGTTTGTGGCTCTGCTGG
TGTTCTACAACAC

[symbol]

[symbol]

[symbol]

[symbol]

[symbol]

[symbol]

[symbol]

AGAGTGAAATTCAGCCGCAGCGCCGATGCTCCTGCCTATAAGCAGGGACAGAACC
AGCTGTACAACGAGCTGAATCTGGGGCGCAGAGAAGAGTACGATGTGCTGGACAAGCGGAGA
GGCAGAGATCCTGAGATGGGCGGCAAGCCCAGACGGAAGAATCCTCAAGAGGGCCTGTATAA
TGAGCTGCAGAAAGACAAGATGGCCGAGGCCTACAGCGAGATCGGAATGAAGGGCGAGCGCA
GAAGAGGCAAGGGACACGATGGACTGTATCAGGGCCTGAGCACCGCCACCAAGGATACCTAT
GATGCCCTGCACATGCAGGCCCTGCCTCCAAGA
CD8a Signal peptide:
(SEQ ID NO: 111)
ATGGCTCTGCCTGTGACAGCTCTGCTGCTGCCTCTGGCTCTGCTTCTGCATGCCGCCAGACCT
CD2 Extracellular domain with D111H mutation within
the CD2 Extracellular domain:
(SEQ ID NO: 106)
CD2 Transmembrane domain:
(SEQ ID NO: 102)
IL15RA Intracellular domain:
(SEQ ID NO: 133)

[symbol]

[symbol]

[symbol]

[symbol]

[symbol]

[symbol]

CD3z Intracellular domain:
(SEQ ID NO: 94)

An exemplary mutant CSR 06 CD2.DH.21z protein of the disclosure comprises or consists of the amino acid sequence of (CD2 Signal peptide, CD2 Extracellular domain with D111H mutation within the CD2 Extracellular domain, CD2 Transmembrane domain, IL21R Intracellular domain, CD3z Intracellular domain):

(SEQ ID NO: 136)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYLIIGICGGGSLLMVFVALLVFYIT

[symbol]

[symbol]

[symbol]

[symbol]

[symbol]

[symbol]

[symbol]

[symbol]

[symbol]

[symbol]

[symbol]

[symbol]

[symbol]

PPRSYLRQWVVIPPPLSSPGPQASRVKFSRSADAPAYKQGQNQLYNELNLGRREEYDVLDKR
RGRDPEMGGKPRRKNPQEGLYNELQKDKMAEAYSEIGMKGERRRGKGHDGLYQGLSTATKDT
YDALHMQALPPR
CD2 Signal peptide:
(SEQ ID NO: 9)
CD2 Extracellular domain with D111H mutation within the CD2
Extracellular domain:
(SEQ ID NO: 4)
CD2 Transmembrane domain:
(SEQ ID NO: 14)
IL21R Intracellular domain:
(SEQ ID NO: 137)

[symbol]

[symbol]

[symbol]

[symbol]

[symbol]

[symbol]

[symbol]

[symbol]

[symbol]

[symbol]

[symbol]

[symbol]

[symbol]

[symbol]

CD3z Intracellular domain:
(SEQ ID NO: 5)

An exemplary nucleotide sequence encoding a mutant CSR 06 CD2.DH.21z protein of the disclosure comprises or consists of the nucleotide sequence of (CD2 Signal peptide, CD2 Extracellular domain with D111H mutation within the CD2 Extracellular domain, CD2 Transmembrane domain, IL21R Intracellular domain, CD3z Intracellular domain):

(SEQ ID NO: 138)
ATGAGCTTCCCTTGCAAGTTCGTGGCCAGCTTCCTGCTGATCTTCAACGTGTCCTCTAAGGG
CGCCGTGTCCAAAGAAATCACGAATGCATTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGATCCCTGCTGATGGTGTTTGTGG
CTCTGCTGGTGTTCTAGATCACC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

AGAGTGAAATTCAGCCGCAGCGCCGATGCTCCTGCCTATAAGCAGGGACAGA
ACCAGCTGTACAACGAGCTGAATCTGGGGCGCAGAGAAGAGTACGATGTGCTGGACAAGCGG
AGAGGCAGAGATCCTGAGATGGGCGGCAAGCCCAGACGGAAGAATCCTCAAGAGGGCCTGTA
TAATGAGCTGCAGAAAGACAAGATGGCCGAGGCCTACAGCGAGATCGGAATGAAGGGCGAGC
GCAGAAGAGGCAAGGGACACGATGGACTGTATCAGGGCCTGAGCACCGCCACCAAGGATACC
TATGATGCCCTGCACATGCAGGCCCTGCCTCCAAGA
CD2 Signal peptide:
(SEQ ID NO: 100)
CD2 Extracellular domain with D111H mutation within the CD2
Extracellular domain:
(SEQ ID NO: 106)
CD2 Transmembrane domain:
(SEQ ID NO: 102)
IL21R Intracellular domain:
(SEQ ID NO: 139)

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

CD3z Intracellular domain:
(SEQ ID NO: 94)

An exemplary mutant CSR 06b CD2.8.DH.21z protein of the disclosure comprises or consists of the amino acid sequence of (CD8a Signal peptide, CD2 Extracellular domain with D111H mutation within the CD2 Extracellular domain, CD2 Transmembrane domain, IL21R Intracellular domain, CD3z Intracellular domain):

(SEQ ID NO: 140)
MALPVTALLLPLALLLHAARPKEITNALETWGALGQDINLDIPSFQMSDDIDDIKWEKTSDK
KKIAQFRKEKETFKEKDTYKLFKNGTLKIKHLKTDDQDIYKVSIYHTKGKNVLEKIFDLKIQ
ERVSKPKISWTCINTTLTCEVMNGTDPELNLYQDGKHLKLSQRVITHKWTTSLSAKFKCTAG
NKVSKESSVEPVSCPEKGLDIYLIIGICGGGSLLMVFVALLVFYIT

[symbol]

[symbol]

[symbol]

[symbol]

[symbol]

[symbol]

[symbol]

[symbol]

[symbol]

[symbol]

[symbol]

[symbol]

[symbol]

[symbol]

RVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKDTYDA
LHMQALPPR
CD8a Signal peptide:
(SEQ ID NO: 12)
CD2 Extracellular domain with D111H mutation within the CD2
Extracellular domain:
(SEQ ID NO: 4)
CD2 Transmembrane domain:
(SEQ ID NO: 14)
IL21R Intracellular domain:
(SEQ ID NO: 137)
CD3z Intracellular domain:
(SEQ ID NO: 5)

An exemplary nucleotide sequence encoding a mutant CSR 06b CD2.8.DH.21z protein of the disclosure comprises or consists of the nucleotide sequence of (CD8a Signal peptide, CD2 Extracellular domain with D111H mutation within the CD2 Extracellular domain, CD2 Transmembrane domain, IL21R Intracellular domain, CD3z Intracellular domain):

(SEQ ID NO: 141)
ATGGCTCTGCCTGTGACAGCTCTGCTGCTGCCTCTGGCTCTGCTTCTGCATGCCGCCAGACC
TAAAGAAATCACGAATGCATTGGAAACCTGGGGAGCCCTCGGCCAGGATATTAACCTGGACA
TCCCCAGCTTCCAGATGAGCGACGACATCGATGACATCAAGTGGGAGAAAACCAGCGACAAG
AAGAAGATCGCCCAGTTCCGGAAAGAGAAAGAGACATTCAAAGAGAAGGACACCTACAAGCT
GTTCAAGAACGGCACCCTGAAGATCAAGCACCTGAAAACCGACGACCAGGACATCTATAAGG
TGTCCATCTACCACACCAAGGGCAAGAACGTGCTGGAAAAGATCTTCGACCTCAAGATCCAA
GAGCGGGTGTCCAAGCCTAAGATCAGCTGGACCTGCATCAACACCACACTGACCTGCGAAGT
GATGAACGGCACAGACCCCGAGCTGAACCTGTACCAGGATGGCAAACACCTGAAGCTGAGCC
AGCGCGTGATCACCCACAAGTGGACAACAAGCCTGAGCGCCAAGTTCAAGTGCACCGCCGGA
AACAAAGTGTCTAAAGAGTCCAGCGTCGAGCCCGTGTCTTGCCCTGAAAAAGGACTGGACAT
CTACCTGATCATCGGCATCTGTGGCGGCGGATCCCTGCTGATGGTGTTTGTGGCTCTGCTGG
TGTTCTAGATCACC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

CAGAGTGAAATTCAGCCGCAGCGCCGATGCTCCTGCCTATAAGCAGGGACAGAACCAGCTGT
ACAACGAGCTGAATCTGGGGCGCAGAGAAGAGTACGATGTGCTGGACAAGCGGAGAGGCAGA
GATCCTGAGATGGGCGGCAAGCCCAGACGGAAGAATCCTCAAGAGGGCCTGTATAATGAGCT
GCAGAAAGACAAGATGGCCGAGGCCTACAGCGAGATCGGAATGAAGGGCGAGCGCAGAAGAG
GCAAGGGACACGATGGACTGTATCAGGGCCTGAGCACCGCCACCAAGGATACCTATGATGCC
CTGCACATGCAGGCCCTGCCTCCAAGA
CD8a Signal peptide:
(SEQ ID NO: 111)
ATGGCTCTGCCTGTGACAGCTCTGCTGCTGCCTCTGGCTCTGCTTCTGCATGCCGCCAGACC
T
CD2 Extracellular domain with D111H mutation within the CD2
Extracellular domain:
(SEQ ID NO: 106)
CD2 Transmembrane domain:
(SEQ ID NO: 102)
IL21R Intracellular domain:
(SEQ ID NO: 139)
CD3z Intracellular domain:
(SEQ ID NO: 94)

An exemplary mutant CSR 07 CD2.DH.Iz protein of the disclosure comprises or consists of the amino acid sequence of (CD2 Signal peptide, CD2 Extracellular domain with D111H mutation within the CD2 Extracellular domain, CD2 Transmembrane domain, ICOS Intracellular domain, CD3z Intracellular domain):

(SEQ ID NO: 142)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGL DIYLIIGICGGGSLLMVFVALLVFYIT [symbol] [symbol]
RVKFSRSADAPAYKQGQNQLYNELNLGRREEYDVLDK
RRGRDPEMGGKPRRKNPQEGLYNELQKDKMAEAYSEIGMKGERRRGKGHDGLYQGLSTATKD
TYDALHMQALPPR
CD2 Signal peptide:
(SEQ ID NO: 9)
CD2 Extracellular domain with D111H mutation within
the CD2 Extracellular domain:
(SEQ ID NO: 4)
CD2 Transmembrane domain:
(SEQ ID NO: 14)
ICOS Intracellular domain:
(SEQ ID NO: 143)

[symbol]

[symbol]

CD3z Intracellular domain:
(SEQ ID NO: 5)

An exemplary nucleotide sequence encoding a mutant CSR 07 CD2.DH.Iz protein of the disclosure comprises or consists of the nucleotide sequence of (CD2 Signal peptide, CD2 Extracellular domain with D111H mutation within the CD2 Extracellular domain, CD2 Transmembrane domain, ICOS Intracellular domain, CD3z Intracellular domain):

(SEQ ID NO: 144)
ATGAGCTTCCCTTGCAAGTTCGTGGCCAGCTTCCTGCTGATCTTCAACGTGTCCTCTAAGGG
CGCCGTGTCCAAAGAAATCACGAATGCATTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGATCCCTGCTGATGGTGTTTGTGG
CTCTGCTGGTGTTCTACATCACC

[symbol]

[symbol]

[symbol]

[symbol]

[symbol]

[symbol]

AGAGTGAAATTCAGCCGCAGCGCCGATGCTCCTGCCTATAAGCAGGGAC
AGAACCAGCTGTACAACGAGCTGAATCTGGGGCGCAGAGAAGAGTACGATGTGCTGGACAAG
CGGAGAGGCAGAGATCCTGAGATGGGCGGCAAGCCCAGACGGAAGAATCCTCAAGAGGGCCT
GTATAATGAGCTGCAGAAAGACAAGATGGCCGAGGCCTACAGCGAGATCGGAATGAAGGGCG
AGCGCAGAAGAGGCAAGGGACACGATGGACTGTATCAGGGCCTGAGCACCGCCACCAAGGAT
ACCTATGATGCCCTGCACATGCAGGCCCTGCCTCCAAGA
CD2 Signal peptide:
(SEQ ID NO: 100)
CD2 Extracellular domain with D111H mutation within the CD2
Extracellular domain:
(SEQ ID NO: 106)
CD2 Transmembrane domain:
(SEQ ID NO: 102)
ICOS Intracellular domain:
(SEQ ID NO: 145)

[symbol]

[symbol]

[symbol]

[symbol]

[symbol]

[symbol]

CD3z Intracellular domain:
(SEQ ID NO: 94)

An exemplary mutant CSR 07b CD2.8.DH.Iz protein of the disclosure comprises or consists of the amino acid sequence of (CD8a Signal peptide, CD2 Extracellular domain with D111H mutation within the CD2 Extracellular domain, CD2 Transmembrane domain, ICOS Intracellular domain, CD3z Intracellular domain):

(SEQ ID NO: 146)
MALPVTALLLPLALLLHAARPKEITNALETWGALGQDINLDIPSFQMSDDIDDIKWEKTSDK
KKIAQFRKEKETFKEKDTYKLFKNGTLKIKHLKTDDQDIYKVSIYHTKGKNVLEKIFDLKIQ
ERVSKPKISWTCINTTLTCEVMNGTDPELNLYQDGKHLKLSQRVITHKWTTSLSAKFKCTAG
NKVSKESSVEPVSCPEKGLDIYLIIGICGGGSLLMVFVALLVFYIT [symbol] [symbol]
RVKFSRSADAPAYKQGQNQLYNELNLGRREEYDVLDKRRG
RDPEMGGKPRRKNPQEGLYNELQKDKMAEAYSEIGMKGERRRGKGHDGLYQGLSTATKDTYD
ALHMQALPPR
CD8a Signal peptide:
(SEQ ID NO: 12)
CD2 Extracellular domain with D111H mutation within the CD2
Extracellular domain:
(SEQ ID NO: 4)
CD2 Transmembrane domain:
(SEQ ID NO: 14)
ICOS Intracellular domain:
(SEQ ID NO: 143)
CD3z Intracellular domain:
(SEQ ID NO: 5)

An exemplary nucleotide sequence encoding a mutant CSR 07b CD2.8.DH.Iz protein of the disclosure comprises or consists of the nucleotide sequence of (CD8a Signal peptide, CD2 Extracellular domain with D111H mutation within the CD2 Extracellular domain, CD2 Transmembrane domain, ICOS Intracellular domain, CD3z Intracellular domain):

(SEQ ID NO: 147)
ATGGCTCTGCCTGTGACAGCTCTGCTGCTGCCTCTGGCTCTGCTTCTGCATGCCGCCAGACC
TAAAGAAATCACGAATGCATTGGAAACCTGGGGAGCCCTCGGCCAGGATATTAACCTGGACA
TCCCCAGCTTCCAGATGAGCGACGACATCGATGACATCAAGTGGGAGAAAACCAGCGACAAG
AAGAAGATCGCCCAGTTCCGGAAAGAGAAAGAGACATTCAAAGAGAAGGACACCTACAAGCT
GTTCAAGAACGGCACCCTGAAGATCAAGCACCTGAAAACCGACGACCAGGACATCTATAAGG
TGTCCATCTACCACACCAAGGGCAAGAACGTGCTGGAAAAGATCTTCGACCTCAAGATCCAA
GAGCGGGTGTCCAAGCCTAAGATCAGCTGGACCTGCATCAACACCACACTGACCTGCGAAGT
GATGAACGGCACAGACCCCGAGCTGAACCTGTACCAGGATGGCAAACACCTGAAGCTGAGCC
AGCGCGTGATCACCCACAAGTGGACAACAAGCCTGAGCGCCAAGTTCAAGTGCACCGCCGGA
AACAAAGTGTCTAAAGAGTCCAGCGTCGAGCCCGTGTCTTGCCCTGAAAAAGGACTGGACAT
CTACCTGATCATCGGCATCTGTGGCGGCGGATCCCTGCTGATGGTGTTTGTGGCTCTGCTGG
TGTTCTAGATCACC

[symbol]

[symbol]

[symbol]

[symbol]

[symbol]

[symbol]

AGAGTGAAATTCAGCCGCAGCGCCGATGCTCCTGCCTATAAGCAGGGACAGAACCAGC
TGTACAACGAGCTGAATCTGGGGCGCAGAGAAGAGTACGATGTGCTGGACAAGCGGAGAGGC
AGAGATCCTGAGATGGGCGGCAAGCCCAGACGGAAGAATCCTCAAGAGGGCCTGTATAATGA
GCTGCAGAAAGACAAGATGGCCGAGGCCTACAGCGAGATCGGAATGAAGGGCGAGCGCAGAA
GAGGCAAGGGACACGATGGACTGTATCAGGGCCTGAGCACCGCCACCAAGGATACCTATGAT
GCCCTGCACATGCAGGCCCTGCCTCCAAGA
CD8a Signal peptide:
(SEQ ID NO: 111)
ATGGCTCTGCCTGTGACAGCTCTGCTGCTGCCTCTGGCTCTGCTTCTGCATGCCGCCAGACC
T
CD2 Extracellular domain with D111H mutation within the CD2
Extracellular domain:
(SEQ ID NO: 106)
CD2 Transmembrane domain:
(SEQ ID NO: 102)
ICOS Intracellular domain:
(SEQ ID NO: 145)
CD3z Intracellular domain:
(SEQ ID NO: 94)

An exemplary mutant CSR 08 CD2.DH.27z protein of the disclosure comprises or consists of the amino acid sequence of (CD2 Signal peptide, CD2 Extracellular domain with D111H mutation within the CD2 Extracellular domain, CD2 Transmembrane domain, CD27 Intracellular domain, CD3z Intracellular domain):

(SEQ ID NO: 148)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYLIIGICGGGSLLMVFVALLVFYIT [symbol] [symbol] [symbol]
RVKFSRSADAPAYKQGQNQLYNELNLG
RREEYDVLDKRRGRDPEMGGKPRRKNPQEGLYNELQKDKMAEAYSEIGMKGERRRGKGHDGL
YQGLSTATKDTYDALHMQALPPR
CD2 Signal peptide:
(SEQ ID NO: 9)
CD2 Extracellular domain with D111H mutation within the CD2
Extracellular domain:
(SEQ ID NO: 4)
CD2 Transmembrane domain:
(SEQ ID NO: 14)
CD27Intracellular domain:
(SEQ ID NO: 149)
QRRKYRSNKGESPVEPAEPCHYSCPREEEGSTIPIQEDYRKPEPACSP
CD3z Intracellular domain:
(SEQ ID NO: 5)

An exemplary nucleotide sequence encoding a mutant CSR 08 CD2.DH.27z protein of the disclosure comprises or consists of the nucleotide sequence of (CD2 Signal peptide, CD2 Extracellular domain with D111H mutation within the CD2 Extracellular domain, CD2 Transmembrane domain, CD27 Intracellular domain, CD3z Intracellular domain):

(SEQ ID NO: 150)
ATGAGCTTCCCTTGCAAGTTCGTGGCCAGCTTCCTGCTGATCTTCAACGTGTCCTCTAAGGG
CGCCGTGTCCAAAGAAATCACGAATGCATTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGATCCCTGCTGATGGTGTTTGTGG
CTCTGCTGGTGTTCTACATCACC

[symbol]

[symbol]

[symbol]

[symbol]

[symbol]

[symbol]

[symbol]

AGAGTGAAATTCAGCCGCA
GCGCCGATGCTCCTGCCTATAAGCAGGGACAGAACCAGCTGTACAACGAGCTGAATCTGGGG
CGCAGAGAAGAGTACGATGTGCTGGACAAGCGGAGAGGCAGAGATCCTGAGATGGGCGGCAA
GCCCAGACGGAAGAATCCTCAAGAGGGCCTGTATAATGAGCTGCAGAAAGACAAGATGGCCG
AGGCCTACAGCGAGATCGGAATGAAGGGCGAGCGCAGAAGAGGCAAGGGACACGATGGACTG
TATCAGGGCCTGAGCACCGCCACCAAGGATACCTATGATGCCCTGCACATGCAGGCCCTGCC
TCCAAGA
CD2 Signal peptide:
(SEQ ID NO: 100)
CD2 Extracellular domain with D111H mutation within the CD2
Extracellular domain:
(SEQ ID NO: 106)
CD2 Transmembrane domain:
(SEQ ID NO: 102)
CD2 7Intracellular domain:
(SEQ ID NO: 151)

[symbol]

[symbol]

[symbol]

[symbol]

[symbol]

[symbol]

[symbol]

CD3z Intracellular domain:
(SEQ ID NO: 94)

An exemplary mutant CSR 08b CD2.8.DH.27z protein of the disclosure comprises or consists of the amino acid sequence of (CD8a Signal peptide, CD2 Extracellular domain with D111H mutation within the CD2 Extracellular domain, CD2 Transmembrane domain, CD27 Intracellular domain, CD3z Intracellular domain):

(SEQ ID NO: 152)
MALPVTALLLPLALLLHAARPKEITNALETWGALGQDINLDIPSFQMSDDIDDIKWEKTSDK
KKIAQFRKEKETFKEKDTYKLFKNGTLKIKHLKTDDQDIYKVSIYHTKGKNVLEKIFDLKIQ
ERVSKPKISWTCINTTLTCEVMNGTDPELNLYQDGKHLKLSQRVITHKWTTSLSAKFKCTAG
NKVSKESSVEPVSCPEKGLDIYLIIGICGGGSLLMVFVALLVFYIT [symbol] [symbol] [symbol]
RVKFSRSADAPAYKQGQNQLYNELNLGRRE
EYDVLDKRRGRDPEMGGKPRRKNPQEGLYNELQKDKMAEAYSEIGMKGERRRGKGHDGLYQG
LSTATKDTYDALHMQALPPR
CD8a Signal peptide:
(SEQ ID NO: 12)
CD2 Extracellular domain with D111H mutation within the CD2
Extracellular domain:
(SEQ ID NO: 4)
CD2 Transmembrane domain:
(SEQ ID NO: 14)
CD27 Intracellular domain:
(SEQ ID NO: 149)
CD3z Intracellular domain:
(SEQ ID NO: 5)

An exemplary nucleotide sequence encoding a mutant CSR 08b CD2.8.DH.27z protein of the disclosure comprises or consists of the nucleotide sequence of (CD8a Signal peptide, CD2 Extracellular domain with D111H mutation within the CD2 Extracellular domain, CD2 Transmembrane domain, CD27 Intracellular domain, CD3z Intracellular domain):

(SEQ ID NO: 153)
ATGGCTCTGCCTGTGACAGCTCTGCTGCTGCCTCTGGCTCTGCTTCTGCATGCCGCCAGACC
TAAAGAAATCACGAATGCATTGGAAACCTGGGGAGCCCTCGGCCAGGATATTAACCTGGACA
TCCCCAGCTTCCAGATGAGCGACGACATCGATGACATCAAGTGGGAGAAAACCAGCGACAAG
AAGAAGATCGCCCAGTTCCGGAAAGAGAAAGAGACATTCAAAGAGAAGGACACCTACAAGCT
GTTCAAGAACGGCACCCTGAAGATCAAGCACCTGAAAACCGACGACCAGGACATCTATAAGG
TGTCCATCTACCACACCAAGGGCAAGAACGTGCTGGAAAAGATCTTCGACCTCAAGATCCAA
GAGCGGGTGTCCAAGCCTAAGATCAGCTGGACCTGCATCAACACCACACTGACCTGCGAAGT
GATGAACGGCACAGACCCCGAGCTGAACCTGTACCAGGATGGCAAACACCTGAAGCTGAGCC
AGCGCGTGATCACCCACAAGTGGACAACAAGCCTGAGCGCCAAGTTCAAGTGCACCGCCGGA
AACAAAGTGTCTAAAGAGTCCAGCGTCGAGCCCGTGTCTTGCCCTGAAAAAGGACTGGACAT
CTACCTGATCATCGGCATCTGTGGCGGCGGATCCCTGCTGATGGTGTTTGTGGCTCTGCTGG
TGTTCTACATCACC

[symbol]

[symbol]

[symbol]

[symbol]

[symbol]

[symbol]

[symbol]

[symbol]

AGAGTGAAATTGAGCCGGAGCGCCGATG
CTCCTGCCTATAAGCAGGGACAGAACCAGCTGTACAACGAGCTGAATCTGGGGCGCAGAGAA
GAGTACGATGTGCTGGACAAGCGGAGAGGCAGAGATCCTGAGATGGGCGGCAAGCCCAGACG
GAAGAATCCTCAAGAGGGCCTGTATAATGAGCTGCAGAAAGACAAGATGGCCGAGGCCTACA
GCGAGATCGGAATGAAGGGCGAGCGCAGAAGAGGCAAGGGACACGATGGACTGTATCAGGGC
CTGAGCACCGCCACCAAGGATACCTATGATGCCCTGCACATGCAGGCCCTGCCTCCAAGA
CD8a Signal peptide:
(SEQ ID NO: 111)
ATGGCTCTGCCTGTGACAGCTCTGCTGCTGCCTCTGGCTCTGCTTCTGCATGCCGCCAGACCT
CD2 Extracellular domain with D111H mutation within the CD2
Extracellular domain:
(SEQ ID NO: 106)
CD2 Transmembrane domain:
(SEQ ID NO: 102)
CD27 Intracellular domain:
(SEQ ID NO: 151)
CD3z Intracellular domain:
(SEQ ID NO: 94)

An exemplary mutant CSR 09 CD2.DH.Oxz protein of the disclosure comprises or consists of the amino acid sequence of (CD2 Signal peptide, CD2 Extracellular domain with D111H mutation within the CD2 Extracellular domain, CD2 Transmembrane domain, OX40 Intracellular domain, CD3z Intracellular domain):

(SEQ ID NO: 154)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYLIIGICGGGSLLMVFVALLVFYIY [symbol] [symbol] [symbol]
RVKFSRSADAPAYKQGQNQLYNELNLGRREEYD
VLDKRRGRDPEMGGKPRRKNPQEGLYNELQKDKMAEAYSEIGMKGERRRGKGHDGLYQGLST
ATKDTYDALHMQALPPR
CD2 Signal peptide:
(SEQ ID NO: 9)
CD2 Extracellular domain with D111H mutation within the CD2
Extracellular domain
(SEQ ID NO: 4)
CD2 Transmembrane domain:
(SEQ ID NO: 14)
OX40 Intracellular domain:
(SEQ ID NO: 155)

[symbol]

[symbol]

CD3z Intracellular domain:
(SEQ ID NO: 5)

An exemplary nucleotide sequence encoding a mutant CSR 09 CD2.DH.Oxz protein of the disclosure comprises or consists of the nucleotide sequence of (CD2 Signal peptide, CD2 Extracellular domain with D111H mutation within the CD2 Extracellular domain, CD2 Transmembrane domain, OX40 Intracellular domain, CD3z Intracellular domain):

(SEQ ID NO: 156)
ATGAGCTTCCCTTGCAAGTTCGTGGCCAGCTTCCTGCTGATCTTCAACGTGTCCTCTAAGGG
CGCCGTGTCCAAAGAAATCACGAATGCATTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGATCCCTGCTGATGGTGTTTGTGG
CTCTGCTGGTGTTCTACATCACC

[symbol]

[symbol]

[symbol]

[symbol]

[symbol]

[symbol]

[symbol]

AGAGTGAAATTCAGCCGCAGCGCCGATGCTCCTGCCT
ATAAGCAGGGACAGAACCAGCTGTACAACGAGCTGAATCTGGGGCGCAGAGAAGAGTACGAT
GTGCTGGACAAGCGGAGAGGCAGAGATCCTGAGATGGGCGGCAAGCCCAGACGGAAGAATCC
TCAAGAGGGCCTGTATAATGAGCTGCAGAAAGACAAGATGGCCGAGGCCTACAGCGAGATCG
GAATGAAGGGCGAGCGCAGAAGAGGCAAGGGACACGATGGACTGTATCAGGGCCTGAGCACC
GCCACCAAGGATACCTATGATGCCCTGCACATGCAGGCCCTGCCTCCAAGA
CD2 Signal peptide:
(SEQ ID NO: 100)
CD2 Extracellular domain with D111H mutation within the CD2
Extracellular domain:
(SEQ ID NO: 106)
CD2 Transmembrane domain:
(SEQ ID NO: 102)
OX40 Intracellular domain:
(SEQ ID NO: 157)

[symbol]

[symbol]

[symbol]

[symbol]

[symbol]

[symbol]

[symbol]

CD3z Intracellular domain:
(SEQ ID NO: 94)

An exemplary mutant CSR 09b CD2.8.DH.Oxz protein of the disclosure comprises or consists of the amino acid sequence of (CD8a Signal peptide, CD2 Extracellular domain with D111H mutation within the CD2 Extracellular domain, CD2 Transmembrane domain, OX40 Intracellular domain, CD3z Intracellular domain):

(SEQ ID NO: 158)
MALPVTALLLPLALLLHAARPKEITTNALETWGALGQDINLDIPSFQMSDDIDDIKWEKTSDK
KKIAQFRKEKETFKEKDTYKLFKNGTLKIKHLKTDDQDIYKVSIYHTKGKNVLEKIFDLKIQ
ERVSKPKISWTCINTTLTCEVMNGTDPELNLYQDGKHLKLSQRVITHKWTTSLSAKFKCTAG
NKVSKESSVEPVSCPEKGL DIYLIIGICGGGSLLMVFVALLVFYIT [symbol] [symbol] [symbol]
RVKFSRSADAPAYKQGQNQLYNELNLGRREEYDVLD
KRRGRDPEMGGKPRRKNPQEGLYNELQKDKMAEAYSEIGMKGERRRGKGHDGLYQGLSTATK
DTYDALHMQALPPR
CD8a Signal peptide:
(SEQ ID NO: 12)
CD2 Extracellular domain with D111H mutation
within the CD2 Extracellular domain:
(SEQ ID NO: 4)
CD2 Transmembrane domain:
(SEQ ID NO: 14)
OX40 Intracellular domain:
(SEQ ID NO: 155)
CD3z Intracellular domain:
(SEQ ID NO: 5)

An exemplary nucleotide sequence encoding a mutant CSR 09b CD2.8.DH.Oxz protein of the disclosure comprises or consists of the nucleotide sequence of (CD8a Signal peptide, CD2 Extracellular domain with D111H mutation within the CD2 Extracellular domain, CD2 Transmembrane domain, OX40 Intracellular domain, CD3z Intracellular domain):

(SEQ ID NO: 159)
ATGGCTCTGCCTGTGACAGCTCTGCTGCTGCCTCTGGCTCTGCTTCTGCATGCCGCCAGACC
TAAAGAAATCACGAATGCATTGGAAACCTGGGGAGCCCTCGGCCAGGATATTAACCTGGACA
TCCCCAGCTTCCAGATGAGCGACGACATCGATGACATCAAGTGGGAGAAAACCAGCGACAAG
AAGAAGATCGCCCAGTTCCGGAAAGAGAAAGAGACATTCAAAGAGAAGGACACCTACAAGCT
GTTCAAGAACGGCACCCTGAAGATCAAGCACCTGAAAACCGACGACCAGGACATCTATAAGG
TGTCCATCTACCACACCAAGGGCAAGAACGTGCTGGAAAAGATCTTCGACCTCAAGATCCAA
GAGCGGGTGTCCAAGCCTAAGATCAGCTGGACCTGCATCAACACCACACTGACCTGCGAAGT
GATGAACGGCACAGACCCCGAGCTGAACCTGTACCAGGATGGCAAACACCTGAAGCTGAGCC
AGCGCGTGATCACCCACAAGTGGACAACAAGCCTGAGCGCCAAGTTCAAGTGCACCGCCGGA
AACAAAGTGTCTAAAGAGTCCAGCGTCGAGCCCGTGTCTTGCCCTGAAAAAGGACTGGACAT
CTACCTGATCATCGGCATCTGTGGCGGCGGATCCCTGCTGATGGTGTTTGTGGCTCTGCTGG
TGTTCTACATCACC

[symbol]

[symbol]

[symbol]

[symbol]

[symbol]

[symbol]

[symbol]

[symbol]

AGAGTGAAATTCAGCCGCAGCGCCGATGCTCCTGCCTATAAGCAGG
GACAGAACCAGCTGTACAACGAGCTGAATCTGGGGCGCAGAGAAGAGTACGATGTGCTGGAC
AAGCGGAGAGGCAGAGATCCTGAGATGGGCGGCAAGCCCAGACGGAAGAATCCTCAAGAGGG
CCTGTATAATGAGCTGCAGAAAGACAAGATGGCCGAGGCCTACAGCGAGATCGGAATGAAGG
GCGAGCGCAGAAGAGGCAAGGGACACGATGGACTGTATCAGGGCCTGAGCACCGCCACCAAG
GATACCTATGATGCCCTGCACATGCAGGCCCTGCCTCCAAGA
CD8a Signal peptide:
(SEQ ID NO: 111)
ATGGCTCTGCCTGTGACAGCTCTGCTGCTGCCTCTGGCTCTGCTTCTGCATGCCGCCAGACC
T
CD2 Extracellular domain with D111H mutation
within the CD2 Extracellular domain:
(SEQ ID NO: 106)
CD2 Transmembrane domain:
(SEQ ID NO: 102)
OX40 Intracellular domain:
(SEQ ID NO: 157)
CD3z Intracellular domain:
(SEQ ID NO: 94)

An exemplary mutant CSR 10 CD2.DH.Gz protein of the disclosure comprises or consists of the amino acid sequence of (CD2 Signal peptide, CD2 Extracellular domain with D111H mutation within the CD2 Extracellular domain, CD2 Transmembrane domain, GITR Intracellular domain, CD3z Intracellular domain):

(SEQ ID NO: 160)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGL DIYLIIGICGGGSLLMVFVALLVFYIT

[symbol]

[symbol]

[symbol]

[symbol]

NQLYNELNLGRREEYDVLDKRRGRDPEMGGKPRRKNPQEGLYNELQKDKMAEAYSEIGMKGE
RRRGKGHDGLYQGLSTATKDTYDALHMQALPPR
CD2 Signal peptide:
(SEQ ID NO: 9)
CD2 Extracellular domain with D111H
mutation within the CD2 Extracellular domain:
(SEQ ID NO: 4)
CD2 Transmembrane domain:
(SEQ ID NO: 14)
GITR Intracellular domain:

[symbol]

[symbol]

[symbol]

(SEQ ID NO: 161)
CD3z Intracellular domain:
(SEQ ID NO: 5)

An exemplary nucleotide sequence encoding a mutant CSR 10 CD2.DH.Gz protein of the disclosure comprises or consists of the nucleotide sequence of (CD2 Signal peptide, CD2 Extracellular domain with D111H mutation within the CD2 Extracellular domain, CD2 Transmembrane domain,

GITR Intracellular domain, CD3z Intracellular
domain):
(SEQ ID NO: 162)
ATGAGCTTCCCTTGCAAGTTCGTGGCCAGCTTCCTGCTGATCTTCAAC
GTGTCCTCTAAGGGCGCCGTGTCCAAAGAAATCACGAATGCATTGGAA
ACCTGGGGAGCCCTCGGCCAGGATATTAACCTGGACATCCCCAGCTTC
CAGATGAGCGACGACATCGATGACATCAAGTGGGAGAAAACCAGCGAC
AAGAAGAAGATCGCCCAGTTCCGGAAAGAGAAAGAGACATTCAAAGAG
AAGGACACCTACAAGCTGTTCAAGAACGGCACCCTGAAGATCAAGCAC
CTGAAAACCGACGACCAGGACATCTATAAGTGTCCATCTACCACACCA
AGGGCAAGAACGTGCTGGAAAAGATCTTCGACCTCAAGATCCAAGAGC
GGGTGTCCAAGCCTAAGATCAGCTGGACCTGCATCAACACCACACTGA
CCTGCGAAGTGATGAACGGCACAGACCCCGAGCTGAACCTGTACCAGG
ATGGCAAACACCTGAAGCTGAGCCAGCGCGTGATCACCCACAAGTGGA
CAACAAGCCTGAGCGCCAAGTTCAAGTGCACCGCCGGAAACAAAGTGT
CTAAAGAGTCCAGCGTCGAGCCCGTGTCTTGCCCTGAAAAAGGACTGG
ACATCTACCTGATCATCGGCATCTGTGGCGGCGGATCCCTGCTGATGG
TGTTTGTGGCTCTGCTGGTGTTCTACATCACA

[symbol]

[symbol]

[symbol]

[symbol]

[symbol]

[symbol]

[symbol]

[symbol]

[symbol]

AAATTCAGCCGCAGCGCCGATGCTCCTGCC
TATAAGCAGGGACAGAACCAGCTGTACAACGAGCTGAATCTGGGGCGC
AGAGAAGAGTACGATGTGCTGGACAAGCGGAGAGGCAGAGATCCTGAG
ATGGGCGGCAAGCCCAGACGGAAGAATCCTCAAGAGGGCCTGTATAAT
GAGCTGCAGAAAGACAAGATGGCCGAGGCCTACAGCGAGATCGGAATG
AAGGGCGAGCGCAGAAGAGGCAAGGGACACGATGGACTGTATCAGGGC
CTGAGCACCGCCACCAAGGATACCTATGATGCCCTGCACATGCAGGCC
CTGCCTCCAAGA
CD2 Signal peptide:
(SEQ ID NO: 100)
CD2 Extracellular domain with D111H mutation
within the CD2 Extracellular domain:
(SEQ ID NO: 106)
CD2 Transmembrane domain:
(SEQ ID NO: 102)
GITR Intracellular domain:
(SEQ ID NO: 163)

[symbol]

[symbol]

[symbol]

[symbol]

[symbol]

[symbol]

[symbol]

[symbol]

[symbol]

CD3z Intracellular domain:
(SEQ ID NO: 94)

An exemplary mutant CSR 10b CD2.8.DH.Gz protein of the disclosure comprises or consists of the amino acid sequence of (CD8a Signal peptide, CD2 Extracellular domain with D111H mutation within the CD2 Extracellular domain, CD2 Transmembrane domain,

GITR Intracellular domain, CD3z Intracellular
domain):
(SEQ ID NO: 164)
MALPVTALLLPLALLLHAARPKEITNALETWGALGQDINLDIPSFQMS
DDIDDIKWEKTSDKKKIAQFRKEKETFKEKDTYKLFKNGTLKIKHLKT
DDQDIYKVSIYHTKGKNVLEKIFDLKIQERVSKPKISWTCINTTLTCE
VMNGTDPELNLYQDGKHLKLSQRVITHKWTTSLSAKFKCTAGNKVSKE
SSVEPVSCPEKGLDIYLIIGICGGGSLLMVFVALLVFYIT

[symbol]

[symbol]

[symbol]

RVKFSRSADAPAYKQGQNQLYNEL
NLGRREEYDVLDKRRGRDPEMGGKPRRKNPQEGLYNELQKDKMAEAYS
EIGMKGERRRGKGHDGLYQGLSTATKDTYDALHMQALPRR
CD8 Signal peptide:
(SEQ ID NO: 12)
CD2 Extracellular domain with D111H mutation
within the CD2 Extracellular domain:
(SEQ ID NO: 4)
CD2 Transmembrane domain:
(SEQ ID NO: 14)
GITR Intracellular domain:
(SEQ ID NO: 161)
CD3z Intracelluar domain:
(SEQ ID NO: 5)

An exemplary nucleotide sequence encoding a mutant CSR 10b CD2.8.DH.Gz protein of the disclosure comprises or consists of the nucleotide sequence of (CD8a Signal peptide, CD2 Extracellular domain with D111H mutation within the CD2 Extracellular domain, CD2 Transmembrane domain,

GITR Intracellular domain, CD3z Intracellular
domain):
(SEQ ID NO: 165)
ATGGCTCTGCCTGTGACAGCTCTGCTGCTGCCTCTGGCTCTGCTTCTG
CATGCCGCCAGACCTAAAGAAATCACGAATGCATTGGAAACCTGGGGA
GCCCTCGGCCAGGATATTAACCTGGACATCCCCAGCTTCCAGATGAGC
GACGACATCGATGACATCAAGTGGGAGAAAACCAGCGACAAGAAGAAG
ATCGCCCAGTTCCGGAAAGAGAAAGAGACATTCAAAGAGAAGGACACC
TACAAGCTGTTCAAGAACGGCACCCTGAAGATCAAGCACCTGAAAACC
GACGACCAGGACATCTATAAGGTGTCCATCTACCACACCAAGGGCAAG
AACGTGCTGGAAAAGATCTTCGACCTCAAGATCCAAGAGCGGGTGTCC
AAGCCTAAGATCAGCTGGACCTGCATCAACACCACACTGACCTGCGAA
GTGATGAACGGCACAGACCCCGAGCTGAACCTGTACCAGGATGGCAAA
CACCTGAAGCTGAGCCAGCGCGTGATCACCCACAAGTGGACAACAAGC
CTGAGCGCCAAGTTCAAGTGCACCGCCGGAAACAAAGTGTCTAAAGAG
TCCAGCGTCGAGCCCGTGTCTTGCCCTGAAAAAGGACTGGACATCTAC
CTGATCATCGGCATCTGTGGCGGCGGATCCCTGCTGATGGTGTTTGTG
GCTCTGCTGGTGTTCTACATCACA

[symbol]

[symbol]

[symbol]

[symbol]

[symbol]

[symbol]

[symbol]

[symbol]

[symbol]

[symbol]

AAATTCAGCCGCAGCGCCGATGCTCCTGCCTATAAGCAGGGACAGAAC
CAGCTGTACAACGAGCTGAATCTGGGGCGCAGAGAAGAGTACGATGTG
CTGGACAAGCGGAGAGGCAGAGATCCTGAGATGGGCGGCAAGCCCAGA
CGGAAGAATCCTCAAGAGGGCCTGTATAATGAGCTGCAGAAAGACAAG
ATGGCCGAGGCCTACAGCGAGATCGGAATGAAGGGCGAGCGCAGAAGA
GGCAAGGGACACGATGGACTGTATCAGGGCCTGAGCACCGCCACCAAG
GATACCTATGATGCCCTGCACATGCAGGCCCTGCCTCCAAGA
CD8a Signal peptide
(SEQ ID NO: 111)
ATGGCTCTGCCTGTGACAGCTCTGCTGCTGCCTCTGGCTCTGCTTCTG
CATGCCGCCAGACCT
CD2 Extracellular doamin with D111H mutation
within the CD2 Extracellular domain:
(SEQ ID NO: 106)
CD2 Transmembrane domain:
(SEQ ID NO: 102)
GITR Intracellular domain:
(SEQ ID NO: 163)
CD3z Intracellular domain:
(SEQ ID NO: 94

In some embodiments, the disclosure provides a Chimeric Stimulatory Receptor (CSR) to deliver CD3z primary stimulation to Treg cells when stimulated using standard activation/stimulation reagents, including agonist anti-CD3 mAb.

In some embodiments, Chimeric Stimulatory Receptors (CSRs) of the disclosure provide a CD3 stimulus to enhance activation and expansion of regulatory T cells. In some embodiments, CSRs of the disclosure comprise an CD2, IL2RB or IL2RG extracellular domain that binds to an agonistic mAb and a TNFR2, IL2RB, IL2RG and/or CD3t intracellular stimulatory domain, which functionally, converts an anti-CD28 or anti-CD2 binding event on the surface into a TNFR2, IL2RB, IL2RG or CD3ζ signaling event in an T cell modified to express the CSR. In some embodiments, a CSR comprises a wild type CD2 extracellular domain and a TNFR2, IL2RB, IL2RG and/or a CD3z intracellular stimulatory domain. In preferred embodiments, the CSR express a non-naturally occurring antigen receptor and/or a therapeutic protein. In preferred embodiments, a CSR comprises a truncated or mutated extracellular domain CD2 protein and a CD3z intracellular stimulation domain. In some embodiments, the non-naturally occurring antigen receptor comprises a Chimeric Antigen Receptor.

The data provided herein demonstrate that modified T cells of the disclosure comprising/expressing a CSR of the disclosure improve the expansion of regulatory T cells when compared to those cells that do not comprise/express a CSR of the disclosure.

An exemplary protein sequence encoding a wild type CSR BBB.WT protein of the disclosure comprises or consists of the amino acid sequence of (IL2RB signal peptide, IL2RB extra cellular domain, IL2RB transmembrane domain, IL2RB intracellular domain):

(SEQ ID NO: 166)
MAAPALSWRLPLLILLLPLATSWASAAVNGTSQFTCFYNSRANISCVWS
QDTSCQVHAWPDRRRWNQTCELLPVSQASWACNLILGAPDSQKLTTVDI
VTLRVLCREGVRWRVMAIQDFKPFENLRLMAPISLQVVHVETHRCNISW
WISQASHYFERHLEFEARTLSPGHTWEEAPLLTLKQKQEWICLETLTPD
TQYEFQVRVKPLQGEFTTWSPWSQPLAFRTKPAALGKDTIPWLGHLLVG
LSGAFGFIIVYLLINCRNTGPWLKKVLKCNTPDPSKFFSQLSSEHGGDV
QKWLSSPFPSSSFSPGGLAPEISPLEVLERDKVTQLLLQQDKVPEPASL
SSNHSLTSCFTNQGYFFFHLPDALEIEACQVYFTYDPYSEEDPDEGVAG
APTGSSPQPLQPLSGEDDAYCRFPSRDDLLLFSPSLLGGPSPPSTAPGG
SGAGEERMPPSLQERVPRDWDPQPLGPPTPGVPDLVDFQPPPELVLREA
GEEVPDAGPREGVSFPWSRPPGQGEFRALNARLPLNTDAYLSLQELQGQ
DPTHLV

An exemplary nucleotide sequence encoding a wild type CSR BBB.WT protein of the disclosure comprises or consists of the amino acid sequence of (IL2RB signal peptide, IL2RB extra cellular domain, IL2RB transmembrane domain, IL2RB intracellular domain):

(SEQ ID NO: 167)
ATGGCTGCCCCTGCTCTGTCTTGGAGACTGCCTCTGCTGATCCTGCTGC
TGCCTCTGGCTACATCTTGGGCCTCTGCCGCCGTGAATGGCACCAGCCA
GTTCACCTGTTTCTACAACAGCCGGGCCAACATCAGCTGCGTGTGGTCA
CAAGATGGCGCCCTGCAGGATACCAGCTGTCAGGTTCACGCCTGGCCTG
ATCGGAGAAGATGGAACCAGACATGCGAGCTGCTGCCCGTGTCTCAGGC
TAGCTGGGCCTGTAATCTGATCCTGGGAGCCCCTGACAGCCAGAAACTG
ACCACAGTGGACATCGTGACCCTGCGGGTGCTGTGTAGAGAAGGCGTTC
GTTGGAGAGTGATGGCCATTCAGGACTTCAAGCCCTTCGAGAACCTGCG
GCTGATGGCCCCAATTAGCCTGCAGGTTGTGCACGTGGAAACCCACCGG
TGCAATATCAGCTGGGAGATCAGCCAGGCCAGCCACTACTTCGAGCGGC
ACCTGGAATTCGAGGCCAGAACACTGAGCCCTGGCCACACCTGGGAAGA
AGCTCCTCTGCTCACCCTGAAGCAGAAACAAGAGTGGATCTGCCTGGAA
ACCCTGACACCTGACACACAGTACGAGTTCCAAGTGCGCGTGAAGCCTC
TCCAGGGCGAGTTTACAACATGGTCCCCTTGGAGTCAGCCCCTGGCCTT
TAGAACAAAGCCTGCCGCTCTGGGCAAAGACACAATCCCTTGGCTGGGC
CATCTGCTCGTTGGACTGTCTGGCGCCTTCGGCTTCATCATCCTGGTGT
ACCTGCTGATCAACTGCCGGAACACAGGCCCCTGGCTGAAGAAAGTGCT
GAAGTGCAACACCCCTGATCCGAGCAAGTTCTTTAGCCAGCTGAGCAGC
GAGCATGGCGGCGACGTTCAGAAATGGCTGTCTAGCCCATTTCCTAGCA
GCAGCTTCAGCCCTGGTGGACTGGCCCCTGAGATTAGCCCTCTGGAAGT
GCTGGAACGGGACAAAGTGACCCAGCTGCTTCTGCAGCAGGACAAGGTG
CCAGAACCTGCCAGCCTGTCCAGCAATCACAGCCTGACCAGCTGTTTTA
CAAACCAGGGCTACTTCTTCTTCCATCTGCCTGACGCACTGGAAATTGA
GGCCTGCCAGGTGTACTTCACCTACGATCCCTACAGCGAAGAGGACCCC
GATGAAGGTGTTGCCGGTGCTCCTACCGGAAGCTCTCCTCAACCTCTGC
AACCACTGAGCGGCGAGGATGACGCCTACTGCACATTCCCCAGCAGAGA
TGACCTGCTCCTGTTCAGCCCTTCTCTGCTCGGCGGACCTTCTCCACCA
TCTACAGCTCCAGGTGGAAGCGGAGCCGGCGAGGAAAGAATGCCTCCAA
GCCTGCAAGAGCGGGTGCCCAGAGATTGGGATCCTCAACCACTGGGCCC
TCCAACACCTGGCGTGCCAGATCTCGTGGATTTCCAGCCTCCTCCAGAG
CTGGTGCTGAGAGAAGCTGGCGAAGAAGTGCCAGACGCTGGCCCTAGAG
AGGGCGTTAGCTTTCCTTGGAGCAGACCTCCTGGACAGGGCGAATTCAG
AGCCCTGAATGCCAGACTGCCCCTGAACACCGATGCCTACCTGTCTCTG
CAAGAACTGCAGGGACAAGACCCCACACACCTGGTG

An exemplary protein sequence encoding a wild type CSR GGG.WT protein of the disclosure comprises or consists of the amino acid sequence of (IL2RG signal peptide, IL2RG extra cellular domain, IL2RG transmembrane domain, IL2RG intracellular domain):

(SEQ ID NO: 168)
MLKPSLPFTSLLFLQLPLLGVGLNTTILTPNGNEDTTADFFLTTMPTDS
LSVSTLPLPEVQCFVFNVEYMNCTWNSSSEPQPTNLTLHYQYKNSDNDK
VQKCSHYLFSEEITSGCQLQKKEIHLYQTFVVQLQDPREPRRQATQMLK
LQNLVIPWAPENLTLHKLSESQLELNWNNRFLNHCLEHLVQYRTDWDHS
WTEQSVDYRHKFSLPSVDGQKRYTFRVRSRFNPLCGSAQHWSEWSHPIH
WGSNTSKENPFLFALEAVVISVGSMGLIISLLCVYFWLERTMPRIPTLK
NLEDLVTEYHGNFSAWSGVSKGLAESLQPDYSERLCLVSEIPPKGGALG
EGPGASPCNQHSPYWAPPCYTLKPET

An exemplary nucleotide sequence encoding a wild type CSR GGG.WT protein of the disclosure comprises or consists of the amino acid sequence of (IL2RG signal peptide, IL2RG extra cellular domain, IL2RG transmembrane domain, IL2RG intracellular domain):

(SEQ ID NO: 169)
ATGCTGAAGCCCAGCCTGCCTTTTACCAGCCTGCTGTTCCTGCAGCTGC
CTCTGCTTGGCGTGGGCCTGAATACCACCATCCTGACACCTAACGGCAA
CGAGGATACAACCGCCGACTTCTTCCTGACCACCATGCCTACCGATAGC
CTGAGCGTGTCCACACTGCCACTGCCTGAGGTGCAGTGCTTCGTGTTCA
ACGTCGAGTACATGAACTGCACCTGGAACAGCTCCAGCGAGCCCCAGCC
TACCAATCTGACACTGCACTACTGGTACAAGAACAGCGACAACGACAAG
GTGCAGAAGTGCAGCCACTACCTGTTCAGCGAGGAAATCACCAGCGGCT
GCCAGCTGCAGAAGAAAGAGATCCACCTGTACCAGACCTTCGTGGTGCA
GCTCCAGGATCCTAGAGAGCCTAGAAGGCAGGCCACACAGATGCTGAAA
CTGCAGAACCTGGTCATCCCCTGGGCTCCCGAAAACCTGACTCTGCACA
AGCTGAGCGAGAGCCAGCTGGAACTGAACTGGAACAACCGGTTCCTGAA
TCACTGCCTGGAACACCTGGTGCAGTACCGGACCGATTGGGATCACAGC
TGGACAGAGCAGAGCGTGGACTACCGGCACAAGTTCAGCCTGCCATCTG
TGGACGGCCAGAAGCGGTACACCTTTAGAGTGCGGAGCCGGTTCAATCC
CCTGTGTGGATCTGCTCAGCATTGGAGCGAGTGGTCACACCCTATCCAC
TGGGGCAGCAACACCAGCAAAGAGAACCCCTTCCTGTTCGCCCTGGAAG
CCGTGGTTATCAGCGTGGGCTCTATGGGCCTGATCATCTCCCTGCTGTG
CGTGTACTTCTGGCTGGAAAGAACCATGCCTCGGATCCCCACACTGAAG
AATCTGGAAGATCTGGTCACCGAGTACCACGGCAACTTCAGTGCTTGGA
GCGGCGTGTCAAAAGGCCTGGCCGAATCTCTGCAGCCCGACTACTCTGA
GAGACTGTGCCTGGTGTCTGAGATCCCTCCTAAAGGCGGAGCCCTCGGA
GAAGGACCTGGCGCCTCTCCATGTAATCAGCACAGCCCTTATTGGGCCC
CTCCTTGCTACACCCTGAAGCCTGAGACA

An exemplary protein sequence encoding a CSR 2DH.TF2z protein of the disclosure comprises or consists of the amino acid sequence of (CD2 signal peptide, CD2 extra cellular domain (DI 11H), TNFR2 transmembrane domain, TNFR2 intracellular domain #1, CD3z intracellular domain #2):

(SEQ ID NO: 17)
MSFPCKFVASFLLIFNVSSKGAVSKEITNALETWGALGQDINLDIPSFQ
MSDDIDDIKWEKTSDKKKIAQFRKEKETFKEKDTYKLFKNGTLKIKHLK
TDDQDIYKVSIYHTKGKNVLEKIFDLKIQERVSKPKISWTCINTTLTCE
VMNGTDPELNLYQDGKHLKLSQRVITHKWTTSLSAKFKCTAGNKVSKES
SVEPVSCPEKGLDFALPVGLIVGVTALGLLIIGVVNCVIMTQVKKKPLC
LQREAKVPHLPADKARGTQGPEQQHLLITAPSSSSSLESSASALDRRAP
TRNQPQAPGVEASGAGEARASTGSSDSSPGGHGTQVNVTCIVNVCSSSD
HSSQCSSQASSTMGDTDSSPSESPKDEQVPFSKEECAFRSQLETPETLL
GSTEEKPLPLGVPDAGMKPSRVKFSRSADAPAYKQGQNQLYNELNLGRR
EEYDVLDKRRGRDPEMGGKPRRKNPQEGLYNELQKDKMAEAYSEIGMKG
ERRRGKGHDGLYQGLSTATKDTYDALHMQALPPR

An exemplary nucleotide sequence encoding a CSR 2DH.TF2z protein of the disclosure comprises or consists of the amino acid sequence of (CD2 signal peptide, CD2 extra cellular domain (D111H), TNFR2 transmembrane domain, TNFR2 intracellular domain #1, CD3z intracellular domain #2):

(SEQ ID NO: 170)
ATGAGCTTCCCTTGCAAGTTCGTGGCCAGCTTCCTGCTGATCTTCAACG
TGTCCTCTAAGGGCGCCGTGTCCAAAGAGATCACAAACGCCCTGGAAAC
CTGGGGAGCCCTCGGCCAGGATATTAACCTGGACATCCCCAGCTTCCAG
ATGAGCGACGACATCGATGACATCAAGTGGGAGAAAACCAGCGACAAGA
AGAAGATCGCCCAGTTCCGGAAAGAGAAAGAGACATTCAAAGAGAAGGA
CACCTACAAGCTGTTCAAGAACGGCACCCTGAAGATCAAGCACCTGAAA
ACCGACGACCAGGACATCTATAAGGTGTCCATCTACCACACCAAGGGCA
AGAACGTGCTGGAAAAGATCTTCGACCTCAAGATCCAAGAGCGGGTGTC
CAAGCCTAAGATCAGCTGGACCTGCATCAACACCACTGACCTGCGAAGT
GATGAACGGCACAGACCCCGAGCTGAACCTGTACCAGGATGGCAAACAC
CTGAAGCTGAGCCAGCGCGTGATCACCCACAAGTGGACAACAAGCCTGA
GCGCCAAGTTCAAGTGCACCGCCGGAAACAAAGTGTCTAAAGAGTCCAG
CGTCGAGCCCGTGTCTTGCCCTGAGAAAGGCCTGGATTTTGCCCTGCCT
GTGGGCCTGATTGTGGGAGTGACAGCTCTGGGACTGCTGATCATCGGCG
TGGTCAACTGCGTGATCATGACCCAAGTGAAGAAAAAGCCCCTGTGCCT
GCAGCGCGAGGCCAAAGTTCCTCATCTGCCTGCCGATAAGGCCAGAGGC
ACACAGGGACCTGAACAGCAGCATCTGCTGATTACAGCCCCTAGCAGCA
GCAGCTCCAGCCTGGAATCTTCTGCCAGCGCTCTGGATCGGAGAGCCCC
TACCAGAAATCAGCCTCAGGCTCCTGGCGTGGAAGCTAGCGGAGCTGGC
GAAGCTAGAGCCTCTACAGGCAGCAGCGATTCTTCTCCTGGCGGCCACG
GCACACAAGTGAACGTGACCTGTATCGTGAACGTGTGCTCCAGCAGCGA
CCACAGCAGCCAGTGTAGCTCTCAGGCCAGCAGCACCATGGGCGATACA
GATAGCAGCCCTAGCGAGAGCCCCAAGGATGAACAGGTGCCCTTCAGCA
AAGAGGAATGCGCCTTCAGAAGCCAGCTGGAAACCCCTGAGACACTGCT
GGGCAGCACCGAGGAAAAACCTCTGCCTCTGGGAGTGCCCGACGCCGGA
ATGAAGCCTAGCAGAGTGAAGTTCAGCCGCAGCGCCGATGCTCCTGCCT
ATAAGCAGGGACAGAACCAGCTGTACAACGAGCTGAATCTGGGGCGCAG
AGAAGAGTACGATGTGCTGGACAAGCGGAGAGGCAGGGATCCTGAGATG
GGCGGCAAGCCCAGACGGAAGAATCCTCAAGAGGGCCTGTATAATGAGC
TGCAGAAAGACAAGATGGCCGAGGCCTACAGCGAGATCGGCATGAAGGG
CGAGCGCAGAAGAGGCAAGGGACACGATGGACTGTATCAGGGCCTGAGC
ACCGCCACCAAGGATACCTATGATGCCCTGCACATGCAGGCCCTGCCTC
CAAGA

An exemplary protein sequence encoding a CSR 2DH.2TF2z WT protein of the disclosure comprises or consists of the amino acid sequence of (CD2 signal peptide, CD2 extra cellular domain (D111H), CD2 transmembrane domain, TNFR2 intracellular domain #1, CD3z intracellular domain #2):

(SEQ ID NO: 18)
MSFPCKFVASFLLIFNVSSKGAVSKEITNALETWGALGQDINLDIPSFQ
MSDDIDDIKWEKTSDKKKIAQFRKEKETFKEKDTYKLFKNGTLKIKHLK
TDDQDIYKVSIYHTKGKNVLEKIFDLKIQERVSKPKISWTCINTTLTCE
VMNGTDPELNLYQDGKHLKLSQRVITHKWTTSLSAKFKCTAGNKVSKES
SVEPVSCPEKGLDIYLIIGICGGGSLLMVFVALLVFYITKKKPLCLQRE
AKVPHLPADKARGTQGPEQQHLLITAPSSSSSLESSASALDRRAPTRNQ
PQAPGVEASGAGEARASTGSSDSSPGGHGTQVNVTCIVNVCSSSDHSSQ
CSSQASSTMGDTDSSPSESPKDEQVPFSKEECAFRSQLETPETLLGSTE
EKPLPLGVPDAGMKPSRVKFSRSADAPAYKQGQNQLYNELNLGRREEYD
VLDKRRGRDPEMGGKPRRKNPQEGLYNELQKDKMAEAYSEIGMKGERRR
GKGHDGLYQGLSTATKDTYDALHMQALPPR

An exemplary nucleotide sequence encoding a CSR 2DH.2TF2z WT protein of the disclosure comprises or consists of the amino acid sequence of (CD2 signal peptide, CD2 extra cellular domain (D111H), CD2 transmembrane domain, TNFR2 intracellular domain #1, CD3z intracellular domain #2):

(SEQ ID NO: 171)
ATGAGCTTCCCTTGCAAGTTCGTGGCCAGCTTCCTGCTGATCTTCAACG
TGTCCTCTAAGGGCGCCGTGTCCAAAGAGATCACAAACGCCCTGGAAAC
CTGGGGAGCCCTCGGCCAGGATATTAACCTGGACATCCCCAGCTTCCAG
ATGAGCGACGACATCGATGACATCAAGTGGGAGAAAACCAGCGACAAGA
AGAAGATCGCCCAGTTCCGGAAAGAGAAAGAGACATTCAAAGAGAAGGA
CACCTACAAGCTGTTCAAGAACGGCACCCTGAAGATCAAGCACCTGAAA
ACCGACGACCAGGACATCTATAAGGTGTCCATCTACCACACCAAGGGCA
AGAACGTGCTGGAAAAGATCTTCGACCTCAAGATCCAAGAGCGGGTGTC
CAAGCCTAAGATCAGCTGGACCTGCATCAACACCACACTGACCTGCGAA
GTGATGAACGGCACAGACCCCGAGCTGAACCTGTACCAGGATGGCAAAC
ACCTGAAGCTGAGCCAGCGCGTGATCACCCACAAGTGGACAACAAGCCT
GAGCGCCAAGTTCAAGTGCACCGCCGGAAACAAAAGTGTCTAAAGAGTC
CAGCGTCGAGCCCGTGTCTTGCCCTGAAAAAGGACTGGACATCTACCTG
ATCATCGGCATCTGTGGCGGCGGAAGCCTGCTGATGGTGTTTGTGGCTC
TGCTGGTGTTCTACATCACCAAGAAAAAGCCCCTGTGCCTGCAGCGCGA
GGCCAAAGTTCCTCATCTGCCTGCCGATAAGGCCAGAGGCACAGGGACC
TGAACAGCAGCATCTGCTGATCACAGCCCCTAGCAGCAGCAGCTCTAGC
CTGGAATCTAGCGCCAGCGCTCTGGATAGAAGGGCCCCTACCAGAAACC
AGCCTCAGGCTCCTGGTGTTGAAGCTAGCGGAGCTGGCGAAGCCAGAGC
CTCTACAGGCAGCAGCGATTCTTCTCCTGGCGGCCACGGCACCCAAGTG
AACGTGACCTGTATCGTGAACGTGTGCAGCTCCAGCGACCACAGCAGCC
AGTGTAGCTCTCAGGCCAGCAGCACCATGGGCGATACAGATAGCAGCCC
TAGCGAGAGCCCCAAGGATGAACAGGTGCCCTTCAGCAAAGAGGAATGC
GCCTTCAGAAGCCAGCTGGAAACCCCTGAGACACTGCTGGGCAGCACCG
AGGAAAAACCTCTGCCTCTGGGAGTGCCCGACGCCGGAATGAAGCCTAG
CAGAGTGAAGTTCAGCCGCAGCGCCGATGCTCCTGCCTATAAGCAGGGA
CAGAACCAGCTGTACAACGAGCTGAATCTGGGGCGCAGAGAAGAGTACG
ATGTGCTGGACAAGCGGAGAGGCAGGGATCCTGAGATGGGCGGCAAGCC
CAGACGGAAGAATCCTCAAGAGGGCCTGTATAATGAGCTGCAGAAAGAC
AAGATGGCCGAGGCCTACAGCGAGATCGGCATGAAGGGCGAGCGCAGAA
GAGGCAAGGGACACGATGGACTGTATCAGGGCCTGAGCACCGCCACCAA
GGATACCTATGATGCCCTGCACATGCAGGCCCTGCCTCCAAGA

An exemplary protein sequence encoding a CSR 82DH.TF2z protein of the disclosure comprises or consists of the amino acid sequence of (CD8 signal peptide, CD2 extra cellular domain (D111H), TNFR2 transmembrane domain, TNFR2 intracellular domain #1, CD3z intracellular domain #2):

(SEQ ID NO: 37)
MALPVTALLLPLALLLHAARPKEITNALETWGALGQDINLDIPSFQMSD
DIDDIKWEKTSDKKKIAQFRKEKETFKEKDTYKLFKNGTLKIKHLKTDD
QDIYKVSIYHTKGKNVLEKIFDLKIQERVSKPKISWTCINTTLTCEVMN
GTDPELNLYQDGKHLKLSQRVITHKWTTSLSAKFKCTAGNKVSKESSVE
PVSCPEKGLDFALPVGLIVGVTALGLLIIGVVNCVIMTQVKKKPLCLQR
EAKVPHLPADKARGTQGPEQQHLLITAPSSSSSSLESSASALDRRAPTR
NQPQAPGVEASGAGEARASTGSSDSSPGGHGTQVNVTCIVNVCSSSDHS
SQCSSQASSTMGDTDSSPSESPKDEQVPFSKEECAFRSQLETPETLLGS
TEEKPLPLGVPDAGMKPSRVKFSRSADAPAYKQGQNQLYNELNLGRREE
YDVLDKRRGRDPEMGGKPRRKNPQEGLYNELQKDKMAEAYSEIGMKGER
RRGKGHDGLYQGLSTATKDTYDALHMQALPPR

An exemplary nucleotide sequence encoding a CSR 82DH.TF2z protein of the disclosure comprises or consists of the amino acid sequence of (CD8 signal peptide, CD2 extra cellular domain (D111H), TNFR2 transmembrane domain, TNFR2 intracellular domain #1, CD3z intracellular domain #2):

(SEQ ID NO: 172)
ATGGCTCTGCCTGTGACAGCTCTGCTGCTGCCTCTGGCTCTGCTTCTGC
ATGCCGCCAGACCTAAAGAGATCACAAACGCCCTGGAAACCTGGGGAGC
CCTCGGCCAGGATATTAACCTGGACATCCCCAGCTTCCAGATGAGCGAC
GACATCGATGACATCAAGTGGGAGAAAACCAGCGACAAGAAGAAGATCG
CCCAGTTCCGGAAAGAGAAAGAGACATTCAAAGAGAAGGACACCTACAA
GCTGTTCAAGAACGGCACCCTGAAGATCAAGCACCTGAAAACCGACGAC
CAGGACATCTATAAGGTGTCCATCTACCACACCAAGGGCAAGAACGTGC
TGGAAAAGATCTTCGACCTCAAGATCCAAGAGCGGGTGTCCAAGCCTAA
GATCAGCTGGACCTGCATCAACACCACACTGACCTGCGAAGTGATGAAC
GGCACAGACCCCGAGCTGAACCTGTACCAGGATGGCAAACACCTGAAGC
TGAGCCAGCGCGTGATCACCCACAAGTGGACAACAAGCCTGAGCGCCAA
GTTCAAGTGCACCGCCGGAAACAAAGTGTCCAAAGAAAGCAGCGTGGAA
CCCGTGTCTTGCCCCGAGAAAGGCCTGGATTTTGCCCTGCCTGTGGGCC
TGATTGTGGGAGTGACTGCTCTGGGCCTGCTGATCATCGGCGTGGTCAA
CTGCGTGATCATGACCCAAGTGAAGAAAAAGCCCCTGTGCCTGCAGCGC
GAGGCCAAAGTTCCTCATCTGCCTGCCGATAAGGCCAGGGGAACACAGG
GACCTGAACAGCAGCATCTGCTGATTACAGCCCCTAGCAGCAGCTCCAG
CAGCCTGGAATCTTCTGCCAGCGCTCTGGATCGGAGAGCCCCTACCAGA
AATCAGCCTCAGGCTCCTGGCGTGGAAGCTAGCGGAGCTGGCGAAGCTA
GAGCCTCTACAGGCAGCTCTGATAGCTCTCCTGGCGGCCACGGCACACA
AGTGAACGTGACCTGTATCGTGAACGTGTGCAGCTCCTCCGACCACAGC
AGCCAGTGTAGCTCTCAGGCCAGCAGCACCATGGGCGATACAGATAGCA
GCCCTAGCGAGAGCCCCAAGGATGAACAGGTGCCCTTCAGCAAAGAGGA
ATGCGCCTTCAGAAGCCAGCTGGAAACCCCTGAGACACTGCTGGGCAGC
ACCGAGGAAAAACCTCTGCCACTGGGAGTGCCCGACGCCGGAATGAAGC
CTAGCAGAGTGAAGTTCAGCAGAAGCGCCGACGCTCCCGCCTATAAGCA
GGGACAGAACCAGCTGTACAACGAGCTGAATCTGGGGCGCAGAGAAGAG
TACGATGTGCTGGACAAGCGGAGAGGCAGAGATCCTGAGATGGGCGGCA
AGCCCAGACGGAAGAATCCTCAAGAGGGCCTGTATAATGAGCTGCAGAA
AGACAAGATGGCCGAGGCCTACAGCGAGATCGGCATGAAGGGCGAGCGC
AGAAGAGGCAAGGGACACGATGGACTGTATCAGGGCCTGAGCACCGCCA
CCAAGGATACCTATGATGCCCTGCACATGCAGGCCCTGCCTCCAAGA

An exemplary protein sequence encoding a CSR 82DH.2TF2z WT protein of the disclosure comprises or consists of the amino acid sequence of (CD8 signal peptide, CD2 extra cellular domain (D111H), CD2 transmembrane domain, TNFR2 intracellular domain #1, CD3z intracellular domain #2):

(SEQ ID NO: 38)
MALPVTALLLPLALLLHAARPKEITNALETWGALGQDINLDIPSFQMSD
DIDDIKWEKTSDKKKIAQFRKEKETFKEKDTYKLFKNGTLKIKHLKTDD
QDIYKVSIYHTKGKNVLEKIFDLKIQERVSKPKISWTCINTTLTCEVMN
GTDPELNLYQDGKHLKLSQRVITHKWTTSLSAKFKCTAGNKVSKESSVE
PVSCPEKGLDIYLIIGICGGGSLLMVFVALLVFYITKKKPLCLQREAKV
PHLPADKARGTQGPEQQHLLITAPSSSSSSLESSASALDRRAPTRNQPQ
APGVEASGAGEARASTGSSDSSPGGHGTQVNVTCIVNVCSSSDHSSQCS
SQASSTMGDTDSSPSESPKDEQVPFSKEECAFRSQLETPETLLGSTEEK
PLPLGVPDAGMKPSRVKFSRSADAPAYKQGQNQLYNELNLGRREEYDVL
DKRRGRDPEMGGKPRRKNPQEGLYNELQKDKMAEAYSEIGMKGERRRGK
GHDGLYQGLSTATKDTYDALHMQALPRR

An exemplary nucleotide sequence encoding a CSR 82DH.2TF2z WT protein of the disclosure comprises or consists of the amino acid sequence of (CD8 signal peptide, CD2 extra cellular domain (D111H), CD2 transmembrane domain, TNFR2 intracellular domain #1, CD3z intracellular domain #2):

(SEQ ID NO: 173)
ATGGCTCTGCCTGTGACAGCTCTGCTGCTGCCTCTGGCTCTGCTTCTGC
ATGCCGCCAGACCTAAAGAGATCACAAACGCCCTGGAAACCTGGGGAGC
CCTCGGCCAGGATATTAACCTGGACATCCCCAGCTTCCAGATGAGCGAC
GACATCGATGACATCAAGTGGGAGAAAACCAGCGACAAGAAGAAGATCG
CCCAGTTCCGGAAAGAGAAAGAGACATTCAAAGAGAAGGACACCTACAA
GCTGTTCAAGAACGGCACCCTGAAGATCAAGCACCTGAAAACCGACGAC
CAGGACATCTATAAGGTGTCCATCTACCACACCAAGGGCAAGAACGTGC
TGGAAAAGATCTTCGACTTCAAGATCCAAGAGCGGGTGTCCAAGCCTAA
GATCAGCTGGACCTGCATCAACACCACACTGACCTGCGAAGTGATGAAC
GGCACAGACCCCGAGCTGAACCTGTACCAGGATGGCAAACACCTGAAGC
TGAGCCAGCGCGTGATCACCCACAAGTGGACAACAAGCCTGAGCGCCAA
GTTCAAGTGCACCGCCGGAAACAAAGTGTCCAAAGAAAGCAGCGTGGAA
CCCGTGTCTTGCCCCGAGAAAGGACTGGACATCTACCTGATCATCGGCA
TCTGTGGCGGCGGAAGCCTGCTGATGGTGTTTGTTGCCCTGCTGGTGTT
CTACATCACCAAGAAAAAGCCCCTGTGCCTGCAGCGCGAGGCCAAAGTT
CCTCATCTGCCTGCCGATAAGGCCAGGGGAACACAGGGACCTGAACAGC
AGCATCTGCTGATCACAGCCCCTAGCAGCAGCTCCAGCAGCCTGGAATC
TTCTGCCAGCGCTCTGATCGGAGAGCCCCTACCAGAAATCAGCCTCAGG
CTCCTGGCGTGGAAGCTAGCGGAGCTGGCGAAGCTAGAGCCTCTACAGG
CAGCTCTGATAGCTCTCCTGGCGGCCACGGCACCCAAGTGAATGTGACC
TGTATCGTGAACGTGTGCAGCTCCTCCGACCACAGCAGCCAGTGTAGCT
CTCAGGCCAGCAGCACCATGGGCGATACAGATAGCAGCCCTAGCGAGAG
CCCCAAGGATGAACAGGTGCCCTTCAGCAAAGAGGAATGCGCCTTCAGA
AGCCAGCTGGAAACCCCTGAGACACTGCTGGGCAGCACCGAGGAAAAAC
CTCTGCCACTGGGAGTGCCCCGACGCCGGAATGAAGCCTAGCAGAGTGA
AGTTCAGCAGAAGCGCCGACGCTCCCGCCTATAAGCAGGGACAGAACCA
GCTGTACAACGAGCTGAATCTGGGGCGCAGAGAAGAGTACGATGTGCTG
GACAAGCGGAGAGGCAGAGATCCTGAGATGGGCGGCAAGCCCAGACGGA
AGAATCCTCAAGAGGGCCTGTATAATGAGCTGCAGAAAGACAAGATGGC
CGAGGCCTACAGCGAGATCGGCATGAAGGGCGAGCGCAGAAGAGGCAAG
GGACACGATGGACTGTATCAGGGCCTGAGCACCGCCACCAAGGATACCT
ATGATGCCCTGCACATGCAGGCCCTGCCTCCAAGA

An exemplary protein sequence encoding a CSR BBBz protein of the disclosure comprises or consists of the amino acid sequence of (IL2RB signal peptide, IL2RB extra cellular domain, IL2RB transmembrane domain, IL2RB intracellular domain #1, CD3z intracellular domain #2):

(SEQ ID NO: 19)
MAAPALSWRLPLLILLLPLATSWASAAVNGTSQFTCFYNSRANISCVWSQDGALQDTSCQVH
AWPDRRRWNQTCELLPVSQASWACNLILGAPDSQKLTTVDIVTLRVLCREGVRWRVMAIQDF
KPFENLRLMAPISLQVVHVETHRCNISWEISQASHYFERHLEFEARTLSPGHTWEEAPLLTL
KQKQEWICLETLTPDTQYEFQVRVKPLQGEFTTWSPWSQPLAFRTKPAALGKDTIPWLGHLL
VGLSGAFGFIILVYLLINCRNTGPWLKKVLKCNTPDPSKFFSQLSSEHGGDVQKWLSSPFPS
SSFSPGGLAPEISPLEVLERDKVTQLLLQQDKVPEPASLSSNHSLTSCFTNQGYFFFHLPDA
LEIEACQVYFTYDPYSEEDPDEGVAGAPTGSSPQPLQPLSGEDDAYCTFPSRDDLLLFSPSL
LGGPSPPSTAPGGSGAGEERMPPSLQERVPRDWDPQPLGPPTPGVPDLVDFQPPPELVLREA
GEEVPDAGPREGVSFPWSRPPGQGEFRALNARLPLNTDAYLSLQELQGQDPTHLVRVKFSRS
ADAPAYKQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR

An exemplary nucleotide sequence encoding a CSR BBBz protein of the disclosure comprises or consists of the amino acid sequence of (IL2RB signal peptide, IL2RB extra cellular domain, IL2RB transmembrane domain, IL2RB intracellular domain #1, CD3z intracellular domain #2):

(SEQ ID NO: 174)
ATGGCTGCCCCTGCTCTGTCTTGGAGACTGCCTCTGCTGATCCTGCTGCTGCCTCTGGCTAC
ATCTTGGGCCTCTGCCGCCGTGAATGGCACCAGCCAGTTCACCTGTTTCTACAACAGCCGGG
CCAACATCAGCTGCGTGTGGTCACAAGATGGCGCCCTGCAGGATACCAGCTGTCAGGTTCAC
GCCTGGCCTGATCGGAGAAGATGGAACCAGACATGCGAGCTGCTGCCCGTGTCTCAGGCTAG
CTGGGCCTGTAATCTGATCCTGGGAGCCCCTGACAGCCAGAAACTGACCACAGTGGACATCG
TGACCCTGCGGGTGCTGTGTAGAGAAGGCGTTCGTTGGAGAGTGATGGCCATTCAGGACTTC
AAGCCCTTCGAGAACCTGCGGCTGATGGCCCCAATTAGCCTGCAGGTTGTGCACGTGGAAAC
CCACCGGTGCAATATCAGCTGGGAGATCAGCCAGGCCAGCCACTACTTCGAGCGGCACCTGG
AATTCGAGGCCAGAACACTGAGCCCTGGCCACACCTGGGAAGAAGCTCCTCTGCTCACCCTG
AAGCAGAAACAAGAGTGGATCTGCCTGGAAACCCTGACACCTGACACACAGTACGAGTTCCA
AGTGCGCGTGAAGCCTCTCCAGGGCGAGTTTACAACATGGTCCCCTTGGAGTCAGCCCCTGG
CCTTTAGAACAAAGCCTGCCGCTCTGGGCAAAGACACAATCCCGGGGCGGGGCCAACGGCGC
GTTGGACTGTCTGGCGCCTTCGGCTTCATCATCCTGGTGTACCTGCTGATCAACTGCCGGAA
CACAGGCCCCTGGCTGAAGAAAGTGCTGAAGTGCAACACCCCTGATCCGAGCAAGTTCTTTA
GCCAGCTGAGCAGCGAGCATGGCGGCGACGTTCAGAAATGGCTGTCTAGCCCATTTCCTAGC
AGCAGCTTCAGCCCTGGTGGACTGGCCCCTGAGATTAGCCCTCTGGAAGTGCTGGAACGGGA
CAAAGTGACCCAGCTGCTTCTGCAGCAGGACAAGGTGCCAGAACCTGCCAGCCTGTCCAGCA
ATCACAGCCTGACCAGCTGTTTTACAAACCAGGGCTACTTCTTCTTCCATCTGCCTGACGCA
CTGGAAATTGAGGCCTGCCAGGTGTACTTCACCTACGATCCCTACAGCGAAGAGGACCCCGA
TGAAGGTGTTGCCGGTGCTCCTACCGGAAGCTCTCCTCAACCTCTGCAACCACTGAGCGGCG
AGGATGACGCCTACTGCACATTCCCCAGCAGAGATGACCTGCTCCTGTTCAGCCCTTCTCTG
CTCGGCGGACCTTCTCCACCATCTACAGCTCCAGGTGGAAGCGGAGCCGGCGAGGAAAGAAT
GCCTCCAAGCCTGCAAGAGCGGGTGCCCAGAGATTGGGATCCTCAACCACTGGGCCCTCCAA
CACCTGGCGTGCCAGATCTCGTGGATTTCCAGCCTCCTCCAGAGCTGGTGCTGAGAGAAGCT
GGCGAAGAAGTGCCAGACGCTGGCCCTAGAGAGGGCGTTAGCTTTCCTTGGAGCAGACCTCC
TGGACAGGGCGAATTCAGAGCCCTGAATGCCAGACTGCCCCTGAACACCGATGCCTACCTGT
CTCTGCAAGAACTGCAGGGACAAGACCCCACACACCTCGTGCGAGTGAAGTTCAGCAGAAGC
GCTGACGCCCCTGCCTACAAGCAGGGACAGAACCAGCTGTACAACGAGCTGAACCTGGGGAG
AAGAGAAGAGTACGACGTGCTGGACAAGCGGAGAGGCAGAGATCCTGAGATGGGCGGCAAGC
CCAGACGGAAGAATCCTCAAGAGGGCCTGTATAATGAGCTGCAGAAAGATAAGATGGCCGAG
GCCTACTCCGAGATCGGCATGAAGGGCGAGCGCAGAAGAGGCAAGGGACACGATGGACTGTA
CCAGGGCCTGAGCACCGCCACCAAGGATACCTATGATGCCCTGCACATGCAGGCCCTGCCAC
CTAGA

An exemplary protein sequence encoding a CSR 2BB protein of the disclosure comprises or consists of the amino acid sequence of (CD2 signal peptide, CD2 extra cellular domain (D111H), IL2RB transmembrane domain, IL2RB intracellular domain):

(SEQ ID NO: 20)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIPWLGHLLVGLSGAFGFIILVYLLINCRNTGPWLKKVLK
CNTPDPSKFFSQLSSEHGGDVQKWLSSPFPSSSFSPGGLAPEISPLEVLERDKVTQLLLQQD
KVPEPASLSSNHSLTSCFTNQGYFFFHLPDALEIEACQVYFTYDPYSEEDPDEGVAGAPTGS
SPQPLQPLSGEDDAYCTFPSRDDLLLFSPSLLGGPSPPSTAPGGSGAGEERMPPSLQERVPR
DWDPQPLGPPTPGVPDLVDFQPPPELVLREAGEEVPDAGPREGVSFPWSRPPGQGEFRALNA
RLPLNTDAYLSLQELQGQDPTHLV

An exemplary nucleotide sequence encoding a CSR 2BB protein of the disclosure comprises or consists of the amino acid sequence of (CD2 signal peptide, CD2 extra cellular domain (D111H), IL2RB transmembrane domain, IL2RB intracellular domain):

(SEQ ID NO: 175)
ATGAGCTTCCCTTGCAAGTTCGTGGCCAGCTTCCTGCTGATCTTCAACGTGTCCTCTAAGGG
CGCCGTGTCCAAAGAGATCACAAACGCCCTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
CCTGGACATTCCCTGGCTGGGCCATCTGCTTGTTGGACTGTCTGGCGCCTTCGGCTTCATCA
TCCTGGTGTATCTGCTGATCAACTGCCGGAACACAGGCCCCTGGCTGAAGAAAGTGCTGAAG
TGCAACACCCCTGATCCGAGCAAGTTCTTTAGCCAGCTGAGCAGCGAGCATGGCGGCGACGT
TCAGAAATGGCTGTCTAGCCCATTTCCTAGCAGCAGCTTCAGCCCTGGTGGACTGGCCCCTG
AGATTAGCCCTCTGGAAGTGCTGGAACGGGACAAAGTGACCCAGCTGCTCCTGCAGCAGGAT
AAGGTGCCAGAACCTGCCAGCCTGTCCAGCAATCACAGCCTGACCAGCTGCTTTACCAACCA
GGGCTACTTCTTCTTCCATCTGCCTGACGCTCTGGAAATCGAGGCCTGCCAGGTGTACTTCA
CCTACGATCCCTACAGCGAAGAGGACCCCGATGAAGGTGTTGCCGGTGCTCCTACCGGAAGC
TCTCCTCAACCTCTGCAACCACTGAGCGGCGAGGATGACGCCTACTGCACATTCCCCAGCAG
AGATGACCTGCTGCTGTTCAGCCCTTCTCTGCTCGGTGGACCTTCTCCACCTTCTACAGCAC
CTGGCGGATCTGGCGCAGGCGAGGAAAGAATGCCTCCTAGCCTGCAAGAGAGAGTGCCCAGA
GACTGGGATCCTCAGCCTCTCGGACCTCCTACACCAGGCGTGCCAGACCTGGTGGATTTTCA
GCCTCCTCCAGAGCTGGTGCTGAGAGAAGCTGGCGAAGAAGTGCCAGACGCCGGACCTAGAG
AGGGCGTTAGCTTTCCTTGGAGCAGACCTCCTGGACAGGGCGAGTTCAGAGCCCTGAATGCT
AGACTGCCCCTGAACACCGATGCCTACCTGTCTCTGCAAGAGCTGCAGGGACAAGACCCCAC
ACACCTGGTG

An exemplary protein sequence encoding a CSR 2BBz protein of the disclosure comprises or consists of the amino acid sequence of (CD2 signal peptide, CD2 extra cellular domain (D111H), IL2RB transmembrane domain, IL2RB intracellular domain #1, CD3z intracellular domain #2):

(SEQ ID NO: 21)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIPWLGHLLVGLSGAFGFIILVYLLINCRNTGPWLKKVLK
CNTPDPSKFFSQLSSEHGGDVQKWLSSPFPSSSFSPGGLAPEISPLEVLERDKVTQLLLQQD
KVPEPASLSSNHSLTSCFTNQGYFFFHLPDALEIEACQVYFTYDPYSEEDPDEGVAGAPTGS
SPQPLQPLSGEDDAYCTFPSRDDLLLFSPSLLGGPSPPSTAPGGSGAGEERMPPSLQERVPR
DWDPQPLGPPTPGVPDLVDFQPPPELVLREAGEEVPDAGPREGVSFPWSRPPGQGEFRALNA
RLPLNTDAYLSLQELQGQDPTHLVRVKFSRSADAPAYKQGQNQLYNELNLGRREEYDVLDKR
RGRDPEMGGKPRRKNPQEGLYNELQKDKMAEAYSEIGMKGERRRGKGHDGLYQGLSTATKDT
YDALHMQALPPR

An exemplary nucleotide sequence encoding a CSR 2BBz protein of the disclosure comprises or consists of the amino acid sequence of (CD2 signal peptide, CD2 extra cellular domain (D111H), IL2RB transmembrane domain, IL2RB intracellular domain #1, CD3z intracellular domain #2):

(SEQ ID NO: 176)
ATGAGCTTCCCTTGCAAGTTCGTGGCCAGCTTCCTGCTGATCTTCAACGTGTCCTCTAAGGG
CGCCGTGTCCAAAGAGATCACAAACGCCCTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
CCTGGACATTCCCTGGCTGGGCCATCTGCTTGTTGGACTGTCTGGCGCCTTCGGCTTCATCA
TCCTGGTGTATCTGCTGATCAACTGCCGGAACACAGGCCCCTGGCTGAAGAAAGTGCTGAAG
TGCAACACCCCTGATCCGAGCAAGTTCTTTAGCCAGCTGAGCAGCGAGCATGGCGGCGACGT
TCAGAAATGGCTGTCTAGCCCATTTCCTAGCAGCAGCTTCAGCCCTGGTGGACTGGCCCCTG
AGATTAGCCCTCTGGAAGTGCTGGAACGGGACAAAGTGACCCAGCTGCTCCTGCAGCAGGAT
AAGGTGCCAGAACCTGCCAGCCTGTCCAGCAATCACAGCCTGACCAGCTGCTTTACCAACCA
GGGCTACTTCTTCTTCCATCTGCCTGACGCTCTGGAAATCGAGGCCTGCCAGGTGTACTTCA
CCTACGATCCCTACAGCGAAGAGGACCCCGATGAAGGTGTTGCCGGTGCTCCTACCGGAAGC
TCTCCTCAACCTCTGCAACCACTGAGCGGCGAGGATGACGCCTACTGCACATTCCCCAGCAG
AGATGACCTGCTGCTGTTCAGCCCTTCTCTGCTCGGTGGACCTTCTCCACCTTCTACAGCAC
CTGGCGGATCTGGCGCAGGCGAGGAAAGAATGCCTCCTAGCCTGCAAGAGAGAGTGCCCAGA
GACTGGGATCCTCAGCCTCTCGGACCTCCTACACCAGGCGTGCCAGACCTGGTGGATTTTCA
GCCTCCTCCAGAGCTGGTGCTGAGAGAAGCTGGCGAAGAAGTGCCAGACGCCGGACCTAGAG
AGGGCGTTAGCTTTCCTTGGAGCAGACCTCCTGGACAGGGCGAGTTCAGAGCCCTGAATGCT
AGACTGCCCCTGAACACCGATGCCTACCTGTCTCTGCAAGAGCTGCAGGGACAAGACCCCAC
ACACCTCGTCAGAGTGAAGTTCAGCCGCAGCGCTGATGCCCCTGCCTACAAGCAGGGACAGA
ACCAGCTGTACAACGAGCTGAATCTGGGGCGCAGAGAAGAGTACGATGTGCTGGACAAGCGG
AGAGGCAGAGATCCTGAGATGGGCGGCAAGCCCAGACGGAAGAATCCTCAAGAGGGCCTGTA
TAATGAGCTGCAGAAAGACAAGATGGCCGAGGCCTACTCCGAGATCGGAATGAAGGGCGAGC
GCAGAAGAGGCAAGGGACACGATGGACTGTATCAGGGCCTGAGCACCGCCACCAAGGATACC
TATGATGCCCTGCACATGCAGGCCCTGCCTCCAAGA

An exemplary protein sequence encoding a CSR 22B protein of the disclosure comprises or consists of the amino acid sequence of (CD2 signal peptide, CD2 extra cellular domain (D111H), CD2 transmembrane domain, IL2RB intracellular domain):

(SEQ ID NO: 22)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYLIIGICGGGSLLMVFVALLVFYITNCRNTGPWLKKVL
KCNTPDPSKFFSQLSSEHGGDVQKWLSSPFPSSSFSPGGLAPEISPLEVLERDKVTQLLLQQ
DKVPEPASLSSNHSLTSCFTNQGYFFFHLPDALEIEACQVYFTYDPYSEEDPDEGVAGAPTG
SSPQPLQPLSGEDDAYCTFPSRDDLLLFSPSLLGGPSPPSTAPGGSGAGEERMPPSLQERVP
RDWDPQPLGPPTPGVPDLVDFQPPPELVLREAGEEVPDAGPREGVSFPWSRPPGQGEFRALN
ARLPLNTDAYLSLQELQGQDPTHLV

An exemplary nucleotide sequence encoding a CSR 22B protein of the disclosure comprises or consists of the amino acid sequence of (CD2 signal peptide, CD2 extra cellular domain (D111H), CD2 transmembrane domain, IL2RB intracellular domain):

(SEQ ID NO: 177)
ATGAGCTTCCCTTGCAAGTTCGTGGCCAGCTTCCTGCTGATCTTCAACGTGTCCTCTAAGGG
CGCCGTGTCCAAAGAGATCACAAACGCCCTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGAAGCCTGCTGATGGTGTTTGTGG
CTCTGCTGGTGTTCTACATCACCAACTGCCGGAACACAGGCCCCTGGCTGAAGAAAGTGCTG
AAGTGCAACACCCCTGATCCGAGCAAGTTCTTTAGCCAGCTGAGCAGCGAGCATGGCGGCGA
CGTTCAGAAATGGCTGTCTAGCCCATTTCCTAGCAGCAGCTTCAGCCCTGGTGGACTGGCCC
CTGAGATTAGCCCTCTGGAAGTGCTGGAACGGGACAAAGTGACCCAGCTGCTCCTGCAGCAG
GATAAGGTGCCAGAACCTGCCAGCCTGTCCAGCAATCACAGCCTGACCAGCTGCTTTACCAA
CCAGGGCTACTTCTTCTTCCATCTGCCTGACGCTCTGGAAATCGAGGCCTGCCAGGTGTACT
TCACCTACGATCCCTACAGCGAAGAGGACCCCGATGAAGGTGTTGCCGGTGCTCCTACCGGA
AGCTCTCCTCAACCTCTGCAACCACTGAGCGGCGAGGATGACGCCTACTGCACATTCCCCAG
CAGAGATGACCTGCTGCTGTTCAGCCCTTCTCTGCTCGGTGGACCTTCTCCACCTTCTACAG
CACCTGGCGGATCTGGCGCAGGCGAGGAAAGAATGCCTCCTAGCCTGCAAGAGAGAGTGCCC
AGAGACTGGGATCCTCAGCCTCTCGGACCTCCTACACCAGGCGTGCCAGACCTGGTGGATTT
TCAGCCTCCTCCAGAGCTGGTGCTGAGAGAAGCTGGCGAAGAAGTGCCAGACGCCGGACCTA
GAGAGGGCGTTAGCTTTCCTTGGAGCAGACCTCCTGGACAGGGCGAGTTCAGAGCCCTGAAT
GCTAGACTGCCCCTGAACACCGATGCCTACCTGTCTCTGCAAGAGCTGCAGGGACAAGACCC
CACACACCTGGTG

An exemplary protein sequence encoding a CSR 22Bz protein of the disclosure comprises or consists of the amino acid sequence of (CD2 signal peptide, CD2 extra cellular domain (D111H), CD2 transmembrane domain, IL2RB intracellular domain #1, CD3z intracellular domain #2):

(SEQ ID NO: 23)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYLIIGICGGGSLLMVFVALLVFYITNCRNTGPWLKKVL
KCNTPDPSKFFSQLSSEHGGDVQKWLSSPFPSSSFSPGGLAPEISPLEVLERDKVTQLLLQQ
DKVPEPASLSSNHSLTSCFTNQGYFFFHLPDALEIEACQVYFTYDPYSEEDPDEGVAGAPTG
SSPQPLQPLSGEDDAYCTFPSRDDLLLFSPSLLGGPSPPSTAPGGSGAGEERMPPSLQERVP
RDWDPQPLGPPTPGVPDLVDFQPPPELVLREAGEEVPDAGPREGVSFPWSRPPGQGEFRALN
ARLPLNTDAYLSLQELQGQDPTHLVRVKFSRSADAPAYKQGQNQLYNELNLGRREEYDVLDK
RRGRDPEMGGKPRRKNPQEGLYNELQKDKMAEAYSEIGMKGERRRGKGHDGLYQGLSTATKD
TYDALHMQALPPR

An exemplary nucleotide sequence encoding a CSR 22Bz protein of the disclosure comprises or consists of the amino acid sequence of (CD2 signal peptide, CD2 extra cellular domain (D111H), CD2 transmembrane domain, IL2RB intracellular domain #1, CD3z intracellular domain #2):

(SEQ ID NO: 178)
ATGAGCTTCCCTTGCAAGTTCGTGGCCAGCTTCCTGCTGATCTTCAACGTGTCCTCTAAGGG
CGCCGTGTCCAAAGAGATCACAAACGCCCTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGAAGCCTGCTGATGGTGTTTGTGG
CTCTGCTGGTGTTCTACATCACCAACTGCCGGAACACAGGCCCCTGGCTGAAGAAAGTGCTG
AAGTGCAACACCCCTGATCCGAGCAAGTTCTTTAGCCAGCTGAGCAGCGAGCATGGCGGCGA
CGTTCAGAAATGGCTGTCTAGCCCATTTCCTAGCAGCAGCTTCAGCCCTGGTGGACTGGCCC
CTGAGATTAGCCCTCTGGAAGTGCTGGAACGGGACAAAGTGACCCAGCTGCTCCTGCAGCAG
GATAAGGTGCCAGAACCTGCCAGCCTGTCCAGCAATCACAGCCTGACCAGCTGCTTTACCAA
CCAGGGCTACTTCTTCTTCCATCTGCCTGACGCTCTGGAAATCGAGGCCTGCCAGGTGTACT
TCACCTACGATCCCTACAGCGAAGAGGACCCCGATGAAGGTGTTGCCGGTGCTCCTACCGGA
AGCTCTCCTCAACCTCTGCAACCACTGAGCGGCGAGGATGACGCCTACTGCACATTCCCCAG
CAGAGATGACCTGCTGCTGTTCAGCCCTTCTCTGCTCGGTGGACCTTCTCCACCTTCTACAG
CACCTGGCGGATCTGGCGCAGGCGAGGAAAGAATGCCTCCTAGCCTGCAAGAGAGAGTGCCC
AGAGACTGGGATCCTCAGCCTCTCGGACCTCCTACACCAGGCGTGCCAGACCTGGTGGATTT
TCAGCCTCCTCCAGAGCTGGTGCTGAGAGAAGCTGGCGAAGAAGTGCCAGACGCCGGACCTA
GAGAGGGCGTTAGCTTTCCTTGGAGCAGACCTCCTGGACAGGGCGAGTTCAGAGCCCTGAAT
GCTAGACTGCCCCTGAACACCGATGCCTACCTGTCTCTGCAAGAGCTGCAGGGACAAGACCC
CACACACCTCGTCAGAGTGAAGTTCAGCCGCAGCGCTGATGCCCCTGCCTACAAGCAGGGAC
AGAACCAGCTGTACAACGAGCTGAATCTGGGGCGCAGAGAAGAGTACGATGTGCTGGACAAG
CGGAGAGGCAGAGATCCTGAGATGGGCGGCAAGCCCAGACGGAAGAATCCTCAAGAGGGCCT
GTATAATGAGCTGCAGAAAGACAAGATGGCCGAGGCCTACTCCGAGATCGGAATGAAGGGCG
AGCGCAGAAGAGGCAAGGGACACGATGGACTGTATCAGGGCCTGAGCACCGCCACCAAGGAT
ACCTATGATGCCCTGCACATGCAGGCCCTGCCTCCAAGA

An exemplary protein sequence encoding a CSR GGGz protein of the disclosure comprises or consists of the amino acid sequence of (IL2RG signal peptide, IL2RG extra cellular domain, IL2RG transmembrane domain, IL2RG intracellular domain #1, CD3z intracellular domain #2):

(SEQ ID NO: 24)
MLKPSLPFTSLLFLQLPLLGVGLNTTTLTPNGNEDTTADFFLTTMPTDSLSVSTLPLPEVQC
FVFNVEYMNCTWNSSSEPQPTNLTLHYWYKNSDNDKVQKCSHYLFSEEITSGCQLQKKEIHL
YQTFVVQLQDPREPRRQATQMLKLQNLVIPWAPENLTLHKLSESQLELNWNNRFLNHCLEHL
VQYRTDWDHSWTEQSVDYRHKFSLPSVDGQKRYTFRVRSRFNPLCGSAQHWSEWSHPIHWGS
NTSKENPFLFALEAVVISVGSMGLIISLLCVYFWLERTMPRIPTLKNLEDLVTEYHGNFSAW
SGVSKGLAESLQPDYSERLCLVSEIPPKGGALGEGPGASPCNQHSPYWAPPCYTLKPETRVK
FSRSADAPAYKQGQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR

An exemplary nucleotide sequence encoding a CSR GGGz protein of the disclosure comprises or consists of the amino acid sequence of (IL2RG signal peptide, IL2RG extra cellular domain, IL2RG transmembrane domain, IL2RG intracellular domain #1, CD3z intracellular domain #2):

(SEQ ID NO: 179)
ATGCTGAAGCCCAGCCTGCCTTTTACCAGCCTGCTGTTCCTGCAGCTGCCTCTGCTTGGCGT
GGGCCTGAATACCACCATCCTGACACCTAACGGCAACGAGGATACAACCGCCGACTTCTTCC
TGACCACCATGCCTACCGATAGCCTGAGCGTGTCCACACTGCCACTGCCTGAGGTGCAGTGC
TTCGTGTTCAACGTCGAGTACATGAACTGCACCTGGAACAGCTCCAGCGAGCCCCAGCCTAC
CAATCTGACACTGCACTACTGGTACAAGAACAGCGACAACGACAAGGTGCAGAAGTGCAGCC
ACTACCTGTTCAGCGAGGAAATCACCAGCGGCTGCCAGCTGCAGAAGAAAGAGATCCACCTG
TACCAGACCTTCGTGGTGCAGCTCCAGGATCCTAGAGAGCCTAGAAGGCAGGCCACACAGAT
GCTGAAACTGCAGAACCTGGTCATCCCCTGGGCTCCCGAAAACCTGACTCTGCACAAGCTGA
GCGAGAGCCAGCTGGAACTGAACTGGAACAACCGGTTCCTGAATCACTGCCTGGAACACCTG
GTGCAGTACCGGACCGATTGGGATCACAGCTGGACAGAGCAGAGCGTGGACTACCGGCACAA
GTTCAGCCTGCCATCTGTGGACGGCCAGAAGCGGTACACCTTTAGAGTGCGGAGCCGGTTCA
ATCCCCTGTGTGGATCTGCTCAGCATTGGAGCGAGTGGTCACACCCTATCCACTGGGGCAGC
AACACCAGCAAAGAGAACCCCTTCCTGTTCGCCCTGGAAGCCGTGGTTATCAGCGTGGGCTC
TATGGGCCTGATCATCTCCCTGCTGTGCGTGTACTTCTGGCTGGAAAGAACCATGCCTCGGA
TCCCCACACTGAAGAATCTGGAAGATCTGGTCACCGAGTACCACGGCAACTTCAGTGCTTGG
AGCGGCGTGTCAAAAGGCCTGGCCGAATCTCTGCAGCCCGACTACTCTGAGAGACTGTGCCT
GGTGTCTGAGATCCCTCCTAAAGGCGGAGCCCTCGGAGAAGGACCTGGCGCCTCTCCATGTA
ATCAGCACAGCCCTTATTGGGCCCCTCCTTGCTACACCCTGAAGCCTGAGACAAGAGTGAAG
TTCAGCAGAAGCGCCGACGCTCCCGCCTATAAGCAGGGACAGAACCAGCTGTACAACGAGCT
GAACCTGGGGAGAAGAGAAGAGTACGACGTGCTGGACAAGCGGAGAGGCAGAGATCCTGAGA
TGGGCGGCAAGCCCAGACGGAAGAATCCTCAAGAGGGCCTGTATAATGAGCTGCAAAAGGAC
AAGATGGCCGAGGCCTACAGCGAGATCGGAATGAAGGGCGAGCGCAGAAGAGGCAAGGGACA
CGATGGACTGTACCAGGGCCTGAGCACCGCCACCAAGGATACCTATGATGCCCTGCACATGC
AGGCCCTGCCTCCAAGA

An exemplary protein sequence encoding a CSR 2GG protein of the disclosure comprises or consists of the amino acid sequence of (CD2 signal peptide, CD2 extra cellular domain (D111H), IL2RG transmembrane domain, IL2RG intracellular domain):

(SEQ ID NO: 25)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDAVVISVGSMGLIISLLCVYFWLERTMPRIPTLKNLEDLV
TEYHGNFSAWSGVSKGLAESLQPDYSERLCLVSEIPPKGGALGEGPGASPCNQHSPYWAPPC
YTLKPET

An exemplary nucleotide sequence encoding a CSR 2GG protein of the disclosure comprises or consists of the amino acid sequence of (CD2 signal peptide, CD2 extra cellular domain (D111H), IL2RG transmembrane domain, IL2RG intracellular domain)

(SEQ ID NO: 180)
ATGAGCTTCCCTTGCAAGTTCGTGGCCAGCTTCCTGCTGATCTTCAACGTGTCCTCTAAGGG
CGCCGTGTCCAAAGAGATCACAAACGCCCTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAGAAAGG
ACTGGATGCCGTGGTCATCAGCGTGGGCTCTATGGGCCTGATCATCAGCCTGCTGTGCGTGT
ACTTCTGGCTGGAACGGACCATGCCTCGGATCCCCACACTGAAGAACCTGGAAGATCTGGTC
ACCGAGTACCACGGCAACTTCTCTGCTTGGAGCGGCGTCAGTAAAGGCCTGGCCGAATCTCT
GCAGCCCGACTACTCTGAGAGACTGTGCCTGGTGTCTGAGATCCCTCCTAAAGGCGGAGCCC
TTGGAGAAGGACCTGGCGCCTCTCCATGTAACCAGCACAGCCCTTATTGGGCCCCTCCTTGC
TACACTCTGAAGCCCGAGACA

An exemplary protein sequence encoding a CSR 2GGz protein of the disclosure comprises or consists of the amino acid sequence of (CD2 signal peptide, CD2 extra cellular domain (D111H), IL2RG transmembrane domain, IL2RG intracellular domain #1, CD3z intracellular domain #2):

(SEQ ID NO: 26)
MSFPCKFVASFLLIFNVSSKGAVSKEITNALETWGALGQDINLDTPSFQ
MSDDIDDTKWEKTSDKKKIAQFRKEKETFKEKDTYKLFKNGTLKIKHLK
TDDQDIYKVSIYHTKGKNVLEKIFDLKIQERVSKPKISWTCINTTLTCE
VMNGTDPELNLYQDGKHLKLSQRVITHKWTTSLSAKFKCTAGNKVSKES
SVEPVSCPEKGLDAVVISVGSMGLIISLLCVYFWLERTMPRIPTLKNLE
DLVTEYHGNFSAWSGVSKGLAESLQPDYSERLCLVSEIPPKGGALGEGP
GASPCNQHSPYWAPPCYTLKPETRVKFSRSADAPAYKQGQNQLYNELNL
GRREEYDVLDKRRGRDPEMGGKPRRKNPQEGLYNELQKDKMAEAYSEIG
MKGERRRGKGHDGLYQGLSTATKDTYDALHMQALPPR

An exemplary nucleotide sequence encoding a CSR 2GGz protein of the disclosure comprises or consists of the amino acid sequence of (CD2 signal peptide, CD2 extra cellular domain (D111H), IL2RG transmembrane domain, IL2RG intracellular domain #1, CD3z intracellular domain #2):

(SEQ ID NO: 181)
ATGAGCTTCCCTTGCAAGTTCGTGGCCAGCTTCCTGCTGATCTTCAACGTGTCCTCTAAGGG
CGCCGTGTCCAAAGAGATCACAAACGCCCTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAGAAAGG
ACTGGATGCCGTGGTCATCAGCGTGGGCTCTATGGGCCTGATCATCAGCCTGCTGTGCGTGT
ACTTCTGGCTGGAACGGACCATGCCTCGGATCCCCACACTGAAGAACCTGGAAGATCTGGTC
ACCGAGTACCACGGCAACTTCTCTGCTTGGAGCGGCGTCAGTAAAGGCCTGGCCGAATCTCT
GCAGCCCGACTACTCTGAGAGACTGTGCCTGGTGTCTGAGATCCCTCCTAAAGGCGGAGCCC
TTGGAGAAGGACCTGGCGCCTCTCCATGTAACCAGCACAGCCCTTATTGGGCCCCTCCTTGC
TACACTCTGAAGCCCGAGACAAGAGTGAAGTTCAGCAGATCCGCCGAGGCTCCCGCCTATAA
GCAGGGACAGAACCAGCTGTACAACGAGCTGAATCTGGGGCGCAGAGAAGAGTACGATGTGC
TGGACAAGCGGAGAGGCAGAGATCCTGAGATGGGCGGCAAGCCCAGACGGAAGAATCCTCAA
GAGGGCCTGTATAATGAGCTGCAGAAAGACAAGATGGCCGAGGCCTACAGCGAGATCGGAAT
GAAGGGCGAGCGCAGAAGAGGCAAGGGACACGATGGACTGTATCAGGGCCTGAGCACCGCCA
CCAAGGATACCTATGATGCCCTGCACATGCAGGCCCTGCCTCCAAGA

An exemplary protein sequence encoding a CSR 22G protein of the disclosure comprises or consists of the amino acid sequence of (CD2 signal peptide, CD2 extra cellular domain (D111H), CD2 transmembrane domain, IL2RG intracellular domain):

(SEQ ID NO: 27)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYEIIGICGGGSLLMVFVALLVFYITERTMPRIPTLKNL
EDLVTEYHGNFSAWSGVSKGLAESLQPDYSERLCLVSEIPPKGGALGEGPGASPCNQHSPYW
APPCYTLKPET

An exemplary nucleotide sequence encoding a CSR 22G protein of the disclosure comprises or consists of the amino acid sequence of (CD2 signal peptide, CD2 extra cellular domain (D111H), CD2 transmembrane domain, IL2RG intracellular domain):

(SEQ ID NO: 182)
ATGAGCTTCCCTTGCAAGTTCGTGGCCAGCTTCCTGCTGATCTTCAACGTGTCCTCTAAGGG
CGCCGTGTCCAAAGAGATCACAAACGCCCTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGAAGCCTGCTGATGGTGTTTGTGG
CTCTGCTGGTGTTCTACATCACCGAGCGGACCATGCCTCGGATCCCCACACTGAAGAACCTG
GAAGATCTGGTCACCGAGTACCACGGCAACTTCTCTGCTTGGAGCGGCGTCAGTAAAGGCCT
GGCCGAATCTCTGCAGCCCGACTACTCTGAGAGACTGTGCCTGGTGTCTGAGATCCCTCCTA
AAGGCGGAGCCCTTGGAGAAGGACCTGGCGCCTCTCCATGTAACCAGCACAGCCCTTATTGG
GCCCCTCCTTGCTACACTCTGAAGCCCGAGACA

An exemplary protein sequence encoding a CSR 22 Gz protein of the disclosure comprises or consists of the amino acid sequence of (CD2 signal peptide, CD2 extra cellular domain (D111H), CD2 transmembrane domain, IL2RG intracellular domain #1, CD3z intracellular domain #2):

(SEQ ID NO: 28)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYEIIGICGGGSLLMVFVALLVFYITERTMPRIPTLKNL
EDLVTEYHGNFSAWSGVSKGLAESLQPDYSERLCLVSEIPPKGGALGEGPGASPCNQHSPYW
APPCYTLKPETRVKFSRSADAPAYKQGQNQLYNELNLGRREEYDVLDKRRGRDPEMGGKPRR
KNPQEGLYNELQKDKMAEAYSEIGMKGERRRGKGHDGLYQGLSTATKDTYDALHMQALPPR

An exemplary nucleotide sequence encoding a CSR 22 Gz protein of the disclosure comprises or consists of the amino acid sequence of (CD2 signal peptide, CD2 extra cellular domain (D111H), CD2 transmembrane domain, IL2RG intracellular domain #1, CD3z intracellular domain #2):

(SEQ ID NO: 183)
ATGAGCTTCCCTTGCAAGTTCGTGGCCAGCTTCCTGCTGATCTTCAACGTGTCCTCTAAGGG
CGCCGTGTCCAAAGAGATCACAAACGCCCTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGAAGCCTGCTGATGGTGTTTGTGG
CTCTGCTGGTGTTCTACATCACCGAGCGGACCATGCCTCGGATCCCCACACTGAAGAACCTG
GAAGATCTGGTCACCGAGTACCACGGCAACTTCTCTGCTTGGAGCGGCGTCAGTAAAGGCCT
GGCCGAATCTCTGCAGCCCGACTACTCTGAGAGACTGTGCCTGGTGTCTGAGATCCCTCCTA
AAGGCGGAGCCCTTGGAGAAGGACCTGGCGCCTCTCCATGTAACCAGCACAGCCCTTATTGG
GCCCCTCCTTGCTACACTCTGAAGCCCGAGACAAGAGTGAAGTTCAGCAGATCCGCCGAGGC
TCCCGCCTATAAGCAGGGACAGAACCAGCTGTACAACGAGCTGAATCTGGGGCGCAGAGAAG
AGTACGATGTGCTGGACAAGCGGAGAGGCAGAGATCCTGAGATGGGCGGCAAGCCCAGACGG
AAGAATCCTCAAGAGGGCCTGTATAATGAGCTGCAGAAAGACAAGATGGCCGAGGCCTACAG
CGAGATCGGAATGAAGGGCGAGCGCAGAAGAGGCAAGGGACACGATGGACTGTATCAGGGCC
TGAGCACCGCCACCAAGGATACCTATGATGCCCTGCACATGCAGGCCCTGCCTCCAAGA

An exemplary protein sequence encoding a CSR 2BBG protein of the disclosure comprises or consists of the amino acid sequence of (CD2 signal peptide, CD2 extra cellular domain (D111H), IL2RB transmembrane domain, IL2RB intracellular domain #1, IL2RG intracellular domain #2):

(SEQ ID NO: 29)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIPWLGHLLVGLSGAFGFIILVYLLINCRNTGPWLKKVLK
CNTPDPSKFFSQLSSEHGGDVQKWLSSPFPSSSFSPGGLAPEISPLEVLERDKVTQLLLQQD
KVPEPASLSSNHSLTSCFTNQGYFFFHLPDALEIEACQVYFTYDPYSEEDPDEGVAGAPTGS
SPQPLQPLSGEDDAYCTFPSRDDLLLFSPSLLGGPSPPSTAPGGSGAGEERMPPSLQERVPR
DWDPQPLGPPTPGVPDLVDFQPPPELVLREAGEEVPDAGPREGVSFPWSRPPGQGEFRALNA
RLPLNTDAYLSLQELQGQDPTHLVERTMPRIPTLKNLEDLVTEYHGNFSAWSGVSKGLAESL
QPDYSERLCLVSEIPPKGGALGEGPGASPCNQHSPYWAPPCYTLKPET

An exemplary nucleotide sequence encoding a CSR 2BBG protein of the disclosure comprises or consists of the amino acid sequence of (CD2 signal peptide, CD2 extra cellular domain (D111H), IL2RB transmembrane domain, IL2RB intracellular domain #1, IL2RG intracellular domain #2):

(SEQ ID NO: 184)
ATGAGCTTCCCTTGCAAGTTCGTGGCCAGCTTCCTGCTGATCTTCAACGTGTCCTCTAAGGG
CGCCGTGTCCAAAGAGATCACAAACGCCCTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
CCTGGACATTCCCTGGCTGGGCCATCTGCTTGTTGGACTGTCTGGCGCCTTCGGCTTCATCA
TCCTGGTGTATCTGCTGATCAACTGCCGGAACACAGGCCCCTGGCTGAAGAAAGTGCTGAAG
TGCAACACCCCTGATCCGAGCAAGTTCTTTAGCCAGCTGAGCAGCGAGCATGGCGGCGACGT
TCAGAAATGGCTGTCTAGCCCATTTCCTAGCAGCAGCTTCAGCCCTGGTGGACTGGCCCCTG
AGATTAGCCCTCTGGAAGTGCTGGAACGGGACAAAGTGACCCAGCTGCTCCTGCAGCAGGAT
AAGGTGCCAGAACCTGCCAGCCTGTCCAGCAATCACAGCCTGACCAGCTGCTTTACCAACCA
GGGCTACTTCTTCTTCCATCTGCCTGACGCTCTGGAAATCGAGGCCTGCCAGGTGTACTTCA
CCTACGATCCCTACAGCGAAGAGGACCCCGATGAAGGTGTTGCCGGTGCTCCTACCGGAAGC
TCTCCTCAACCTCTGCAACCACTGAGCGGCGAGGATGACGCCTACTGCACATTCCCCAGCAG
AGATGACCTGCTGCTGTTCAGCCCTTCTCTGCTCGGTGGACCTTCTCCACCTTCTACAGCAC
CTGGCGGATCTGGCGCAGGCGAGGAAAGAATGCCTCCTAGCCTGCAAGAGAGAGTGCCCAGA
GACTGGGATCCTCAGCCTCTCGGACCTCCTACACCAGGCGTGCCAGACCTGGTGGATTTTCA
GCCTCCTCCAGAGCTGGTGCTGAGAGAAGCTGGCGAAGAAGTGCCAGACGCCGGACCTAGAG
AGGGCGTTAGCTTTCCTTGGAGCAGACCTCCTGGACAGGGCGAGTTCAGAGCCCTGAATGCT
AGACTGCCCCTGAACACCGATGCCTACCTGTCTCTGCAAGAGCTGCAGGGACAAGACCCCAC
ACATCTGGTGGAACGGAGCATGCCTAGAATGCCGAGACTGAAGAACCTGGAAGATCTGGTCA
CCGAGTACCACGGCAACTTCAGTGCTTGGAGCGGCGTCAGCAAAGGACTGGCCGAATCTCTG
CAGCCCGACTACAGCGAGAGACTGTGCCTGGTGTCTGAGATCCCTCCTAAAGGCGGAGCCCT
TGGAGAAGGACCTGGCGCCTCTCCATGTAACCAGCACAGCCCTTATTGGGCCCCTCCTTGCT
ACACTCTGAAGCCCGAGACA

An exemplary protein sequence encoding a CSR 2BBGz protein of the disclosure comprises or consists of the amino acid sequence of (CD2 signal peptide, CD2 extra cellular domain (D111H), IL2RB transmembrane domain, IL2RB intracellular domain #1, IL2RG intracellular domain #2, CD3z intracellular domain #3):

(SEQ ID NO: 30)
MSFPCKFVASFLLIFNVSSKGAVSKEITNALETWGALGQDINLDTPSFQMSDDIDDTKWEKT
SDKKKPAQFRKEKETFKEKDTYKEFKNGTEKPKHEKTDDQDPYKVSPYHTKGKNVEEKPFDP
KIQERVSKPKISWTCINTTLTCEVMNGTDPELNLYQDGKHLKLSQRVITHKWTTSLSAKFKC
TAGNKVSKESSVEPVSCPEKGLDIPWLGHLLVGLSGAFGFIILVYLLINCRNTGPWLKKVLK
CNTPDPSKFFSQLSSEHGGDVQKWLSSPFPSSSFSPGGLAPEISPLEVLERDKVTQLLLQQD
KVPEPASLSSNHSLTSCFTNQGYFFFHLPDALEIEACQVYFTYDPYSEEDPDEGVAGAPTGS
SPQPLQPLSGEDDAYCTFPSRDDLLLFSPSLLGGPSPPSTAPGGSGAGEERMPPSLQERVPR
DWDPQPLGPPTPGVPDLVDFQPPPELVLREAGEEVPDAGPREGVSFPWSRPPGQGEFRALNA
RLPLNTDAYLSLQELQGQDPTHLVERTMPRIPTLKNLEDLVTEYHGNFSAWSGVSKGLAESL
QPDYSERLCLVSEIPPKGGALGEGPGASPCNQHSPYWAPPCYTLKPETRVKFSRSADAPAYK
QGQNQLYNELNLGRREEYDVLDKRRGRDPEMGGKPRRKNPQEGLYNEEQKDKMAEAYSEPGM
KGERRRGKGHDGLYQGLSTATKDTYDALHMQALPPR

An exemplary nucleotide sequence encoding a CSR 2BBGz protein of the disclosure comprises or consists of the amino acid sequence of (CD2 signal peptide, CD2 extra cellular domain (D111H), IL2RB transmembrane domain, IL2RB intracellular domain #1, IL2RG intracellular domain #2, CD3z intracellular domain #3):

(SEQ ID NO: 185)
ATGAGCTTCCCTTGCAAGTTCGTGGCCAGCTTCCTGCTGATCTTCAACGTGTCCTCTAAGGG
CGCCGTGTCCAAAGAGATCACAAACGCCCTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
CCTGGACATTCCCTGGCTGGGCCATCTGCTTGTTGGACTGTCTGGCGCCTTCGGCTTCATCA
TCCTGGTGTATCTGCTGATCAACTGCCGGAACACAGGCCCCTGGCTGAAGAAAGTGCTGAAG
TGCAACACCCCTGATCCGAGCAAGTTCTTTAGCCAGCTGAGCAGCGAGCATGGCGGCGACGT
TCAGAAATGGCTGTCTAGCCCATTTCCTAGCAGCAGCTTCAGCCCTGGTGGACTGGCCCCTG
AGATTAGCCCTCTGGAAGTGCTGGAACGGGACAAAGTGACCCAGCTGCTCCTGCAGCAGGAT
AAGGTGCCAGAACCTGCCAGCCTGTCCAGCAATCACAGCCTGACCAGCTGCTTTACCAACCA
GGGCTACTTCTTCTTCCATCTGCCTGACGCTCTGGAAATCGAGGCCTGCCAGGTGTACTTCA
CCTACGATCCCTACAGCGAAGAGGACCCCGATGAAGGTGTTGCCGGTGCTCCTACCGGAAGC
TCTCCTCAACCTCTGCAACCACTGAGCGGCGAGGATGACGCCTACTGCACATTCCCCAGCAG
AGATGACCTGCTGCTGTTCAGCCCTTCTCTGCTCGGTGGACCTTCTCCACCTTCTACAGCAC
CTGGCGGATCTGGCGCAGGCGAGGAAAGAATGCCTCCTAGCCTGCAAGAGAGAGTGCCCAGA
GACTGGGATCCTCAGCCTCTCGGACCTCCTACACCAGGCGTGCCAGACCTGGTGGATTTTCA
GCCTCCTCCAGAGCTGGTGCTGAGAGAAGCTGGCGAAGAAGTGCCAGACGCCGGACCTAGAG
AGGGCGTTAGCTTTCCTTGGAGCAGACCTCCTGGACAGGGCGAGTTCAGAGCCCTGAATGCT
AGACTGCCCCTGAACACCGATGCCTACCTGTCTCTGCAAGAGCTGCAGGGACAAGACCCCAC
ACATCTGGTGGAACGGAGCATGCCTAGAATGCCGAGACTGAAGAACCTGGAAGATCTGGTCA
CCGAGTACCACGGCAACTTCAGTGCTTGGAGCGGCGTCAGCAAAGGACTGGCCGAATCTCTG
CAGCCCGACTACAGCGAGAGACTGTGCCTGGTGTCTGAGATCCCTCCTAAAGGCGGAGCCCT
TGGAGAAGGACCTGGCGCCTCTCCATGTAACCAGCACAGCCCTTATTGGGCCCCTCCTTGCT
ACACTCTGAAGCCCGAGACAAGAGTGAAGTTCAGCCGCAGCGCTGATGCCCCTGCCTACAAG
CAGGGACAGAACCAGCTGTACAACGAGCTGAATCTGGGGCGCAGAGAAGAGTACGATGTGCT
GGACAAGCGGAGAGGCAGAGATCCTGAGATGGGCGGCAAGCCCAGACGGAAGAATCCTCAAG
AGGGCCTGTATAATGAGCTGCAGAAAGACAAGATGGCCGAGGCCTACTCCGAGATCGGAATG
AAGGGCGAGCGCAGAAGAGGCAAGGGACACGATGGACTGTATCAGGGCCTGAGCACCGCCAC
CAAGGATACCTATGATGCCCTGCACATGCAGGCCCTGCCTCCAAGA

An exemplary protein sequence encoding a CSR 22BG protein of the disclosure comprises or consists of the amino acid sequence of (CD2 signal peptide, CD2 extra cellular domain (D111H), CD2 transmembrane domain, IL2RB intracellular domain #1, IL2RG intracellular domain #2):

(SEQ ID NO: 31)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYLIIGICGGGSLLMVFVALLVFYITNCRNTGPWLKKVL
KCNTPDPSKFFSQLSSEHGGDVQKWLSSPFPSSSFSPGGLAPEISPLEVLERDKVTQLLLQQ
DKVPEPASLSSNHSLTSCFTNQGYFFFHLPDALEIEACQVYFTYDPYSEEDPDEGVAGAPTG
SSPQPLQPLSGEDDAYCTFPSRDDLLLFSPSLLGGPSPPSTAPGGSGAGEERMPPSLQERVP
RDWDPQPLGPPTPGVPDLVDFQPPPELVLREAGEEVPDAGPREGVSFPWSRPPGQGEFRALN
ARLPLNTDAYLSLQELQGQDPTHLVERTMPRIPTLKNLEDLVTEYHGNFSAWSGVSKGLAES
LQPDYSERLCLVSEIPPKGGALGEGPGASPCNQHSPYWAPPCYTLKPET

An exemplary nucleotide sequence encoding a CSR 22BG protein of the disclosure comprises or consists of the amino acid sequence of (CD2 signal peptide, CD2 extra cellular domain (D111H), CD2 transmembrane domain, IL2RB intracellular domain #1, IL2RG intracellular domain #2):

(SEQ ID NO: 186)
ATGAGCTTCCCTTGCAAGTTCGTGGCCAGCTTCCTGCTGATCTTCAACGTGTCCTCTAAGGG
CGCCGTGTCCAAAGAGATCACAAACGCCCTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGAAGCCTGCTGATGGTGTTTGTGG
CTCTGCTGGTGTTCTACATCACCAACTGCCGGAACACAGGCCCCTGGCTGAAGAAAGTGCTG
AAGTGCAACACCCCTGATCCGAGCAAGTTCTTTAGCCAGCTGAGCAGCGAGCATGGCGGCGA
CGTTCAGAAATGGCTGTCTAGCCCATTTCCTAGCAGCAGCTTCAGCCCTGGTGGACTGGCCC
CTGAGATTAGCCCTCTGGAAGTGCTGGAACGGGACAAAGTGACCCAGCTGCTCCTGCAGCAG
GATAAGGTGCCAGAACCTGCCAGCCTGTCCAGCAATCACAGCCTGACCAGCTGCTTTACCAA
CCAGGGCTACTTCTTCTTCCATCTGCCTGACGCTCTGGAAATCGAGGCCTGCCAGGTGTACT
TCACCTACGATCCCTACAGCGAAGAGGACCCCGATGAAGGTGTTGCCGGTGCTCCTACCGGA
AGCTCTCCTCAACCTCTGCAACCACTGAGCGGCGAGGATGACGCCTACTGCACATTCCCCAG
CAGAGATGACCTGCTGCTGTTCAGCCCTTCTCTGCTCGGTGGACCTTCTCCACCTTCTACAG
CACCTGGCGGATCTGGCGCAGGCGAGGAAAGAATGCCTCCTAGCCTGCAAGAGAGAGTGCCC
AGAGACTGGGATCCTCAGCCTCTCGGACCTCCTACACCAGGCGTGCCAGACCTGGTGGATTT
TCAGCCTCCTCCAGAGCTGGTGCTGAGAGAAGCTGGCGAAGAAGTGCCAGACGCCGGACCTA
GAGAGGGCGTTAGCTTTCCTTGGAGCAGACCTCCTGGACAGGGCGAGTTCAGAGCCCTGAAT
GCTAGACTGCCCCTGAACACCGATGCCTACCTGTCTCTGCAAGAGCTGCAGGGACAAGACCC
CACACATCTGGTGGAACGGAGCATGCCTAGAATCCCGAGACTGAAGAACCTGGAAGATCTGG
TCACCGAGTACCACGGCAACTTCAGTGCTTGGAGCGGCGTCAGTAAAGGCCTGGCCGAATCT
CTGCAGCCCGACTACTCTGAGAGACTGTGCCTGGTGTCTGAGATCCCTCCTAAAGGCGGAGC
CCTTGGAGAAGGACCTGGCGCCTCTCCATGTAACCAGCACAGCCCTTATTGGGCCCCTCCTT
GCTACACTCTGAAGCCCGAGACA

An exemplary protein sequence encoding a CSR 22BGz protein of the disclosure comprises or consists of the amino acid sequence of (CD2 signal peptide, CD2 extra cellular domain (D111H), CD2 transmembrane domain, IL2RB intracellular domain #1, IL2RG intracellular domain #2, CD3z intracellular domain #3):

(SEQ ID NO: 32)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYLIIGICGGGSLLMVFVALLVFYITNCRNTGPWLKKVL
KCNTPDPSKFFSQLSSEHGGDVQKWLSSPFPSSSFSPGGLAPEISPLEVLERDKVTQLLLQQ
DKVPEPASLSSNHSLTSCFTNQGYFFFHLPDALEIEACQVYFTYDPYSEEDPDEGVAGAPTG
SSPQPLQPLSGEDDAYCTFPSRDDLLLFSPSLLGGPSPPSTAPGGSGAGEERMPPSLQERVP
RDWDPQPLGPPTPGVPDLVDFQPPPELVLREAGEEVPDAGPREGVSFPWSRPPGQGEFRALN
ARLPLNTDAYLSLQELQGQDPTHLVERTMPRIPTLKNLEDLVTEYHGNFSAWSGVSKGLAES
LQPDYSERLCLVSEIPPKGGALGEGPGASPCNQHSPYWAPPCYTLKPETRVKFSRSADAPAY
KQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR

An exemplary nucleotide sequence encoding a CSR 22BGz protein of the disclosure comprises or consists of the amino acid sequence of (CD2 signal peptide, CD2 extra cellular domain (D111H), CD2 transmembrane domain, IL2RB intracellular domain #1, IL2RG intracellular domain #2, CD3z intracellular domain #3):

(SEQ ID NO: 187)
ATGAGCTTCCCTTGCAAGTTCGTGGCCAGCTTCCTGCTGATCTTCAACGTGTCCTCTAAGGG
CGCCGTGTCCAAAGAGATCACAAACGCCCTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGAAGCCTGCTGATGGTGTTTGTGG
CTCTGCTGGTGTTCTACATCACCAACTGCCGGAACACAGGCCCCTGGCTGAAGAAAGTGCTG
AAGTGCAACACCCCTGATCCGAGCAAGTTCTTTAGCCAGCTGAGCAGCGAGCATGGCGGCGA
CGTTCAGAAATGGCTGTCTAGCCCATTTCCTAGCAGCAGCTTCAGCCCTGGTGGACTGGCCC
CTGAGATTAGCCCTCTGGAAGTGCTGGAACGGGACAAAGTGACCCAGCTGCTCCTGCAGCAG
GATAAGGTGCCAGAACCTGCCAGCCTGTCCAGCAATCACAGCCTGACCAGCTGCTTTACCAA
CCAGGGCTACTTCTTCTTCCATCTGCCTGACGCTCTGGAAATCGAGGCCTGCCAGGTGTACT
TCACCTACGATCCCTACAGCGAAGAGGACCCCGATGAAGGTGTTGCCGGTGCTCCTACCGGA
AGCTCTCCTCAACCTCTGCAACCACTGAGCGGCGAGGATGACGCCTACTGCACATTCCCCAG
CAGAGATGACCTGCTGCTGTTCAGCCCTTCTCTGCTCGGTGGACCTTCTCCACCTTCTACAG
CACCTGGCGGATCTGGCGCAGGCGAGGAAAGAATGCCTCCTAGCCTGCAAGAGAGAGTGCCC
AGAGACTGGGATCCTCAGCCTCTCGGACCTCCTACACCAGGCGTGCCAGACCTGGTGGATTT
TCAGCCTCCTCCAGAGCTGGTGCTGAGAGAAGCTGGCGAAGAAGTGCCAGACGCCGGACCTA
GAGAGGGCGTTAGCTTTCCTTGGAGCAGACCTCCTGGACAGGGCGAGTTCAGAGCCCTGAAT
GCTAGACTGCCCCTGAACACCGATGCCTACCTGTCTCTGCAAGAGCTGCAGGGACAAGACCC
CACACATCTGGTGGAACGGAGCATGCCTAGAATCCCCACACTGAAGAACCTGGAAGATCTGG
TCACCGAGTACCACGGCAACTTCAGTGCTTGGAGCGGCGTCAGTAAAGGCCTGGCCGAATCT
CTGCAGCCCGACTACTCTGAGAGACTGTGCCTGGTGTCTGAGATCCCTCCTAAAGGCGGAGC
CCTTGGAGAAGGACCTGGCGCCTCTCCATGTAACCAGCACAGCCCTTATTGGGCCCCTCCTT
GCGACACGCGGAAGCCCGAGACAAGAGTGAAGTTCAGCCGCAGCGCTGATGCCCCTGCCTAC
AAGCAGGGACAGAACCAGCTGTACAACGAGCTGAATCTGGGGCGCAGAGAAGAGTACGATGT
GCTGGACAAGCGGAGAGGCAGAGATCCTGAGATGGGCGGCAAGCCCAGACGGAAGAATCCTC
AAGAGGGCCTGTATAATGAGCTGCAGAAAGACAAGATGGCCGAGGCCTACTCCGAGATCGGA
ATGAAGGGCGAGCGCAGAAGAGGCAAGGGACACGATGGACTGTATCAGGGCCTGAGCACCGC
CACCAAGGATACCTATGATGCCCTGCACATGCAGGCCCTGCCTCCAAGA

An exemplary protein sequence encoding a CSR 2GGB protein of the disclosure comprises or consists of the amino acid sequence of (CD2 signal peptide, CD2 extra cellular domain (D111H), IL2RG transmembrane domain, IL2RG intracellular domain #1, IL2RB intracellular domain #2):

(SEQ ID NO: 33)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDAVVISVGSMGLIISLLCVYFWLERTMPRIPTLKNLEDLV
TEYHGNFSAWSGVSKGLAESLQPDYSERLCLVSEIPPKGGALGEGPGASPCNQHSPYWAPPC
YTLKPETNCRNTGPWLKKVLKCNTPDPSKFFSQLSSEHGGDVQKWLSSPFPSSSFSPGGLAP
EISPLEVLERDKVTQLLLQQDKVPEPASLSSNHSLTSCFTNQGYFFFHLPDALEIEACQVYF
TYDPYSEEDPDEGVAGAPTGSSPQPLQPLSGEDDAYCTFPSRDDLLLFSPSLLGGPSPPSTA
PGGSGAGEERMPPSLQERVPRDWDPQPLGPPTPGVPDLVDFQPPPELVLREAGEEVPDAGPR
EGVSFPWSRPPGQGEFRALNARLPLNTDAYLSLQELQGQDPTHLV

An exemplary nucleotide sequence encoding a CSR 2GGB protein of the disclosure comprises or consists of the amino acid sequence of (CD2 signal peptide, CD2 extra cellular domain (D111H), IL2RG transmembrane domain, IL2RG intracellular domain #1, IL2RB intracellular domain #2):

(SEQ ID NO: 188)
ATGAGCTTCCCTTGCAAGTTCGTGGCCAGCTTCCTGCTGATCTTCAACGTGTCCTCTAAGGG
CGCCGTGTCCAAAGAGATCACAAACGCCCTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAGAAAGG
ACTGGATGCCGTGGTCATCAGCGTGGGCTCTATGGGCCTGATCATCAGCCTGCTGTGCGTGT
ACTTCTGGCTGGAACGGACCATGCCTCGGATCCCCACACTGAAGAACCTGGAAGATCTGGTC
ACCGAGTACCACGGCAACTTCTCTGCTTGGAGCGGCGTCAGTAAAGGCCTGGCCGAATCTCT
GCAGCCCGACTACTCTGAGAGACTGTGCCTGGTGTCTGAGATCCCTCCTAAAGGCGGAGCCC
TTGGAGAAGGACCTGGCGCCTCTCCATGTAACCAGCACAGCCCTTATTGGGCCCCTCCTTGC
TACACTCTGAAGCCCGAGACAAACTGCCGGAACACAGGCCCCTGGCTGAAGAAAGTGCTGAA
GTGCAACACCCCTGATCCGAGCAAGTTCTTTAGCCAGCTGAGCAGCGAGCATGGCGGCGACG
TTCAGAAATGGCTGTCTAGCCCATTTCCTAGCAGCAGCTTCAGCCCTGGTGGACTGGCCCCT
GAGATTAGCCCTCTGGAAGTGCTCGAGCGGGACAAAGTGACACAGCTGCTCCTCCAGCAGGA
CAAGGTGCCAGAACCTGCCAGCCTGTCCAGCAATCACAGCCTGACCAGCTGCTTTACCAACC
AGGGCTACTTCTTCTTCCATCTGCCTGACGCTCTGGAAATCGAGGCCTGCCAGGTGTACTTC
ACCTACGATCCCTACAGCGAAGAGGACCCCGATGAAGGTGTTGCCGGTGCTCCTACCGGAAG
CTCTCCTCAACCTCTCCAACCTCTGAGCGGCGAGGATGACGCCTACTGCACATTCCCCAGCA
GAGATGACCTGCTGCTGTTCAGCCCTAGCCTGCTCGGAGGACCTTCTCCACCATCTACAGCT
CCAGGTGGAAGCGGAGCCGGCGAGGAAAGAATGCCTCCAAGCCTGCAAGAGAGAGTGCCCAG
AGACTGGGATCCTCAGCCTCTCGGACCTCCTACACCAGGCGTGCCAGACCTGGTGGATTTTC
AGCCTCCTCCAGAGCTGGTGCTGAGAGAAGCTGGCGAAGAAGTGCCAGACGCCGGACCTAGA
GAGGGCGTTAGCTTTCCTTGGAGCAGACCTCCTGGACAGGGCGAGTTCAGAGCCCTGAATGC
TAGACTGCCCCTGAACACCGATGCCTACCTGTCTCTGCAAGAGCTGCAGGGACAAGACCCCA
CACACCTGGTG

An exemplary protein sequence encoding a CSR 2GGBz protein of the disclosure comprises or consists of the amino acid sequence of (CD2 signal peptide, CD2 extra cellular domain (D111H), IL2RG transmembrane domain, IL2RG intracellular domain #1, IL2RB intracellular domain #2, CD3z intracellular domain #3):

(SEQ ID NO: 34)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDAVVISVGSMGLIISLLCVYFWLERTMPRIPTLKNLEDLV
TEYHGNFSAWSGVSKGLAESLQPDYSERLCLVSEIPPKGGALGEGPGASPCNQHSPYWAPPC
YTLKPETNCRNTGPWLKKVLKCNTPDPSKFFSQLSSEHGGDVQKWLSSPFPSSSFSPGGLAP
EISPLEVLERDKVTQLLLQQDKVPEPASLSSNHSLTSCFTNQGYFFFHLPDALEIEACQVYF
TYDPYSEEDPDEGVAGAPTGSSPQPLQPLSGEDDAYCTFPSRDDLLLFSPSLLGGPSPPSTA
PGGSGAGEERMPPSLQERVPRDWDPQPLGPPTPGVPDLVDFQPPPELVLREAGEEVPDAGPR
EGVSFPWSRPPGQGEFRALNARLPLNTDAYLSLQELQGQDPTHLVRVKFSRSADAPAYKQGQ
NQLYNELNLGRREEYDVLDKRRGRDPEMGGKPRRKNPQEGLYNELQKDKMAEAYSEIGMKGE
RRRGKGHDGLYQGLSTATKDTYDALHMQALPPR

An exemplary nucleotide sequence encoding a CSR 2GGBz protein of the disclosure comprises or consists of the amino acid sequence of (CD2 signal peptide, CD2 extra cellular domain (D111H), IL2RG transmembrane domain, IL2RG intracellular domain #1, IL2RB intracellular domain #2, CD3z intracellular domain #3):

(SEQ ID NO: 189)
ATGAGCTTCCCTTGCAAGTTCGTGGCCAGCTTCCTGCTGATCTTCAACGTGTCCTCTAAGGG
CGCCGTGTCCAAAGAGATCACAAACGCCCTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAGAAAGG
ACTGGATGCCGTGGTCATCAGCGTGGGCTCTATGGGCCTGATCATCAGCCTGCTGTGCGTGT
ACTTCTGGCTGGAACGGACCATGCCTCGGATCCCCACACTGAAGAACCTGGAAGATCTGGTC
ACCGAGTACCACGGCAACTTCTCTGCTTGGAGCGGCGTCAGTAAAGGCCTGGCCGAATCTCT
GCAGCCCGACTACTCTGAGAGACTGTGCCTGGTGTCTGAGATCCCTCCTAAAGGCGGAGCCC
TTGGAGAAGGACCTGGCGCCTCTCCATGTAACCAGCACAGCCCTTATTGGGCCCCTCCTTGC
TACACTCTGAAGCCCGAGACAAACTGCCGGAACACAGGCCCCTGGCTGAAGAAAGTGCTGAA
GTGCAACACCCCTGATCCGAGCAAGTTCTTTAGCCAGCTGAGCAGCGAGCATGGCGGCGACG
TTCAGAAATGGCTGTCTAGCCCATTTCCTAGCAGCAGCTTCAGCCCTGGTGGACTGGCCCCT
GAGATTAGCCCTCTGGAAGTGCTCGAGCGGGACAAAGTGACACAGCTGCTCCTCCAGCAGGA
CAAGGTGCCAGAACCTGCCAGCCTGTCCAGCAATCACAGCCTGACCAGCTGCTTTACCAACC
AGGGCTACTTCTTCTTCCATCTGCCTGACGCTCTGGAAATCGAGGCCTGCCAGGTGTACTTC
ACCTACGATCCCTACAGCGAAGAGGACCCCGATGAAGGTGTTGCCGGTGCTCCTACCGGAAG
CTCTCCTCAACCTCTCCAACCTCTGAGCGGCGAGGATGACGCCTACTGCACATTCCCCAGCA
GAGATGACCTGCTGCTGTTCAGCCCTAGCCTGCTCGGAGGACCTTCTCCACCATCTACAGCT
CCAGGTGGAAGCGGAGCCGGCGAGGAAAGAATGCCTCCAAGCCTGCAAGAGAGAGTGCCCAG
AGACTGGGATCCTCAGCCTCTCGGACCTCCTACACCAGGCGTGCCAGACCTGGTGGATTTTC
AGCCTCCTCCAGAGCTGGTGCTGAGAGAAGCTGGCGAAGAAGTGCCAGACGCCGGACCTAGA
GAGGGCGTTAGCTTTCCTTGGAGCAGACCTCCTGGACAGGGCGAGTTCAGAGCCCTGAATGC
TAGACTGCCCCTGAACACCGATGCCTACCTGTCTCTGCAAGAGCTGCAGGGACAAGACCCCA
CACACCGCGGCAGAGTGAAGTTCAGCAGATCCGCCGACGCTCCCGCCTATAAGCAGGGACAG
AACCAGCTGTACAACGAGCTGAATCTGGGGCGCAGAGAAGAGTACGATGTGCTGGACAAGCG
GAGAGGCAGAGATCCTGAGATGGGCGGCAAGCCCAGACGGAAGAATCCTCAAGAGGGCCTGT
ATAATGAGCTGCAGAAAGACAAGATGGCCGAGGCCTACTCCGAGATCGGAATGAAGGGCGAG
CGCAGAAGAGGCAAGGGACACGATGGACTGTATCAGGGCCTGAGCACCGCCACCAAGGATAC
CTATGATGCCCTGCACATGCAGGCCCTGCCACCTAGA

An exemplary protein sequence encoding a CSR 22 GB protein of the disclosure comprises or consists of the amino acid sequence of (CD2 signal peptide, CD2 extra cellular domain (D111H), CD2 transmembrane domain, IL2RG intracellular domain #1, IL2RB intracellular domain #2):

(SEQ ID NO: 35)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYEIIGICGGGSLLMVFVALLVFYITERTMPRIPTLKNL
EDLVTEYHGNFSAWSGVSKGLAESLQPDYSERLCLVSEIPPKGGALGEGPGASPCNQHSPYW
APPCYTLKPETNCRNTGPWLKKVLKCNTPDPSKFFSQLSSEHGGDVQKWLSSPFPSSSFSPG
GLAPEISPLEVLERDKVTQLLLQQDKVPEPASLSSNHSLTSCFTNQGYFFFHLPDALEIEAC
QVYFTYDPYSEEDPDEGVAGAPTGSSPQPLQPLSGEDDAYCTFPSRDDLLLFSPSLLGGPSP
PSTAPGGSGAGEERMPPSLQERVPRDWDPQPLGPPTPGVPDLVDFQPPPELVLREAGEEVPD
AGPREGVSFPWSRPPGQGEFRALNARLPLNTDAYLSLQELQGQDPTHLV

An exemplary nucleotide sequence encoding a CSR 22 GB protein of the disclosure comprises or consists of the amino acid sequence of (CD2 signal peptide, CD2 extra cellular domain (D111H), CD2 transmembrane domain, IL2RG intracellular domain #1, IL2RB intracellular domain #2):

(SEQ ID NO: 190)
ATGAGCTTCCCTTGCAAGTTCGTGGCCAGCTTCCTGCTGATCTTCAACGTGTCCTCTAAGGG
CGCCGTGTCCAAAGAGATCACAAACGCCCTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGAAGCCTGCTGATGGTGTTTGTGG
CTCTGCTGGTGTTCTACATCACCGAGCGGACCATGCCTCGGATCCCCACACTGAAGAACCTG
GAAGATCTGGTCACCGAGTACCACGGCAACTTCTCTGCTTGGAGCGGCGTCAGTAAAGGCCT
GGCCGAATCTCTGCAGCCCGACTACTCTGAGAGACTGTGCCTGGTGTCTGAGATCCCTCCTA
AAGGCGGAGCCCTTGGAGAAGGACCTGGCGCCTCTCCATGTAACCAGCACAGCCCTTATTGG
GCCCCTCCTTGCTACACTCTGAAGCCCGAGACAAACTGCCGGAACACAGGCCCCTGGCTGAA
GAAAGTGCTGAAGTGCAACACCCCTGATCCGAGCAAGTTCTTTAGCCAGCTGAGCAGCGAGC
ATGGCGGCGACGTTCAGAAATGGCTGTCTAGCCCATTTCCTAGCAGCAGCTTCAGCCCTGGT
GGACTGGCCCCTGAGATTAGCCCTCTGGAAGTGCTGGAACGGGACAAAGTGACCCAGCTGCT
CCTGCAGCAGGATAAGGTGCCAGAACCTGCCAGCCTGTCCAGCAATCACAGCCTGACCAGCT
GCTTTACCAACCAGGGCTACTTCTTCTTCCATCTGCCTGACGCTCTGGAAATCGAGGCCTGC
CAGGTGTACTTCACCTACGATCCCTACAGCGAAGAGGACCCCGATGAAGGTGTTGCCGGTGC
TCCTACCGGAAGCTCTCCTCAACCTCTCCAACCTCTGAGCGGCGAGGATGACGCCTACTGCA
CATTCCCCAGCAGAGATGACCTGCTGCTGTTCAGCCCTAGCCTGCTCGGAGGACCTTCTCCA
CCATCTACAGCTCCAGGTGGAAGCGGAGCCGGCGAGGAAAGAATGCCTCCAAGCCTGCAAGA
GAGAGTGCCCAGAGACTGGGATCCTCAGCCTCTCGGACCTCCTACACCAGGCGTGCCAGACC
TGGTGGATTTTCAGCCTCCTCCAGAGCTGGTGCTGAGAGAAGCTGGCGAAGAAGTGCCAGAC
GCCGGACCTAGAGAGGGCGTTAGCTTTCCTTGGAGCAGACCTCCTGGACAGGGCGAGTTCAG
AGCCCTGAATGCTAGACTGCCCCTGAACACCGATGCCTACCTGTCTCTGCAAGAGCTGCAGG
GACAAGACCCCACACACCTGGTG

An exemplary protein sequence encoding a CSR 22GBz protein of the disclosure comprises or consists of the amino acid sequence of (CD2 signal peptide, CD2 extra cellular domain (D111H), CD2 transmembrane domain, IL2RG intracellular domain #1, IL2RB intracellular domain #2, CD3z intracellular domain #3):

(SEQ ID NO: 36)
MSFPCKFVASFLLIFNVSSKGAVSKEITNALETWGALGQDINLDTPSFQMSDDIDDTKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYEIIGICGGGSLLMVFVALLVFYITERTMPRIPTLKNL
EDLVTEYHGNFSAWSGVSKGLAESLQPDYSERLCLVSEIPPKGGALGEGPGASPCNQHSPYW
APPCYTLKPETNCRNTGPWLKKVLKCNTPDPSKFFSQLSSEHGGDVQKWLSSPFPSSSFSPG
GLAPEISPLEVLERDKVTQLLLQQDKVPEPASLSSNHSLTSCFTNQGYFFFHLPDALEIEAC
QVYFTYDPYSEEDPDEGVAGAPTGSSPQPLQPLSGEDDAYCTFPSRDDLLLFSPSLLGGPSP
PSTAPGGSGAGEERMPPSLQERVPRDWDPQPLGPPTPGVPDLVDFQPPPELVLREAGEEVPD
AGPREGVSFPWSRPPGQGEFRALNARLPLNTDAYLSLQELQGQDPTHLVRVKFSRSADAPAY
KQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR

An exemplary nucleotide sequence encoding a CSR 22GBz protein of the disclosure comprises or consists of the amino acid sequence of (CD2 signal peptide, CD2 extra cellular domain (D111H), CD2 transmembrane domain, IL2RG intracellular domain #1, IL2RB intracellular domain #2, CD3z intracellular domain #3):

(SEQ ID NO: 191)
ATGAGCTTCCCTTGCAAGTTCGTGGCCAGCTTCCTGCTGATCTTCAACGTGTCCTCTAAGGG
CGCCGTGTCCAAAGAGATCACAAACGCCCTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGAAGCCTGCTGATGGTGTTTGTGG
CTCTGCTGGTGTTCTACATCACCGAGCGGACCATGCCTCGGATCCCCACACTGAAGAACCTG
GAAGATCTGGTCACCGAGTACCACGGCAACTTCTCTGCTTGGAGCGGCGTCAGTAAAGGCCT
GGCCGAATCTCTGCAGCCCGACTACTCTGAGAGACTGTGCCTGGTGTCTGAGATCCCTCCTA
AAGGCGGAGCCCTTGGAGAAGGACCTGGCGCCTCTCCATGTAACCAGCACAGCCCTTATTGG
GCCCCTCCTTGCTACACTCTGAAGCCCGAGACAAACTGCCGGAACACAGGCCCCTGGCTGAA
GAAAGTGCTGAAGTGCAACACCCCTGATCCGAGCAAGTTCTTTAGCCAGCTGAGCAGCGAGC
ATGGCGGCGACGTTCAGAAATGGCTGTCTAGCCCATTTCCTAGCAGCAGCTTCAGCCCTGGT
GGACTGGCCCCTGAGATTAGCCCTCTGGAAGTGCTGGAACGGGACAAAGTGACCCAGCTGCT
CCTGCAGCAGGATAAGGTGCCAGAACCTGCCAGCCTGTCCAGCAATCACAGCCTGACCAGCT
GCTTTACCAACCAGGGCTACTTCTTCTTCCATCTGCCTGACGCTCTGGAAATCGAGGCCTGC
CAGGTGTACTTCACCTACGATCCCTACAGCGAAGAGGACCCCGATGAAGGTGTTGCCGGTGC
TCCTACCGGAAGCTCTCCTCAACCTCTCCAACCTCTGAGCGGCGAGGATGACGCCTACTGCA
CATTCCCCAGCAGAGATGACCTGCTGCTGTTCAGCCCTAGCCTGCTCGGAGGACCTTCTCCA
CCATCTACAGCTCCAGGTGGAAGCGGAGCCGGCGAGGAAAGAATGCCTCCAAGCCTGCAAGA
GAGAGTGCCCAGAGACTGGGATCCTCAGCCTCTCGGACCTCCTACACCAGGCGTGCCAGACC
TGGTGGATTTTCAGCCTCCTCCAGAGCTGGTGCTGAGAGAAGCTGGCGAAGAAGTGCCAGAC
GCCGGACCTAGAGAGGGCGTTAGCTTTCCTTGGAGCAGACCTCCTGGACAGGGCGAGTTCAG
AGCCCTGAATGCTAGACTGCCCCTGAACACCGATGCCTACCTGTCTCTGCAAGAGCTGCAGG
GACAAGACCCCACACACCTCGTCAGAGTGAAGTTCAGCAGATCCGCCGACGCTCCCGCCTAT
AAGCAGGGACAGAACCAGCTGTACAACGAGCTGAATCTGGGGCGCAGAGAAGAGTACGATGT
GCTGGACAAGCGGAGAGGCAGAGATCCTGAGATGGGCGGCAAGCCCAGACGGAAGAATCCTC
AAGAGGGCCTGTATAATGAGCTGCAGAAAGACAAGATGGCCGAGGCCTACTCCGAGATCGGA
ATGAAGGGCGAGCGCAGAAGAGGCAAGGGACACGATGGACTGTATCAGGGCCTGAGCACCGC
CACCAAGGATACCTATGATGCCCTGCACATGCAGGCCCTGCCACCTAGA

An exemplary protein sequence encoding a CSR 8BBB protein of the disclosure comprises or consists of the amino acid sequence of (CD8 signal peptide, IL2RB extra cellular domain, IL2RB transmembrane domain, IL2RB intracellular domain):

(SEQ ID NO: 39)
MALPVTALLLPLALLLHAARPAVNGTSQFTCFYNSRANISCVWSQDGALQDTSCQVHAWPDR
RRWNQTCELLPVSQASWACNLILGAPDSQKLTTVDIVTLRVLCREGVRWRVMAIQDFKPFEN
LRLMAPISLQVVHVETHRCNISWEISQASHYFERHLEFEARTLSPGHTWEEAPLLTLKQKQE
WICLETLTPDTQYEFQVRVKPLQGEFTTWSPWSQPLAFRTKPAALGKDTIPWEGHLLVGLSG
AFGFIILVYLLINCRNTGPWLKKVLKCNTPDPSKFFSQLSSEHGGDVQKWLSSPFPSSSFSP
GGLAPEISPLEVLERDKVTQLLLQQDKVPEPASLSSNHSLTSCFTNQGYFFFHLPDALEIEA
CQVYFTYDPYSEEDPDEGVAGAPTGSSPQPLQPLSGEDDAYCTFPSRDDLLLFSPSLLGGPS
PPSTAPGGSGAGEERMPPSLQERVPRDWDPQPLGPPTPGVPDLVDFQPPPELVLREAGEEVP
DAGPREGVSFPWSRPPGQGEFRALNARLPLNTDAYLSLQELQGQDPTHLV

An exemplary nucleotide sequence encoding a CSR 8BBB protein of the disclosure comprises or consists of the amino acid sequence of (CD8 signal peptide, IL2RB extra cellular domain, IL2RB transmembrane domain, IL2RB intracellular domain):

(SEQ ID NO: 192)
ATGGCTCTGCCTGTGACAGCTCTGCTGCTGCCTCTGGCTCTGCTTCTGCATGCTGCTAGACC
TGCCGTGAATGGCACCAGCCAGTTCACCTGTTTCTACAACAGCCGGGCCAACATCAGCTGCG
TGTGGTCACAAGATGGCGCCCTGCAGGATACCAGCTGTCAGGTTCACGCCTGGCCTGATCGG
AGAAGATGGAACCAGACATGCGAGCTGCTGCCCGTGTCTCAGGCTAGCTGGGCCTGTAATCT
GATCCTGGGAGCCCCTGACAGCCAGAAACTGACCACAGTGGACATCGTGACCCTGCGGGTGC
TGTGTAGAGAAGGCGTTCGTTGGAGAGTGATGGCCATTCAGGACTTCAAGCCCTTCGAGAAC
CTGCGGCTGATGGCCCCAATTAGCCTGCAGGTTGTGCACGTGGAAACCCACCGGTGCAATAT
CAGCTGGGAGATCAGCCAGGCCAGCCACTACTTCGAGCGGCACCTGGAATTCGAGGCCAGAA
CACTGAGCCCTGGCCACACCTGGGAAGAAGCTCCTCTGCTGACCCTGAAGCAGAAACAAGAG
TGGATCTGCCTGGAAACCCTGACACCTGACACACAGTACGAGTTCCAAGTGCGCGTGAAGCC
TCTCCAGGGCGAGTTTACAACATGGTCCCCTTGGAGTCAGCCCCTGGCCTTTAGAACAAAGC
CTGCCGCTCTGGGCAAAGACACAATCCCTTGGCTGGGCCATCTGCTCGTTGGACTGTCTGGC
GCCTTCGGCTTCATCATCCTGGTGTACCTGCTGATCAACTGCCGGAACACAGGCCCCTGGCT
GAAGAAAGTGCTGAAGTGCAACACCCCTGATCCGAGCAAGTTCTTTAGCCAGCTGAGCAGCG
AGCATGGCGGCGACGTTCAGAAATGGCTGTCTAGCCCATTTCCTAGCAGCAGCTTCAGCCCT
GGTGGACTGGCCCCTGAGATTAGCCCTCTGGAAGTGCTGGAACGGGACAAAGTGACCCAGCT
GCTCCTGCAGCAGGATAAGGTGCCAGAACCTGCCAGCCTGTCCAGCAATCACAGCCTGACCA
GCTGCTTTACCAACCAGGGCTACTTCTTCTTCCATCTGCCTGACGCACTGGAAATTGAGGCC
TGCCAGGTGTACTTCACCTACGATCCCTACAGCGAAGAGGACCCCGATGAAGGTGTTGCCGG
TGCTCCTACCGGAAGCTCTCCTCAACCTCTGCAACCACTGAGCGGCGAGGATGACGCCTACT
GCACATTCCCCAGCAGAGATGACCTGCTGCTGTTCAGCCCTTCTCTGCTCGGCGGACCTTCT
CCACCATCTACAGCTCCAGGTGGAAGCGGAGCCGGCGAGGAAAGAATGCCTCCAAGCCTGCA
AGAGCGGGTGCCCAGAGATTGGGATCCTCAACCACTGGGCCCTCCAACACCTGGCGTGCCAG
ATCTCGTGGATTTCCAGCCTCCTCCAGAGCTGGTGCTGAGAGAAGCTGGCGAAGAAGTGCCA
GACGCTGGCCCTAGAGAGGGCGTTAGCTTTCCTTGGAGCAGACCTCCTGGACAGGGCGAATT
CAGAGCCCTGAATGCCAGACTGCCCCTGAACACCGATGCCTACCTGTCTCTGCAAGAACTGC
AGGGACAAGACCCCACACACCTGGTG

An exemplary protein sequence encoding a CSR 8BBBz protein of the disclosure comprises or consists of the amino acid sequence of (CD8 signal peptide, IL2RB extra cellular domain, IL2RB transmembrane domain, IL2RB intracellular domain #1, CD3z intracellular domain #2):

(SEQ ID NO: 40)
MALPVTALLLPLALLLHAARPAVNGTSQFTCFYNSRANISCVWSQDGALQDTSCQVHA
WPDRRRWNQTCELLPVSQASWACNLILGAPDSQKLTTVDIVTLRVLCREGVRWRVMAIQD
FKPFENLRLMAPISLQVVHVETHRCNISWEISQASHYFERHLEFEARTLSPGHTWEEAPLL
TLKQKQEWICLETLTPDTQYEFQVRVKPLQGEFTTWSPWSQPLAFRTKPAALGKDTIPWL
GHLLVGLSGAFGFIILVYLLINCRNTGPWLKKVLKCNTPDPSKFFSQLSSEHGGDV
QKWLSSPFPSSSFSPGGLAPEISPLEVLERDKVTQLLLQQDKVPEPASLSSNHSLT
SCFTNQGYFFFHLPDALEIEACQVYFTYDPYSEEDPDEGVAGAPTGSSPQPLQPL
SGEDDAYCTFPSRDDLLLFSPSLLGGPSPPSTAPGGSGAGEERMPPSLQERVPRD
WDPQPLGPPTPGVPDLVDFQPPPELVLREAGEEVPDAGPREGVSFPWSRPPGQG
EFRALNARLPLNTDAYLSLQELQGQDPTHLVRVKFSRSADAPAYKQGQNQLYNEL
NLGRREEYDVLDKRRGRDPEMGGKPRRKNPQEGLYNELQKDKMAEAYSEIGMKGE
RRRGKGHDGLYQGLSTATKDTYDALHMQALPPR

An exemplary nucleotide sequence encoding a CSR 8BBBz protein of the disclosure comprises or consists of the amino acid sequence of (CD8 signal peptide, IL2RB extra cellular domain, IL2RB transmembrane domain, IL2RB intracellular domain #1, CD3z intracellular domain #2):

(SEQ ID NO: 193)
ATGGCTCTGCCTGTGACAGCTCTGCTGCTGCCTCTGGCTCTGCTTCTGCATGCTGCTAGACC
TGCCGTGAATGGCACCAGCCAGTTCACCTGTTTCTACAACAGCCGGGCCAACATCAGCTGCG
TGTGGTCACAAGATGGCGCCCTGCAGGATACCAGCTGTCAGGTTCACGCCTGGCCTGATCGG
AGAAGATGGAACCAGACATGCGAGCTGCTGCCCGTGTCTCAGGCTAGCTGGGCCTGTAATCT
GATCCTGGGAGCCCCTGACAGCCAGAAACTGACCACAGTGGACATCGTGACCCTGCGGGTGC
TGTGTAGAGAAGGCGTTCGTTGGAGAGTGATGGCCATTCAGGACTTCAAGCCCTTCGAGAAC
CTGCGGCTGATGGCCCCAATTAGCCTGCAGGTTGTGCACGTGGAAACCCACCGGTGCAATAT
CAGCTGGGAGATCAGCCAGGCCAGCCACTACTTCGAGCGGCACCTGGAATTCGAGGCCAGAA
CACTGAGCCCTGGCCACACCTGGGAAGAAGCTCCTCTGCTGACCCTGAAGCAGAAACAAGAG
TGGATCTGCCTGGAAACCCTGACACCTGACACACAGTACGAGTTCCAAGTGCGCGTGAAGCC
TCTCCAGGGCGAGTTTACAACATGGTCCCCTTGGAGTCAGCCCCTGGCCTTTAGAACAAAGC
CTGCCGCTCTGGGCAAAGACACAATCCCTTGGCTGGGCCATCTGCTCGTTGGACTGTCTGGC
GCCTTCGGCTTCATCATCCTGGTGTACCTGCTGATCAACTGCCGGAACACAGGCCCCTGGCT
GAAGAAAGTGCTGAAGTGCAACACCCCTGATCCGAGCAAGTTCTTTAGCCAGCTGAGCAGCG
AGCATGGCGGCGACGTTCAGAAATGGCTGTCTAGCCCATTTCCTAGCAGCAGCTTCAGCCCT
GGTGGACTGGCCCCTGAGATTAGCCCTCTGGAAGTGCTGGAACGGGACAAAGTGACCCAGCT
GCTCCTGCAGCAGGATAAGGTGCCAGAACCTGCCAGCCTGTCCAGCAATCACAGCCTGACCA
GCTGCTTTACCAACCAGGGCTACTTCTTCTTCCATCTGCCTGACGCACTGGAAATTGAGGCC
TGCCAGGTGTACTTCACCTACGATCCCTACAGCGAAGAGGACCCCGATGAAGGTGTTGCCGG
TGCTCCTACCGGAAGCTCTCCTCAACCTCTGCAACCACTGAGCGGCGAGGATGACGCCTACT
GCACATTCCCCAGCAGAGATGACCTGCTGCTGTTCAGCCCTTCTCTGCTCGGCGGACCTTCT
CCACCATCTACAGCTCCAGGTGGAAGCGGAGCCGGCGAGGAAAGAATGCCTCCAAGCCTGCA
AGAGCGGGTGCCCAGAGATTGGGATCCTCAACCACTGGGCCCTCCAACACCTGGCGTGCCAG
ATCTCGTGGATTTCCAGCCTCCTCCAGAGCTGGTGCTGAGAGAAGCTGGCGAAGAAGTGCCA
GACGCTGGCCCTAGAGAGGGCGTTAGCTTTCCTTGGAGCAGACCTCCTGGACAGGGCGAATT
CAGAGCCCTGAATGCCAGACTGCCCCTGAACACCGATGCCTACCTGTCTCTGCAAGAACTGC
AGGGACAAGACCCCACACACCTCGTGCGAGTGAAGTTCAGCAGAAGCGCTGACGCCCCTGCC
TACAAGCAGGGACAGAACCAGCTGTACAACGAGCTGAACCTGGGGAGAAGAGAAGAGTACGA
CGTGCTGGACAAGCGGAGAGGCAGAGATCCTGAGATGGGCGGCAAGCCCAGACGGAAGAATC
CTCAAGAGGGCCTGTATAATGAGCTGCAGAAAGATAAGATGGCCGAGGCCTACTCCGAGATC
GGCATGAAGGGCGAGCGCAGAAGAGGCAAGGGACACGATGGACTGTACCAGGGCCTGAGCAC
CGCCACCAAGGATACCTATGATGCCCTGCACATGCAGGCCCTGCCACCTAGA

An exemplary protein sequence encoding a CSR 82BB protein of the disclosure comprises or consists of the amino acid sequence of (CD8 signal peptide, CD2 extra cellular domain (D111H), IL2RB transmembrane domain, IL2RB intracellular domain):

(SEQ ID NO: 41)
MALPVTALLLPLALLLHAARPKEITNALETWGALGQDINLDIPSFQMSDDIDDIKWEKTSDK
KKIAQFRKEKETFKEKDTYKLFKNGTLKIKHLKTDDQDIYKVSIYHTKGKNVLEKIFDLKIQ
ERVSKPKISWTCINTTLTCEVMNGTDPELNLYQDGKHLKLSQRVITHKWTTSLSAKFKCTAG
NKVSKESSVEPVSCPEKGLDIPWLGHLLVGLSGAFGFIILVYLLINCRNTGPWLKKVLKCNT
PDPSKFFSQLSSEHGGDVQKWLSSPFPSSSFSPGGLAPEISPLEVLERDKVTQLLLQQDKVP
EPASLSSNHSLTSCFTNQGYFFFHLPDALEIEACQVYFTYDPYSEEDPDEGVAGAPTGSSPQ
PLQPLSGEDDAYCTFPSRDDLLLFSPSLLGGPSPPSTAPGGSGAGEERMPPSLQERVPRDWD
PQPLGPPTPGVPDLVDFQPPPELVLREAGEEVPDAGPREGVSFPWSRPPGQGEFRALNARLP
LNTDAYLSLQELQGQDPTHLV

An exemplary nucleotide sequence encoding a CSR 82BB protein of the disclosure comprises or consists of the amino acid sequence of (CD8 signal peptide, CD2 extra cellular domain (D111H), IL2RB transmembrane domain, IL2RB intracellular domain):

(SEQ ID NO: 194)
ATGGCTCTGCCTGTGACAGCTCTGCTGCTGCCTCTGGCTCTGCTTCTGCATGCCGCCAGACC
TAAAGAGATCACAAACGCCCTGGAAACCTGGGGAGCCCTCGGCCAGGATATTAACCTGGACA
TCCCCAGCTTCCAGATGAGCGACGACATCGATGACATCAAGTGGGAGAAAACCAGCGACAAG
AAGAAGATCGCCCAGTTCCGGAAAGAGAAAGAGACATTCAAAGAGAAGGACACCTACAAGCT
GTTCAAGAACGGCACCCTGAAGATCAAGCACCTGAAAACCGACGACCAGGACATCTATAAGG
TGTCCATCTACCACACCAAGGGCAAGAACGTGCTGGAAAAGATCTTCGACCTCAAGATCCAA
GAGCGGGTGTCCAAGCCTAAGATCAGCTGGACCTGCATCAACACCACACTGACCTGCGAAGT
GATGAACGGCACAGACCCCGAGCTGAACCTGTACCAGGATGGCAAACACCTGAAGCTGAGCC
AGCGCGTGATCACCCACAAGTGGACAACAAGCCTGAGCGCCAAGTTCAAGTGCACCGCCGGA
AACAAAGTGTCCAAAGAAAGCAGCGTGGAACCCGTGTCTTGCCCCGAGAAAGGCCTGGACAT
TCCTTGGCTGGGACATCTGCTCGTGGGACTGTCTGGCGCCTTCGGCTTTATCATCCTGGTGT
ACCTGCTGATCAACTGCCGGAACACAGGCCCCTGGCTGAAGAAAGTGCTGAAGTGCAACACC
CCTGATCCGAGCAAGTTCTTTAGCCAGCTGAGCAGCGAGCATGGCGGCGACGTTCAGAAATG
GCTGTCTAGCCCATTTCCTAGCAGCAGCTTCAGCCCTGGTGGACTGGCCCCTGAGATTAGCC
CTCTGGAAGTGCTGGAACGGGACAAAGTGACCCAGCTGCTCCTGCAGCAGGATAAGGTGCCA
GAACCTGCCAGCCTGTCCAGCAATCACAGCCTGACCAGCTGCTTTACCAACCAGGGCTACTT
CTTCTTCCATCTGCCTGACGCTCTGGAAATCGAGGCCTGCCAGGTGTACTTCACCTACGATC
CCTACAGCGAAGAGGACCCCGATGAAGGTGTTGCCGGTGCTCCTACCGGAAGCTCTCCTCAA
CCTCTGCAACCACTGAGCGGCGAGGATGACGCCTACTGCACATTCCCCAGCAGAGATGACCT
GCTGCTGTTCAGCCCTTCTCTGCTCGGTGGACCTTCTCCACCTTCTACAGCACCTGGCGGAT
CTGGCGCAGGCGAGGAAAGAATGCCTCCTAGCCTGCAAGAGAGAGTGCCCAGAGACTGGGAT
CCTCAGCCTCTCGGACCTCCTACACCAGGCGTGCCAGACCTGGTGGATTTTCAGCCTCCTCC
AGAGCTGGTGCTGAGAGAAGCTGGCGAAGAAGTGCCAGACGCCGGACCTAGAGAGGGCGTTA
GCTTTCCTTGGAGCAGACCTCCTGGACAGGGCGAGTTCAGAGCCCTGAATGCTAGACTGCCC
CTGAACACCGATGCCTACCTGTCTCTGCAAGAGCTGCAGGGACAAGACCCCACACACCTGGT
G

An exemplary protein sequence encoding a CSR 82BBz protein of the disclosure comprises or consists of the amino acid sequence of (CD8 signal peptide, CD2 extra cellular domain (D111H), IL2RB transmembrane domain, IL2RB intracellular domain #1, CD3z intracellular domain #2):

(SEQ ID NO: 42)
MALPVTALLLPLALLLHAARPKEITNALETWGALGQDINLDIPSFQMSDDIDDIKWEKTSDK
KKIAQFRKEKETFKEKDTYKLFKNGTLKIKHLKTDDQDIYKVSIYHTKGKNVLEKIFDLKIQ
ERVSKPKISWTCINTTLTCEVMNGTDPELNLYQDGKHLKLSQRVITHKWTTSLSAKFKCTAG
NKVSKESSVEPVSCPEKGLDIPWLGHLLVGLSGAFGFIILVYLLINCRNTGPWLKKVLKCNT
PDPSKFFSQLSSEHGGDVQKWLSSPFPSSSFSPGGLAPEISPLEVLERDKVTQLLLQQDKVP
EPASLSSNHSLTSCFTNQGYFFFHLPDALEIEACQVYFTYDPYSEEDPDEGVAGAPTGSSPQ
PLQPLSGEDDAYCTFPSRDDLLLFSPSLLGGPSPPSTAPGGSGAGEERMPPSLQERVPRDWD
PQPLGPPTPGVPDLVDFQPPPELVLREAGEEVPDAGPREGVSFPWSRPPGQGEFRALNARLP
LNTDAYLSLQELQGQDPTHLVRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKDTYDA
LHMQALPPR

An exemplary nucleotide sequence encoding a CSR 82BBz protein of the disclosure comprises or consists of the amino acid sequence of (CD8 signal peptide, CD2 extra cellular domain (D111H), IL2RB transmembrane domain, IL2RB intracellular domain #1, CD3z intracellular domain #2):

(SEQ ID NO: 195)
ATGGCTCTGCCTGTGACAGCTCTGCTGCTGCCTCTGGCTCTGCTTCTGCATGCCGCCAGACC
TAAAGAGATCACAAACGCCCTGGAAACCTGGGGAGCCCTCGGCCAGGATATTAACCTGGACA
TCCCCAGCTTCCAGATGAGCGACGACATCGATGACATCAAGTGGGAGAAAACCAGCGACAAG
AAGAAGATCGCCCAGTTCCGGAAAGAGAAAGAGACATTCAAAGAGAAGGACACCTACAAGCT
GTTCAAGAACGGCACCCTGAAGATCAAGCACCTGAAAACCGACGACCAGGACATCTATAAGG
TGTCCATCTACCACACCAAGGGCAAGAACGTGCTGGAAAAGATCTTCGACCTCAAGATCCAA
GAGCGGGTGTCCAAGCCTAAGATCAGCTGGACCTGCATCAACACCACACTGACCTGCGAAGT
GATGAACGGCACAGACCCCGAGCTGAACCTGTACCAGGATGGCAAACACCTGAAGCTGAGCC
AGCGCGTGATCACCCACAAGTGGACAACAAGCCTGAGCGCCAAGTTCAAGTGCACCGCCGGA
AACAAAGTGTCCAAAGAAAGCAGCGTGGAACCCGTGTCTTGCCCCGAGAAAGGCCTGGACAT
TCCTTGGCTGGGACATCTGCTCGTGGGACTGTCTGGCGCCTTCGGCTTTATCATCCTGGTGT
ACCTGCTGATCAACTGCCGGAACACAGGCCCCTGGCTGAAGAAAGTGCTGAAGTGCAACACC
CCTGATCCGAGCAAGTTCTTTAGCCAGCTGAGCAGCGAGCATGGCGGCGACGTTCAGAAATG
GCTGTCTAGCCCATTTCCTAGCAGCAGCTTCAGCCCTGGTGGACTGGCCCCTGAGATTAGCC
CTCTGGAAGTGCTGGAACGGGACAAAGTGACCCAGCTGCTCCTGCAGCAGGATAAGGTGCCA
GAACCTGCCAGCCTGTCCAGCAATCACAGCCTGACCAGCTGCTTTACCAACCAGGGCTACTT
CTTCTTCCATCTGCCTGACGCTCTGGAAATCGAGGCCTGCCAGGTGTACTTCACCTACGATC
CCTACAGCGAAGAGGACCCCGATGAAGGTGTTGCCGGTGCTCCTACCGGAAGCTCTCCTCAA
CCTCTGCAACCACTGAGCGGCGAGGATGACGCCTACTGCACATTCCCCAGCAGAGATGACCT
GCTGCTGTTCAGCCCTTCTCTGCTCGGTGGACCTTCTCCACCTTCTACAGCACCTGGCGGAT
CTGGCGCAGGCGAGGAAAGAATGCCTCCTAGCCTGCAAGAGAGAGTGCCCAGAGACTGGGAT
CCTCAGCCTCTCGGACCTCCTACACCAGGCGTGCCAGACCTGGTGGATTTTCAGCCTCCTCC
AGAGCTGGTGCTGAGAGAAGCTGGCGAAGAAGTGCCAGACGCCGGACCTAGAGAGGGCGTTA
GCTTTCCTTGGAGCAGACCTCCTGGACAGGGCGAGTTCAGAGCCCTGAATGCTAGACTGCCC
CTGAACACCGATGCCTACCTGTCTCTGCAAGAGCTGCAGGGACAAGACCCCACACACCTCGT
CAGAGTGAAGTTCAGCAGAAGCGCCGACGCTCCCGCCTATAAGCAGGGACAGAACCAGCTGT
ACAACGAGCTGAATCTGGGGCGCAGAGAAGAGTACGATGTGCTGGACAAGCGGAGAGGCAGA
GATCCTGAGATGGGCGGCAAGCCCAGACGGAAGAATCCTCAAGAGGGCCTGTATAATGAGCT
GCAGAAAGACAAGATGGCCGAGGCCTACTCCGAGATCGGAATGAAGGGCGAGCGCAGAAGAG
GCAAGGGACACGATGGACTGTATCAGGGCCTGAGCACCGCCACCAAGGATACCTATGATGCC
CTGCACATGCAGGCCCTGCCTCCAAGA

An exemplary protein sequence encoding a CSR 822B protein of the disclosure comprises or consists of the amino acid sequence of (CD8 signal peptide, CD2 extra cellular domain (D111H), CD2 transmembrane domain, IL2RB intracellular domain):

(SEQ ID NO: 43)
MALPVTALLLPLALLLHAARPKEITNALETWGALGQDINLDIPSFQMSDDIDDIKWEKTSDK
KKIAQFRKEKETFKEKDTYKLFKNGTLKIKHLKTDDQDIYKVSIYHTKGKNVLEKIFDLKIQ
ERVSKPKISWTCINTTLTCEVMNGTDPELNLYQDGKHLKLSQRVITHKWTTSLSAKFKCTAG
NKVSKESSVEPVSCPEKGLDIYLIIGICGGGSLLMVFVALLVFYITNCRNTGPWLKKVLKCN
TPDPSKFFSQLSSEHGGDVQKWLSSPFPSSSFSPGGLAPEISPLEVLERDKVTQLLLQQDKV
PEPASLSSNHSLTSCFTNQGYFFFHLPDALEIEACQVYFTYDPYSEEDPDEGVAGAPTGSSP
QPLQPLSGEDDAYCTFPSRDDLLLFSPSLLGGPSPPSTAPGGSGAGEERMPPSLQERVPRDW
DPQPLGPPTPGVPDLVDFQPPPELVLREAGEEVPDAGPREGVSFPWSRPPGQGEFRALNARL
PLNTDAYLSLQELQGQDPTHLV

An exemplary nucleotide sequence encoding a CSR 822B protein of the disclosure comprises or consists of the amino acid sequence of (CD8 signal peptide, CD2 extra cellular domain (D111H), CD2 transmembrane domain, IL2RB intracellular domain):

(SEQ ID NO: 196)
ATGGCTCTGCCTGTGACAGCTCTGCTGCTGCCTCTGGCTCTGCTTCTGC
ATGCCGCCAGACCTAAAGAGATCACAAACGCCCTGGAAACCTGGGGAGC
CCTCGGCCAGGATATTAACCTGGACATCCCCAGCTTCCAGATGAGCGAC
GACATCGATGACATCAAGTGGGAGAAAACCAGCGACAAGAAGAAGATCG
CCCAGTTCCGGAAAGAGAAAGAGACATTCAAAGAGAAGGACACCTACAA
GCTGTTCAAGAACGGCACCCTGAAGATCAAGCACCTGAAAACCGACGAC
CAGGACATCTATAAGGTGTCCATCTACCACACCAAGGGCAAGAACGTGC
TGGAAAAGATCTTCGACCTCAAGATCCAAGAGCGGGTGTCCAAGCCTAA
GATCAGCTGGACCTGCATCAACACCACACTGACCTGCGAAGTGATGAAC
GGCACAGACCCCGAGCTGAACCTGTACCAGGATGGCAAACACCTGAAGC
TGAGCCAGCGCGTGATCACCCACAAGTGGACAACAAGCCTGAGCGCCAA
GTTCAAGTGCACCGCCGGAAACAAAGTGTCCAAAGAAAGCAGCGTGGAA
CCCGTGTCTTGCCCCGAGAAAGGACTGGACATCTACCTGATCATCGGCA
TCTGTGGCGGCGGAAGCCTGCGATGGTGTTTGTTGCCCTGCTGGTGTTC
TACATCACCAACTGCAGAAACACAGGCCCCTGGCTGAAGAAAGTGCTGA
AGTGCAACACCCCTGATCCGAGCAAGTTCTTTAGCCAGCTGAGCAGCGA
GCATGGCGGCGACGTTCAGAAATGGCTGTCTAGCCCATTTCCTAGCAGC
AGCTTCAGCCCTGGTGGACTGGCCCCTGAGATTAGCCCTCTGGAAGTGC
TGGAACGGGACAAAGTGACCCAGCTGCTCCTGCAGCAGGATAAGGTGCC
AGAACCTGCCAGCCTGTCCAGCAATCACAGCCTGACCAGCTGCTTTACC
AACCAGGGCTACTTCTTCTTCCATCTGCCTGACGCTCTGGAAATCGAGG
CCTGCCAGGTGTACTTCACCTACGATCCCTACAGCGAAGAGGACCCCGA
TGAAGGTGTTGCCGGTGCTCCTACCGGAAGCTCTCCTCAACCTCTGCAA
CCACTGAGCGGCGAGGATGACGCCTACTGCACATTCCCCAGCAGAGATG
ACCTGCTGCTGTTCAGCCCTTCTCTGCTCGGTGGACCTTCTCCACCTTC
TACAGCACCTGGCGGATCTGGCGCAGGCGAGGAAAGAATGCCTCCTAGC
CTGCAAGAGAGAGTGCCCAGGAGACTGGGATCCTCAGCCTCTCGGACCT
CCTACACCAGGCGTGCCAGACCTGGTGGATTTTCAGCCTCCTCCAGAGC
TGGTGCTGAGAGAAGCTGGCGAAGAAGTGCCAGACGCCGGACCTAGAGA
GGGCGTTAGCTTTCCTTGGAGCAGACCTCCTGGACAGGGCGAGTTCAGA
GCCCTGAATGCTAGACTGCCCCTGAACACCGATGCCTACCTGTCTCTGC
AAGAGCTGCAGGGACAAGACCCCACACACCTGGTG

An exemplary protein sequence encoding a CSR 822Bz protein of the disclosure comprises or consists of the amino acid sequence of (CD8 signal peptide, CD2 extra cellular domain (D111H), CD2 transmembrane domain, IL2RB intracellular domain #1, CD3z intracellular domain #2):

(SEQ ID NO: 44)
MALPVTALLLPLALLLHAARPKEITNALETWGALGQDINLDIPSFQMSD
DIDDIKWEKTSDKKKIAQFRKEKETFKEKDTYKLFKNGTLKIKHLKTDD
QDIYKVSIYHTKGKNVLEKIFDLKIQERVSKPKISWTCINTTLTCEVMN
GTDPELNLYQDGKHLKLSQRVITHKWTTSLSAKFKCTAGNKVSKESSVE
PVSCPEKGLDIYLIIGICGGGSLLMVFVALLVFYITNCRNTGPWLKKVL
KCNTPDPSKFFSQLSSEHGGDVQKWLSSPFPSSSFSPGGLAPEISPLEV
LERDKVTQLLLQQQDKVPEPASLSSNHSLTCFTNQGYFFFHLPDALEIE
ACQVYFTYDPYSEEDPDEGVAGAPTGSSPQPLQPLSGEDDAYCTFPSRD
DLLLFSPSLLGGPSPPSTAPGGSGAGEERMPPSLQERVPRDWDPQPLGP
PTPGVPDLVDFQPPPELVLREAGEEVPDAGPREGVSFPWSRPPGQEFRA
LNARLPLNTDAYLSLQELQGQDPTHLVRVKFSRSADAPAYKQGQNQLYN
ELNLGRREEYDVLDKRRGRDPEMGGKPRRKNPQEGLYNELQKDKMAEAY
SEIGMKGERRRGKGHDGLYQGLSTATKDTYDALHNQALPPR

An exemplary nucleotide sequence encoding a CSR 822Bz protein of the disclosure comprises or consists of the amino acid sequence of (CD8 signal peptide, CD2 extra cellular domain (D111H), CD2 transmembrane domain, IL2RB intracellular domain #1, CD3z intracellular domain #2):

(SEQ ID NO: 197)
ATGGCTCTGCCTGTGACAGCTCTGCTGCTGCCTCTGGCTCTGCTTCTGC
ATGCCGCCAGACCTAAAGAGATCACAAACGCCCTGGAAACCTGGGGAGC
CCTCGGCCAGGATATTAACCTGGACATCCCCAGCTTCCAGATGAGCGAC
GACATCGATGACATCAAGTGGGAGAAAACCAGCGACAAGAAGAAGATCG
CCCAGTTCCGGAAAGAGAAAGAGACATTCAAAGAGAAGGACACCTACAA
GCTGTTCAAGAACGGCACCCTGAAGATCAAGCACCTGAAAACCGACGAC
CAGGACATCTATAAGGTGTCCATCTACCACACCAAGGGCAAGAACGTGC
TGGAAAAGATCTTCGACCTCAAGATCCAAGAGCGGGTGTCCAAGCCTAA
GATCAGCTGGACCTGCATCAACACCACACTGACCTGCGAAGTGATGAAC
GGCACAGACCCCGAGCTGAACCTGTACCAGGATGGCAAACACCTGAAGC
TGAGCCAGCGCGTGATCACCCACAAGTGGACAACAAGCCTGAGCGCCAA
GTTCAAGTGCACCGCCGGAAACAAAGTGTCCAAAGAAAGCAGCGTGGAA
CCCGTGTCTTGCCCCGAGAAAGGACTGGACATCTACCTGATCATCGGCA
TCTGTGGCGGCGGAAGCCTGCTGATGGTGTTTGTTGCCCTGCTGGTGTT
CTACATCACCAACTGCAGAAACACAGGCCCCTGGCTGAAGAAAGTGCTG
AAGTGCAACACCCCTGATCCGAGCAAGTTCTTTAGCCAGCTGAGCAGCG
AGCATGGCGGCGACGTTCAGAAATGGCTGTCTAGCCCATTTCCTAGCAG
CAGCTTCAGCCCTGGTGGACTGGCCCCTGAGATTAGCCCTCTGGAAGTG
CTGGAACGGGACAAAGTGACCCAGCTGCTCCTGCAGCAGGATAAGGTGC
CAGAACCTGCCAGCCTGTCCAGCAATCACAGCCTGACCAGCTGCTTTAC
CAACCAGGGCTACTTCTTCTTCCATCTGCCTGACGCTCTGGAAATCGAG
GCCTGCCAGGTGTACTTCACCTACGATCCCTACAGCGAAGAGGACCCCG
ATGAAGGTGTTGCCGGTGCTCCTACCGGAAGCTCTCCTCAACCTCTGCA
ACCACTGAGCGGCGAGGATGACGCCTACTGCACATTCCCCAGCAGAGAT
GACCTGCTGCTGTTCAGCCCTTCTCTGCTCGGTGGACCTTCTCCACCTT
CTACAGCACCTGGCGGATCTGGCGCAGGCGAGGAAAGAATGCCTCCTAG
CCTGCAAGAGAGAGTGCCCAGAGACTGGGATCCTCAGCCTCTCGGACCT
CCTACACCAGGCGTGCCAGACCTGGTGGATTTTCAGCCTCCTCCAGAGC
TGGTGCTGAGAGAAGCTGGCGAAGAAGTGCCAGACGCCGGACCTAGAGA
GGGCGTTAGCTTTCCTTGGAGCAGACCTCCTGGACAGGGCGAGTTCAGA
GCCCTGAATGCTAGACTGCCCCTGAACACCGATGCCTACCTGTCTCTGC
AAGAGCTGCAGGGACAAGACCCCACACACCTCGTCAGAGTGAAGTTCAG
CAGAAGCGCCGACGCTCCCGCCTATAAGCAGGGACAGAACCAGCTGTAC
AACGAGCTGAATCTGGGGCGCAGAGAAGAGTACGATGTGCTGGACAAGC
GGAGAGGCTGTACAACGAGCTGAATCTGGGGCGCAGAGAAGAGTACGAT
GTGCTGGACAAGCGGAGAGGCAGAGATCCTGAGATGGGCGGCAAGCCCA
GACGGAAGAATCCTCAAGAGGGCCTGTATAATGAGCTGCAGAAAGACAA
GATGGCCGAGGCCTACTCCGAGATCGGAATGAAGGGCGAGCGCAGAAGA
GGCAAGGGACACGATGGACTGTATCAGGGCCTGAGCACCGCCACCAAGG
ATACCTATGATGCCCTGCACATGCAGGCCCTGCCTCCAAGA

An exemplary protein sequence encoding a CSR 8GGG protein of the disclosure comprises or consists of the amino acid sequence of (CD8 signal peptide, IL2RG extra cellular domain, IL2RG transmembrane domain, IL2RG intracellular domain):

(SEQ ID NO: 45)
MALPVTALLLPLALLLHAARPLNTTLILPNGNEDTTADFFLTTMPTDSL
SVSTLPLPEVQCFVFNVEYMNCTWNSSSEPQPTNLTLHYWYKNSDNDKV
QKCSHYLFSEEITSGCQLQKKEIHLYQTFVVQLQDPREPRRQATQMLKL
QNLVIPWAPENLTLHKLSESQLELNWNNRFLNHCLEHLVQYRTDWDHSW
TEQSVDYRHKFSLPSVDGQKRYTFRVRSRFNPLCGSAQHWSEWSHPIHW
GSNTSKENPFLFALEAVVISVGSMGLIISLLCVYFWLERTMPRIPTLKN
LEDLVTEYHGNFSAWSGVSKGLAESLQPDYSERLCLVSEIPPKGGALGE
GPGASPCNQHSPYWAPPCYTLKPET

An exemplary nucleotide sequence encoding a CSR 8GGG protein of the disclosure comprises or consists of the amino acid sequence of (CD8 signal peptide, IL2RG extra cellular domain, IL2RG transmembrane domain, IL2RG intracellular domain):

(SEQ ID NO: 198)
ATGGCTCTGCCTGTGACAGCTCTGCTGCTGCCTCTGGCTCTGCTTCTGC
ATGCTGCCAGACCTCTGAACACCACCATCCTGACACCTAACGGCAACGA
GGATACAACCGCCGACTTCTTCCTGACCACCATGCCTACCGATAGCCTG
AGCGTGTCCACACTGCCTCTGCCTGAGGTGCAGTGCTTCGTGTTCAACG
TCGAGTACATGAACTGCACCTGGAACAGCAGCAGCGAGCCCCAGCCTAC
CAATCTGACACTGCACTACTGGTACAAGAACAGCGACAACGACAAGGTG
CAGAAGTGCAGCCACTACCTGTTCAGCGAGGAAATCACCAGCGGCTGCC
AGCTGCAGAAGAAAGAGATCCACCTGTACCAGACCTTCGTGGTTCAGCT
GCAGGACCCCAGAGAGCCTAGAAGGCAGGCTACCCAGATGCTGAAACTG
CAGAACCTGGTCATCCCCTGGGCTCCCGAAAACCTGACTCTGCACAAGC
TGAGCGAGAGCCAGCTGGAACTGAACTGGAACAACCGGTTCCTGAACCA
CTGCCTGGAACACCTGGTGCAGTACAGAACCGACTGGGACCACAGCTGG
ACAGAGCAGAGCGTGGACTACCGGCACAAGTTCAGCCTGCCATCTGTGG
ACGGCCAGAAGCGGTACACCTTTAGAGTGCGGAGCCGGTTCAATCCCCT
GTGTGGATCTGCTCAGCATTGGAGCGAGTGGTCACACCCTATCCACTGG
GGCAGCAACACCAGCAAAGAGAACCCCTTCCTGTTCGCCCTGGAAGCCG
TGGTTATCAGCGTGGGCTCTATGGGCCTGATCATCAGCCTGCTGTGCGT
GTACTTCTGGCTGGAAAGAACCATGCCTCGGATCCCCACACTGAAGAAT
CTGGAAGATCTGGTCACCGAGTACCACGGCAACTTCTCTGCTTGGAGCG
GCGTGTCAAAAGGCCTGGCCGAATCTCTGCAGCCCGACTACTCTGAGAG
ACTGTGCCTGGTGTCTGAGATCCCTCCTAAAGGCGGAGCCCTCGGAGAA
GGACCTGGCGCCTCTCCATGTAATCAGCACAGCCCTTATTGGGCCCCTC
CTTGCTACACCCTGAAGCCTGAGACA

An exemplary protein sequence encoding a CSR 8GGGz protein of the disclosure comprises or consists of the amino acid sequence of (CD8 signal peptide, IL2RG extra cellular domain, IL2RG transmembrane domain, IL2RG intracellular domain #1, CD3z intracellular domain #2):

(SEQ ID NO: 46)
MALPVTALLLPLALLLHAARPLNTTILTPNGNEDTTADFFLTTMPTDSL
SVSTLPLPEVQCFVFNVEYMNCTWNSSSEPQPTNLTLHYWYKNSDNDKV
QKCSHYLFSEEITSGCQLQKKEIHLYQTFVVQLQDPREPRRQATQMLKL
QNLVIPWAPENLTLHKLSESQLELNWNNRFLNHCLEHLVQYRTDWDHSW
TEQSVDYRHKFSLPSVDGQKRYTFRVRSRFNPLCGSAQHWSEWSHPIHW
GSNTSKENPFLFALEAVVISVGSMGLIISLLCVYFWLERTMPRIPTLKN
LEDLVTEYHGNFSAWSGVSKGLAESLQPDYSERLCLVSEIPPKGGALGE
GPGASPCNQHSPYWAPPCYTLKPETRVKFSRSADAPAYKQGQNQLYNEL
NLGRREEYDVLDKRRGRDPEMGGKPRRKNPQEGLYNELQKDKMAEAYSE
IGMKGERRRGKGHDGLYQGLSTATKDTYDALHMQALPRR

An exemplary nucleotide sequence encoding a CSR 8GGGz protein of the disclosure comprises or consists of the amino acid sequence of (CD8 signal peptide, IL2RG extra cellular domain, IL2RG transmembrane domain, IL2RG intracellular domain #1, CD3z intracellular domain #2):

(SEQ ID NO: 199)
ATGGCTCTGCCTGTGACAGCTCTGCTGCTGCCTCTGGCTCTGCTTCTGC
ATGCTGCCAGACCTCTGAACACCACCATCCTGACACCTAACGGCAACGA
GGATACAACCGCCGACTTCTTCCTGACCACCATGCCTACCGATAGCCTG
AGCGTGTCCACACTGCCTCTGCCTGAGGTGCAGTGCTTCGTGTTCAACG
TCGAGTACATGAACTGCACCTGGAACAGCAGCAGCGAGCCCCAGCCTAC
CAATCTGACACTGCACTACTGGTACAAGAACAGCGACAACGACAAGGTG
CAGAAGTGCAGCCACTACCTGTTCAGCGAGGAAATCACCAGCGGCTGCC
AGCTGCAGAAGAAAGAGATCCACCTGTACCAGACCTTCGTGGTTCAGCT
GCAGGACCCCAGAGAGCCTAGAAGGCAGGCTACCCAGATGCTGAAACTG
CAGAACCTGGTCATCCCCTGGGCTCCCGAAAACCTGACTCTGCACAAGC
TGAGCGAGAGCCAGCTGGAACTGAACTGGAACAACCGGTTCCTGAACCA
CTGCCTGGAACACCTGGTGCAGTACAGAACCGACTGGGACCACAGCTGG
ACAGAGCAGAGCGTGGACTACCGGCACAAGTTCAGCCTGCCATCTGTGG
ACGGCCAGAAGCGGTACACCTTTAGAGTGCGGAGCCGGTTCAATCCCCT
GTGTGGATCTGCTCAGCATTGGAGCGAGTGGTCACACCCTATCCACTGG
GGCAGCAACACCAGCAAAGAGAACCCCTTCCTGTTCGCCCTGGAAGCCG
TGGTTATCAGCGTGGGCTCTATGGGCCTGATCATCAGCCTGCTGTGCGT
GTACTTCTGGCTGGAAAGAACCATGCCTCGGATCCCCACACTGAAGAAT
CTGGAAGATCTGGTCACCGAGTACCACGGCAACTTCTCTGCTTGGAGCG
GCGTGTCAAAAGGCCTGGCCGAATCTCTGCAGCCCGACTACTCTGAGAG
ACTGTGCCTGGTGTCTGAGATCCCTCCTAAAGGCGGAGCCCTCGGAGAA
GGACCTGGCGCCTCTCCATGTAATCAGCACAGCCCTTATTGGGCCCCTC
CTTGCTACACCCTGAAGCCTGAGACAAGAGTGAAGTTCAGCAGAAGCGC
CGACGCTCCCGCCTATAAGCAGGGACAGAACCAGCTGTACAACGAGCTG
AACCTGGGGAGAAGAGAAGAGTACGACGTGCTGGACAAGCGGAGAGGCA
GAGATCCTGAGATGGGCGGCAAGCCCAGACGGAAGAATCCTCAAGAGGG
CCTGTATAATGAGCTGCAAAAGGACAAGATGGCCGAGGCTACAGCGAGA
TCGGAATGAAGGGCGAGCGCAGAAGAGGCAAGGGACACGATGGACTGTA
CCAGGGCCTGAGCACCGCCACCAAGGATACCTATGATGCCCTGCACATG
CAGGCCCTGCCTCCAAGA

An exemplary protein sequence encoding a CSR 82GG protein of the disclosure comprises or consists of the amino acid sequence of (CD8 signal peptide, CD2 extra cellular domain (D111H), IL2RG transmembrane domain, IL2RG intracellular domain):

(SEQ ID NO: 47)
MALPVTALLLPLALLLHAARPKEITNALETWGALGQDINLDIPSFQMSD
DIDDIKWEKTSDKKKIAQFRKEKETFKEKDTYKLFKNGTLKIKHLKTDD
QDIYKVSIYHTKGKNVLEKIFDLKIQERVSKPKISWTCINTTLTCEVMN
GTDPELNLYQDGKHLKLSQRVITHKWTTSLSAKFKCTAGNKVSKESSVE
PVSCPEKGLDAVVISVGSMGLIISLLCVYFWLERTMPRIPTLKNLEDLV
TEYHGNFSAWSGVSKGLAESLQPDYSERLCLVSEIPPKGGALGEGPGAS
PCNQHSPYWAPPCYTLKPET

An exemplary nucleotide sequence encoding a CSR 82GG protein of the disclosure comprises or consists of the amino acid sequence of (CD8 signal peptide, CD2 extra cellular domain (D111H), IL2RG transmembrane domain, IL2RG intracellular domain):

(SEQ ID NO: 200)
ATGGCTCTGCCTGTGACAGCTCTGCTGCTGCCTCTGGCTCTGCTTCTGC
ATGCCGCCAGACCTAAAGAGATCACAAACGCCCTGGAAACCTGGGGAGC
CCTCGGCCAGGATATTAACCTGGACATCCCCAGCTTCCAGATGAGCGAC
GACATCGATGACATCAAGTGGGAGAAAACCAGCGACAAGAAGAAGATCG
CCCAGTTCCGGAAAGAGAAAGAGACATTCAAAGAGAAGGACACCTACAA
GCTGTTCAAGAACGGCACCCTGAAGATCAAGCACCTGAAAACCGACGAC
CAGGACATCTATAAGGTGTCCATCTACCACACCAAGGGCAAGAACGTGC
TGGAAAAGATCTTCGACCTCAAGATCCAAGAGCGGGTGTCCAAGCCTAA
GATCAGCTGGACCTGCATCAACACCACACTGACCTGCGAAGTGATGAAC
GGCACAGACCCCGAGCTGAACCTGTACCAGGATGGCAAACACCTGAAGC
TGAGCCAGCGCGTGATCACCCACAAGTGGACAACAAGCCTGAGCGCCAA
GTTCAAGTGCACCGCCGGAAACAAAGTGTCCAAAGAAAGCAGCGTGGAA
CCCGTGTCTTGCCCCGAGAAAGGACTGGATGCCGTGGTCATCAGCGTGG
GCTCTATGGGCCTGATCATCAGCCTGCTGTGCGTGTACTTCTGGCTGGA
ACGGACCATGCCTCGGATCCCCACACTGAAGAACCTGGAAGATCTGGTC
ACCGAGTACCACGGCAACTTCTCTGCTTGGAGTGGCGTGTCAAAAGGCC
TGGCCGAAAGCCTGCAGCCTGACTACTCTGAGAGACTGTGCCTGGTGTC
TGAGATCCCTCCTAAAGGCGGAGCCCTTGGAGAAGGACCTGGCGCCTCT
CCATGTAACCAGCACAGCCCTTATTGGGCCCCTCCTTGCTACACTCTGA
AGCCCGAGACA

An exemplary protein sequence encoding a CSR 82GGz protein of the disclosure comprises or consists of the amino acid sequence of (CD8 signal peptide, CD2 extra cellular domain (D111H), IL2RG transmembrane domain, IL2RG intracellular domain #1, CD3z intracellular domain #2).

(SEQ ID NO: 48)
MALPVTALLLPLALLLHAARPKEITNALETWGALGQDINLDIPSFQMSD
DIDDIKWEKTSDKKKIAQFRKEKETFKEKDTYKLFKNGTLKIKHLKTDD
QDIYKVSIYHTKGKNVLEKIFDLKIQERVSKPKISWTCINTTLTCEVMN
GTDPELNLYQDGKHLKLSQRVITHKWTTSLSAKFKCTAGNKVSKESSVE
PVSCPEKGLDAVVISVGSMGLIISLLCVYFWLERTMPRIPTLKNLEDLV
TEYHGNFSAWSGVSKGLAESLQPDYSERLCLVSEIPPKGGALGEGPGAS
PCNQHSPYWAPPCYTLKPETRVKFSRSADAPAYKQGQNQLYNELNLGRR
EEYDVLDKRRGRDPEMGGKPRRKNPQEGLYNELQKDKMAEAYSEIGMKG
ERRRGKGHDGLYQGLSTATKDTYDALHMQALPPR

An exemplary nucleotide sequence encoding a CSR 82GGz protein of the disclosure comprises or consists of the amino acid sequence of (CD8 signal peptide, CD2 extra cellular domain (D111H), IL2RG transmembrane domain, IL2RG intracellular domain #1, CD3z intracellular domain #2):

(SEQ ID NO: 201)
ATGGCTCTGCCTGTGACAGCTCTGCTGCTGCCTCTGGCTCTGCTTCTGC
ATGCCGCCAGACCTAAAGAGATCACAAACGCCCTGGAAACCTGGGGAGC
CCTCGGCCAGGATATTAACCTGGACATCCCCAGCTTCCAGATGAGCGAC
GACATCGATGACATCAAGTGGGAGAAAACCAGCGACAAGAAGAAGATCG
CCCAGTTCCGGAAAGAGAAAGAGACATTCAAAGAGAAGGACACCTACAA
GCTGTTCAAGAACGGCACCCTGAAGATCAAGCACCTGAAAACCGACGAC
CAGGACATCTATAAGGTGTCCATCTACCACACCAAGGGCAAGAACGTGC
TGGAAAAGATCTTCGACCTCAAGATCCAAGAGCGGGTGTCCAAGCCTAA
GATCAGCTGGACCTGCATCAACACCACACTGACCTGCGAAGTGATGAAC
GGCACAGACCCCGAGCTGAACCTGTACCAGGATGGCAAACACCTGAAGC
TGAGCCAGCGCGTGATCACCCACAAGTGGACAACAAGCCTGAGCGCCAA
GTTCAAGTGCACCGCCGGAAACAAAGTGTCCAAAGAAAGCAGCGTGGAA
CCCGTGTCTTGCCCCGAGAAAGGACTGGATGCCGTGGTCATCAGCGTGG
GCTCTATGGGCCTGATCATCAGCCTGCTGTGCGTGTACTTCTGGCTGGA
ACGGACCATGCCTCGGATCCCCACACTGAAGAACCTGGAAGATCTGGTC
ACCGAGTACCACGGCAACTTCTCTGCTTGGAGTGGCGTGTCAAAAGGCC
TGGCCGAAAGCCTGCAGCCTGACTACTCTGAGAGACTGTGCCTGGTGTC
TGAGATCCCTCCTAAAGGCGGAGCCCTTGGAGAAGGACCTGGCGCCTCT
CCATGTAACCAGCACAGCCCTTATTGGGCCCCTCCTTGCTACACTCTGA
AGCCCGAGACAAGAGTGAAGTTCAGCAGAAGCGCCGACGCTCCCGCCTA
TAAGCAGGGACAGAACCAGCTGTACAACGAGCTGAATCTGGGGCGCAGA
GAAGAGTACGATGTGCTGGACAAGCGGAGAGGCAGAGATCCTGAGATGG
GCGGCAAGCCCAGACGGAAGAATCCTCAAGAGGGCCTGTATAATGAGCT
GCAGAAAGACAAGATGGCCGAGGCCTACAGCGAGATCGGAATGAAGGGC
GAGCGCAGAAGAGGCAAGGGACACGATGGACTGTATACAGGGCCTGAGC
ACCGCCACCAAGGATACCTATGATGCCCTGCACATGCAGGCCCTGCCTC
CAAGA

An exemplary protein sequence encoding a CSR 822G protein of the disclosure comprises or consists of the amino acid sequence of (CD8 signal peptide, CD2 extra cellular domain (D111H), CD2 transmembrane domain, IL2RG intracellular domain):

(SEQ ID NO: 49)
MALPVTALLLPLALLLHAARPKEITNALETWGALGQDINLDIPSFQMSD
DIDDIKWEKTSDKKKIAQFRKEKETFKEKDTYKLFKNGTLKIKHLKTDD
QDIYKVSIYHTKGKNVLEKIFDLKIQERVSKPKISWTCINTTLTCEVMN
GTDPELNLYQDGKGHLKLSQRVITHKWTTSLSAKFKCTAGNKVSKESSV
EPVSCPEKGLDIYLIIGICGGGSLLMVFVALLVFYITERTMPRIPTLKN
LEDLVTEYHGNFSAWSGVSKGLAESLQPDYSERLCLVSEIPPKGGALGE
GPGASPCNQHSPYWAPPCYTLKPET

An exemplary nucleotide sequence encoding a CSR 822G protein of the disclosure comprises or consists of the amino acid sequence of (CD8 signal peptide, CD2 extra cellular domain (D111H), CD2 transmembrane domain, IL2RG intracellular domain):

(SEQ ID NO: 202)
ATGGCTCTGCCTGTGACAGCTCTGCTGCTGCCTCTGGCTCTGCTTCTG
CATGCCGCCAGACCTAAAGAGATCACAAACGCCCTGGAAACCTGGGGA
GCCCTCGGCCAGGATATTAACCTGGACATCCCCAGCTTCCAGATGAGC
GACGACATCGATGACATCAAGTGGGAGAAAACCAGCGACAAGAAGAAG
ATCGCCCAGTTCCGGAAAGAGAAAGAGACATTCAAAGAGAAGGACACC
TACAAGCTGTTCAAGAACGGCACCCTGAAGATCAAGCACCTGAAAACC
GACGACCAGGACATCTATAAGGTGTCCATCTACCACACCAAGGGCAAG
AACGTGCTGGAAAAGATCTTCGACCTCAAGATCCAAGAGCGGGTGTCC
AAGCCTAAGATCAGCTGGACCTGCATCAACACCACACTGACCTGCGAA
GTGATGAACGGCACAGACCCCGAGCTGAACCTGTACCAGGATGGCAAA
CACCTGAAGCTGAGCCAGCGCGTGATCACCCACAAGTGGACAACAAGC
CTGAGCGCCAAGTTCAAGTGCACCGCCGGAAACAAAGTGTCCAAAGAA
AGCAGCGTGGAACCCGTGTCTTGCCCCGAGAAAGGACTGGACATCTAC
CTGATCATCGGCATCTGTGGCGGCGGAAGCCTGCTGATGGTGTTTGTT
GCCCTGCTGGTGTTCTACATCACCGAGCGGACCATGCCTCGGATCCCC
ACACTGAAGAACCTGGAAGATCTGGTCACCGAGTACCACGGCAACTTC
TCTGCTTGGAGTGGCGTGTCAAAAGGCCTGGCCGAAAGCCTGCAGCCT
GACTACTCTGAGAGACTGTGCCTGGTGTCTGAGATCCCTCCTAAAGGC
GGAGCCCTTGGAGAAGGACCTGGCGCCTCTCCATGTAACCAGCACAGC
CCTTATTGGGCCCCTCCTTGCTACACTCTGAAGCCCGAGACA

An exemplary protein sequence encoding a CSR 822 Gz protein of the disclosure comprises or consists of the amino acid sequence of (CD8 signal peptide, CD2 extra cellular domain (D111H), CD2 transmembrane domain, IL2RG intracellular domain #1, CD3z intracellular domain #2):

(SEQ ID NO: 50)
MALPVTALLLPLALLLHAARPKEITNALETWGALGQDINLDIPSFQMSD
DIDDIKWEKTSDKKKIAQFRKEKETFKEKDTYKLFKNGTLKIKHLKTDD
QDIYKVSIYHTKGKNVLEKIFDLKIQERVSKPKISWTCINTTLTCEVMN
GTDPELNLYQDGKHLKLSQRVITHKWTTSLSAKFKCTAGNKVSKESSVE
PVSCPEKGLDIYLIIGICGGGSLLMVFVALLVFYITERTMPRIPTLKNL
EDLVTEYHGNFSAWSGVSKGLAESLQPDYSERLCLVSEIPPKGGALGEG
PGASPCNQHSPYWAPPCYTLKPETRVKFSRSADAPAYKQGQNQLYNELN
LGRREEYDVLDKRRGRDPEMGGKPRRKNPQEGLYNELQKDKMAEAYSEI
GMKGERRRGKGHDGLYQGLSTATKDTYDALHMQALPPR

An exemplary nucleotide sequence encoding a CSR 822 Gz protein of the disclosure comprises or consists of the amino acid sequence of (CD8 signal peptide, CD2 extra cellular domain (D111H), CD2 transmembrane domain, IL2RG intracellular domain #1, CD3z intracellular domain #2):

(SEQ ID NO: 203)
ATGGCTCTGCCTGTGACAGCTCTGCTGCTGCCTCTGGCTCTGCTTCTGC
ATGCCGCCAGACCTAAAGAGATCACAAACGCCCTGGAAACCTGGGGAGC
CCTCGGCCAGGATATTAACCTGGACATCCCCAGCTTCCAGATGAGCGAC
GACATCGATGACATCAAGTGGGAGAAAACCAGCGACAAGAAGAAGATCG
CCCAGTTCCGGAAAGAGAAAGAGACATTCAAAGAGAAGGACACCTACAA
GCTGTTCAAGAACGGCACCCTGAAGATCAAGCACCTGAAAACCGACGAC
CAGGACATCTATAAGGTGTCCATCTACCACACCAAGGGCAAGAACGTGC
TGGAAAAGATCTTCGACCTCAAGATCCAAGAGCGGGTGTCCAAGCCTAA
GATCAGCTGGACCTGCATCAACACCACACTGACCTGCGAAGTGATGAAC
GGCACAGACCCCGAGCTGAACCTGTACCAGGATGGCAAACACCTGAAGC
TGAGCCAGCGCGTGATCACCCACAAGTGGACAACAAGCCTGAGCGCCAA
GTTCAAGTGCACCGCCGGAAACAAAGTGTCCAAAGAAAGCAGCGTGGAA
CCCGTGTCTTGCCCCGAGAAAGGACTGGACATCTACCTGATCATCGGCA
TCTGTGGCGGCGGAAGCCTGCTGATGGTGTTTGTTGCCCTGCTGGTGTT
CTACATCACCGAGCGGACCATGCCTCGGATCCCCACACTGAAGAACCTG
GAAGATCTGGTCACCGAGTACCACGGCAACTTCTCTGCTTGGAGTGGCG
TGTCAAAAGGCCTGGCCGAAAGCCTGCAGCCTGACTACTCTGAGAGACT
GTGCCTGGTGTCTGAGATCCCTCCTAAAGGCGGAGCCCTTGGAGAAGGA
CCTGGCGCCTCTCCATGTAACCAGCACAGCCCTTATTGGGCCCCTCCTT
GCTACACTCTGAAGCCCGAGACAAGAGTGAAGTTCAGCAGAAGCGCCGA
CGCTCCCGCCTATAAGCAGGGACAGAACCAGCTGTACAACGAGCTGAAT
CTGGGGCGCAGAGAAGAGTACGATGTGCTGGACAAGCGGAGAGGCAGAG
ATCCTGAGATGGGCGGCAAGCCCAGACGGAAGAATCCTCAAGAGGGCCT
GTATAATGAGCTGCAGAAAGACAAGATGGCCGAGGCCTACAGCGAGATC
GGAATGAAGGGCGAGCGCAGAAGAGGCAAGGGACACGATGGACTGTATC
AGGGCCTGAGCACCGCCACCAAGGATACCTATGATGCCCTGCACATGCA
GGCCCTGCCTCCAAGA

An exemplary protein sequence encoding a CSR 82BBG protein of the disclosure comprises or consists of the amino acid sequence of (CD8 signal peptide, CD2 extra cellular domain (D111H), IL2RB transmembrane domain, IL2RB intracellular domain #1, IL2RG intracellular domain #2):

(SEQ ID NO: 51)
MALPVTALLLPLALLLHAARPKEITNALETWGALGQDINLDIPSFQMSDD
IDDIKWEKTSDKKKIAQFRKEKETFKEKDTYKLFKNGTLKIKHLKTDDQD
IYKVSIYHTKGKNVLEKIFDLKIQERVSKPKISWTCINTTLTCEVMNGTD
PELNLYQDGKHLKLSQRVITHKWTTSLSAKFKCTAGNKVSKESSVEPVSC
PEKGLDIPWLGHLLVGLSGAFGFIILVYLLINCRNTGPWLKKVLKCNTPD
PSKFFSQLSSEHGGDVQKWLSSPFPSSSFSPGGLAPEISPLEVLERDKVT
QLLLQQDKVPEPASLSSNHSLTSCFTNQGYFFFHLPDALEIEACQVYFTY
DPYSEEDPDEGVAGAPTGSSPQPLQPLSGEDDAYCTFPSRDDLLLFSPSL
LGGPSPPSTAPGGSGAGEERMPPSLQERVPRDWDPQPLGPPTPGVPDLVD
FQPPPELVLREAGEEVPDAGPREGVSFPWSRPPGQGEFRALNARLPLNTD
AYLSLQELQGQDPTHLVERTMPRIPTLKNLEDLVTEYHGNFSAWSGVSKG
LAESLQPDYSERLCLVSEIPPKGGALGEGPGASPCNQHSPYWAPPCYTLK
PET

An exemplary nucleotide sequence encoding a CSR 82BBG protein of the disclosure comprises or consists of the amino acid sequence of (CD8 signal peptide, CD2 extra cellular domain (D111H), IL2RB transmembrane domain, IL2RB intracellular domain #1, IL2RG intracellular domain #2):

(SEQ ID NO: 204)
ATGGCTCTGCCTGTGACAGCTCTGCTGCTGCCTCTGGCTCTGCTTCTGCA
TGCCGCCAGACCTAAAGAGATCACAAACGCCCTGGAAACCTGGGGAGCCC
TCGGCCAGGATATTAACCTGGACATCCCCAGCTTCCAGATGAGCGACGAC
ATCGATGACATCAAGTGGGAGAAAACCAGCGACAAGAAGAAGATCGCCCA
GTTCCGGAAAGAGAAAGAGACATTCAAAGAGAAGGACACCTACAAGCTGT
TCAAGAACGGCACCCTGAAGATCAAGCACCTGAAAACCGACGACCAGGAC
ATCTATAAGGTGTCCATCTACCACACCAAGGGCAAGAACGTGCTGGAAAA
GATCTTCGACCTCAAGATCCAAGAGCGGGTGTCCAAGCCTAAGATCAGCT
GGACCTGCATCAACACCACACTGACCTGCGAAGTGATGAACGGCACAGAC
CCCGAGCTGAACCTGTACCAGGATGGCAAACACCTGAAGCTGAGCCAGCG
CGTGATCACCCACAAGTGGACAACAAGCCTGAGCGCCAAGTTCAAGTGCA
CCGCCGGAAACAAAGTGTCCAAAGAAAGCAGCGTGGAACCCGTGTCTTGC
CCCGAGAAAGGCCTGGACATTCCTTGGCTGGGACATCTGCTCGTGGGACT
GTCTGGCGCCTTCGGCTTTATCATCCTGGTGTACCTGCTGATCAACTGCC
GGAACACAGGCCCCTGGCTGAAGAAAGTGCTGAAGTGCAACACCCCTGAT
CCGAGCAAGTTCTTTAGCCAGCTGAGCAGCGAGCATGGCGGCGACGTTCA
GAAATGGCTGTCTAGCCCATTTCCTAGCAGCAGCTTCAGCCCTGGTGGAC
TGGCCCCTGAGATTAGCCCTCTGGAAGTGCTGGAACGGGACAAAGTGACC
CAGCTGCTCCTGCAGCAGGATAAGGTGCCAGAACCTGCCAGCCTGTCCAG
CAATCACAGCCTGACCAGCTGCTTTACCAACCAGGGCTACTTCTTCTTCC
ATCTGCCTGACGCTCTGGAAATCGAGGCCTGCCAGGTGTACTTCACCTAC
GATCCCTACAGCGAAGAGGACCCCGATGAAGGTGTTGCCGGTGCTCCTAC
CGGAAGCTCTCCTCAACCTCTGCAACCACTGAGCGGCGAGGATGACGCCT
ACTGCACATTCCCCAGCAGAGATGACCTGCTGCTGTTCAGCCCTTCTCTG
CTCGGTGGACCTTCTCCACCTTCTACAGCACCTGGCGGATCTGGCGCAGG
CGAGGAAAGAATGCCTCCTAGCCTGCAAGAGAGAGTGCCCAGAGACTGGG
ATCCTCAGCCTCTCGGACCTCCTACACCAGGCGTGCCAGACCTGGTGGAT
TTTCAGCCTCCTCCAGAGCTGGTGCTGAGAGAAGCTGGCGAAGAAGTGCC
AGACGCCGGACCTAGAGAGGGCGTTAGCTTTCCTTGGAGCAGACCTCCTG
GACAGGGCGAGTTCAGAGCCCTGAATGCTAGACTGCCCCTGAACACCGAT
GCCTACCTGTCTCTGCAAGAGCTGCAGGGACAAGACCCCACACATCTGGT
GGAACGGAGCATGCCTAGAATCCCCACACTGAAGAACCTGGAAGATCTGG
TCACCGAGTACCACGGCAACTTCAGTGCTTGGAGTGGCGTGTCAAAAGGA
CTGGCCGAGAGCCTGCAGCCTGACTACTCTGAGAGACTGTGCCTGGTGTC
TGAGATCCCTCCTAAAGGCGGAGCCCTTGGAGAAGGACCTGGCGCCTCTC
CATGTAACCAGCACAGCCCTTATTGGGCCCCTCCTTGCTACACTCTGAAG
CCCGAGACA

An exemplary protein sequence encoding a CSR 82BBGz protein of the disclosure comprises or consists of the amino acid sequence of (CD8 signal peptide, CD2 extra cellular domain (D111H), IL2RB transmembrane domain, IL2RB intracellular domain #1, IL2RG intracellular domain #2, CD3z intracellular domain #3):

(SEQ ID NO: 52)
MALPVTALLLPLALLLHAARPKEITNALETWGALGQDINLDIPSFQMSDD
IDDIKWEKTSDKKKIAQFRKEKETFKEKDTYKLFKNGTLKIKHLKTDDQD
IYKVSIYHTKGKNVLEKIFDLKIQERVSKPKISWTCINTTLTCEVMNGTD
PELNLYQDGKHLKLSQRVITHKWTTSLSAKFKCTAGNKVSKESSVEPVSC
PEKGLDIPWLGHLLVGLSGAFGFIILVYLLINCRNTGPWLKKVLKCNTPD
PSKFFSQLSSEHGGDVQKWLSSPFPSSSFSPGGLAPEISPLEVLERDKVT
QLLLQQDKVPEPASLSSNHSLTSCFTNQGYFFFHLPDALEIEACQVYFTY
DPYSEEDPDEGVAGAPTGSSPQPLQPLSGEDDAYCTFPSRDDLLLFSPSL
LGGPSPPSTAPGGSGAGEERMPPSLQERVPRDWDPQPLGPPTPGVPDLVD
FQPPPELVLREAGEEVPDAGPREGVSFPWSRPPGQGEFRALNARLPLNTD
AYLSLQELQGQDPTHLVERTMPRIPTLKNLEDLVTEYHGNFSAWSGVSKG
LAESLQPDYSERLCLVSEIPPKGGALGEGPGASPCNQHSPYWAPPCYTLK
PET

[symbol]

[symbol]

[symbol]

[symbol]

[symbol]

An exemplary nucleotide sequence encoding a CSR 82BBGz protein of the disclosure comprises or consists of the amino acid sequence of (CD8 signal peptide, CD2 extra cellular domain (D111H), IL2RB transmembrane domain, IL2RB intracellular domain #1, IL2RG intracellular domain #2, CD3z intracellular domain #3):

(SEQ ID NO: 205)
ATGGCTCTGCCTGTGACAGCTCTGCTGCTGCCTCTGGCTCTGCTTCTGCA
TGCCGCCAGACCTAAAGAGATCACAAACGCCCTGGAAACCTGGGGAGCCC
TCGGCCAGGATATTAACCTGGACATCCCCAGCTTCCAGATGAGCGACGAC
ATCGATGACATCAAGTGGGAGAAAACCAGCGACAAGAAGAAGATCGCCCA
GTTCCGGAAAGAGAAAGAGACATTCAAAGAGAAGGACACCTACAAGCTGT
TCAAGAACGGCACCCTGAAGATCAAGCACCTGAAAACCGACGACCAGGAC
ATCTATAAGGTGTCCATCTACCACACCAAGGGCAAGAACGTGCTGGAAAA
GATCTTCGACCTCAAGATCCAAGAGCGGGTGTCCAAGCCTAAGATCAGCT
GGACCTGCATCAACACCACACTGACCTGCGAAGTGATGAACGGCACAGAC
CCCGAGCTGAACCTGTACCAGGATGGCAAACACCTGAAGCTGAGCCAGCG
CGTGATCACCCACAAGTGGACAACAAGCCTGAGCGCCAAGTTCAAGTGCA
CCGCCGGAAACAAAGTGTCCAAAGAAAGCAGCGTGGAACCCGTGTCTTGC
CCCGAGAAAGGCCTGGACATTCCTTGGCTGGGACATCTGCTCGTGGGACT
GTCTGGCGCCTTCGGCTTTATCATCCTGGTGTACCTGCTGATCAACTGCC
GGAACACAGGCCCCTGGCTGAAGAAAGTGCTGAAGTGCAACACCCCTGAT
CCGAGCAAGTTCTTTAGCCAGCTGAGCAGCGAGCATGGCGGCGACGTTCA
GAAATGGCTGTCTAGCCCATTTCCTAGCAGCAGCTTCAGCCCTGGTGGAC
TGGCCCCTGAGATTAGCCCTCTGGAAGTGCTGGAACGGGACAAAGTGACC
CAGCTGCTCCTGCAGCAGGATAAGGTGCCAGAACCTGCCAGCCTGTCCAG
CAATCACAGCCTGACCAGCTGCTTTACCAACCAGGGCTACTTCTTCTTCC
ATCTGCCTGACGCTCTGGAAATCGAGGCCTGCCAGGTGTACTTCACCTAC
GATCCCTACAGCGAAGAGGACCCCGATGAAGGTGTTGCCGGTGCTCCTAC
CGGAAGCTCTCCTCAACCTCTGCAACCACTGAGCGGCGAGGATGACGCCT
ACTGCACATTCCCCAGCAGAGATGACCTGCTGCTGTTCAGCCCTTCTCTG
CTCGGTGGACCTTCTCCACCTTCTACAGCACCTGGCGGATCTGGCGCAGG
CGAGGAAAGAATGCCTCCTAGCCTGCAAGAGAGAGTGCCCAGAGACTGGG
ATCCTCAGCCTCTCGGACCTCCTACACCAGGCGTGCCAGACCTGGTGGAT
TTTCAGCCTCCTCCAGAGCTGGTGCTGAGAGAAGCTGGCGAAGAAGTGCC
AGACGCCGGACCTAGAGAGGGCGTTAGCTTTCCTTGGAGCAGACCTCCTG
GACAGGGCGAGTTCAGAGCCCTGAATGCTAGACTGCCCCTGAACACCGAT
GCCTACCTGTCTCTGCAAGAGCTGCAGGGACAAGACCCCACACATCTGGT
GGAACGGAGCATGCCTAGAATCCCCACACTGAAGAACCTGGAAGATCTGG
TCACCGAGTACCACGGCAACTTCAGTGCTTGGAGTGGCGTGTCAAAAGGA
CTGGCCGAGAGCCTGCAGCCTGACTACTCTGAGAGACTGTGCCTGGTGTC
TGAGATCCCTCCTAAAGGCGGAGCCCTTGGAGAAGGACCTGGCGCCTCTC
CATGTAACCAGCACAGCCCTTATTGGGCCCCTCCTTGCTACACTCTGAAG
CCCGAGACA

[symbol]

[symbol]

[symbol]

[symbol]

[symbol]

[symbol]

[symbol]

[symbol]

[symbol]

[symbol]

[symbol]

[symbol]

An exemplary protein sequence encoding a CSR 822BG protein of the disclosure comprises or consists of the amino acid sequence of (CD8 signal peptide, CD2 extra cellular domain (D111H), CD2 transmembrane domain, IL2RB intracellular domain #1, IL2RG intracellular domain #2):

(SEQ ID NO: 53)
MALPVTALLLPLALLLHAARPKEITNALETWGALGQDINLDIPSFQMSDD
IDDIKWEKTSDKKKIAQFRKEKETFKEKDTYKLFKNGTLKIKHLKTDDQD
IYKVSIYHTKGKNVLEKIFDLKIQERVSKPKISWTCINTTLTCEVMNGTD
PELNLYQDGKHLKLSQRVITHKWTTSLSAKFKCTAGNKVSKESSVEPVSC
PEKGLDIYLIIGICGGGSLLMVFVALLVFYITNCRNTGPWLKKVLKCNTP
DPSKFFSQLSSEHGGDVQKWLSSPFPSSSFSPGGLAPEISPLEVLERDKV
TQLLLQQDKVPEPASLSSNHSLTSCFTNQGYFFFHLPDALEIEACQVYFT
YDPYSEEDPDEGVAGAPTGSSPQPLQPLSGEDDAYCTFPSRDDLLLFSPS
LLGGPSPPSTAPGGSGAGEERMPPSLQERVPRDWDPQPLGPPTPGVPDLV
DFQPPPELVLREAGEEVPDAGPREGVSFPWSRPPGQGEFRALNARLPLNT
DAYLSLQELQGQDPTHLVERTMPRIPTLKNLEDLVTEYHGNFSAWSGVSK
GLAESLQPDYSERLCLVSEIPPKGGALGEGPGASPCNQHSPYWAPPCYTL
KPET

An exemplary nucleotide sequence encoding a CSR 822BG protein of the disclosure comprises or consists of the amino acid sequence of (CD8 signal peptide, CD2 extra cellular domain (D111H), CD2 transmembrane domain, IL2RB intracellular domain #1, IL2RG intracellular domain #2):

(SEQ ID NO: 206)
ATGGCTCTGCCTGTGACAGCTCTGCTGCTGCCTCTGGCTCTGCTTCTGCA
TGCCGCCAGACCTAAAGAGATCACAAACGCCCTGGAAACCTGGGGAGCCC
TCGGCCAGGATATTAACCTGGACATCCCCAGCTTCCAGATGAGCGACGAC
ATCGATGACATCAAGTGGGAGAAAACCAGCGACAAGAAGAAGATCGCCCA
GTTCCGGAAAGAGAAAGAGACATTCAAAGAGAAGGACACCTACAAGCTGT
TCAAGAACGGCACCCTGAAGATCAAGCACCTGAAAACCGACGACCAGGAC
ATCTATAAGGTGTCCATCTACCACACCAAGGGCAAGAACGTGCTGGAAAA
GATCTTCGACCTCAAGATCCAAGAGCGGGTGTCCAAGCCTAAGATCAGCT
GGACCTGCATCAACACCACACTGACCTGCGAAGTGATGAACGGCACAGAC
CCCGAGCTGAACCTGTACCAGGATGGCAAACACCTGAAGCTGAGCCAGCG
CGTGATCACCCACAAGTGGACAACAAGCCTGAGCGCCAAGTTCAAGTGCA
CCGCCGGAAACAAAGTGTCCAAAGAAAGCAGCGTGGAACCCGTGTCTTGC
CCCGAGAAAGGACTGGACATCTACCTGATCATCGGCATCTGTGGCGGCGG
AAGCCTGCTGATGGTGTTTGTTGCCCTGCTGGTGTTCTACATCACCAACT
GCAGAAACACAGGCCCCTGGCTGAAGAAAGTGCTGAAGTGCAACACCCCT
GATCCGAGCAAGTTCTTTAGCCAGCTGAGCAGCGAGCATGGCGGCGACGT
TCAGAAATGGCTGTCTAGCCCATTTCCTAGCAGCAGCTTCAGCCCTGGTG
GACTGGCCCCTGAGATTAGCCCTCTGGAAGTGCTGGAACGGGACAAAGTG
ACCCAGCTGCTCCTGCAGCAGGATAAGGTGCCAGAACCTGCCAGCCTGTC
CAGCAATCACAGCCTGACCAGCTGCTTTACCAACCAGGGCTACTTCTTCT
TCCATCTGCCTGACGCTCTGGAAATCGAGGCCTGCCAGGTGTACTTCACC
TACGATCCCTACAGCGAAGAGGACCCCGATGAAGGTGTTGCCGGTGCTCC
TACCGGAAGCTCTCCTCAACCTCTGCAACCACTGAGCGGCGAGGATGACG
CCTACTGCACATTCCCCAGCAGAGATGACCTGCTGCTGTTCAGCCCTTCT
CTGCTCGGTGGACCTTCTCCACCTTCTACAGCACCTGGCGGATCTGGCGC
AGGCGAGGAAAGAATGCCTCCTAGCCTGCAAGAGAGAGTGCCCAGAGACT
GGGATCCTCAGCCTCTCGGACCTCCTACACCAGGCGTGCCAGACCTGGTG
GATTTTCAGCCTCCTCCAGAGCTGGTGCTGAGAGAAGCTGGCGAAGAAGT
GCCAGACGCCGGACCTAGAGAGGGCGTTAGCTTTCCTTGGAGCAGACCTC
CTGGACAGGGCGAGTTCAGAGCCCTGAATGCTAGACTGCCCCTGAACACC
GATGCCTACCTGTCTCTGCAAGAGCTGCAGGGACAAGACCCCACACATCT
GGTGGAACGGACCATGCCTAGAATCCCCACACTGAAGAACCTGGAAGATC
TGGTCACCGAGTACCACGGCAACTTCAGTGCTTGGAGTGGCGTGTCAAAA
GGCCTGGCCGAAAGCCTGCAGCCTGACTACTCTGAGAGACTGTGCCTGGT
GTCTGAGATCCCTCCTAAAGGCGGAGCCCTTGGAGAAGGACCTGGCGCCT
CTCCATGTAACCAGCACAGCCCTTATTGGGCCCCTCCTTGCTACACTCTG
AAGCCCGAGACA

An exemplary protein sequence encoding a CSR 822BGz protein of the disclosure comprises or consists of the amino acid sequence of (CD8 signal peptide, CD2 extra cellular domain (D111H), CD2 transmembrane domain, IL2RB intracellular domain #1, IL2RG intracellular domain #2, CD3z intracellular domain #3):

(SEQ ID NO: 54)
MALPVTALLLPLALLLHAARPKEITNALETWGALGQDINLDIPSFQMSDD
IDDIKWEKTSDKKKIAQFRKEKETFKEKDTYKLFKNGTLKIKHLKTDDQD
IYKVSIYHTKGKNVLEKIFDLKIQERVSKPKISWTCINTTLTCEVMNGTD
PELNLYQDGKHLKLSQRVITHKWTTSLSAKFKCTAGNKVSKESSVEPVSC
PEKGLDIYLIIGICGGGSLLMVFVALLVFYITNCRNTGPWLKKVLKCNTP
DPSKFFSQLSSEHGGDVQKWLSSPFPSSSFSPGGLAPEISPLEVLERDKV
TQLLLQQDKVPEPASLSSNHSLTSCFTNQGYFFFHLPDALEIEACQVYFT
YDPYSEEDPDEGVAGAPTGSSPQPLQPLSGEDDAYCTFPSRDDLLLFSPS
LLGGPSPPSTAPGGSGAGEERMPPSLQERVPRDWDPQPLGPPTPGVPDLV
DFQPPPELVLREAGEEVPDAGPREGVSFPWSRPPGQGEFRALNARLPLNT
DAYLSLQELQGQDPTHLVERTMPRIPTLKNLEDLVTEYHGNFSAWSGVSK
GLAESLQPDYSERLCLVSEIPPKGGALGEGPGASPCNQHSPYWAPPCYTL
KPET

[symbol]

[symbol]

[symbol]

[symbol]

[symbol]

An exemplary nucleotide sequence encoding a CSR 822BGz protein of the disclosure comprises or consists of the amino acid sequence of (CD8 signal peptide, CD2 extra cellular domain (D111H), CD2 transmembrane domain, IL2RB intracellular domain #1, IL2RG intracellular domain #2, CD3z intracellular domain #3):

(SEQ ID NO: 207)
ATGGCTCTGCCTGTGACAGCTCTGCTGCTGCCTCTGGCTCTGCTTCTGCA
TGCCGCCAGACCTAAAGAGATCACAAACGCCCTGGAAACCTGGGGAGCCC
TCGGCCAGGATATTAACCTGGACATCCCCAGCTTCCAGATGAGCGACGAC
ATCGATGACATCAAGTGGGAGAAAACCAGCGACAAGAAGAAGATCGCCCA
GTTCCGGAAAGAGAAAGAGACATTCAAAGAGAAGGACACCTACAAGCTGT
TCAAGAACGGCACCCTGAAGATCAAGCACCTGAAAACCGACGACCAGGAC
ATCTATAAGGTGTCCATCTACCACACCAAGGGCAAGAACGTGCTGGAAAA
GATCTTCGACCTCAAGATCCAAGAGCGGGTGTCCAAGCCTAAGATCAGCT
GGACCTGCATCAACACCACACTGACCTGCGAAGTGATGAACGGCACAGAC
CCCGAGCTGAACCTGTACCAGGATGGCAAACACCTGAAGCTGAGCCAGCG
CGTGATCACCCACAAGTGGACAACAAGCCTGAGCGCCAAGTTCAAGTGCA
CCGCCGGAAACAAAGTGTCCAAAGAAAGCAGCGTGGAACCCGTGTCTTGC
CCCGAGAAAGGACTGGACATCTACCTGATCATCGGCATCTGTGGCGGCGG
AAGCCTGCTGATGGTGTTTGTTGCCCTGCTGGTGTTCTACATCACCAACT
GCAGAAACACAGGCCCCTGGCTGAAGAAAGTGCTGAAGTGCAACACCCCT
GATCCGAGCAAGTTCTTTAGCCAGCTGAGCAGCGAGCATGGCGGCGACGT
TCAGAAATGGCTGTCTAGCCCATTTCCTAGCAGCAGCTTCAGCCCTGGTG
GACTGGCCCCTGAGATTAGCCCTCTGGAAGTGCTGGAACGGGACAAAGTG
ACCCAGCTGCTCCTGCAGCAGGATAAGGTGCCAGAACCTGCCAGCCTGTC
CAGCAATCACAGCCTGACCAGCTGCTTTACCAACCAGGGCTACTTCTTCT
TCCATCTGCCTGACGCTCTGGAAATCGAGGCCTGCCAGGTGTACTTCACC
TACGATCCCTACAGCGAAGAGGACCCCGATGAAGGTGTTGCCGGTGCTCC
TACCGGAAGCTCTCCTCAACCTCTGCAACCACTGAGCGGCGAGGATGACG
CCTACTGCACATTCCCCAGCAGAGATGACCTGCTGCTGTTCAGCCCTTCT
CTGCTCGGTGGACCTTCTCCACCTTCTACAGCACCTGGCGGATCTGGCGC
AGGCGAGGAAAGAATGCCTCCTAGCCTGCAAGAGAGAGTGCCCAGAGACT
GGGATCCTCAGCCTCTCGGACCTCCTACACCAGGCGTGCCAGACCTGGTG
GATTTTCAGCCTCCTCCAGAGCTGGTGCTGAGAGAAGCTGGCGAAGAAGT
GCCAGACGCCGGACCTAGAGAGGGCGTTAGCTTTCCTTGGAGCAGACCTC
CTGGACAGGGCGAGTTCAGAGCCCTGAATGCTAGACTGCCCCTGAACACC
GATGCCTACCTGTCTCTGCAAGAGCTGCAGGGACAAGACCCCACACATCT
GGTGGAACGGACCATGCCTAGAATCCCCACACTGAAGAACCTGGAAGATC
TGGTCACCGAGTACCACGGCAACTTCAGTGCTTGGAGTGGCGTGTCAAAA
GGCCTGGCCGAAAGCCTGCAGCCTGACTACTCTGAGAGACTGTGCCTGGT
GTCTGAGATCCCTCCTAAAGGCGGAGCCCTTGGAGAAGGACCTGGCGCCT
CTCCATGTAACCAGCACAGCCCTTATTGGGCCCCTCCTTGCTACACTCTG
AAGCCCGAGACA

[symbol]

[symbol]

[symbol]

[symbol]

[symbol]

[symbol]

[symbol]

[symbol]

[symbol]

[symbol]

[symbol]

[symbol]

An exemplary protein sequence encoding a CSR 82GGB protein of the disclosure comprises or consists of the amino acid sequence of (CD8 signal peptide, CD2 extra cellular domain (D111H), IL2RG transmembrane domain, IL2RG intracellular domain #1, IL2RB intracellular domain #2):

(SEQ ID NO: 55)
MALPVTALLLPLALLLHAARPKEITNALETWGALGQDINLDIPSFQMSDD
IDDIKWEKTSDKKKIAQFRKEKETFKEKDTYKLFKNGTLKIKHLKTDDQD
IYKVSIYHTKGKNVLEKIFDLKIQERVSKPKISWTCINTTLTCEVMNGTD
PELNLYQDGKHLKLSQRVITHKWTTSLSAKFKCTAGNKVSKESSVEPVSC
PEKGLDAVVISVGSMGLIISLLCVYFWLERTMPRIPTLKNLEDLVTEYHG
NFSAWSGVSKGLAESLQPDYSERLCLVSEIPPKGGALGEGPGASPCNQHS
PYWAPPCYTLKPETNCRNTGPWLKKVLKCNTPDPSKFFSQLSSEHGGDVQ
KWLSSPFPSSSFSPGGLAPEISPLEVLERDKVTQLLLQQDKVPEPASLSS
NHSLTSCFTNQGYFFFHLPDALEIEACQVYFTYDPYSEEDPDEGVAGAPT
GSSPQPLQPLSGEDDAYCTFPSRDDLLLFSPSLLGGPSPPSTAPGGSGAG
EERMPPSLQERVPRDWDPQPLGPPTPGVPDLVDFQPPPELVLREAGEEVP
DAGPREGVSFPWSRPPGQGEFRALNARLPLNTDAYLSLQELQGQDPTHLV

An exemplary nucleotide sequence encoding a CSR 82GGB protein of the disclosure comprises or consists of the amino acid sequence of (CD8 signal peptide, CD2 extra cellular domain (D111H), IL2RG transmembrane domain, IL2RG intracellular domain #1, IL2RB intracellular domain #2):

(SEQ ID NO: 208)
ATGGCTCTGCCTGTGACAGCTCTGCTGCTGCCTCTGGCTCTGCTTCTGCA
TGCCGCCAGACCTAAAGAGATCACAAACGCCCTGGAAACCTGGGGAGCCC
TCGGCCAGGATATTAACCTGGACATCCCCAGCTTCCAGATGAGCGACGAC
ATCGATGACATCAAGTGGGAGAAAACCAGCGACAAGAAGAAGATCGCCCA
GTTCCGGAAAGAGAAAGAGACATTCAAAGAGAAGGACACCTACAAGCTGT
TCAAGAACGGCACCCTGAAGATCAAGCACCTGAAAACCGACGACCAGGAC
ATCTATAAGGTGTCCATCTACCACACCAAGGGCAAGAACGTGCTGGAAAA
GATCTTCGACCTCAAGATCCAAGAGCGGGTGTCCAAGCCTAAGATCAGCT
GGACCTGCATCAACACCACACTGACCTGCGAAGTGATGAACGGCACAGAC
CCCGAGCTGAACCTGTACCAGGATGGCAAACACCTGAAGCTGAGCCAGCG
CGTGATCACCCACAAGTGGACAACAAGCCTGAGCGCCAAGTTCAAGTGCA
CCGCCGGAAACAAAGTGTCCAAAGAAAGCAGCGTGGAACCCGTGTCTTGC
CCCGAGAAAGGACTGGATGCCGTGGTCATCAGCGTGGGCTCTATGGGCCT
GATCATCAGCCTGCTGTGCGTGTACTTCTGGCTGGAACGGACCATGCCTC
GGATCCCCACACTGAAGAACCTGGAAGATCTGGTCACCGAGTACCACGGC
AACTTCTCTGCTTGGAGTGGCGTGTCAAAAGGCCTGGCCGAAAGCCTGCA
GCCTGACTACTCTGAGAGACTGTGCCTGGTGTCTGAGATCCCTCCTAAAG
GCGGAGCCCTTGGAGAAGGACCTGGCGCCTCTCCATGTAACCAGCACAGC
CCTTATTGGGCCCCTCCTTGCTACACTCTGAAGCCCGAGACAAACTGCCG
GAACACAGGCCCCTGGCTGAAGAAAGTGCTGAAGTGCAACACCCCTGATC
CGAGCAAGTTCTTTAGCCAGCTGAGCAGCGAGCATGGCGGCGACGTTCAG
AAATGGCTGTCTAGCCCATTTCCTAGCAGCAGCTTCAGCCCTGGTGGACT
GGCCCCTGAGATTAGCCCTCTGGAAGTGCTCGAGCGGGACAAAGTGACAC
AGCTGCTCCTCCAGCAGGACAAGGTGCCAGAACCTGCCAGCCTGTCCAGC
AATCACAGCCTGACCAGCTGCTTTACCAACCAGGGCTACTTCTTCTTCCA
TCTGCCTGACGCTCTGGAAATCGAGGCCTGCCAGGTGTACTTCACCTACG
ATCCCTACAGCGAAGAGGACCCCGATGAAGGTGTTGCCGGTGCTCCTACC
GGAAGCTCTCCTCAACCTCTGCAACCACTGAGCGGCGAGGATGACGCCTA
CTGCACATTCCCCAGCAGAGATGACCTGCTGCTGTTCAGCCCTAGCCTGC
TCGGAGGACCTTCTCCACCATCTACAGCTCCAGGTGGAAGCGGAGCCGGC
GAGGAAAGAATGCCTCCAAGCCTGCAAGAGAGAGTGCCCAGAGACTGGGA
TCCTCAGCCTCTCGGACCTCCTACACCAGGCGTGCCAGACCTGGTGGATT
TTCAGCCTCCTCCAGAGCTGGTGCTGAGAGAAGCTGGCGAAGAAGTGCCA
GACGCCGGACCTAGAGAGGGCGTTAGCTTTCCTTGGAGCAGACCTCCTGG
ACAGGGCGAGTTCAGAGCCCTGAATGCTAGACTGCCCCTGAACACCGATG
CCTACCTGTCTCTGCAAGAGCTGCAGGGACAAGACCCCACACACCTGGTG

An exemplary protein sequence encoding a CSR 82GGBz protein of the disclosure comprises or consists of the amino acid sequence of (CD8 signal peptide, CD2 extra cellular domain (D111H), IL2RG transmembrane domain, IL2RG intracellular domain #1, IL2RB intracellular domain #2, CD3z intracellular domain #3):

(SEQ ID NO: 56)
MALPVTALLLPLALLLHAARPKEITNALETWGALGQDINLDIPSFQMSDD
IDDIKWEKTSDKKKIAQFRKEKETFKEKDTYKLFKNGTLKIKHLKTDDQD
IYKVSIYHTKGKNVLEKIFDLKIQERVSKPKISWTCINTTLTCEVMNGTD
PELNLYQDGKHLKLSQRVITHKWTTSLSAKFKCTAGNKVSKESSVEPVSC
PEKGLDAVVISVGSMGLIISLLCVYFWLERTMPRIPTLKNLEDLVTEYHG
NFSAWSGVSKGLAESLQPDYSERLCLVSEIPPKGGALGEGPGASPCNQHS
PYWAPPCYTLKPETNCRNTGPWLKKVLKCNTPDPSKFFSQLSSEHGGDVQ
KWLSSPFPSSSFSPGGLAPEISPLEVLERDKVTQLLLQQDKVPEPASLSS
NHSLTSCFTNQGYFFFHLPDALEIEACQVYFTYDPYSEEDPDEGVAGAPT
GSSPQPLQPLSGEDDAYCTFPSRDDLLLFSPSLLGGPSPPSTAPGGSGAG
EERMPPSLQERVPRDWDPQPLGPPTPGVPDLVDFQPPPELVLREAGEEVP
DAGPREGVSFPWSRPPGQGEFRALNARLPLNTDAYLSLQELQGQDPTHLV

[symbol]

[symbol]

[symbol]

[symbol]

An exemplary nucleotide sequence encoding a CSR 82GGBz protein of the disclosure comprises or consists of the amino acid sequence of (CD8 signal peptide, CD2 extra cellular domain (D111H), IL2RG transmembrane domain, IL2RG intracellular domain #1, IL2RB intracellular domain #2, CD3z intracellular domain #3):

(SEQ ID NO: 209)
ATGGCTCTGCCTGTGACAGCTCTGCTGCTGCCTCTGGCTCTGCTTCTGCA
TGCCGCCAGACCTAAAGAGATCACAAACGCCCTGGAAACCTGGGGAGCCC
TCGGCCAGGATATTAACCTGGACATCCCCAGCTTCCAGATGAGCGACGAC
ATCGATGACATCAAGTGGGAGAAAACCAGCGACAAGAAGAAGATCGCCCA
GTTCCGGAAAGAGAAAGAGACATTCAAAGAGAAGGACACCTACAAGCTGT
TCAAGAACGGCACCCTGAAGATCAAGCACCTGAAAACCGACGACCAGGAC
ATCTATAAGGTGTCCATCTACCACACCAAGGGCAAGAACGTGCTGGAAAA
GATCTTCGACCTCAAGATCCAAGAGCGGGTGTCCAAGCCTAAGATCAGCT
GGACCTGCATCAACACCACACTGACCTGCGAAGTGATGAACGGCACAGAC
CCCGAGCTGAACCTGTACCAGGATGGCAAACACCTGAAGCTGAGCCAGCG
CGTGATCACCCACAAGTGGACAACAAGCCTGAGCGCCAAGTTCAAGTGCA
CCGCCGGAAACAAAGTGTCCAAAGAAAGCAGCGTGGAACCCGTGTCTTGC
CCCGAGAAAGGACTGGATGCCGTGGTCATCAGCGTGGGCTCTATGGGCCT
GATCATCAGCCTGCTGTGCGTGTACTTCTGGCTGGAACGGACCATGCCTC
GGATCCCCACACTGAAGAACCTGGAAGATCTGGTCACCGAGTACCACGGC
AACTTCTCTGCTTGGAGTGGCGTGTCAAAAGGCCTGGCCGAAAGCCTGCA
GCCTGACTACTCTGAGAGACTGTGCCTGGTGTCTGAGATCCCTCCTAAAG
GCGGAGCCCTTGGAGAAGGACCTGGCGCCTCTCCATGTAACCAGCACAGC
CCTTATTGGGCCCCTCCTTGCTACACTCTGAAGCCCGAGACAAACTGCCG
GAACACAGGCCCCTGGCTGAAGAAAGTGCTGAAGTGCAACACCCCTGATC
CGAGCAAGTTCTTTAGCCAGCTGAGCAGCGAGCATGGCGGCGACGTTCAG
AAATGGCTGTCTAGCCCATTTCCTAGCAGCAGCTTCAGCCCTGGTGGACT
GGCCCCTGAGATTAGCCCTCTGGAAGTGCTCGAGCGGGACAAAGTGACAC
AGCTGCTCCTCCAGCAGGACAAGGTGCCAGAACCTGCCAGCCTGTCCAGC
AATCACAGCCTGACCAGCTGCTTTACCAACCAGGGCTACTTCTTCTTCCA
TCTGCCTGACGCTCTGGAAATCGAGGCCTGCCAGGTGTACTTCACCTACG
ATCCCTACAGCGAAGAGGACCCCGATGAAGGTGTTGCCGGTGCTCCTACC
GGAAGCTCTCCTCAACCTCTGCAACCACTGAGCGGCGAGGATGACGCCTA
CTGCACATTCCCCAGCAGAGATGACCTGCTGCTGTTCAGCCCTAGCCTGC
TCGGAGGACCTTCTCCACCATCTACAGCTCCAGGTGGAAGCGGAGCCGGC
GAGGAAAGAATGCCTCCAAGCCTGCAAGAGAGAGTGCCCAGAGACTGGGA
TCCTCAGCCTCTCGGACCTCCTACACCAGGCGTGCCAGACCTGGTGGATT
TTCAGCCTCCTCCAGAGCTGGTGCTGAGAGAAGCTGGCGAAGAAGTGCCA
GACGCCGGACCTAGAGAGGGCGTTAGCTTTCCTTGGAGCAGACCTCCTGG
ACAGGGCGAGTTCAGAGCCCTGAATGCTAGACTGCCCCTGAACACCGATG
CCTACCTGTCTCTGCAAGAGCTGCAGGGACAAGACCCCACACACCTCGTC

[symbol]

[symbol]

[symbol]

[symbol]

[symbol]

[symbol]

[symbol]

[symbol]

[symbol]

[symbol]

[symbol]

An exemplary protein sequence encoding a CSR 822 GB protein of the disclosure comprises or consists of the amino acid sequence of (CD8 signal peptide, CD2 extra cellular domain (D111H), CD2 transmembrane domain, IL2RG intracellular domain #1, IL2RB intracellular domain #2):

(SEQ ID NO: 57)
MALPVTALLLPLALLLHAARPKEITNALETWGALGQDINLDIPSFQMSDD
IDDIKWEKTSDKKKIAQFRKEKETFKEKDTYKLFKNGTLKIKHLKTDDQD
IYKVSIYHTKGKNVLEKIFDLKIQERVSKPKISWTCINTTLTCEVMNGTD
PELNLYQDGKHLKLSQRVITHKWTTSLSAKFKCTAGNKVSKESSVEPVSC
PEKGLDIYLIIGICGGGSLLMVFVALLVFYITERTMPRIPTLKNLEDLVT
EYHGNFSAWSGVSKGLAESLQPDYSERLCLVSEIPPKGGALGEGPGASPC
NQHSPYWAPPCYTLKPETNCRNTGPWLKKVLKCNTPDPSKFFSQLSSEHG
GDVQKWLSSPFPSSSFSPGGLAPEISPLEVLERDKVTQLLLQQDKVPEPA
SLSSNHSLTSCFTNQGYFFFHLPDALEIEACQVYFTYDPYSEEDPDEGVA
GAPTGSSPQPLQPLSGEDDAYCTFPSRDDLLLFSPSLLGGPSPPSTAPGG
SGAGEERMPPSLQERVPRDWDPQPLGPPTPGVPDLVDFQPPPELVLREAG
EEVPDAGPREGVSFPWSRPPGQGEFRALNARLPLNTDAYLSLQELQGQDP
THLV

An exemplary nucleotide sequence encoding a CSR 822 GB protein of the disclosure comprises or consists of the amino acid sequence of (CD8 signal peptide, CD2 extra cellular domain (D111H), CD2 transmembrane domain, IL2RG intracellular domain #1, IL2RB intracellular domain #2):

(SEQ ID NO: 210)
ATGGCTCTGCCTGTGACAGCTCTGCTGCTGCCTCTGGCTCTGCTTCTGCA
TGCCGCCAGACCTAAAGAGATCACAAACGCCCTGGAAACCTGGGGAGCCC
TCGGCCAGGATATTAACCTGGACATCCCCAGCTTCCAGATGAGCGACGAC
ATCGATGACATCAAGTGGGAGAAAACCAGCGACAAGAAGAAGATCGCCCA
GTTCCGGAAAGAGAAAGAGACATTCAAAGAGAAGGACACCTACAAGCTGT
TCAAGAACGGCACCCTGAAGATCAAGCACCTGAAAACCGACGACCAGGAC
ATCTATAAGGTGTCCATCTACCACACCAAGGGCAAGAACGTGCTGGAAAA
GATCTTCGACCTCAAGATCCAAGAGCGGGTGTCCAAGCCTAAGATCAGCT
GGACCTGCATCAACACCACACTGACCTGCGAAGTGATGAACGGCACAGAC
CCCGAGCTGAACCTGTACCAGGATGGCAAACACCTGAAGCTGAGCCAGCG
CGTGATCACCCACAAGTGGACAACAAGCCTGAGCGCCAAGTTCAAGTGCA
CCGCCGGAAACAAAGTGTCCAAAGAAAGCAGCGTGGAACCCGTGTCTTGC
CCCGAGAAAGGACTGGACATCTACCTGATCATCGGCATCTGTGGCGGCGG
AAGCCTGCTGATGGTGTTTGTTGCCCTGCTGGTGTTCTACATCACCGAGC
GGACCATGCCTCGGATCCCCACACTGAAGAACCTGGAAGATCTGGTCACC
GAGTACCACGGCAACTTCTCTGCTTGGAGTGGCGTGTCAAAAGGCCTGGC
CGAAAGCCTGCAGCCTGACTACTCTGAGAGACTGTGCCTGGTGTCTGAGA
TCCCTCCTAAAGGCGGAGCCCTTGGAGAAGGACCTGGCGCCTCTCCATGT
AACCAGCACAGCCCTTATTGGGCCCCTCCTTGCTACACTCTGAAGCCCGA
GACAAACTGCCGGAACACAGGCCCCTGGCTGAAGAAAGTGCTGAAGTGCA
ACACCCCTGATCCGAGCAAGTTCTTTAGCCAGCTGAGCAGCGAGCATGGC
GGCGACGTTCAGAAATGGCTGTCTAGCCCATTTCCTAGCAGCAGCTTCAG
CCCTGGTGGACTGGCCCCTGAGATTAGCCCTCTGGAAGTGCTGGAACGGG
ACAAAGTGACCCAGCTGCTCCTGCAGCAGGATAAGGTGCCAGAACCTGCC
AGCCTGTCCAGCAATCACAGCCTGACCAGCTGCTTTACCAACCAGGGCTA
CTTCTTCTTCCATCTGCCTGACGCTCTGGAAATCGAGGCCTGCCAGGTGT
ACTTCACCTACGATCCCTACAGCGAAGAGGACCCCGATGAAGGTGTTGCC
GGTGCTCCTACCGGAAGCTCTCCTCAACCTCTGCAACCACTGAGCGGCGA
GGATGACGCCTACTGCACATTCCCCAGCAGAGATGACCTGCTGCTGTTCA
GCCCTAGCCTGCTCGGAGGACCTTCTCCACCATCTACAGCTCCAGGTGGA
AGCGGAGCCGGCGAGGAAAGAATGCCTCCAAGCCTGCAAGAGAGAGTGCC
CAGAGACTGGGATCCTCAGCCTCTCGGACCTCCTACACCAGGCGTGCCAG
ACCTGGTGGATTTTCAGCCTCCTCCAGAGCTGGTGCTGAGAGAAGCTGGC
GAAGAAGTGCCAGACGCCGGACCTAGAGAGGGCGTTAGCTTTCCTTGGAG
CAGACCTCCTGGACAGGGCGAGTTCAGAGCCCTGAATGCTAGACTGCCCC
TGAACACCGATGCCTACCTGTCTCTGCAAGAGCTGCAGGGACAAGACCCC
ACACACCTGGTG

An exemplary protein sequence encoding a CSR 822GBz protein of the disclosure comprises or consists of the amino acid sequence of (CD8 signal peptide, CD2 extra cellular domain (D111H), CD2 transmembrane domain, IL2RG intracellular domain #1, IL2RB intracellular domain #2, CD3z intracellular domain #3):

(SEQ ID NO: 58)
MALPVTALLLPLALLLHAARPKEITNALETWGALGQDINLDIPSFQMSDD
IDDIKWEKTSDKKKIAQFRKEKETFKEKDTYKLFKNGTLKIKHLKTDDQD
IYKVSIYHTKGKNVLEKIFDLKIQERVSKPKISWTCINTTLTCEVMNGTD
PELNLYQDGKHLKLSQRVITHKWTTSLSAKFKCTAGNKVSKESSVEPVSC
PEKGLDIYLIIGICGGGSLLMVFVALLVFYITERTMPRIPTLKNLEDLVT
EYHGNFSAWSGVSKGLAESLQPDYSERLCLVSEIPPKGGALGEGPGASPC
NQHSPYWAPPCYTLKPETNCRNTGPWLKKVLKCNTPDPSKFFSQLSSEHG
GDVQKWLSSPFPSSSFSPGGLAPEISPLEVLERDKVTQLLLQQDKVPEPA
SLSSNHSLTSCFTNQGYFFFHLPDALEIEACQVYFTYDPYSEEDPDEGVA
GAPTGSSPQPLQPLSGEDDAYCTFPSRDDLLLFSPSLLGGPSPPSTAPGG
SGAGEERMPPSLQERVPRDWDPQPLGPPTPGVPDLVDFQPPPELVLREAG
EEVPDAGPREGVSFPWSRPPGQGEFRALNARLPLNTDAYLSLQELQGQDP
THLV

[symbol]

[symbol]

[symbol]

[symbol]

[symbol]

An exemplary nucleotide sequence encoding a CSR 822GBz protein of the disclosure comprises or consists of the amino acid sequence of (CD8 signal peptide, CD2 extra cellular domain (D111H), CD2 transmembrane domain, IL2RG intracellular domain #1, IL2RB intracellular domain #2, CD3z intracellular domain #3):

(SEQ ID NO: 211)
ATGGCTCTGCCTGTGACAGCTCTGCTGCTGCCTCTGGCTCTGCTTCTGCA
TGCCGCCAGACCTAAAGAGATCACAAACGCCCTGGAAACCTGGGGAGCCC
TCGGCCAGGATATTAACCTGGACATCCCCAGCTTCCAGATGAGCGACGAC
ATCGATGACATCAAGTGGGAGAAAACCAGCGACAAGAAGAAGATCGCCCA
GTTCCGGAAAGAGAAAGAGACATTCAAAGAGAAGGACACCTACAAGCTGT
TCAAGAACGGCACCCTGAAGATCAAGCACCTGAAAACCGACGACCAGGAC
ATCTATAAGGTGTCCATCTACCACACCAAGGGCAAGAACGTGCTGGAAAA
GATCTTCGACCTCAAGATCCAAGAGCGGGTGTCCAAGCCTAAGATCAGCT
GGACCTGCATCAACACCACACTGACCTGCGAAGTGATGAACGGCACAGAC
CCCGAGCTGAACCTGTACCAGGATGGCAAACACCTGAAGCTGAGCCAGCG
CGTGATCACCCACAAGTGGACAACAAGCCTGAGCGCCAAGTTCAAGTGCA
CCGCCGGAAACAAAGTGTCCAAAGAAAGCAGCGTGGAACCCGTGTCTTGC
CCCGAGAAAGGACTGGACATCTACCTGATCATCGGCATCTGTGGCGGCGG
AAGCCTGCTGATGGTGTTTGTTGCCCTGCTGGTGTTCTACATCACCGAGC
GGACCATGCCTCGGATCCCCACACTGAAGAACCTGGAAGATCTGGTCACC
GAGTACCACGGCAACTTCTCTGCTTGGAGTGGCGTGTCAAAAGGCCTGGC
CGAAAGCCTGCAGCCTGACTACTCTGAGAGACTGTGCCTGGTGTCTGAGA
TCCCTCCTAAAGGCGGAGCCCTTGGAGAAGGACCTGGCGCCTCTCCATGT
AACCAGCACAGCCCTTATTGGGCCCCTCCTTGCTACACTCTGAAGCCCGA
GACAAACTGCCGGAACACAGGCCCCTGGCTGAAGAAAGTGCTGAAGTGCA
ACACCCCTGATCCGAGCAAGTTCTTTAGCCAGCTGAGCAGCGAGCATGGC
GGCGACGTTCAGAAATGGCTGTCTAGCCCATTTCCTAGCAGCAGCTTCAG
CCCTGGTGGACTGGCCCCTGAGATTAGCCCTCTGGAAGTGCTGGAACGGG
ACAAAGTGACCCAGCTGCTCCTGCAGCAGGATAAGGTGCCAGAACCTGCC
AGCCTGTCCAGCAATCACAGCCTGACCAGCTGCTTTACCAACCAGGGCTA
CTTCTTCTTCCATCTGCCTGACGCTCTGGAAATCGAGGCCTGCCAGGTGT
ACTTCACCTACGATCCCTACAGCGAAGAGGACCCCGATGAAGGTGTTGCC
GGTGCTCCTACCGGAAGCTCTCCTCAACCTCTGCAACCACTGAGCGGCGA
GGATGACGCCTACTGCACATTCCCCAGCAGAGATGACCTGCTGCTGTTCA
GCCCTAGCCTGCTCGGAGGACCTTCTCCACCATCTACAGCTCCAGGTGGA
AGCGGAGCCGGCGAGGAAAGAATGCCTCCAAGCCTGCAAGAGAGAGTGCC
CAGAGACTGGGATCCTCAGCCTCTCGGACCTCCTACACCAGGCGTGCCAG
ACCTGGTGGATTTTCAGCCTCCTCCAGAGCTGGTGCTGAGAGAAGCTGGC
GAAGAAGTGCCAGACGCCGGACCTAGAGAGGGCGTTAGCTTTCCTTGGAG
CAGACCTCCTGGACAGGGCGAGTTCAGAGCCCTGAATGCTAGACTGCCCC
TGAACACCGATGCCTACCTGTCTCTGCAAGAGCTGCAGGGACAAGACCCC
ACACACCTCGTCAGAGTGAAGTTCAGCAGAAGCGCCGACGCTCCCGCCTA
TAAGCAGGGACAGAACCAGCTGTACAACGAGCTGAATCTGGGGCGCAGAG
AAGAGTACGATGTGCTGGACAAGCGGAGAGGCAGAGATCCTGAGATGGGC
GGCAAGCCCAGACGGAAGAATCCTCAAGAGGGCCTGTATAATGAGCTGCA
GAAAGACAAGATGGCCGAGGCCTACTCCGAGATCGGAATGAAGGGCGAGC
GCAGAAGAGGCAAGGGACACGATGGACTGTATCAGGGCCTGAGCACCGCC
ACCAAGGATACCTATGATGCCCTGCACATGCAGGCCCTGCCACCTAGA

Chimeric Stimulator Receptors and Recombinant HLA-E Polypeptides

Adoptive cell compositions that are “universally” safe for administration to any patient requires a significant reduction or elimination of alloreactivity. Towards this end, cells of the disclosure (e.g., allogenic cells) can be modified to interrupt expression or function of a T-cell Receptor (TCR) and/or a class of Major Histocompatibility Complex (MHC). The TCR mediates graft vs host (GvH) reactions whereas the MHC mediates host vs graft (HvG) reactions. In preferred aspects, any expression and/or function of the TCR is eliminated to prevent T-cell mediated GvH that could cause death to the subject. Thus, in a preferred aspect, the disclosure provides a pure TCR-negative allogeneic T-cell composition (e.g., each cell of the composition expresses at a level so low as to either be undetectable or non-existent).

Expression and/or function of MHC class I (MHC-I, specifically, HLA-A, HLA-B, and HLA-C) is reduced or eliminated to prevent HvG and, consequently, to improve engraftment of cells in a subject. Improved engraftment results in longer persistence of the cells, and, therefore, a larger therapeutic window for the subject. Specifically, expression and/or function of a structural element of MHC-I, Beta-2-Microglobulin (B2M), is reduced or eliminated.

The above strategies induce further challenges. T Cell Receptor (TCR) knockout (KO) in T cells results in loss of expression of CD3-zeta (CD3z or CD3ζ), which is part of the TCR complex. The loss of CD3λ in TCR-KO T-cells dramatically reduces the ability of optimally activating and expanding these cells using standard stimulation/activation reagents, including, but not limited to, agonist anti-CD3 mAb. When the expression or function of any one component of the TCR complex is interrupted, all components of the complex are lost, including TCR-alpha (TCRα), TCR-beta (TCRβ), CD3-gamma (CD3γ), CD3-epsilon (CD3 ε), CD3-delta (CD3 δ), and CD3-zeta (CD3ζ). Both CD3ε and CD3 are required for T cell activation and expansion. Agonist anti-CD3 mAbs typically recognize CD3ε and possibly another protein within the complex which, in turn, signals to CD3. CD3ζ provides the primary stimulus for T cell activation (along with a secondary co-stimulatory signal) for optimal activation and expansion. Under normal conditions, full T-cell activation depends on the engagement of the TCR in conjunction with a second signal mediated by one or more co-stimulatory receptors (e.g., CD28, CD2, 4-1BBL) that boost the immune response. However, when the TCR is not present, T cell expansion is severely reduced when stimulated using standard activation/stimulation reagents, including agonist anti-CD3 mAb. In fact, T cell expansion is reduced to only 20-40% of the normal level of expansion when stimulated using standard activation/stimulation reagents, including agonist anti-CD3 mAb.

The present disclosure provides a nucleic acid sequence encoding any CSR disclosed herein. The present disclosure provides a transposon or a vector comprising a nucleic acid sequence encoding any CSR disclosed herein.

The present disclosure provides a cell comprising any CSR disclosed herein. The present disclosure provides a cell comprising a nucleic acid sequence encoding any CSR disclosed herein. The present disclosure provides a cell comprising a vector comprising a nucleic acid sequence encoding any CSR disclosed herein. The present disclosure provides a cell comprising a transposon comprising a nucleic acid sequence encoding any CSR disclosed herein.

A modified cell disclosed herein can be an allogeneic cell or an autologous cell. In some preferred aspects, the modified cell is an allogeneic cell. In some aspects, the modified cell is an autologous T-cell or a modified autologous CSR T-cell. In some preferred aspects, the modified cell is an allogeneic T-cell or a modified allogeneic CSR T-cell.

The present disclosure provides a composition comprising any CSR disclosed herein. The present disclosure provides a composition comprising a nucleic acid sequence encoding any CSR disclosed herein. The present disclosure provides a composition comprising a vector comprising a nucleic acid sequence encoding any CSR disclosed herein. The present disclosure provides a composition comprising a transposon comprising a nucleic acid sequence encoding any CSR disclosed herein. The present disclosure provides a composition comprising a modified cell disclosed herein or a composition comprising a plurality of modified cells disclosed herein.

The present disclosure provides a modified T lymphocyte (T-cell), comprising: (a) a modification of an endogenous sequence encoding a T-cell Receptor (TCR), wherein the modification reduces or eliminates a level of expression or activity of the TCR; and (b) a chimeric stimulatory receptor (CSR) comprising: (i) an ectodomain comprising an activation component, wherein the activation component is isolated or derived from a first protein; (ii) a transmembrane domain; and (iii) an endodomain comprising at least one signal transduction domain, wherein the at least one signal transduction domain is isolated or derived from a second protein; wherein the first protein and the second protein are not identical.

The modified T-cell can further comprise an inducible proapoptotic polypeptide. The modified T-cell can further comprise a modification of an endogenous sequence encoding Beta-2-Microglobulin (B2M), wherein the modification reduces or eliminates a level of expression or activity of a major histocompatibility complex (WIC) class I (MHC-I).

The modified T-cell can further comprise a non-naturally occurring polypeptide comprising an HLA class I histocompatibility antigen, alpha chain E (HLA-E) polypeptide. The non-naturally occurring polypeptide comprising a HLA-E polypeptide can further comprise a B2M signal peptide. The non-naturally occurring polypeptide comprising a HLA-E polypeptide can further comprise a B2M polypeptide. The non-naturally occurring polypeptide comprising an HLA-E polypeptide can further comprise a linker, wherein the linker is positioned between the B2M polypeptide and the HLA-E polypeptide. The non-naturally occurring polypeptide comprising an HLA-E polypeptide can further comprise a peptide and a B2M polypeptide. The non-naturally occurring polypeptide comprising an HLA-E can further comprise a first linker positioned between the B2M signal peptide and the peptide, and a second linker positioned between the B2M polypeptide and the peptide encoding the HLA-E.

The modified T-cell can further comprise a non-naturally occurring antigen receptor, a sequence encoding a therapeutic polypeptide, or a combination thereof. The non-naturally occurring antigen receptor can comprise a chimeric antigen receptor (CAR).

The CSR can be transiently expressed in the modified T-cell. The CSR can be stably expressed in the modified T-cell. The polypeptide comprising the HLA-E polypeptide can be transiently expressed in the modified T-cell. The polypeptide comprising the HLA-E polypeptide can be stably expressed in the modified T-cell. The inducible proapoptotic polypeptide can be transiently expressed in the modified T-cell. The inducible proapoptotic polypeptide can be stably expressed in the modified T-cell. The non-naturally occurring antigen receptor or a sequence encoding a therapeutic protein can be transiently expressed in the modified T-cell. The non-naturally occurring antigen receptor or a sequence encoding a therapeutic protein can be stably expressed in the modified T-cell.

Gene editing compositions, including but not limited to, RNA-guided fusion proteins comprising dCas9-Clo051, as described in detail herein, can be used to target and decrease or eliminate expression of an endogenous T-cell receptor. In preferred aspects, the gene editing compositions target and delete a gene, a portion of a gene, or a regulatory element of a gene (such as a promoter) encoding an endogenous T-cell receptor. Non-limiting examples of primers (including a T7 promoter, genome target sequence, and gRNA scaffold) for the generation of guide RNA (gRNA) templates for targeting and deleting TCR-alpha (TCR-a), targeting and deleting TCR-beta (TCR-β), and targeting and deleting beta-2-microglobulin (β2M) are disclosed in PCT Application No. PCT/US2019/049816.

Gene editing compositions, including but not limited to, RNA-guided fusion proteins comprising dCas9-Clo051, can be used to target and decrease or eliminate expression of an endogenous MHCI, MHCII, or MHC activator. In preferred aspects, the gene editing compositions target and delete a gene, a portion of a gene, or a regulatory element of a gene (such as a promoter) encoding one or more components of an endogenous MHO, MHCII, or MHC activator. Non-limiting examples of guide RNAs (gRNAs) for targeting and deleting MHC activators are disclosed in PCT Application No. PCT/US2019/049816.

A detailed description of non-naturally occurring chimeric stimulatory receptors, genetic modifications of endogenous sequences encoding TCR-alpha (TCR-α), TCR-beta (TCR-β), and/or Beta-2-Microglobulin (β2M), and non-naturally occurring polypeptides comprising an HLA class I histocompatibility antigen, alpha chain E (HLA-E) polypeptide is disclosed in PCT Application No. PCT/US2019/049816.

### Cells and Modified Cells of the Disclosure

Regulatory T cells

“Regulatory T lymphocyte” or “Treg cell” or “Treg,” as used in the present specification and claims are synonymous and are intended to have its standard definition as used in the art. Treg cells are a specialized subpopulation of T cells that act in a “regulatory” way to suppress activation of the immune system and thereby maintain immune system homeostasis and tolerance to self-antigens. Tregs have sometimes been referred to suppressor T cells. Treg cells are characterized by expression of the forkhead family transcription factor Foxp3 (forkhead box p3). They are characterized by expression of CD4. They may express CD25 (also known as Interleukin 2 receptor subunit alpha, or IL2RA).

In general, T regulatory cells have been identified as a CD4+CD25+ T cell population capable of suppressing an immune response. The identification of Foxp3 as a “master-regulator” of Tregs helped define Tregs as a distinct T cell lineage. The identification of additional antigenic markers on the surface of Tregs has enabled identification and FACS sorting of viable Tregs to greater purity, resulting in a more highly-enriched and suppressive Treg population. In addition to CD4 and CD25, both mouse and human Tregs express GITR/AITR, CTLA-4, and express low levels of CD127 (IL-7Ra).

Moreover, Tregs can exist in different states which can be identified based on their expression of surface markers. Tregs which develop in the thymus from CD4+ thymocytes are known as “natural” Tregs, however Tregs can also be induced in the periphery from naive CD4+ T cells in response to low-dose engagement of the TCR, TGF beta and IL-2. These “induced” Tregs secrete the immunosuppressive cytokine IL-10. The phenotype of Tregs changes again as they become activated, and markers including GARP in mouse and human, CD45RA in human, and CD103 in mouse have been shown to be useful for the identification of activated Tregs. Tregs are important in the maintenance of immune cell homeostasis as evidenced by undesirable consequences of genetic or physical ablation of the Treg population. Treg cells generally maintain order in the immune system by enforcing a dominant negative regulation on other immune cells. Broadly classified into natural or adaptive (induced) Tregs; natural Tregs are CD4⁺CD25⁺ T cells which develop, and emigrate from the thymus to play a role in immune homeostasis. Adaptive Tregs are non-regulatory CD4⁺ T cells which acquire CD25 (IL-2R alpha) expression outside of the thymus, and may be induced by inflammation and disease processes, such as autoimmunity and cancer. Functional Tregs can also be forced through the overexpression of either of the two common human FoxP3 isoforms in CD4+CD25-cells. These are classified as “forced Tregs”.

There is increasing evidence that Tregs acquire their function through a myriad of mechanisms that may include the secretion of immunosuppressive soluble factors such as IL-10 and TGF beta, cell contact mediated regulation via the high affinity TCR and other costimulatory molecules such as CTLA-4 and GITR. Under the influence of TGF beta, adaptive Treg cells mature in peripheral sites, including mucosa-associated lymphoid tissue (MALT), from CD4+ Treg precursors, where they acquire the expression of markers typical of Tregs, including CD25, CTLA4 and GITR/AITR. Upon up-regulation of the transcription factor Foxp3, Treg cells begin their suppressive effect. This includes the secretion of cytokines including IL-10 and TGF beta which may induce cell-cycle arrest or apoptosis in effector T cells, and blocking co-stimulation and maturation of dendritic cells.

In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% or any percentage in between of the plurality of modified T cells in the population expresses one or more cell-surface marker(s) of a regulatory T cell (Treg); and wherein the one or more cell-surface marker(s) comprise CD4, CD25, FOXP3 or CD127. In certain embodiments, the Treg cell is CD4+CD25+ and FOXP3+. In certain embodiments, the Treg cell is CD4+CD25+ FOXP3+ and CD127^{low}. In certain embodiments, the Treg cell is CD4+CD25+CD127^{low}. In certain embodiments, the Treg cell is CD4+CD25+ FoxP3+CD127^{low} and CD45RA+.

A plurality of modified cells of the population comprise a transgene or a sequence encoding the transgene (e.g., a CSR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells of the population expresses one or more cell-surface marker(s) of a regulatory T cell (Treg).

Effector T cells

“Effector T cells” or “Teff cell” or “Teff” or “immune effector T cell” as used in the present specification and claims are synonymous and are intended to have its standard definition as used in the art. Effector T cells (Teffs) describes a group of cells that includes several T cell types that actively respond to a stimulus. Teff cells may refer to a cell that is involved with an immune response, e.g., in promotion of an immune effector response. Examples of effector cells include alpha/beta T cells and gamma/delta T cells, They are characterized by expression of CD3. They may express CD4. They may express CD8.

“Immune effector function or immune effector response,” as used in the present specific, refers to function or response, e.g., of an immune effector cell, that enhances or promotes an immune attack of a target cell. E.g., an immune effector function or response refers a property of a T cell that promotes killing or the inhibition of growth or proliferation, of a target cell. In the case of a T cell, primary stimulation and co-stimulation are examples of immune effector function or response. The term “effector function” refers to a specialized function of a cell. Effector function of a T cell, for example, may be cytolytic activity or helper activity including the secretion of cytokines. In some embodiments, the effector T cells is a CD8+ T cell. In some embodiments the effector T cell is an effector cytotoxic T cell (effector CTL). In some embodiments the effector T cells is a memory cytotoxic T cell (memory CTL).

One major difference between regulatory T cells and effectors cells is that regulatory T cells typically serve to modulate and deactivate the immune response, while effector T cells usually begin with immune-promoting cytokines and then switch to inhibitory cytokines later in their life cycle.

In certain embodiments, the Teff cell is CD4+CD3+. In certain embodiments, the Teff cell is CD8+CD3+. In certain embodiments the Teff is CD4+, CD3+, CD62L−, CCR7−, CD45RA+ and CD45RO−. In certain embodiments the Teff is CD8+, CD3+, CD62L−, CCR7-, CD45RA+ and CD45RO−.

In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% or any percentage in between of the plurality of modified T cells in the population expresses one or more cell-surface marker(s) of a effector T cell (Teff); and wherein the one or more cell-surface marker(s) comprise CD8+CD3+. The cell-surface markers can comprise one or more of CD4+, CD3+, CD62L−, CCR7−, CD45RA+ and CD45RO−. The cell-surface markers can comprise one or more of CD8+, CD3+, CD62L−, CCR7−, CD45RA+ and CD45RO−.

A plurality of modified cells of the population comprise a transgene or a sequence encoding the transgene (e.g., a CSR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells of the population expresses one or more cell-surface marker(s) of a effector T cell (Teff).

### T Cells

Modified T cells of the disclosure may be derived from modified hematopoietic stem and progenitor cells (HSPCs) or modified HSCs. Unlike traditional biologics and chemotherapeutics, the disclosed modified-T cells the capacity to rapidly reproduce upon antigen recognition, thereby potentially obviating the need for repeat treatments. To achieve this, in some embodiments, modified-T cells not only drive an initial response, but also persist in the patient as a stable population of viable memory T cells to prevent potential relapses. Alternatively, in some aspects, when it is not desired, the modified-T cells do not persist in the patient.

Intensive efforts have been focused on the development of antigen receptor molecules that do not cause T cell exhaustion through antigen-independent (tonic) signaling, as well as of a modified-T cell product containing early memory T cells, especially stem cell memory (T_{SCM}) or stem cell-like T cells. Stem cell-like modified-T cells of the disclosure exhibit the greatest capacity for self-renewal and multipotent capacity to derive central memory (T_{CM}) T cells or T_{CM} like cells, effector memory (T_{EM}) and effector T cells (T_{E}), thereby producing better tumor eradication and long-term modified-T cell engraftment. A linear pathway of differentiation may be responsible for generating these cells: Naive T cells (TN)>T_{SCM}>T_{CM}>T_{EM}>T_{E}>T_{TE}, whereby TN is the parent precursor cell that directly gives rise to T_{SCM}, which then, in turn, directly gives rise to T_{CM}, etc. Compositions of T cells of the disclosure can comprise one or more of each parental T cell subset with T_{SCM} cells being the most abundant (e.g., T_{SCM}>T_{CM}>T_{EM}>T_{E}>T_{TE}).

The immune cell precursor can be differentiated into or is capable of differentiating into an early memory T cell, a stem cell like T-cell, a Naive T cells (T_{N}), a T_{SCM}, a T_{CM}, a T_{EM}, a T_{E}, or a T_{TE}. The immune cell precursor can be a primitive HSC, an HSC, or a HSC descendent cell of the disclosure. The immune cell can be an early memory T cell, a stem cell like T-cell, a Naive T cells (T_{N}), a T_{SCM}, a T_{CM}, a T_{EM}, a T_{E}, or a T_{TE}.

The methods of the disclosure can modify and/or produce a population of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of a plurality of modified T cells in the population expresses one or more cell-surface marker(s) of an early memory T cell. The population of modified early memory T cells comprises a plurality of modified stem cell-like T cells. The population of modified early memory T cells comprises a plurality of modified T_{SCM} cells. The population of modified early memory T cells comprises a plurality of modified T_{CM} cells.

The methods of the disclosure can modify and/or produce a population of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells in the population expresses one or more cell-surface marker(s) of a stem cell-like T cell. The population of modified stem cell-like T cells comprises a plurality of modified T_{SCM} cells. The population of modified stem cell-like T cells comprises a plurality of modified T_{CM} cells.

In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% or any percentage in between of the plurality of modified T cells in the population expresses one or more cell-surface marker(s) of a stem memory T cell (T_{SCM}) or a T_{SCM}-like cell; and wherein the one or more cell-surface marker(s) comprise CD45RA and CD62L. The cell-surface markers can comprise one or more of CD62L, CD45RA, CD28, CCR7, CD127, CD45RO, CD95, CD95 and IL-2Rβ. The cell-surface markers can comprise one or more of CD45RA, CD95, IL-2Rβ, CCR7, and CD62L.

In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the plurality of modified T cells in the population expresses one or more cell-surface marker(s) of a central memory T cell (T_{CM}) or a T_{CM}-like cell; and wherein the one or more cell-surface marker(s) comprise CD45RO and CD62L. The cell-surface markers can comprise one or more of CD45RO, CD95, IL-2Rβ, CCR7, and CD62L.

The methods of the disclosure can modify and/or produce a population of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells in the population expresses one or more cell-surface marker(s) of a naive T cell (T_{N}). The cell-surface markers can comprise one or more of CD45RA, CCR7 and CD62L.

The methods of the disclosure can modify and/or produce a population of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells in the population expresses one or more cell-surface marker(s) of an effector T-cell (modified T_{EFF}). The cell-surface markers can comprise one or more of CD45RA, CD95, and IL-2Rβ.

The methods of the disclosure can modify and/or produce a population of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells of the population expresses one or more cell-surface marker(s) of a stem cell-like T cell, a stem memory T cell (T_{SCM}) or a central memory T cell (T_{CM}).

A plurality of modified cells of the population comprise a transgene or a sequence encoding the transgene (e.g., a CSR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells of the population expresses one or more cell-surface marker(s) of a stem cell-like T cell, a stem memory T cell (T_{SCM}) or a central memory T cell (T_{CM}).

A plurality of modified cells of the population comprise a transgene or a sequence encoding the transgene (e.g., a CSR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34 or wherein at least about 70% to about 99%, about 75% to about 95% or about 85% to about 95% of the population of modified cells express one or more cell-surface marker(s) comprising CD34 (e.g., comprise the cell-surface marker phenotype CD34+).

A plurality of modified cells of the population comprise a transgene or a sequence encoding the transgene (e.g., a CSR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34 and do not express one or more cell-surface marker(s) comprising CD38, or wherein at least about 45% to about 90%, about 50% to about 80% or about 65% to about 75% of the population of modified cells express one or more cell-surface marker(s) comprising CD34 and do not express one or more cell-surface marker(s) comprising CD38 (e.g., comprise the cell-surface marker phenotype CD34+ and CD38−).

A plurality of modified cells of the population comprise a transgene or a sequence encoding the transgene (e.g., a CSR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 0.1%, at least 0.2%, at least 0.3%, at least 0.4%, at least 0.5%, at least 0.6%, at least 0.7%, at least 0.8%, at least 0.9%, at least 1%, at least 1.5%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34 and CD90 and do not express one or more cell-surface marker(s) comprising CD38, or wherein at least about 0.2% to about 40%, about 0.2% to about 30%, about 0.2% to about 2% or 0.5% to about 1.5% of the population of modified cells express one or more cell-surface marker(s) comprising CD34 and CD90 and do not express one or more cell-surface marker(s) comprising CD38 (e.g., comprise the cell-surface marker phenotype CD34+, CD38- and CD90+).

A plurality of modified cells of the population comprise a transgene or a sequence encoding the transgene (e.g., a CSR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 0.1%, at least 0.2%, at least 0.3%, at least 0.4%, at least 0.5%, at least 0.6%, at least 0.7%, at least 0.8%, at least 0.9%, at least 1%, at least 1.5%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34 and CD90 and do not express one or more cell-surface marker(s) comprising CD38 and CD45RA, or wherein at least about 0.2% to about 40%, about 0.2% to about 30%, about 0.2% to about 2% or 0.5% to about 1.5% of the population of modified cells express one or more cell-surface marker(s) comprising CD34 and CD90 and do not express one or more cell-surface marker(s) comprising CD38 and CD45RA (e.g., comprise the cell-surface marker phenotype CD34+, CD38−, CD90+, CD45RA−).

A plurality of modified cells of the population comprise a transgene or a sequence encoding the transgene (e.g., a CSR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, at least 1.5%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34, CD90 and CD49f and do not express one or more cell-surface marker(s) comprising CD38 and CD45RA, or wherein at least about 0.02% to about 30%, about 0.02% to about 2%, about 0.04% to about 2% or about 0.04% to about 1% of the population of modified cells express one or more cell-surface marker(s) comprising CD34, CD90 and CD49f and do not express one or more cell-surface marker(s) comprising CD38 and CD45RA (e.g., comprise the cell-surface marker phenotype CD34+, CD38−, CD90+, CD45RA- and CD49f+).

A plurality of modified cells of the population comprise a transgene or a sequence encoding the transgene (e.g., a CSR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, at least 1.5%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34 and CD90 and do not express one or more cell-surface marker(s) comprising CD45RA, or wherein at least about 0.2% to about 5%, about 0.2% to about 3% or about 0.4% to about 3% of the population of modified cells express one or more cell-surface marker(s) comprising CD34 and CD90 and do not express one or more cell-surface marker(s) comprising CD45RA (e.g., comprise the cell-surface marker phenotype CD34+, CD90+ and CD45RA−).

Immune Precursor Cells, NK cells and B cells

The immune precursor cells can comprise any cells which can differentiate into one or more types of immune cells. The immune precursor cells can comprise multipotent stem cells that can self-renew and develop into immune cells. The immune precursor cells can comprise hematopoietic stem cells (HSCs) or descendants thereof. The immune precursor cells can comprise precursor cells that can develop into immune cells. The immune precursor cells can comprise hematopoietic progenitor cells (HPCs).

Hematopoietic stem cells (HSCs) are multipotent, self-renewing cells. All differentiated blood cells from the lymphoid and myeloid lineages arise from HSCs. HSCs can be found in adult bone marrow, peripheral blood, mobilized peripheral blood, peritoneal dialysis effluent and umbilical cord blood.

HSCs can be isolated or derived from a primary or cultured stem cell. HSCs can be isolated or derived from an embryonic stem cell, a multipotent stem cell, a pluripotent stem cell, an adult stem cell, or an induced pluripotent stem cell (iPSC).

Immune precursor cells can comprise an HSC or an HSC descendent cell. Non-limiting examples of HSC descendent cells include multipotent stem cells, lymphoid progenitor cells, natural killer (NK) cells, T lymphocyte cells (T-cells), B lymphocyte cells (B-cells), myeloid progenitor cells, neutrophils, basophils, eosinophils, monocytes and macrophages.

HSCs produced by the disclosed methods can retain features of “primitive” stem cells that, while isolated or derived from an adult stem cell and while committed to a single lineage, share characteristics of embryonic stem cells. For example, the “primitive” HSCs produced by the disclosed methods retain their “stemness” following division and do not differentiate. Consequently, as an adoptive cell therapy, the “primitive” HSCs produced by the disclosed methods not only replenish their numbers, but expand in vivo. “Primitive” HSCs produced by disclosed the methods can be therapeutically-effective when administered as a single dose.

Primitive HSCs can be CD34+. Primitive HSCs can be CD34+ and CD38−. Primitive HSCs can be CD34+, CD38- and CD90+. Primitive HSCs can be CD34+, CD38−, CD90+ and CD45RA−. Primitive HSCs can be CD34+, CD38−, CD90+, CD45RA−, and CD49f+. Primitive HSCs can be CD34+, CD38−, CD90+, CD45RA−, and CD49f+.

Primitive HSCs, HSCs, and/or HSC descendent cells can be modified according to the disclosed methods to express an exogenous sequence (e.g., a chimeric antigen receptor or therapeutic protein). Modified primitive HSCs, modified HSCs, and/or modified HSC descendent cells can be forward differentiated to produce a modified immune cell including, but not limited to, a modified T cell, a modified natural killer cell and/or a modified B-cell.

The modified immune or immune precursor cells can be NK cells. The NK cells can be cytotoxic lymphocytes that differentiate from lymphoid progenitor cells. Modified NK cells can be derived from modified hematopoietic stem and progenitor cells (HSPCs) or modified HSCs. In some aspects, non-activated NK cells are derived from CD3-depleted leukapheresis (containing CD14/CD19/CD56+ cells).

The modified immune or immune precursor cells can be B cells. B cells are a type of lymphocyte that express B cell receptors on the cell surface. B cell receptors bind to specific antigens. Modified B cells can be derived from modified hematopoietic stem and progenitor cells (HSPCs) or modified HSCs.

Modified T cells of the disclosure may be derived from modified hematopoietic stem and progenitor cells (HSPCs) or modified HSCs. Unlike traditional biologics and chemotherapeutics, the disclosed modified-T cells the capacity to rapidly reproduce upon antigen recognition, thereby potentially obviating the need for repeat treatments. To achieve this, in some embodiments, modified-T cells not only drive an initial response, but also persist in the patient as a stable population of viable memory T cells to prevent potential relapses. Alternatively, in some aspects, when it is not desired, the modified-T cells do not persist in the patient.

Compositions and Methods of Producing, Activating and/or Expanding T cells

In some embodiments of the methods of the disclosure, a buffer comprises the immune cell or precursor thereof. The buffer maintains or enhances a level of cell viability and/or a Treg phenotype of the immune cell or precursor thereof, including T cells. In certain embodiments, the buffer maintains or enhances a level of cell viability and/or a Treg phenotype of the primary human T cells prior to the nucleofection. In certain embodiments, the buffer maintains or enhances a level of cell viability and/or a Treg phenotype of the primary human T cells during the nucleofection. In certain embodiments, the buffer maintains or enhances a level of cell viability and/or a Treg phenotype of the primary human T cells following the nucleofection. In certain embodiments, the buffer comprises one or more of KCl, MgCl₂, ClNa, Glucose and Ca(NO₃)₂ in any absolute or relative abundance or concentration, and, optionally, the buffer further comprises a supplement selected from the group consisting of HEPES, Tris/HCl, and a phosphate buffer. In certain embodiments, the buffer comprises 5 mM KCl, 15 mM MgCl₂, 90 mM ClNa, 10 mM Glucose and 0.4 mM Ca(NO₃)₂. In certain embodiments, the buffer comprises 5 mM KCl, 15 mM MgCl₂, 90 mM ClNa, 10 mM Glucose and 0.4 mM Ca(NO₃)₂ and a supplement comprising 20 mM HEPES and 75 mM Tris/HCl. In certain embodiments, the buffer comprises 5 mM KCl, 15 mM MgCl₂, 90 mM ClNa, 10 mM Glucose and 0.4 mM Ca(NO₃)₂ and a supplement comprising 40 mM Na₂HPO₄/NaH₂PO₄ at pH 7.2. In certain embodiments, the composition comprising primary human T cells comprises 100 μl of the buffer and between 5×10⁶ and 25×10⁶ cells. In certain embodiments, the composition comprises a scalable ratio of 250×10⁶ primary human T cells per milliliter of buffer or other media during the introduction step.

In some embodiments of the methods of the disclosure, the methods comprise contacting an immune cell of the disclosure, including a T cell of the disclosure, and a T cell expansion composition. In some embodiments of the methods of the disclosure, the step of introducing a transposon and/or transposase of the disclosure into an immune cell of the disclosure may further comprise contacting the immune cell and a T cell expansion composition. In some embodiments, including those in which the introducing step of the methods comprises an electroporation or a nucleofection step, the electroporation or a nucleofection step may be performed with the immune cell contacting T cell expansion composition of the disclosure.

In some embodiments of the methods of the disclosure, the T cell expansion composition comprises, consists essentially of or consists of phosphorus; one or more of an octanoic acid, a palmitic acid, a linoleic acid, and an oleic acid; a sterol; and an alkane.

In certain embodiments of the methods of producing a modified T cell of the disclosure, the expansion supplement comprises one or more cytokine(s). The one or more cytokine(s) may comprise any cytokine, including but not limited to, lymphokines. Exemplary lympokines include, but are not limited to, interleukin-2 (IL-2), interleukin-3 (IL-3), interleukin-4 (IL-4), interleukin-5 (IL-5), interleukin-6 (IL-6), interleukin-7 (IL-7), interleukin-15 (IL-15), interleukin-21 (IL-21), granulocyte-macrophage colony-stimulating factor (GM-CSF) and interferon-gamma (INFγ). The one or more cytokine(s) may comprise IL-2.

In some embodiments of the methods of the disclosure, the T cell expansion composition comprises human serum albumin, recombinant human insulin, human transferrin, 2-Mercaptoethanol, and an expansion supplement. In certain embodiments of this method, the T cell expansion composition further comprises one or more of octanoic acid, nicotinamide, 2,4,7,9-tetramethyl-5-decyn-4,7-diol (TMDD), diisopropyl adipate (DIPA), n-butyl-benzenesulfonamide, 1,2-benzenedicarboxylic acid, bis(2-methylpropyl) ester, palmitic acid, linoleic acid, oleic acid, stearic acid hydrazide, oleamide, a sterol and an alkane. In certain embodiments of this method, the T cell expansion composition further comprises one or more of octanoic acid, palmitic acid, linoleic acid, oleic acid and a sterol. In certain embodiments of this method, the T cell expansion composition further comprises one or more of octanoic acid at a concentration of between 0.9 mg/kg to 90 mg/kg, inclusive of the endpoints; palmitic acid at a concentration of between 0.2 mg/kg to 20 mg/kg, inclusive of the endpoints; linoleic acid at a concentration of between 0.2 mg/kg to 20 mg/kg, inclusive of the endpoints; oleic acid at a concentration of 0.2 mg/kg to 20 mg/kg, inclusive of the endpoints; and a sterol at a concentration of about 0.1 mg/kg to 10 mg/kg, inclusive of the endpoints. In certain embodiments of this method, the T cell expansion composition further comprises one or more of octanoic acid at a concentration of about 9 mg/kg, palmitic acid at a concentration of about 2 mg/kg, linoleic acid at a concentration of about 2 mg/kg, oleic acid at a concentration of about 2 mg/kg and a sterol at a concentration of about 1 mg/kg. In certain embodiments of this method, the T cell expansion composition further comprises one or more of octanoic acid at a concentration of between 6.4 μmol/kg and 640 μmol/kg, inclusive of the endpoints; palmitic acid at a concentration of between 0.7 μmol/kg and 70 μmol/kg, inclusive of the endpoints; linoleic acid at a concentration of between 0.75 μmol/kg and 75 μmol/kg, inclusive of the endpoints; oleic acid at a concentration of between 0.75 μmol/kg and 75 μmol/kg, inclusive of the endpoints; and a sterol at a concentration of between 0.25 μmol/kg and 25 μmol/kg, inclusive of the endpoints. In certain embodiments of this method, the T cell expansion composition further comprises one or more of octanoic acid at a concentration of about 64 μmol/kg, palmitic acid at a concentration of about 7 μmol/kg, linoleic acid at a concentration of about 7.5 μmol/kg, oleic acid at a concentration of about 7.5 μmol/kg and a sterol at a concentration of about 2.5 μmol/kg.

In certain embodiments, the T cell expansion composition comprises one or more of human serum albumin, recombinant human insulin, human transferrin, 2-Mercaptoethanol, and an expansion supplement to produce a plurality of expanded modified T cells, wherein at least 2% of the plurality of modified T cells expresses one or more cell-surface marker(s) of a Treg cell. In certain embodiments, the T cell expansion composition comprises or further comprises one or more of octanoic acid, nicotinamide, 2,4,7,9-tetramethyl-5-decyn-4,7-diol (TMDD), diisopropyl adipate (DIPA), n-butyl-benzenesulfonamide, 1,2-benzenedicarboxylic acid, bis(2-methylpropyl) ester, palmitic acid, linoleic acid, oleic acid, stearic acid hydrazide, oleamide, a sterol and an alkane. In certain embodiments, the T cell expansion composition comprises one or more of octanoic acid, palmitic acid, linoleic acid, oleic acid and a sterol (e.g., cholesterol). In certain embodiments, the T cell expansion composition comprises one or more of octanoic acid at a concentration of between 0.9 mg/kg to 90 mg/kg, inclusive of the endpoints; palmitic acid at a concentration of between 0.2 mg/kg to 20 mg/kg, inclusive of the endpoints; linoleic acid at a concentration of between 0.2 mg/kg to 20 mg/kg, inclusive of the endpoints; oleic acid at a concentration of 0.2 mg/kg to 20 mg/kg, inclusive of the endpoints; and a sterol at a concentration of about 0.1 mg/kg to 10 mg/kg, inclusive of the endpoints (wherein mg/kg=parts per million). In certain embodiments, the T cell expansion composition comprises one or more of octanoic acid at a concentration of about 9 mg/kg, palmitic acid at a concentration of about 2 mg/kg, linoleic acid at a concentration of about 2 mg/kg, oleic acid at a concentration of about 2 mg/kg, and a sterol at a concentration of about 1 mg/kg (wherein mg/kg=parts per million). In certain embodiments, the T cell expansion composition comprises one or more of octanoic acid at a concentration of 9.19 mg/kg, palmitic acid at a concentration of 1.86 mg/kg, linoleic acid at a concentration of about 2.12 mg/kg, oleic acid at a concentration of about 2.13 mg/kg, and a sterol at a concentration of about 1.01 mg/kg (wherein mg/kg=parts per million). In certain embodiments, the T cell expansion composition comprises octanoic acid at a concentration of 9.19 mg/kg, palmitic acid at a concentration of 1.86 mg/kg, linoleic acid at a concentration of 2.12 mg/kg, oleic acid at a concentration of about 2.13 mg/kg, and a sterol at a concentration of 1.01 mg/kg (wherein mg/kg=parts per million). In certain embodiments, the T cell expansion composition comprises one or more of octanoic acid at a concentration of between 6.4 mol/kg and 640 μmol/kg, inclusive of the endpoints; palmitic acid at a concentration of between 0.7 μmol/kg and 70 μmol/kg, inclusive of the endpoints; linoleic acid at a concentration of between 0.75 μmol/kg and 75 μmol/kg, inclusive of the endpoints; oleic acid at a concentration of between 0.75 μmol/kg and 75 μmol/kg, inclusive of the endpoints; and a sterol at a concentration of between 0.25 μmol/kg and 25 μmol/kg, inclusive of the endpoints. In certain embodiments, the T cell expansion composition comprises one or more of octanoic acid at a concentration of about 64 μmol/kg, palmitic acid at a concentration of about 7 μmol/kg, linoleic acid at a concentration of about 7.5 μmol/kg, oleic acid at a concentration of about 7.5 μmol/kg and a sterol at a concentration of about 2.5 μmol/kg. In certain embodiments, the T cell expansion composition comprises one or more of octanoic acid at a concentration of about 63.75 μmol/kg, palmitic acid at a concentration of about 7.27 mol/kg, linoleic acid at a concentration of about 7.57 μmol/kg, oleic acid at a concentration of about 7.56 μmol/kg and a sterol at a concentration of about 2.61 μmol/kg. In certain embodiments, the T cell expansion composition comprises octanoic acid at a concentration of about 63.75 μmol/kg, palmitic acid at a concentration of about 7.27 μmol/kg, linoleic acid at a concentration of about 7.57 μmol/kg, oleic acid at a concentration of 7.56 μmol/kg and a sterol at a concentration of 2.61 μmol/kg.

As used herein, the terms “supplemented T cell expansion composition” or “T cell expansion composition” may be used interchangeably with a media comprising one or more of human serum albumin, recombinant human insulin, human transferrin, 2-Mercaptoethanol, and an expansion supplement at 37° C. Alternatively, or in addition, the terms “supplemented T cell expansion composition” or “T cell expansion composition” may be used interchangeably with a media comprising one or more of phosphorus, an octanoic fatty acid, a palmitic fatty acid, a linoleic fatty acid and an oleic acid. In certain embodiments, the media comprises an amount of phosphorus that is 10-fold higher than may be found in, for example, Iscove's Modified Dulbecco's Medium ((IMDM); available at ThermoFisher Scientific as Catalog number 12440053).

As used herein, the terms “supplemented T cell expansion composition” or “T cell expansion composition” may be used interchangeably with a media comprising one or more of human serum albumin, recombinant human insulin, human transferrin, 2-Mercaptoethanol, Iscove's MDM, and an expansion supplement at 37° C. Alternatively, or in addition, the terms “supplemented T cell expansion composition” or “T cell expansion composition” may be used interchangeably with a media comprising one or more of the following elements: boron, sodium, magnesium, phosphorus, potassium, and calcium. In certain embodiments, the terms “supplemented T cell expansion composition” or “T cell expansion composition” may be used interchangeably with a media comprising one or more of the following elements present in the corresponding average concentrations: boron at 3.7 mg/L, sodium at 3000 mg/L, magnesium at 18 mg/L, phosphorus at 29 mg/L, potassium at 15 mg/L and calcium at 4 mg/L.

As used herein, the terms “supplemented T cell expansion composition” or “T cell expansion composition” may be used interchangeably with a media comprising one or more of human serum albumin, recombinant human insulin, human transferrin, 2-Mercaptoethanol, and an expansion supplement at 37° C. Alternatively, or in addition, the terms “supplemented T cell expansion composition” or “T cell expansion composition” may be used interchangeably with a media comprising one or more of the following components: octanoic acid (CAS No. 124-07-2), nicotinamide (CAS No. 98-92-0), 2,4,7,9-tetramethyl-5-decyn-4,7-diol (TMDD) (CAS No. 126-86-3), diisopropyl adipate (DIPA) (CAS No. 6938-94-9), n-butyl-benzenesulfonamide (CAS No. 3622-84-2), 1,2-benzenedicarboxylic acid, bis(2-methylpropyl) ester (CAS No. 84-69-5), palmitic acid (CAS No. 57-10-3), linoleic acid (CAS No. 60-33-3), oleic acid (CAS No. 112-80-1), stearic acid hydrazide (CAS No. 4130-54-5), oleamide (CAS No. 3322-62-1), sterol (e.g., cholesterol) (CAS No. 57-88-5), and alkanes (e.g., nonadecane) (CAS No. 629-92-5). In certain embodiments, the terms “supplemented T cell expansion composition” or “T cell expansion composition” may be used interchangeably with a media comprising one or more of the following components: octanoic acid (CAS No. 124-07-2), nicotinamide (CAS No. 98-92-0), 2,4,7,9-tetramethyl-5-decyn-4,7-diol (TMDD) (CAS No. 126-86-3), diisopropyl adipate (DIPA) (CAS No. 6938-94-9), n-butyl-benzenesulfonamide (CAS No. 3622-84-2), 1,2-benzenedicarboxylic acid, bis(2-methylpropyl) ester (CAS No. 84-69-5), palmitic acid (CAS No. 57-10-3), linoleic acid (CAS No. 60-33-3), oleic acid (CAS No. 112-80-1), stearic acid hydrazide (CAS No. 4130-54-5), oleamide (CAS No. 3322-62-1), sterol (e.g., cholesterol) (CAS No. 57-88-5), alkanes (e.g., nonadecane) (CAS No. 629-92-5), and phenol red (CAS No. 143-74-8). In certain embodiments, the terms “supplemented T cell expansion composition” or “T cell expansion composition” may be used interchangeably with a media comprising one or more of the following components: octanoic acid (CAS No. 124-07-2), nicotinamide (CAS No. 98-92-0), 2,4,7,9-tetramethyl-5-decyn-4,7-diol (TMDD) (CAS No. 126-86-3), diisopropyl adipate (DIPA) (CAS No. 6938-94-9), n-butyl-benzenesulfonamide (CAS No. 3622-84-2), 1,2-benzenedicarboxylic acid, bis(2-methylpropyl) ester (CAS No. 84-69-5), palmitic acid (CAS No. 57-10-3), linoleic acid (CAS No. 60-33-3), oleic acid (CAS No. 112-80-1), stearic acid hydrazide (CAS No. 4130-54-5), oleamide (CAS No. 3322-62-1), phenol red (CAS No. 143-74-8) and lanolin alcohol.

In certain embodiments, the terms “supplemented T cell expansion composition” or “T cell expansion composition” may be used interchangeably with a media comprising one or more of human serum albumin, recombinant human insulin, human transferrin, 2-Mercaptoethanol, and an expansion supplement at 37° C. Alternatively, or in addition, the terms “supplemented T cell expansion composition” or “T cell expansion composition” may be used interchangeably with a media comprising one or more of the following ions: sodium, ammonium, potassium, magnesium, calcium, chloride, sulfate and phosphate.

As used herein, the terms “supplemented T cell expansion composition” or “T cell expansion composition” may be used interchangeably with a media comprising one or more of human serum albumin, recombinant human insulin, human transferrin, 2-Mercaptoethanol, and an expansion supplement at 37° C. Alternatively, or in addition, the terms “supplemented T cell expansion composition” or “T cell expansion composition” may be used interchangeably with a media comprising one or more of the following free amino acids: histidine, asparagine, serine, glutamate, arginine, glycine, aspartic acid, glutamic acid, threonine, alanine, proline, cysteine, lysine, tyrosine, methionine, valine, isoleucine, leucine, phenylalanine and tryptophan. In certain embodiments, the terms “supplemented T cell expansion composition” or “T cell expansion composition” may be used interchangeably with a media comprising one or more of the following free amino acids in the corresponding average mole percentages: histidine (about 1%), asparagine (about 0.5%), serine (about 1.5%), glutamine (about 67%), arginine (about 1.5%), glycine (about 1.5%), aspartic acid (about 1%), glutamic acid (about 2%), threonine (about 2%), alanine (about 1%), proline (about 1.5%), cysteine (about 1.5%), lysine (about 3%), tyrosine (about 1.5%), methionine (about 1%), valine (about 3.5%), isoleucine (about 3%), leucine (about 3.5%), phenylalanine (about 1.5%) and tryptophan (about 0.5%). In certain embodiments, the terms “supplemented T cell expansion composition” or “T cell expansion composition” may be used interchangeably with a media comprising one or more of the following free amino acids in the corresponding average mole percentages: histidine (about 0.78%), asparagine (about 0.4%), serine (about 1.6%), glutamine (about 67.01%), arginine (about 1.67%), glycine (about 1.72%), aspartic acid (about 1.00%), glutamic acid (about 1.93%), threonine (about 2.38%), alanine (about 1.11%), proline (about 1.49%), cysteine (about 1.65%), lysine (about 2.84%), tyrosine (about 1.62%), methionine (about 0.85%), valine (about 3.45%), isoleucine (about 3.14%), leucine (about 3.3%), phenylalanine (about 1.64%) and tryptophan (about 0.37%).

As used herein, the terms “supplemented T cell expansion composition” or “T cell expansion composition” may be used interchangeably with a media comprising one or more of human serum albumin, recombinant human insulin, human transferrin, 2-Mercaptoethanol, Iscove's MDM, and an expansion supplement at 37° C. Alternatively, or in addition, the terms “supplemented T cell expansion composition” or “T cell expansion composition” may be used interchangeably with a media comprising one or more of phosphorus, an octanoic fatty acid, a palmitic fatty acid, a linoleic fatty acid and an oleic acid. In certain embodiments, the media comprises an amount of phosphorus that is 10-fold higher than may be found in, for example, Iscove's Modified Dulbecco's Medium ((IMDM); available at ThermoFisher Scientific as Catalog number 12440053).

In certain embodiments, the terms “supplemented T cell expansion composition” or “T cell expansion composition” may be used interchangeably with a media comprising one or more of octanoic acid, palmitic acid, linoleic acid, oleic acid and a sterol (e.g., cholesterol). In certain embodiments, the terms “supplemented T cell expansion composition” or “T cell expansion composition” may be used interchangeably with a media comprising one or more of octanoic acid at a concentration of between 0.9 mg/kg to 90 mg/kg, inclusive of the endpoints; palmitic acid at a concentration of between 0.2 mg/kg to 20 mg/kg, inclusive of the endpoints; linoleic acid at a concentration of between 0.2 mg/kg to 20 mg/kg, inclusive of the endpoints; oleic acid at a concentration of 0.2 mg/kg to 20 mg/kg, inclusive of the endpoints; and a sterol at a concentration of about 0.1 mg/kg to 10 mg/kg, inclusive of the endpoints (wherein mg/kg=parts per million). In certain embodiments, the terms “supplemented T cell expansion composition” or “T cell expansion composition” may be used interchangeably with a media comprising one or more of octanoic acid at a concentration of about 9 mg/kg, palmitic acid at a concentration of about 2 mg/kg, linoleic acid at a concentration of about 2 mg/kg, oleic acid at a concentration of about 2 mg/kg, and a sterol at a concentration of about 1 mg/kg (wherein mg/kg=parts per million). In certain embodiments, the terms “supplemented T cell expansion composition” or “T cell expansion composition” may be used interchangeably with a media comprising one or more of octanoic acid at a concentration of 9.19 mg/kg, palmitic acid at a concentration of 1.86 mg/kg, linoleic acid at a concentration of about 2.12 mg/kg, oleic acid at a concentration of about 2.13 mg/kg, and a sterol at a concentration of about 1.01 mg/kg (wherein mg/kg=parts per million). In certain embodiments, the terms “supplemented T cell expansion composition” or “T cell expansion composition” may be used interchangeably with a media comprising one or more of octanoic acid at a concentration of 9.19 mg/kg, palmitic acid at a concentration of 1.86 mg/kg, linoleic acid at a concentration of 2.12 mg/kg, oleic acid at a concentration of about 2.13 mg/kg, and a sterol at a concentration of 1.01 mg/kg (wherein mg/kg=parts per million). In certain embodiments, the terms “supplemented T cell expansion composition” or “T cell expansion composition” may be used interchangeably with a media comprising one or more of octanoic acid at a concentration of between 6.4 μmol/kg and 640 μmol/kg, inclusive of the endpoints; palmitic acid at a concentration of between 0.7 μmol/kg and 70 μmol/kg, inclusive of the endpoints; linoleic acid at a concentration of between 0.75 μmol/kg and 75 μmol/kg, inclusive of the endpoints; oleic acid at a concentration of between 0.75 μmol/kg and 75 μmol/kg, inclusive of the endpoints; and a sterol at a concentration of between 0.25 μmol/kg and 25 μmol/kg, inclusive of the endpoints. In certain embodiments, the terms “supplemented T cell expansion composition” or “T cell expansion composition” may be used interchangeably with a media comprising one or more of octanoic acid at a concentration of about 64 μmol/kg, palmitic acid at a concentration of about 7 μmol/kg, linoleic acid at a concentration of about 7.5 μmol/kg, oleic acid at a concentration of about 7.5 μmol/kg and a sterol at a concentration of about 2.5 μmol/kg.

In certain embodiments, the terms “supplemented T cell expansion composition” or “T cell expansion composition” may be used interchangeably with a media comprising one or more of octanoic acid at a concentration of about 63.75 μmol/kg, palmitic acid at a concentration of about 7.27 μmol/kg, linoleic acid at a concentration of about 7.57 μmol/kg, oleic acid at a concentration of about 7.56 μmol/kg and a sterol at a concentration of about 2.61 μmol/kg. In certain embodiments, the terms “supplemented T cell expansion composition” or “T cell expansion composition” may be used interchangeably with a media comprising one or more of octanoic acid at a concentration of about 63.75 μmol/kg, palmitic acid at a concentration of about 7.27 μmol/kg, linoleic acid at a concentration of about 7.57 mol/kg, oleic acid at a concentration of 7.56 μmol/kg and a sterol at a concentration of 2.61 μmol/kg.

In certain embodiments of the methods of producing a modified T cell (e.g., a Treg cell or a Teff cell) of the disclosure, the method comprises contacting a modified T cell and an inhibitor of the P13K-Akt-mTOR pathway. Modified T cells of the disclosure, including modified Treg cells or Teff cells of the disclosure, may be incubated, cultured, grown, stored, or otherwise, combined at any step in the methods of the procedure with a growth medium comprising one or more inhibitors a component of a PI3K pathway. Exemplary inhibitors a component of a PI3K pathway include, but are not limited to, an inhibitor of GSK3β such as TWS119 (also known as GSK 3B inhibitor XII; CAS Number 601514-19-6 having a chemical formula C₁₈H₁₄N₄O₂). Exemplary inhibitors of a component of a PI3K pathway include, but are not limited to, bb007 (BLUEBIRDBIO™). Additional Exemplary inhibitors of a component of a PI3K pathway include, but are not limited to, an allosteric Akt inhibitor VIII (also referred to as Akti-1/2 having Compound number 10196499), ATP competitive inhibitors (Orthosteric inhibitors targeting the ATP-binding pocket of the protein kinase B (Akt)), Isoquinoline-5-sulfonamides (H-8, H-89, and NL-71-101), Azepane derivatives (A series of structures derived from (—)—balanol), Aminofurazans (GSK690693), Heterocyclic rings (7-azaindole, 6-phenylpurine derivatives, pyrrolo[2,3-d]pyrimidine derivatives, CCT128930, 3-aminopyrrolidine, anilinotriazole derivatives, spiroindoline derivatives, AZD5363, ipatasertib (GDC-0068, RG7440), A-674563, and A-443654), Phenylpyrazole derivatives (AT7867 and AT13148), Thiophenecarboxamide derivatives (Afuresertib (GSK2110183), 2-pyrimidyl-5-amidothiophene derivative (DC120), uprosertib (GSK2141795)), Allosteric inhibitors (Superior to orthosteric inhibitors providing greater specificity, reduced side-effects and less toxicity), 2,3-diphenylquinoxaline analogues (2,3-diphenylquinoxaline derivatives, triazolo[3,4-f][1,6]naphthyridin-3(2H)-one derivative (MK-2206)), Alkylphospholipids (Edelfosine (1-O-octadecyl-2-O-methyl-rac-glycero-3-phosphocholine, ET-18—OCH₃) ilmofosine (BM 41.440), miltefosine (hexadecylphosphocholine, HePC), perifosine (D-21266), erucylphosphocholine (ErPC), erufosine (ErPC3, erucylphosphohomocholine), Indole-3-carbinol analogues (Indole-3-carbinol, 3-chloroacetylindole, diindolylmethane, diethyl 6-methoxy-5,7-dihydroindolo [2,3-b]carbazole-2,10-dicarboxylate (SR13668), OSU-A9), Sulfonamide derivatives (PH-316 and PHT-427), Thiourea derivatives (PIT-1, PIT-2, DM-PIT-1, N-[(1-methyl-1H-pyrazol-4-yl)carbonyl]-N′-(3-bromophenyl)-thiourea), Purine derivatives (Triciribine (TCN, NSC 154020), triciribine mono-phosphate active analogue (TCN-P), 4-amino-pyrido[2,3-d]pyrimidine derivative API-1, 3-phenyl-3H-imidazo[4,5-b]pyridine derivatives, ARQ 092), BAY 1125976, 3-methyl-xanthine, quinoline-4-carboxamide and 2-[4-(cyclohexa-1,3-dien-1-yl)-1H-pyrazol-3-yl]phenol, 3-oxo-tirucallic acid, 3α- and 3β-acetoxy-tirucallic acids, acetoxy-tirucallic acid, and irreversible inhibitors (antibiotics, Lactoquinomycin, Frenolicin B, kalafungin, medermycin, Boc-Phe-vinyl ketone, 4-hydroxynonenal (4-FINE), 1,6-naphthyridinone derivatives, and imidazo-1,2-pyridine derivatives).

In certain embodiments of the methods of producing a modified T cell (e.g., a Treg cell or a Teff cell) of the disclosure, the method comprises contacting a modified T cell and an inhibitor of T cell effector differentiation. Exemplary inhibitors of T cell effector differentiation include, but are not limited to, a BET inhibitor (e.g., JQ1, a hienotriazolodiazepine) and/or an inhibitor of the BET family of proteins (e.g., BRD2, BRD3, BRD4, and BRDT).

In certain embodiments of the methods of producing a modified T cell (e.g., a Treg cell or a Teff cell) of the disclosure, the method comprises contacting a modified T cell and an agent that reduces nucleo-cytoplasmic Acetyl-CoA. Exemplary agents that reduce nucleo-cytoplasmic Acetyl-CoA include, but are not limited to, 2-hydroxy-citrate (2-HC) as well as agents that increase expression of Acss1.

In certain embodiments of the methods of producing a modified T cell (e.g., a Treg cell or a Teff cell) of the disclosure, the method comprises contacting a modified T cell and a composition comprising a histone deacetylase (HDAC) inhibitor. In some embodiments, the composition comprising an HDAC inhibitor comprises or consists of valproic acid, Sodium Phenylbutyrate (NaPB) or a combination thereof. In some embodiments, the composition comprising an HDAC inhibitor comprises or consists of valproic acid. In some embodiments, the composition comprising an HDAC inhibitor comprises or consists of Sodium Phenylbutyrate (NaPB).

In certain embodiments of the methods of producing a modified T cell (e.g., a Treg cell or a Teff cell) of the disclosure, the activation supplement may comprise one or more cytokine(s). The one or more cytokine(s) may comprise any cytokine, including but not limited to, lymphokines. Exemplary lympokines include, but are not limited to, interleukin-2 (IL-2), interleukin-3 (IL-3), interleukin-4 (IL-4), interleukin-5 (IL-5), interleukin-6 (IL-6), interleukin-7 (IL-7), interleukin-15 (IL-15), interleukin-21 (IL-21), granulocyte-macrophage colony-stimulating factor (GM-CSF) and interferon-gamma (INFγ). The one or more cytokine(s) may comprise IL-2.

In certain embodiments of the methods of producing a modified T cell (e.g., a Treg cell or a Teff cell) of the disclosure, the activation supplement may comprise one or more activator complexes. Exemplary and nonlimiting activator complexes may comprise a monomeric, dimeric, trimeric or tetrameric antibody complex that binds one or more of CD3, CD28, and CD2. In some embodiments, the activation supplement comprises or consists of an activator complex that comprises a human, a humanized or a recombinant or a chimeric antibody. In some embodiments, the activation supplement comprises or consists of an activator complex that binds CD3 and CD28. In some embodiments, the activation supplement comprises or consists of an activator complex that binds CD3, CD28 and CD2.

Methods of Expressing a Chimeric Stimulatory Receptor

The disclosure provides methods of expressing a CSR on the surface of a cell. The method comprises (a) obtaining a cell population; (b) contacting the cell population to a composition comprising a CSR or a sequence encoding the CSR, under conditions sufficient to transfer the CSR across a cell membrane of at least one cell in the cell population, thereby generating a modified cell population; (c) culturing the modified cell population under conditions suitable for integration of the sequence encoding the CSR; and (d) expanding and/or selecting at least one cell from the modified cell population that express the CSR on the cell surface.

In some aspects, the cell population can comprise leukocytes and/or CD4+ and CD8+ leukocytes. The cell population can comprise CD4+ and CD8+ leukocytes in an optimized ratio. The optimized ratio of CD4+ to CD8+ leukocytes does not naturally occur in vivo. The cell population can comprise a tumor cell.

In some aspects, the conditions sufficient to transfer the CSR or the sequence encoding the CSR, transposon, or vector across a cell membrane of at least one cell in the cell population comprises at least one of an application of one or more pulses of electricity at a specified voltage, a buffer, and one or more supplemental factor(s). In some aspects, the conditions suitable for integration of the sequence encoding the CSR comprise at least one of a buffer and one or more supplemental factor(s).

The buffer can comprise PBS, HBSS, OptiMEM, BTXpress, Amaxa Nucleofector, Human T cell nucleofection buffer or any combination thereof. The one or more supplemental factor(s) can comprise (a) a recombinant human cytokine, a chemokine, an interleukin or any combination thereof; (b) a salt, a mineral, a metabolite or any combination thereof; (c) a cell medium; (d) an inhibitor of cellular DNA sensing, metabolism, differentiation, signal transduction, one or more apoptotic pathway(s) or combinations thereof; and (e) a reagent that modifies or stabilizes one or more nucleic acids. The recombinant human cytokine, the chemokine, the interleukin or any combination thereof can comprise IL2, IL7, IL12, IL15, IL21, ILL IL3, IL4, IL5, IL6, IL8, CXCL8, IL9, IL10, IL11, IL13, IL14, IL16, IL17, IL18, IL19, IL20, IL22, IL23, IL25, IL26, IL27, IL28, IL29, IL30, IL31, IL32, IL33, IL35, IL36, GM-CSF, IFN-gamma, IL-1 alpha/IL-1F1, IL-1 beta/IL-1F2, IL-12 p′70, IL-12/IL-35 p35, IL-13, IL-17/IL-17A, IL-17A/F Heterodimer, IL-17F, IL-18/IL-1F4, IL-23, IL-24, IL-32, IL-32 beta, IL-32 gamma, IL-33, LAP (TGF-beta 1), Lymphotoxin-alpha/TNF-beta, TGF-beta, TNF-alpha, TRANCE/TNFSF11/RANK L or any combination thereof. The salt, the mineral, the metabolite or any combination thereof can comprise HEPES, Nicotinamide, Heparin, Sodium Pyruvate, L-Glutamine, MEM Non-Essential Amino Acid Solution, Ascorbic Acid, Nucleosides, FBS/FCS, Human serum, serum-substitute, antibiotics, pH adjusters, Earle's Salts, 2-Mercaptoethanol, Human transferrin, Recombinant human insulin, Human serum albumin, Nucleofector PLUS Supplement, KCL, MgCl₂, Na₂HPO₄, NAH₂PO₄, Sodium lactobionate, Mannitol, Sodium succinate, Sodium Chloride, CINa, Glucose, Ca(NO₃)₂, Tris/HCl, K₂HPO₄, KH₂PO₄, Polyethylenimine, Poly-ethylene-glycol, Poloxamer 188, Poloxamer 181, Poloxamer 407, Poly-vinylpyrrolidone, Pop313, Crown-5, or any combination thereof. The cell medium can comprise PBS, HBSS, OptiMEM, DMEM, RPMI 1640, AIM-V, X—VIVO 15, CellGro DC Medium, CTS OpTimizer T Cell Expansion SFM, TexMACS Medium, PRIME-XV T Cell Expansion Medium, ImmunoCult-XF T Cell Expansion Medium or any combination thereof. The inhibitor of cellular DNA sensing, metabolism, differentiation, signal transduction, one or more apoptotic pathway(s) or combinations thereof comprise inhibitors of TLR9, MyD88, IRAK, TRAF6, TRAF3, IRF-7, NF-KB, Type 1 Interferons, pro-inflammatory cytokines, cGAS, STING, Sec5, TBK1, IRF-3, RNA pol III, RIG-1, IPS-1, FADD, RIP1, TRAF3, AIM2, ASC, Caspasel, Pro-IL1B, PI3K, Akt, Wnt3A, inhibitors of glycogen synthase kinase-3β (GSK-3β) (e.g. TWS119), or any combination thereof. Examples of such inhibitors can include Bafilomycin, Chloroquine, Quinacrine, AC-YVAD-CMK, Z-VAD-FMK, Z-IETD-FMK or any combination thereof. The reagent that modifies or stabilizes one or more nucleic acids comprises a pH modifier, a DNA-binding protein, a lipid, a phospholipid, CaPO4, a net neutral charge DNA binding peptide with or without a NLS sequence, a TREX1 enzyme or any combination thereof.

The expansion and selection steps can occur concurrently or sequentially. The expansion can occur prior to selection. The expansion can occur following selection, and, optionally, a further (i.e. second) selection can occur following expansion. Concurrent expansion and selection can be simultaneous. The expansion and/or selection steps can proceed for a period of 10 to 14 days, inclusive of the endpoints.

The expansion can comprise contacting at least one cell of the modified cell population with an antigen to stimulate the at least one cell through the CSR, thereby generating an expanded cell population. The antigen can be presented on the surface of a substrate. The substrate can have any form, including, but not limited to a surface, a well, a bead or a plurality thereof, and a matrix. The substrate can further comprise a paramagnetic or magnetic component. The antigen can be presented on the surface of a substrate, wherein the substrate is a magnetic bead, and wherein a magnet can be used to remove or separate the magnetic beads from the modified and expanded cell population. The antigen can be presented on the surface of a cell or an artificial antigen presenting cell. Artificial antigen presenting cells can include, but are not limited to, tumor cells and stem cells.

In some aspects wherein the transposon or vector comprises a selection gene, the selection step comprises contacting at least one cell of the modified cell population with a compound to which the selection gene confers resistance, thereby identifying a cell expressing the selection gene as surviving the selection and identifying a cell failing to express the selection gene as failing to survive the selection step.

The disclosure provides a composition comprising the modified, expanded and selected cell population of the methods described herein.

A more detailed description of methods for expressing a CSR or a CAR on the surface of a cell is disclosed in PCT Publication No. WO 2019/049816 and PCT/US2019/049816.

The present disclosure provides a cell or a population of cells wherein the cell comprises a composition comprising (a) an inducible transgene construct, comprising a sequence encoding an inducible promoter and a sequence encoding a transgene, and (b) a receptor construct, comprising a sequence encoding a constitutive promoter and a sequence encoding an exogenous receptor, such as a CSR, wherein, upon integration of the construct of (a) and the construct of (b) into a genomic sequence of a cell, the exogenous receptor is expressed, and wherein the exogenous receptor, upon binding a ligand or antigen, transduces an intracellular signal that targets directly or indirectly the inducible promoter regulating expression of the inducible transgene (a) to modify gene expression.

The composition can modify gene expression by decreasing gene expression. The composition can modify gene expression by transiently modifying gene expression (e.g., for the duration of binding of the ligand to the exogenous receptor). The composition can modify gene expression acutely (e.g., the ligand reversibly binds to the exogenous receptor). The composition can modify gene expression chronically (e.g., the ligand irreversibly binds to the exogenous receptor).

The exogenous receptor can comprise an endogenous receptor with respect to the genomic sequence of the cell. Exemplary receptors include, but are not limited to, intracellular receptors, cell-surface receptors, transmembrane receptors, ligand-gated ion channels, and G-protein coupled receptors.

The exogenous receptor can comprise a non-naturally occurring receptor. The non-naturally occurring receptor can be a synthetic, modified, recombinant, mutant or chimeric receptor. The non-naturally occurring receptor can comprise one or more sequences isolated or derived from a T-cell receptor (TCR). The non-naturally occurring receptor can comprise one or more sequences isolated or derived from a scaffold protein. In some aspects, including those wherein the non-naturally occurring receptor does not comprise a transmembrane domain, the non-naturally occurring receptor interacts with a second transmembrane, membrane-bound and/or an intracellular receptor that, following contact with the non-naturally occurring receptor, transduces an intracellular signal. The non-naturally occurring receptor can comprise a transmembrane domain. The non-naturally occurring receptor can interact with an intracellular receptor that transduces an intracellular signal. The non-naturally occurring receptor can comprise an intracellular signaling domain. The non-naturally occurring receptor can be a chimeric ligand receptor (CLR). The CLR can be a chimeric antigen receptor (CSR).

The sequence encoding the inducible promoter of comprises a sequence encoding an NFκB promoter, a sequence encoding an interferon (IFN) promoter or a sequence encoding an interleukin-2 promoter. In some aspects, the IFN promoter is an IFNγ promoter. The inducible promoter can be isolated or derived from the promoter of a cytokine or a chemokine. The cytokine or chemokine can comprise IL2, IL3, IL4, IL5, IL6, IL10, IL12, IL13, IL17A/F, IL21, IL22, IL23, transforming growth factor beta (TGF(3), colony stimulating factor 2 (GM-CSF), interferon gamma (IFNγ), Tumor necrosis factor alpha (TNFα), LTα, perforin, Granzyme C (Gzmc), Granzyme B (Gzmb), C—C motif chemokine ligand 5 (CCL5), C—C motif chemokine ligand 4 (Cc14), C—C motif chemokine ligand 3 (Cc13), X—C motif chemokine ligand 1 (Xcl1) or LIF interleukin 6 family cytokine (Lif).

The inducible promoter can be isolated or derived from the promoter of a gene comprising a surface protein involved in cell differentiation, activation, exhaustion and function. In some aspects, the gene comprises CD69, CD71, CTLA4, PD-1, TIGIT, LAG3, TIM-3, GITR, MHCII, COX-2, FASL or 4-1BB.

The inducible promoter can be isolated or derived from the promoter of a gene involved in CD metabolism and differentiation. The inducible promoter can be isolated or derived from the promoter of Nr4a1, Nr4a3, Tnfrsf9 (4-1BB), Sema7α, Zfp3612, Gadd45b, Dusp5, Dusp6 and Neto2.

In some aspects, the inducible transgene construct comprises or drives expression of a signaling component downstream of an inhibitory checkpoint signal, a transcription factor, a cytokine or a cytokine receptor, a chemokine or a chemokine receptor, a cell death or apoptosis receptor/ligand, a metabolic sensing molecule, a protein conferring sensitivity to a cancer therapy, and an oncogene or a tumor suppressor gene. Non-limiting examples of which are disclosed in PCT Publication No. WO 2019/173636 and PCT Application No. PCT/US2019/049816.

The present disclosure provides a method of producing a population of modified T-cells comprising, consisting essential of, or consisting of introducing into a plurality of primary human T-cells a composition comprising the CSR of the present disclosure or a sequence encoding the same to produce a plurality of modified T-cells. The present disclosure provides a composition comprising a population of modified T-cells produced by the method. In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population expresses the CSR of the present disclosure.

### Chimeric Antigen Receptors

In some embodiments of the compositions and methods of the disclosure, a modified Treg or Teff cell comprising a CSR of the disclosure can further comprise a chimeric antigen receptor.

In some embodiments of the compositions and methods of the disclosure, a transposon comprises a sequence encoding a chimeric antigen receptor or a portion thereof.

Chimeric antigen receptors (CARs) of the disclosure may comprise (a) an ectodomain comprising an antigen recognition region, (b) a transmembrane domain, and (c) an endodomain comprising at least one costimulatory domain. In certain embodiments, the ectodomain may further comprise a signal peptide. Alternatively, or in addition, in certain embodiments, the ectodomain may further comprise a hinge between the antigen recognition region and the transmembrane domain. In certain embodiments of the CARs of the disclosure, the signal peptide may comprise a sequence encoding a human CD2, CD3δ, CD3ε, CD3γ, CD3ζ, CD4, CD8α, CD19, CD28, 4-1BB or GM-CSFR signal peptide. In certain embodiments of the CARs of the disclosure, the signal peptide may comprise a sequence encoding a human CD8a signal peptide. In certain embodiments, the transmembrane domain may comprise a sequence encoding a human CD2, CD3δ, CD3ε, CD3γ, CD3ζ, CD4, CD8α, CD19, CD28, 4-1BB or GM-CSFR transmembrane domain. In certain embodiments of the CARs of the disclosure, the transmembrane domain may comprise a sequence encoding a human CD8a transmembrane domain. In certain embodiments of the CARs of the disclosure, the endodomain may comprise a human CD3t endodomain.

In certain embodiments of the CARs of the disclosure, the at least one costimulatory domain may comprise a human 4-1BB, CD28, CD40, ICOS, MyD88, OX-40 intracellular segment, or any combination thereof. In certain embodiments of the CARs of the disclosure, the at least one costimulatory domain may comprise a CD28 and/or a 4-1BB costimulatory domain. In certain embodiments of the CARs of the disclosure, the hinge may comprise a sequence derived from a human CD8α, IgG4, and/or CD4 sequence. In certain embodiments of the CARs of the disclosure, the hinge may comprise a sequence derived from a human CD8a sequence.

The CD28 costimulatory domain may comprise an amino acid sequence comprising RVKFSRSADAPAYKQGQNQLYNELNLGRREEYDVLDKRRGRDPEMGGKPRRKNPQ EGLYNELQKDKMAEAYSEIGMKGERRRGKGHDGLYQGLS TATKDTYDALHMQALP PR (SEQ ID NO: 212) or a sequence having at least 70%, 80%, 90%, 95%, or 99% identity to the amino acid sequence comprising RVKFSRSADAPAYKQGQNQLYNELNLGRREEYDVLDKRRGRDPEMGGKPRRKNPQ EGLYNELQKDKMAEAYSEIGMKGERRRGKGHDGLYQGLS TATKDTYDALHMQALP PR (SEQ ID NO: 212). The CD28 costimulatory domain may be encoded by the nucleic acid sequence comprising cgcgtgaagtttagtcgatcagcagatgccccagatacaaacagggacagaaccagctgtataacgagctgaatctgggccgccga gaggaatatgacgtgctggataageggagaggacgcgaccccgaaatgggaggcaagcccaggcgcaaaaaccctcaggaagg cctgtataacgagctgcagaaggacaaaatggcagaagcctattctgagatcggcatgaagggggagcgacggagaggcaaagg gcacgatgggctgtaccagggactgagcaccgccacaaaggacacctatgatgctctgcatatgcaggcactgcctccaagg (SEQ ID NO: 213). The 4-1BB costimulatory domain may comprise an amino acid sequence comprising KRGRKKLLYIFKQPFMRPVQTTQEEDGCSCRFPEEEEGGCEL (SEQ ID NO: 214) or a sequence having at least 70%, 80%, 90%, 95%, or 99% identity to the amino acid sequence comprising KRGRKKLLYIFKQPFMRPVQTTQEEDGCSCRFPEEEEGGCEL (SEQ ID NO: 214). The 4-1BB costimulatory domain may be encoded by the nucleic acid sequence comprising aagagaggcaggaagaaactgctgtatattricaaacagccatcatgcgccccgtgcagactacccaggaggaagacgggtgctcc tgtcgattccctgaggaagaggaaggcgggtgtgagctg (SEQ ID NO: 215). The 4-1BB costimulatory domain may be located between the transmembrane domain and the CD28 costimulatory domain.

In certain embodiments of the CARs of the disclosure, the hinge may comprise a sequence derived from a human CD8α, IgG4, and/or CD4 sequence. In certain embodiments of the CARs of the disclosure, the hinge may comprise a sequence derived from a human CD8α sequence. The hinge may comprise a human CD8α amino acid sequence comprising TTTPAPRPPTPAPTIASQPLSLRPEACRPAAGGAVHTRGLDFACD (SEQ ID NO: 216) or a sequence having at least 70%, 80%, 90%, 95%, or 99% identity to the amino acid sequence comprising TTTPAPRPPTPAPTIASQPLSLRPEACRPAAGGAVHTRGLDFACD (SEQ ID NO: 216). The human CD8α hinge amino acid sequence may be encoded by the nucleic acid sequence comprising actaccacaccagcacctagaccaccaactccagctccaaccatcgcgagtcagcccctgagtctgagacctgaggcctgcaggcc agctgcaggaggagctgtgcacaccaggggcctggacttcgcctgcgac (SEQ ID NO: 217).

Non-limiting examples of antigen recognition regions include scFvs, centyrins, VH, VHH, single domain antibodies, antibody mimetics, and monobodies, described in more detail in PCT Publication No. WO 2019/173636 and PCT/US2019/049816, which is incorporated by reference herein in its entirety.

Armored Cells

The modified cells of disclosure (e.g., CSR-T cells or CAR T-cells) can be further modified to enhance their therapeutic potential. Alternatively, or in addition, the modified cells may be further modified to render them less sensitive to immunologic and/or metabolic checkpoints. Modifications of this type “armor” the cells, which, following the modification, may be referred to here as “armored” cells (e.g., armored T-cells). Armored cells may be produced by, for example, blocking and/or diluting specific checkpoint signals delivered to the cells (e.g., checkpoint inhibition) naturally, within the tumor immunosuppressive microenvironment.

An armored cell of the disclosure can be derived from any cell, for example, a T cell, a NK cell, a hematopoietic progenitor cell, a peripheral blood (PB) derived T cell (including a T cell isolated or derived from G-CSF-mobilized peripheral blood), or an umbilical cord blood (UCB) derived T cell. An armored cell (e.g., armored T-cell) can comprise one or more of a chimeric ligand receptor (CLR comprising a protein scaffold, an antibody, an ScFv, or an antibody mimetic)/chimeric antigen receptor (CAR comprising a protein scaffold, an antibody, an ScFv, or an antibody mimetic), a CARTyrin (a CAR comprising a Centyrin), and/or a VCAR (a CAR comprising a camelid VHH or a single domain VH). An armored cell (e.g., armored T-cell) can comprise an inducible proapoptotic polypeptide as disclosed herein. An armored cell (e.g., armored T-cell) can comprise an exogenous sequence. The exogenous sequence can comprise a sequence encoding a therapeutic protein. Exemplary therapeutic proteins may be nuclear, cytoplasmic, intracellular, transmembrane, cell-surface bound, or secreted proteins. Exemplary therapeutic proteins expressed by the armored cell (e.g., armored T-cell) may modify an activity of the armored cell or may modify an activity of a second cell. An armored cell (e.g., armored T-cell) can comprise a selection gene or a selection marker. An armored cell (e.g., armored T-cell) can comprise a synthetic gene expression cassette (also referred to herein as an inducible transgene construct).

The modified cells of disclosure (e.g., CSR-T cells or CAR T-cells) can be further modified to silence or reduce expression one or more gene(s) encoding receptor(s) of inhibitory checkpoint signals to produce an armored cell (e.g., armored CAR T-cell). Receptors of inhibitory checkpoint signals are expressed on the cell surface or within the cytoplasm of a cell. Silencing or reducing expressing of the gene encoding the receptor of the inhibitory checkpoint signal results a loss of protein expression of the inhibitory checkpoint receptors on the surface or within the cytoplasm of an armored cell. Thus, armored cells having silenced or reduced expression of one or more genes encoding an inhibitory checkpoint receptor is resistant, non-receptive or insensitive to checkpoint signals. The resistance or decreased sensitivity of the armored cell to inhibitory checkpoint signals enhances the therapeutic potential of the armored cell in the presence of these inhibitory checkpoint signals. Non-limiting examples of inhibitory checkpoint signals (and proteins that induce immunosuppression) are disclosed in PCT Publication No. WO 2019/173636. Preferred examples of inhibitory checkpoint signals that may be silenced include, but are not limited to, PD-1 and TGFβRII.

The modified cells of disclosure (e.g., e.g., CSR-T cells or CAR T-cells) can be further modified to silence or reduce expression of one or more gene(s) encoding intracellular proteins involved in checkpoint signaling to produce an armored cell (e.g., armored CAR T-cell). The activity of the modified cells may be enhanced by targeting any intracellular signaling protein involved in a checkpoint signaling pathway, thereby achieving checkpoint inhibition or interference to one or more checkpoint pathways. Non-limiting examples of intracellular signaling proteins involved in checkpoint signaling are disclosed in PCT Publication No. WO 2019/173636.

The modified cells of disclosure (e.g., CSR-T cells or CAR T-cells) can be further modified to silence or reduce expression of one or more gene(s) encoding a transcription factor that hinders the efficacy of a therapy to produce an armored cell (e.g., armored CAR T-cell). The activity of modified cells may be enhanced or modulated by silencing or reducing expression (or repressing a function) of a transcription factor that hinders the efficacy of a therapy. Non-limiting examples of transcription factors that may be modified to silence or reduce expression or to repress a function thereof include, but are not limited to, the exemplary transcription factors are disclosed in PCT Publication No. WO 2019/173636.

The modified cells of disclosure (e.g., CSR-T cells or CAR T-cells) can be further modified to silence or reduce expression of one or more gene(s) encoding a cell death or cell apoptosis receptor to produce an armored cell (e.g., armored CAR T-cell). Interaction of a death receptor and its endogenous ligand results in the initiation of apoptosis. Disruption of an expression, an activity, or an interaction of a cell death and/or cell apoptosis receptor and/or ligand render a modified cell less receptive to death signals, consequently, making the armored cell more efficacious in a tumor environment. Non-limiting examples of cell death and/or cell apoptosis receptors and ligands are disclosed in PCT Publication No. WO 2019/173636. A preferred example of cell death receptor which may be modified is Fas (CD95).

The modified cells of disclosure (e.g., CSR-T cells or CAR T-cells) can be further modified to silence or reduce expression of one or more gene(s) encoding a metabolic sensing protein to produce an armored cell (e.g., armored CAR T-cell). Disruption to the metabolic sensing of the immunosuppressive tumor microenvironment (characterized by low levels of oxygen, pH, glucose and other molecules) by a modified cell leads to extended retention of T-cell function and, consequently, more tumor cells killed per cell. Non-limiting examples of metabolic sensing genes and proteins are disclosed in PCT Publication No. WO 2019/173636. A preferred example, HIF1a and VHL play a role in T-cell function while in a hypoxic environment. An armored T-cell may have silenced or reduced expression of one or more genes encoding HIF1a or VHL.

The modified cells of disclosure (e.g., CSR-T cells or CAR T-cells) can be further modified to silence or reduce expression of one or more gene(s) encoding proteins that that confer sensitivity to a cancer therapy, including a monoclonal antibody, to produce an armored cell (e.g., armored CAR T-cell). Thus, an armored cell can function and may demonstrate superior function or efficacy whilst in the presence of a cancer therapy (e.g., a chemotherapy, a monoclonal antibody therapy, or another anti-tumor treatment). Non-limiting examples of proteins involved in conferring sensitivity to a cancer therapy are disclosed in PCT Publication No. WO 2019/173636.

The modified cells of disclosure (e.g., CSR-T cells or CAR T-cells) can be further modified to silence or reduce expression of one or more gene(s) encoding a growth advantage factor to produce an armored cell (e.g., armored CAR T-cell). Silencing or reducing expression of an oncogene can confer a growth advantage for the cell. For example, silencing or reducing expression (e.g., disrupting expression) of a TET2 gene during a CAR T-cell manufacturing process results in the generation of an armored CAR T-cell with a significant capacity for expansion and subsequent eradication of a tumor when compared to a non-armored CAR T-cell lacking this capacity for expansion. This strategy may be coupled to a safety switch (e.g., an iC9 safety switch described herein), which permits the targeted disruption of an armored CAR T-cell in the event of an adverse reaction from a subject or uncontrolled growth of the armored CAR T-cell. Non-limiting examples of growth advantage factors are disclosed in PCT Publication No. WO 2019/173636.

The modified cells of disclosure (e.g., CSR-T cells or CAR T-cells) can be further modified to express a modified/chimeric checkpoint receptor to produce an armored T-cell of the disclosure.

The modified/chimeric checkpoint receptor can comprise a null receptor, decoy receptor or dominant negative receptor. A null receptor, decoy receptor or dominant negative receptor can be modified/chimeric receptor/protein. A null receptor, decoy receptor or dominant negative receptor can be truncated for expression of the intracellular signaling domain. Alternatively, or in addition, a null receptor, decoy receptor or dominant negative receptor can be mutated within an intracellular signaling domain at one or more amino acid positions that are determinative or required for effective signaling. Truncation or mutation of null receptor, decoy receptor or dominant negative receptor can result in loss of the receptor's capacity to convey or transduce a checkpoint signal to the cell or within the cell.

For example, a dilution or a blockage of an immunosuppressive checkpoint signal from a PD-L1 receptor expressed on the surface of a tumor cell may be achieved by expressing a modified/chimeric PD-1 null receptor on the surface of an armored cell (e.g., armored CAR T-cell), which effectively competes with the endogenous (non-modified) PD-1 receptors also expressed on the surface of the armored cell to reduce or inhibit the transduction of the immunosuppressive checkpoint signal through endogenous PD-1 receptors of the armored cell. In this non-limiting example, competition between the two different receptors for binding to PD-L1 expressed on the tumor cell reduces or diminishes a level of effective checkpoint signaling, thereby enhancing a therapeutic potential of the armored cell expressing the PD-1 null receptor.

The modified/chimeric checkpoint receptor can comprise a null receptor, decoy receptor or dominant negative receptor that is a transmembrane receptor, a membrane-associated or membrane-linked receptor/protein or an intracellular receptor/protein. Exemplary null, decoy, or dominant negative intracellular receptors/proteins include, but are not limited to, signaling components downstream of an inhibitory checkpoint signal, a transcription factor, a cytokine or a cytokine receptor, a chemokine or a chemokine receptor, a cell death or apoptosis receptor/ligand, a metabolic sensing molecule, a protein conferring sensitivity to a cancer therapy, and an oncogene or a tumor suppressor gene. Non-limiting examples of cytokines, cytokine receptors, chemokines and chemokine receptors are disclosed in PCT Publication No. WO 2019/173636.

The modified/chimeric checkpoint receptor can comprise a switch receptor. Exemplary switch receptors comprise a modified/chimeric receptor/protein wherein a native or wild type intracellular signaling domain is switched or replaced with a different intracellular signaling domain that is either non-native to the protein and/or not a wild-type domain. For example, replacement of an inhibitory signaling domain with a stimulatory signaling domain would switch an immunosuppressive signal into an immunostimulatory signal. Alternatively, replacement of an inhibitory signaling domain with a different inhibitory domain can reduce or enhance the level of inhibitory signaling. Expression or overexpression, of a switch receptor can result in the dilution and/or blockage of a cognate checkpoint signal via competition with an endogenous wild-type checkpoint receptor (not a switch receptor) for binding to the cognate checkpoint receptor expressed within the immunosuppressive tumor microenvironment. Armored cells (e.g., armored CAR T-cells) can comprise a sequence encoding a switch receptor, leading to the expression of one or more switch receptors, and consequently, altering an activity of an armored cell. Armored cells (e.g., armored CAR T-cells) can express a switch receptor that targets an intracellularly expressed protein downstream of a checkpoint receptor, a transcription factor, a cytokine receptor, a death receptor, a metabolic sensing molecule, a cancer therapy, an oncogene, and/or a tumor suppressor protein or gene.

Exemplary switch receptors can comprise or can be derived from a protein including, but are not limited to, the signaling components downstream of an inhibitory checkpoint signal, a transcription factor, a cytokine or a cytokine receptor, a chemokine or a chemokine receptor, a cell death or apoptosis receptor/ligand, a metabolic sensing molecule, a protein conferring sensitivity to a cancer therapy, and an oncogene or a tumor suppressor gene.

The modified cells of disclosure (e.g., CSR-T cells or CAR T-cells) can be further modified to express a CLR/CAR that mediates conditional gene expression to produce an armored T-cell. The combination of the CLR/CAR and the condition gene expression system in the nucleus of the armored T-cell constitutes a synthetic gene expression system that is conditionally activated upon binding of cognate ligand(s) with CLR or cognate antigen(s) with CAR. This system may help to ‘armor’ or enhance therapeutic potential of modified T-cells by reducing or limiting synthetic gene expression at the site of ligand or antigen binding, at or within the tumor environment for example.

Gene Editing Compositions and Methods

A modified cell be produced by introducing a transgene into the cell. The introducing step may comprise delivery of a nucleic acid sequence, a transgene, and/or a genomic editing construct via a non-transposition delivery system.

Introducing a nucleic acid sequence, transgene and/or a genomic editing construct into a cell ex vivo, in vivo, in vitro or in situ can comprise one or more of topical delivery, adsorption, absorption, electroporation, spin-fection, co-culture, transfection, mechanical delivery, sonic delivery, vibrational delivery, magnetofection or by nanoparticle-mediated delivery. Introducing a nucleic acid sequence, a transgene and/or a genomic editing construct into a cell ex vivo, in vivo, in vitro or in situ can comprise liposomal transfection, calcium phosphate transfection, fugene transfection, and dendrimer-mediated transfection. Introducing a nucleic acid sequence, a transgene, and/or a genomic editing construct into a cell ex vivo, in vivo, in vitro or in situ by mechanical transfection can comprise cell squeezing, cell bombardment, or gene gun techniques. Introducing a nucleic acid sequence, transgene and/or a genomic editing construct into a cell ex vivo, in vivo, in vitro or in situ by nanoparticle-mediated transfection can comprise liposomal delivery, delivery by micelles, and delivery by polymerosomes.

Introducing a nucleic acid sequence, transgene and/or a genomic editing construct into a cell ex vivo, in vivo, in vitro or in situ can comprise a non-viral vector. The non-viral vector can comprise a nucleic acid. The non-viral vector can comprise plasmid DNA, linear double-stranded DNA (dsDNA), linear single-stranded DNA (ssDNA), DoggyBone™ DNA, nanoplasmids, minicircle DNA, single-stranded oligodeoxynucleotides (ssODN), DDNA oligonucleotides, single-stranded mRNA (ssRNA), and double-stranded mRNA (dsRNA). The non-viral vector can comprise a transposon as described herein.

Introducing a nucleic acid sequence, transgene and/or a genomic editing construct into a cell ex vivo, in vivo, in vitro or in situ can comprise a viral vector. The viral vector can be a non-integrating non-chromosomal vector. Non-limiting examples of non-integrating non-chromosomal vectors include adeno-associated virus (AAV), adenovirus, and herpes viruses. The viral vector can be an integrating chromosomal vector. Non-limiting examples of integrating chromosomal vectors include adeno-associated vectors (AAV), Lentiviruses, and gamma-retroviruses.

Introducing a nucleic acid sequence, transgene and/or a genomic editing construct into a cell ex vivo, in vivo, in vitro or in situ can comprise a combination of vectors. Non-limiting examples of vector combinations include viral and non-viral vectors, a plurality of non-viral vectors, or a plurality of viral vectors. Non-limiting examples of vector combinations include a combination of a DNA-derived and an RNA-derived vector, a combination of an RNA and a reverse transcriptase, a combination of a transposon and a transposase, a combination of a non-viral vector and an endonuclease, and a combination of a viral vector and an endonuclease.

Genome modification can comprise introducing a nucleic acid sequence, transgene and/or a genomic editing construct into a cell ex vivo, in vivo, in vitro or in situ to stably integrate a nucleic acid sequence, transiently integrate a nucleic acid sequence, produce site-specific integration of a nucleic acid sequence, or produce a biased integration of a nucleic acid sequence. The nucleic acid sequence can be a transgene.

Genome modification can comprise introducing a nucleic acid sequence, transgene and/or a genomic editing construct into a cell ex vivo, in vivo, in vitro or in situ to stably integrate a nucleic acid sequence. The stable chromosomal integration can be a random integration, a site-specific integration, or a biased integration. The site-specific integration can be non-assisted or assisted. The assisted site-specific integration is co-delivered with a site-directed nuclease. The site-directed nuclease comprises a transgene with 5′ and 3′ nucleotide sequence extensions that contain a percentage homology to upstream and downstream regions of the site of genomic integration. The transgene with homologous nucleotide extensions enable genomic integration by homologous recombination, microhomology-mediated end joining, or nonhomologous end-joining. The site-specific integration can occur at a safe harbor site. Genomic safe harbor sites are able to accommodate the integration of new genetic material in a manner that ensures that the newly inserted genetic elements function reliably (for example, are expressed at a therapeutically effective level of expression) and do not cause deleterious alterations to the host genome that cause a risk to the host organism. Non-limiting examples of potential genomic safe harbors include intronic sequences of the human albumin gene, the adeno-associated virus site 1 (AAVS1), a naturally occurring site of integration of AAV virus on chromosome 19, the site of the chemokine (C—C motif) receptor 5 (CCRS) gene and the site of the human ortholog of the mouse Rosa26 locus.

The site-specific transgene integration can occur at a site that disrupts expression of a target gene. Disruption of target gene expression can occur by site-specific integration at introns, exons, promoters, genetic elements, enhancers, suppressors, start codons, stop codons, and response elements. Non-limiting examples of target genes targeted by site-specific integration include TRAC, TRAB, PDI, any immunosuppressive gene, and genes involved in allo-rejection.

The site-specific transgene integration can occur at a site that results in enhanced expression of a target gene. Enhancement of target gene expression can occur by site-specific integration at introns, exons, promoters, genetic elements, enhancers, suppressors, start codons, stop codons, and response elements.

Enzymes can be used to create strand breaks in the host genome to facilitate delivery or integration of the transgene. Enzymes can create single-strand breaks or double-strand breaks. Non-limiting examples of break-inducing enzymes include transposases, integrases, endonucleases, CRISPR-Cas9, transcription activator-like effector nucleases (TALEN), zinc finger nucleases (ZFN), Cas-CLOVER™, and CPF1. Break-inducing enzymes can be delivered to the cell encoded in DNA, encoded in mRNA, as a protein, or as a nucleoprotein complex with a guide RNA (gRNA).

The site-specific transgene integration can be controlled by a vector-mediated integration site bias. Vector-mediated integration site bias can controlled by the chosen lentiviral vector or by the chosen gamma-retroviral vector.

The site-specific transgene integration site can be a non-stable chromosomal insertion. The integrated transgene can be become silenced, removed, excised, or further modified. The genome modification can be a non-stable integration of a transgene. The non-stable integration can be a transient non-chromosomal integration, a semi-stable non chromosomal integration, a semi-persistent non-chromosomal insertion, or a non-stable chromosomal insertion. The transient non-chromosomal insertion can be epi-chromosomal or cytoplasmic. In one aspect, the transient non-chromosomal insertion of a transgene does not integrate into a chromosome and the modified genetic material is not replicated during cell division.

The genome modification can be a semi-stable or persistent non-chromosomal integration of a transgene. A DNA vector encodes a Scaffold/matrix attachment region (S-MAR) module that binds to nuclear matrix proteins for episomal retention of a non-viral vector allowing for autonomous replication in the nucleus of dividing cells.

The genome modification can be a non-stable chromosomal integration of a transgene. The integrated transgene can become silenced, removed, excised, or further modified.

The modification to the genome by transgene insertion can occur via host cell-directed double-strand breakage repair (homology-directed repair) by homologous recombination (HR), microhomology-mediated end joining (MMEJ), nonhomologous end joining (NHEJ), transposase enzyme-mediated modification, integrase enzyme-mediated modification, endonuclease enzyme-mediated modification, or recombinant enzyme-mediated modification. The modification to the genome by transgene insertion can occur via CRISPR-Cas9, TALEN, ZFNs, Cas-CLOVER™, and cpfl.

In gene editing systems that involve inserting new or existing nucleotides/nucleic acids, insertion tools (e.g., DNA template vectors, transposable elements (transposons or retrotransposons) must be delivered to the cell in addition to the cutting enzyme (e.g., a nuclease, recombinase, integrase or transposase). Examples of such insertion tools for a recombinase may include a DNA vector. Other gene editing systems require the delivery of an integrase along with an insertion vector, a transposase along with a transposon/retrotransposon, etc. An example recombinase that may be used as a cutting enzyme is the CRE recombinase. Non-limiting examples of integrases that may be used in insertion tools include viral based enzymes taken from any of a number of viruses including AAV, gamma retrovirus, and lentivirus. Examples transposons/retrotransposons that may be used in insertion tools are described in more detail herein.

A cell with an ex vivo, in vivo, in vitro or in situ genomic modification can be a germline cell or a somatic cell. The modified cell can be a human, non-human, mammalian, rat, mouse, or dog cell. The modified cell can be differentiated, undifferentiated, or immortalized. The modified undifferentiated cell can be a stem cell. The modified undifferentiated cell can be an induced pluripotent stem cell. The modified cell can be an immune cell. The modified cell can be a T cell, a hematopoietic stem cell, a natural killer cell, a macrophage, a dendritic cell, a monocyte, a megakaryocyte, or an osteoclast. The modified cell can be modified while the cell is quiescent, in an activated state, resting, in interphase, in prophase, in metaphase, in anaphase, or in telophase. The modified cell can be fresh, cryopreserved, bulk, sorted into sub-populations, from whole blood, from leukapheresis, or from an immortalized cell line. A detailed description for isolating cells from a leukapheresis product or blood is disclosed in in PCT Publication No. WO 2019/173636 and PCT/US2019/049816.

The present disclosure provides a gene editing composition and/or a cell comprising the gene editing composition. The gene editing composition can comprise a sequence encoding a DNA binding domain and a sequence encoding a nuclease protein or a nuclease domain thereof. The sequence encoding a nuclease protein or the sequence encoding a nuclease domain thereof can comprise a DNA sequence, an RNA sequence, or a combination thereof. The nuclease or the nuclease domain thereof can comprise one or more of a CRISPR/Cas protein, a Transcription Activator-Like Effector Nuclease (TALEN), a Zinc Finger Nuclease (ZFN), and an endonuclease.

The nuclease or the nuclease domain thereof can comprise a nuclease-inactivated Cas (dCas) protein and an endonuclease. The endonuclease can comprise a Clo051 nuclease or a nuclease domain thereof. The gene editing composition can comprise a fusion protein. The fusion protein can comprise a nuclease-inactivated Cas9 (dCas9) protein and a Clo051 nuclease or a Clo051 nuclease domain. The gene editing composition can further comprise a guide sequence. The guide sequence comprises an RNA sequence.

The disclosure provides compositions comprising a small, Cas9 (Cas9) operatively-linked to an effector. The disclosure provides a fusion protein comprising, consisting essentially of or consisting of a DNA localization component and an effector molecule, wherein the effector comprises a small, Cas9 (Cas9). A small Cas9 construct of the disclosure can comprise an effector comprising a type IIS endonuclease. A Staphylococcus aureus Cas9 with an active catalytic site comprises the amino acid sequence of SEQ ID NO: 218.

The disclosure provides compositions comprising an inactivated, small, Cas9 (dSaCas9) operatively-linked to an effector. The disclosure provides a fusion protein comprising, consisting essentially of or consisting of a DNA localization component and an effector molecule, wherein the effector comprises a small, inactivated Cas9 (dSaCas9). A small, inactivated Cas9 (dSaCas9) construct of the disclosure can comprise an effector comprising a type IIS endonuclease. A dSaCas9 comprises the amino acid sequence of SEQ ID NO: 219, which includes a D1OA and a N580A mutation to inactivate the catalytic site.

The disclosure provides compositions comprising an inactivated Cas9 (dCas9) operatively-linked to an effector. The disclosure provides a fusion protein comprising, consisting essentially of or consisting of a DNA localization component and an effector molecule, wherein the effector comprises an inactivated Cas9 (dCas9). An inactivated Cas9 (dCas9) construct of the disclosure can comprise an effector comprising a type IIS endonuclease.

The dCas9 can be isolated or derived from Streptococcus pyogenes. The dCas9 can comprise a dCas9 with substitutions at amino acid positions 10 and 840, which inactivate the catalytic site. In some aspects, these substitutions are D10A and H840A. The dCas9 can comprise the amino acid sequence of SEQ ID NO: 220 or SEQ ID NO: 221.

An exemplary Clo051 nuclease domain comprises, consists essentially of or consists of, the amino acid sequence of SEQ ID NO: 222.

An exemplary dCas9-Clo051 (Cas-CLOVER) fusion protein can comprise, consist essentially of, or consist of, the amino acid sequence of SEQ ID NO: 223. The exemplary dCas9-Clo051 fusion protein can be encoded by a polynucleotide which comprises, consists essentially of, or consists of, the nucleic acid sequence of SEQ ID NO: 224. The nucleic acid encoding the dCas9-Clo051 fusion protein can be DNA or RNA.

An exemplary dCas9-Clo051 (Cas-CLOVER) fusion protein can comprise, consist essentially of, or consist of, the amino acid sequence of SEQ ID NO: 225. The exemplary dCas9-Clo051 fusion protein can be encoded by a polynucleotide which comprises, consists essentially of, or consists of, the nucleic acid sequence of SEQ ID NO: 226. The nucleic acid encoding the dCas9-Clo051 fusion protein can be DNA or RNA.

A cell comprising the gene editing composition can express the gene editing composition stably or transiently. Preferably, the gene editing composition is expressed transiently. The guide RNA can comprise a sequence complementary to a target sequence within a genomic DNA sequence. The target sequence within a genomic DNA sequence can be a target sequence within a safe harbor site of a genomic DNA sequence.

Gene editing compositions, including Cas-CLOVER, and methods of using these compositions for gene editing are described in detail in U.S. Patent Publication Nos. 2017/0107541, 2017/0114149, 2018/0187185 and U.S. Pat. No. 10,415,024.

Gene editing tools can also be delivered to cells using one or more poly(histidine)-based micelles. Poly(histidine) (e.g., poly(L-histidine)), is a pH-sensitive polymer due to the imidazole ring providing an electron lone pair on the unsaturated nitrogen. That is, poly(histidine) has amphoteric properties through protonation-deprotonation. In particular, at certain pHs, poly(histidine)—containing triblock copolymers may assemble into a micelle with positively charged poly(histidine) units on the surface, thereby enabling complexing with the negatively-charged gene editing molecule(s). Using these nanoparticles to bind and release proteins and/or nucleic acids in a pH-dependent manner may provide an efficient and selective mechanism to perform a desired gene modification. In particular, this micelle-based delivery system provides substantial flexibility with respect to the charged materials, as well as a large payload capacity, and targeted release of the nanoparticle payload. In one example, site-specific cleavage of the double stranded DNA is enabled by delivery of a nuclease using the poly(histidine)-based micelles. Without wishing to be bound by a particular theory, it is believed that believed that in the micelles that are formed by the various triblock copolymers, the hydrophobic blocks aggregate to form a core, leaving the hydrophilic blocks and poly(histidine) blocks on the ends to form one or more surrounding layer.

In an aspect, the disclosure provides triblock copolymers made of a hydrophilic block, a hydrophobic block, and a charged block. In some aspects, the hydrophilic block may be poly(ethylene oxide) (PEO), and the charged block may be poly(L-histidine). An example tri-block copolymer that can be used is a PEO-b-PLA-b-PHIS, with variable numbers of repeating units in each block varying by design.

Diblock copolymers that can be used as intermediates for making triblock copolymers can have hydrophilic biocompatible poly(ethylene oxide) (PEO), which is chemically synonymous with PEG, coupled to various hydrophobic aliphatic poly(anhydrides), poly(nucleic acids), poly(esters), poly(ortho esters), poly(peptides), poly(phosphazenes) and poly(saccharides), including but not limited by poly(lactide) (PLA), poly(glycolide) (PLGA), poly(lactic-co-glycolic acid) (PLGA), poly(ε-caprolactone) (PCL), and poly (trimethylene carbonate) (PTMC). Polymeric micelles comprised of 100% PEGylated surfaces possess improved in vitro chemical stability, augmented in vivo bioavailablity, and prolonged blood circulatory half-lives.

Polymeric vesicles, polymersomes and poly(Histidine)-based micelles, including those that comprise triblock copolymers, and methods of making the same, are described in further detail in U.S. Pat. Nos. 7,217,427; 7,868,512; 6,835,394; 8,808,748; 10,456,452; U.S. Publication Nos. 2014/0363496; 2017/0000743; and 2019/0255191; and PCT Publication No. WO 2019/126589.

Transposon and Vector Compositions

The present disclosure provides compositions and methods for delivering a CSR (e.g., comprising an scFv) to a cell or a population of cells. Non-limiting examples of compositions for delivery of a composition of the disclosure to a cell or a population of cells include a transposon or a vector. Thus, the present disclosure provides a transposon comprising a CSR (e.g., comprising an scFv) or a vector comprising an antibody a CSR (e.g., comprising an scFv).

A transposon comprising a CSR of the disclosure or a vector comprising a CSR of the disclosure can further comprise a sequence encoding an inducible proapoptotic polypeptide. Alternatively, or in addition, one transposon or one vector can comprise a CSR of the disclosure and a second transposon or second vector can comprise a sequence encoding an inducible proapoptotic polypeptide of the disclosure. Inducible proapoptotic polypeptides are described in more detail herein.

A transposon comprising a CSR of the disclosure or a vector comprising a CSR of the disclosure can further comprise a sequence encoding a chimeric antigen receptor (CAR). Alternatively, or in addition, one transposon or one vector can comprise a CAR of the disclosure and a second transposon or a second vector can comprise a sequence encoding a CSR of the disclosure. Chimeric stimulatory receptors are described in more detail herein.

A transposon comprising a CSR of the disclosure or a vector comprising a CSR of the disclosure can further comprise a sequence encoding a recombinant HLA-E polypeptide. Alternatively, or in addition, one transposon or one vector can comprise a CSR of the disclosure and a second transposon or a second vector can comprise a sequence encoding a recombinant HLA-E polypeptide. Recombinant HLA-E polypeptide are described in more detail herein.

A transposon comprising a CSR of the disclosure or a vector comprising a CSR of the disclosure can further comprise a selection gene. The selection gene can encode a gene product essential for cell viability and survival. The selection gene can encode a gene product essential for cell viability and survival when challenged by selective cell culture conditions. Selective cell culture conditions may comprise a compound harmful to cell viability or survival and wherein the gene product confers resistance to the compound. Non-limiting examples of selection genes include neo (conferring resistance to neomycin), DHFR (encoding Dihydrofolate Reductase and conferring resistance to Methotrexate), TYMS (encoding Thymidylate Synthetase), MGMT (encoding O(6)-methylguanine-DNA methyltransferase), multidrug resistance gene (MDR1), ALDH1 (encoding Aldehyde dehydrogenase 1 family, member A1), FRANCF, RAD51C (encoding RAD51 Paralog C), GCS (encoding glucosylceramide synthase), NKX2.2 (encoding NK2 Homeobox 2), or any combination thereof.

In a preferred aspect, the selection gene encodes a DHFR mutein enzyme. The DHFR mutein enzyme comprises, consists essentially of, or consists of the amino acid sequence of SEQ ID NO: 227. The DHFR mutein enzyme is encoded by a polynucleotide comprising, consisting essential of, or consisting of the nucleic acid sequence of SEQ ID NO: 228 or SEQ ID NO: 229. The amino acid sequence of the DHFR mutein enzyme can further comprise a mutation at one or more of positions 80, 113, or 153. The amino acid sequence of the DHFR mutein enzyme can comprise one or more of a substitution of a Phenylalanine (F) or a Leucine (L) at position 80, a substitution of a Leucine (L) or a Valine (V) at position 113, and a substitution of a Valine (V) or an Aspartic Acid (D) at position 153.

A transposon comprising a CSR of the disclosure or a vector comprising a CSR of the disclosure can further comprise at least one self-cleaving peptide. For example, a self-cleaving peptide can be located between a CSR (e.g., comprising an scFv) and an inducible proapoptotic polypeptide; or, a self-cleaving peptide can be located between a CSR (e.g., comprising an scFv) and protein encoded by a selection gene.

A transposon comprising a CSR of the disclosure or a vector comprising a CSR of the disclosure can further comprise at least two self-cleaving peptides. For example, a first self-cleaving peptide is located upstream or immediately upstream of a CSR and a second self-cleaving peptide is located downstream or immediately downstream of a CSR; or, the first self-cleaving peptide and the second self-cleaving peptide flank a CSR. For example, a first self-cleaving peptide is located upstream or immediately upstream of an inducible proapoptotic polypeptide and a second self-cleaving peptide is located downstream or immediately downstream of an inducible proapoptotic polypeptide; or, the first self-cleaving peptide and the second self-cleaving peptide flank an inducible proapoptotic polypeptide. For example, a first self-cleaving peptide is located upstream or immediately upstream of protein encoded by a selection gene and a second self-cleaving peptide is located downstream or immediately downstream of a protein encoded by a selection gene; or, the first self-cleaving peptide and the second self-cleaving peptide flank a protein encoded by a selection gene.

Non-limiting examples of self-cleaving peptides include a T2A peptide, GSG-T2A peptide, an E2A peptide, a GSG-E2A peptide, an F2A peptide, a GSG-F2A peptide, a P2A peptide, or a GSG-P2A peptide. A T2A peptide comprises, consists essential of, or consists of, the amino acid sequence at least 75%, 80%, 85%, 90%, 95%, 96%, 97%, 98%, 99% or 100% (or any percentage in between) identical to SEQ ID NO: 230. A GSG-T2A peptide comprises, consists essential of, or consists of, the amino acid sequence at least 75%, 80%, 85%, 90%, 95%, 96%, 97%, 98%, 99% or 100% (or any percentage in between) identical to SEQ ID NO: 231. A GSG-T2A polypeptide is encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 232. A E2A peptide comprises, consists essential of, or consists of, the amino acid sequence at least 75%, 80%, 85%, 90%, 95%, 96%, 97%, 98%, 99% or 100% (or any percentage in between) identical to SEQ ID NO: 233. A GSG-E2A peptide comprises, consists essential of, or consists of, the amino acid sequence at least 75%, 80%, 85%, 90%, 95%, 96%, 97%, 98%, 99% or 100% (or any percentage in between) identical to SEQ ID NO: 234. A F2A peptide comprises, consists essential of, or consists of, the amino acid sequence at least 75%, 80%, 85%, 90%, 95%, 96%, 97%, 98%, 99% or 100% (or any percentage in between) identical to SEQ ID NO: 235. A GSG-F2A peptide comprises, consists essential of, or consists of, the amino acid sequence at least 75%, 80%, 85%, 90%, 95%, 96%, 97%, 98%, 99% or 100% (or any percentage in between) identical to SEQ ID NO: 236. A P2A peptide comprises, consists essential of, or consists of, the amino acid sequence at least 75%, 80%, 85%, 90%, 95%, 96%, 97%, 98%, 99% or 100% (or any percentage in between) identical to SEQ ID NO: 237. A GSG-P2A peptide comprises, consists essential of, or consists of, the amino acid sequence at least 75%, 80%, 85%, 90%, 95%, 96%, 97%, 98%, 99% or 100% (or any percentage in between) identical to SEQ ID NO: 238.

Transposition Systems

The present disclosure provides a transposon comprising a protein scaffold as disclosed herein or the present disclosure provides a transposon comprising a CSR or a CAR (e.g., comprising an scFv) as disclosed herein. In a preferred aspect, the transposon is a plasmid DNA transposon comprising a nucleotide sequence encoding a CSR or CAR (e.g., comprising an scFv) as disclosed herein flanked by two cis-regulatory insulator elements. The present disclosure also provides a composition comprising a transposon. In a preferred aspect, the composition comprising the transposon further comprises a plasmid comprising a nucleotide sequence encoding a transposase. The nucleotide sequence encoding the transposase may be a DNA sequence or an RNA sequence. Preferably, the sequence encoding the transposase is an mRNA sequence.

A transposon of the present disclosure can be a piggyBac™ (PB) transposon. In some aspects when the transposon is a PB transposon, the transposase is a piggyBac™ (PB) transposase a piggyBac-like (PBL) transposase or a Super piggyBac™ (SPB) transposase. The sequence encoding the SPB transposase is an mRNA sequence.

Non-limiting examples of PB transposons and PB, PBL and SPB transposases are described in detail in U.S. Pat. Nos. 6,218,182; 6,962,810; 8,399,643 and PCT Publication No. WO 2010/099296.

The PB, PBL and SPB transposases recognize transposon-specific inverted terminal repeat sequences (ITRs) on the ends of the transposon, and inserts the contents between the ITRs at the sequence 5′-TTAT-3′ within a chromosomal site (a TTAT target sequence) or at the sequence 5′-TTAA-3′ within a chromosomal site (a TTAA target sequence). The target sequence of the PB or PBL transposon can comprise or consist of 5′-CTAA-3′, 5′-TTAG-3′, 5′-ATAA-3′, 5′-TCAA-3′, 5′AGTT-3′, 5′-ATTA-3′, 5′-GTTA-3′, 5′-TTGA-3′, 5′-TTTA-3′, 5′-TTAC-3′, 5′-ACTA-3′, 5′-AGGG-3′, 5′-CTAG-3′, 5′-TGAA-3′, 5′-AGGT-3′, 5′-ATCA-3′, 5′-CTCC-3′, 5′-TAAA-3′, 5′-TCTC-3′, 5′ TGAA-3′, 5′-AAAT-3′, 5′-AATC-3′, 5′-ACAA-3′, 5′-ACAT-3′, 5′-ACTC-3′, 5′-AGTG-3′, 5′-ATAG-3′, 5′-CAAA-3′, 5′-CACA-3′, 5′-CATA-3′, 5′-CCAG-3′, 5′-CCCA-3′, 5′-CGTA-3′, 5′-GTCC-3′, 5′-TAAG-3′, 5′-TCTA-3′, 5′-TGAG-3′, 5′-TGTT-3′, 5′-TTCA-3′5′-TTCT-3′ and 5′-TTTT-3′. The PB or PBL transposon system has no payload limit for the genes of interest that can be included between the ITRs.

Exemplary amino acid sequence for one or more PB, PBL and SPB transposases are disclosed in U.S. Pat. Nos. 6,218,185; 6,962,810 and 8,399,643. In a preferred aspect, the PB transposase comprises or consists of an amino acid sequence at least 75%, 80%, 85%, 90%, 95%, 96%, 97%, 98%, 99% or 100% (or any percentage in between) identical to SEQ ID NO: 239.

The PB or PBL transposase can comprise or consist of an amino acid sequence having an amino acid substitution at two or more, at three or more or at each of positions 30, 165, 282, or 538 of the sequence of SEQ ID NO: 239. The transposase can be a SPB transposase that comprises or consists of the amino acid sequence of the sequence of SEQ ID NO: 239 wherein the amino acid substitution at position 30 can be a substitution of a valine (V) for an isoleucine (I), the amino acid substitution at position 165 can be a substitution of a serine (S) for a glycine (G), the amino acid substitution at position 282 can be a substitution of a valine (V) for a methionine (M), and the amino acid substitution at position 538 can be a substitution of a lysine (K) for an asparagine (N). In a preferred aspect, the SPB transposase comprises or consists of an amino acid sequence at least 75%, 80%, 85%, 90%, 95%, 96%, 97%, 98%, 99% or 100% (or any percentage in between) identical to SEQ ID NO: 240.

In certain aspects wherein the transposase comprises the above-described mutations at positions 30, 165, 282 and/or 538, the PB, PBL and SPB transposases can further comprise an amino acid substitution at one or more of positions 3, 46, 82, 103, 119, 125, 177, 180, 185, 187, 200, 207, 209, 226, 235, 240, 241, 243, 258, 296, 298, 311, 315, 319, 327, 328, 340, 421, 436, 456, 470, 486, 503, 552, 570 and 591 of the sequence of SEQ ID NO: 239 or SEQ ID NO: 240 are described in more detail in PCT Publication No. WO 2019/173636 and PCT/US2019/049816.

The PB, PBL or SPB transposases can be isolated or derived from an insect, vertebrate, crustacean or urochordate as described in more detail in PCT Publication No. WO 2019/173636 and PCT/US2019/049816. In preferred aspects, the PB, PBL or SPB transposases is be isolated or derived from the insect Trichoplusia ni (GenBank Accession No. AAA87375) or Bombyx mori (GenBank Accession No. BAD11135).

A hyperactive PB or PBL transposase is a transposase that is more active than the naturally occurring variant from which it is derived. In a preferred aspect, a hyperactive PB or PBL transposase is isolated or derived from Bombyx mori or Xenopus tropicalis. Examples of hyperactive PB or PBL transposases are disclosed in U.S. Pat. Nos. 6,218,185; 6,962,810, 8,399,643 and WO 2019/173636. A list of hyperactive amino acid substitutions is disclosed in U.S. Pat. No. 10,041,077.

In some aspects, the PB or PBL transposase is integration deficient. An integration deficient PB or PBL transposase is a transposase that can excise its corresponding transposon, but that integrates the excised transposon at a lower frequency than a corresponding wild type transposase. Examples of integration deficient PB or PBL transposases are disclosed in U.S. Pat. Nos. 6,218,185; 6,962,810, 8,399,643 and WO 2019/173636. A list of integration deficient amino acid substitutions is disclosed in U.S. Pat. No. 10,041,077.

In some aspects, the PB or PBL transposase is fused to a nuclear localization signal. Examples of PB or PBL transposases fused to a nuclear localization signal are disclosed in U.S. Pat. Nos. 6,218,185; 6,962,810, 8,399,643 and WO 2019/173636.

A transposon of the present disclosure can be a Sleeping Beauty transposon. In some aspects, when the transposon is a Sleeping Beauty transposon, the transposase is a Sleeping Beauty transposase (for example as disclosed in U.S. Pat. No. 9,228,180) or a hyperactive Sleeping Beauty (SB100X) transposase. In a preferred aspect, the Sleeping Beauty transposase comprises or consists of an amino acid sequence at least 75%, 80%, 85%, 90%, 95%, 96%, 97%, 98%, 99% or 100% (or any percentage in between) identical to SEQ ID NO: 241. In a preferred aspect, hyperactive Sleeping Beauty (SB100X) transposase comprises or consists of an amino acid sequence at least 75%, 80%, 85%, 90%, 95%, 96%, 97%, 98%, 99% or 100% (or any percentage in between) identical to SEQ ID NO: 242.

A transposon of the present disclosure can be a Helraiser transposon. An exemplary Helraiser transposon includes Helibat1, which comprises or consists of a nucleic acid sequence at least 75%, 80%, 85%, 90%, 95%, 96%, 97%, 98%, 99% or 100% (or any percentage in between) identical to SEQ ID NO: 243. In some aspects, when the transposon is a Helraiser transposon, the transposase is a Helitron transposase (for example, as disclosed in WO 2019/173636). In a preferred aspect, Helitron transposase comprises or consists of an amino acid sequence at least 75%, 80%, 85%, 90%, 95%, 96%, 97%, 98%, 99% or 100% (or any percentage in between) identical to SEQ ID NO: 244.

A transposon of the present disclosure can be a To12 transposon. An exemplary To12 transposon, including inverted repeats, subterminal sequences and the To12 transposase, comprises or consists of a nucleic acid sequence at least 75%, 80%, 85%, 90%, 95%, 96%, 97%, 98%, 99% or 100% (or any percentage in between) identical to SEQ ID NO: 245. In some aspects, when the transposon is a To12 transposon, the transposase is a To12 transposase (for example, as disclosed in WO 2019/173636). In a preferred aspect, To12 transposase comprises or consists of an amino acid sequence at least 75%, 80%, 85%, 90%, 95%, 96%, 97%, 98%, 99% or 100% (or any percentage in between) identical to SEQ ID NO: 246.

A transposon of the present disclosure can be a TcBuster transposon. In some aspects, when the transposon is a TcBuster transposon, the transposase is a TcBuster transposase or a hyperactive TcBuster transposase (for example, as disclosed in WO 2019/173636). The TcBuster transposase can comprise or consist of a naturally occurring amino acid sequence or a non-naturally occurring amino acid sequence. In a preferred aspect, a TcBuster transposase comprises or consists of an amino acid sequence at least 75%, 80%, 85%, 90%, 95%, 96%, 97%, 98%, 99% or 100% (or any percentage in between) identical to SEQ ID NO: 247. The polynucleotide encoding a TcBuster transposase can comprise or consist of a naturally occurring nucleic acid sequence or a non-naturally occurring nucleic acid sequence. In a preferred aspect, a TcBuster transposase is encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 248.

In some aspects, a mutant TcBuster transposase comprises one or more sequence variations when compared to a wild type TcBuster transposase as described in more detail in PCT Publication No. WO 2019/173636 and PCT/US2019/049816.

The transposon can be a nanotransposon. A nanotransposon can comprise, consist essential of, or consist of (a) a sequence encoding a transposon insert, comprising a sequence encoding a first inverted terminal repeat (ITR), a sequence encoding a second inverted terminal repeat (ITR), and an intra-ITR sequence; (b) a sequence encoding a backbone, wherein the sequence encoding the backbone comprises a sequence encoding an origin of replication having between 1 and 450 nucleotides, inclusive of the endpoints, and a sequence encoding a selectable marker having between 1 and 200 nucleotides, inclusive of the endpoints, and (c) an inter-ITR sequence. In some aspects, the inter-ITR sequence of (c) comprises the sequence of (b). In some aspects, the intra-ITR sequence of (a) comprises the sequence of (b).

The sequence encoding the backbone can comprise between 1 and 600 nucleotides, inclusive of the endpoints. In some aspects, the sequence encoding the backbone consists of between 1 and 50 nucleotides, between 50 and 100 nucleotides, between 100 and 150 nucleotides, between 150 and 200 nucleotides, between 200 and 250 nucleotides, between 250 and 300 nucleotides, between 300 and 350 nucleotides, between 350 and 400 nucleotides, between 400 and 450 nucleotides, between 450 and 500 nucleotides, between 500 and 550 nucleotides, between 550 and 600 nucleotides, each range inclusive of the endpoints.

The inter-ITR sequence can comprise between 1 and 1000 nucleotides, inclusive of the endpoints. In some aspects, the inter-ITR sequence consists of between 1 and 50 nucleotides, between 50 and 100 nucleotides, between 100 and 150 nucleotides, between 150 and 200 nucleotides, between 200 and 250 nucleotides, between 250 and 300 nucleotides, between 300 and 350 nucleotides, between 350 and 400 nucleotides, between 400 and 450 nucleotides, between 450 and 500 nucleotides, between 500 and 550 nucleotides, between 550 and 600 nucleotides, between 600 and 650 nucleotides, between 650 and 700 nucleotides, between 700 and 750 nucleotides, between 750 and 800 nucleotides, between 800 and 850 nucleotides, between 850 and 900 nucleotides, between 900 and 950 nucleotides, or between 950 and 1000 nucleotides, each range inclusive of the endpoints.

The nanotransposon can be a short nanotransposon (SNT) wherein the inter-ITR sequence comprises between 1 and 200 nucleotides, inclusive of the endpoints. The inter-ITR sequence can consist of between 1 and 10 nucleotides, between 10 and 20 nucleotides, between 20 and 30 nucleotides, between 30 and 40 nucleotides, between 40 and 50 nucleotides, between 50 and 60 nucleotides, between 60 and 70 nucleotides, between 70 and 80 nucleotides, between 80 and 90 nucleotides, or between 90 and 100 nucleotides, each range inclusive of the endpoints.

The selectable marker having between 1 and 200 nucleotides, inclusive of the endpoints, can comprise a sequence encoding a sucrose-selectable marker. The sequence encoding a sucrose-selectable marker can comprise a sequence encoding an RNA-OUT sequence. The sequence encoding an RNA-OUT sequence can comprise or consist of 137 base pairs (bp). The selectable marker having between 1 and 200 nucleotides, inclusive of the endpoints, can comprise a sequence encoding a fluorescent marker. The selectable marker having between 1 and 200 nucleotides, inclusive of the endpoints, can comprise a sequence encoding a cell surface marker.

The sequence encoding an origin of replication having between 1 and 450 nucleotides, inclusive of the endpoints, can comprise a sequence encoding a mini origin of replication. In some aspects, the sequence encoding an origin of replication having between 1 and 450 nucleotides, inclusive of the endpoints, comprises a sequence encoding an R6K origin of replication. The R6K origin of replication can comprise an R6K gamma origin of replication. The R6K origin of replication can comprise an R6K mini origin of replication. The R6K origin of replication can comprise an R6K gamma mini origin of replication. The R6K gamma mini origin of replication can comprise or consist of 281 base pairs (bp).

In some aspects of the nanotransposon, the sequence encoding the backbone does not comprise a recombination site, an excision site, a ligation site or a combination thereof. In some aspects, neither the nanotransposon nor the sequence encoding the backbone comprises a product of a recombination site, an excision site, a ligation site or a combination thereof. In some aspects, neither the nanotransposon nor the sequence encoding the backbone is derived from a recombination site, an excision site, a ligation site or a combination thereof.

In some aspects of the nanotransposon, a recombination site comprises a sequence resulting from a recombination event. In some aspects, a recombination site comprises a sequence that is a product of a recombination event. In some aspects, the recombination event comprises an activity of a recombinase (e.g., a recombinase site).

In some aspects of the nanotransposon, the sequence encoding the backbone does not further comprise a sequence encoding foreign DNA.

In some aspects of the nanotransposon, the inter-ITR sequence does not comprise a recombination site, an excision site, a ligation site or a combination thereof. In some aspects, the inter-ITR sequence does not comprise a product of a recombination event, an excision event, a ligation event or a combination thereof. In some aspects, the inter-ITR sequence is not derived from a recombination event, an excision event, a ligation event or a combination thereof. In some aspects, the inter-ITR sequence comprises a sequence encoding foreign DNA. In some aspects, the intra-ITR sequence comprises at least one sequence encoding an insulator and a sequence encoding a promoter capable of expressing an exogenous sequence in a mammalian cell. The mammalian cell can be a human cell. In some aspects, the intra-ITR sequence comprises a first sequence encoding an insulator, a sequence encoding a promoter capable of expressing an exogenous sequence in a mammalian cell and a second sequence encoding an insulator. In some aspects, the intra-ITR sequence comprises a first sequence encoding an insulator, a sequence encoding a promoter capable of expressing an exogenous sequence in a mammalian cell, a polyadenosine (polyA) sequence and a second sequence encoding an insulator. In some aspects, the intra-ITR sequence comprises a first sequence encoding an insulator, a sequence encoding a promoter capable of expressing an exogenous sequence in a mammalian cell, at least one exogenous sequence, a polyadenosine (polyA) sequence and a second sequence encoding an insulator.

Nanotransposons are described in more detail in PCT/US2019/067758.

Vector Systems

A vector of the present disclose can be a viral vector or a recombinant vector. Viral vectors can comprise a sequence isolated or derived from a retrovirus, a lentivirus, an adenovirus, an adeno-associated virus or any combination thereof. The viral vector may comprise a sequence isolated or derived from an adeno-associated virus (AAV). The viral vector may comprise a recombinant AAV (rAAV). Exemplary adeno-associated viruses and recombinant adeno-associated viruses comprise two or more inverted terminal repeat (ITR) sequences located in cis next to a sequence encoding a CSR or a CAR of the disclosure. Exemplary adeno-associated viruses and recombinant adeno-associated viruses include, but are not limited to all serotypes (e.g., AAV1, AAV2, AAV3, AAV4, AAV5, AAV6, AAV7, AAV8, and AAV9). Exemplary adeno-associated viruses and recombinant adeno-associated viruses include, but are not limited to, self-complementary AAV (scAAV) and AAV hybrids containing the genome of one serotype and the capsid of another serotype (e.g., AAV2/5, AAV-DJ and AAV-DJ8). Exemplary adeno-associated viruses and recombinant adeno-associated viruses include, but are not limited to, rAAV-LK03.

A vector of the present disclose can be a nanoparticle. Non-limiting examples of nanoparticle vectors include nucleic acids (e.g., RNA, DNA, synthetic nucleotides, modified nucleotides or any combination thereof), amino acids (L-amino acids, D-amino acids, synthetic amino acids, modified amino acids, or any combination thereof), polymers (e.g., polymersomes), micelles, lipids (e.g., liposomes), organic molecules (e.g., carbon atoms, sheets, fibers, tubes), inorganic molecules (e.g., calcium phosphate or gold) or any combination thereof. A nanoparticle vector can be passively or actively transported across a cell membrane.

The cell delivery compositions (e.g., transposons, vectors) disclosed herein can comprise a nucleic acid encoding a therapeutic protein or therapeutic agent. Examples of therapeutic proteins include those disclosed in PCT Publication No. WO 2019/173636 and PCT/US2019/049816.

Inducible Proapoptotic Polypeptides

The inducible proapoptotic polypeptides disclosed herein are superior to existing inducible polypeptides because the inducible proapoptotic polypeptides of the disclosure are far less immunogenic. The inducible proapoptotic polypeptides are recombinant polypeptides, and, therefore, non-naturally occurring. Further, the sequences that are recombined to produce inducible proapoptotic polypeptides that do not comprise non-human sequences that the host human immune system could recognize as “non-self” and, consequently, induce an immune response in the subject receiving the inducible proapoptotic polypeptide, a cell comprising the inducible proapoptotic polypeptide or a composition comprising the inducible proapoptotic polypeptide or the cell comprising the inducible proapoptotic polypeptide.

The disclosure provides inducible proapoptotic polypeptides comprising a ligand binding region, a linker, and a proapoptotic peptide, wherein the inducible proapoptotic polypeptide does not comprise a non-human sequence. In certain aspects, the non-human sequence comprises a restriction site. In certain aspects, the ligand binding region can be a multimeric ligand binding region. In certain aspects, the proapoptotic peptide is a caspase polypeptide. Non-limiting examples of caspase polypeptides include caspase 1, caspase 2, caspase 3, caspase 4, caspase 5, caspase 6, caspase 7, caspase 8, caspase 9, caspase 10, caspase 11, caspase 12, and caspase 14. Preferably, the caspase polypeptide is a caspase 9 polypeptide. The caspase 9 polypeptide can be a truncated caspase 9 polypeptide. Inducible proapoptotic polypeptides can be non-naturally occurring. When the caspase is caspase 9 or a truncated caspase 9, the inducible proapoptotic polypeptides can also be referred to as an “iC9 safety switch”.

An inducible caspase polypeptide can comprise (a) a ligand binding region, (b) a linker, and (c) a caspase polypeptide, wherein the inducible proapoptotic polypeptide does not comprise a non-human sequence. In certain aspects, an inducible caspase polypeptide comprises (a) a ligand binding region, (b) a linker, and (c) a truncated caspase 9 polypeptide, wherein the inducible proapoptotic polypeptide does not comprise a non-human sequence.

The ligand binding region can comprise a FK506 binding protein 12 (FKBP12) polypeptide. The amino acid sequence of the ligand binding region that comprises a FK506 binding protein 12 (FKBP12) polypeptide can comprise a modification at position 36 of the sequence. The modification can be a substitution of valine (V) for phenylalanine (F) at position 36 (F36V). The FKBP12 polypeptide can comprise, consist essential of, or consist of, the amino acid sequence at least 75%, 80%, 85%, 90%, 95%, 96%, 97%, 98%, 99% or 100% (or any percentage in between) identical to SEQ ID NO: 249. The FKBP12 polypeptide can be encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 250.

The linker region can comprise, consist essential of, or consist of, the amino acid sequence at least 75%, 80%, 85%, 90%, 95%, 96%, 97%, 98%, 99% or 100% (or any percentage in between) identical to SEQ ID NO: 251 or the linker region can be encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 252. In some aspects, the nucleic acid sequence encoding the linker does not comprise a restriction site.

The truncated caspase 9 polypeptide can comprise an amino acid sequence that does not comprise an arginine (R) at position 87 of the sequence. Alternatively, or in addition, the truncated caspase 9 polypeptide can comprise an amino acid sequence that does not comprise an alanine (A) at position 282 the sequence. The truncated caspase 9 polypeptide can comprise, consist essential of, or consist of, the amino acid sequence at least 75%, 80%, 85%, 90%, 95%, 96%, 97%, 98%, 99% or 100% (or any percentage in between) identical to SEQ ID NO: 253 or the truncated caspase 9 polypeptide can be encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 254.

In certain aspects when the polypeptide comprises a truncated caspase 9 polypeptide, the inducible proapoptotic polypeptide comprises, consists essential of, or consists of, the amino acid sequence at least 75%, 80%, 85%, 90%, 95%, 96%, 97%, 98%, 99% or 100% (or any percentage in between) identical to SEQ ID NO: 255 or the inducible proapoptotic polypeptide is encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 256.

In certain aspects when the polypeptide comprises a truncated caspase 9 polypeptide, the inducible proapoptotic polypeptide comprises, consists essential of, or consists of, the amino acid sequence at least 75%, 80%, 85%, 90%, 95%, 96%, 97%, 98%, 99% or 100% (or any percentage in between) identical to SEQ ID NO: 257 or the inducible proapoptotic polypeptide is encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 258.

Inducible proapoptotic polypeptides can be expressed in a cell under the transcriptional regulation of any promoter known in the art that is capable of initiating and/or regulating the expression of an inducible proapoptotic polypeptide in that cell.

Activation of inducible proapoptotic polypeptides can be accomplished through, for example, chemically induced dimerization (CID) mediated by an induction agent to produce a conditionally controlled protein or polypeptide. Proapoptotic polypeptides not only inducible, but the induction of these polypeptides is also reversible, due to the degradation of the labile dimerizing agent or administration of a monomeric competitive inhibitor.

In certain aspects when the ligand binding region comprises a FKBP12 polypeptide having a substitution of valine (V) for phenylalanine (F) at position 36 (F36V), the induction agent can comprise AP1903, a synthetic drug (CAS Index Name: 2-Piperidinecarboxylic acid, 1-[(2S)-1-oxo-2-(3,4,5-trimethoxyphenyl)butyl]—, 1,2-ethanediylbis[imino(2-oxo-2,1-ethanediyl)oxy-3,1-phenylene[(1R)-3-(3,4-dimethoxyphenyl)propylidene]]ester, [2S-[1(R*),2R*[S*[S*[1(R*),2R*]]]]]—(9C1) CAS Registry Number: 195514-63-7; Molecular Formula: C78H98N4020; Molecular Weight: 1411.65)); AP20187 (CAS Registry Number: 195514-80-8 and Molecular Formula: C82H107N5020) or an AP20187 analog, such as, for example, AP1510. As used herein, the induction agents AP20187, AP1903 and AP1510 can be used interchangeably.

Inducible proapoptotic peptides and methods of inducing these peptides are described in detail in U.S. Patent Publication No. WO 2019/0225667 and PCT Publication No. WO 2018/068022.

Formulations, Dosages and Modes of Administration

The present disclosure provides formulations, dosages and methods for administration of the compositions described herein.

The disclosed compositions and pharmaceutical compositions can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, Pa.) 1990 and in the “Physician's Desk Reference”, 52nd ed., Medical Economics (Montvale, N.J.) 1998. Pharmaceutically acceptable carriers can be routinely selected that are suitable for the mode of administration, solubility and/or stability of the protein scaffold, fragment or variant composition as well known in the art or as described herein.

Non-limiting examples of pharmaceutical excipients and additives suitable for use include proteins, peptides, amino acids, lipids, and carbohydrates (e.g., sugars, including monosaccharides, di—, tri—, tetra—, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Non-limiting examples of protein excipients include serum albumin, such as human serum albumin (HSA), recombinant human albumin (rHA), gelatin, casein, and the like. Representative amino acid/protein components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Non-limiting examples of carbohydrate excipients suitable for use include monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferably, the carbohydrate excipients are mannitol, trehalose, and/or raffinose.

The compositions can also include a buffer or a pH-adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers are organic acid salts, such as citrate.

Additionally, the disclosed compositions can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

Many known and developed modes can be used for administering therapeutically effective amounts of the compositions or pharmaceutical compositions disclosed herein. Non-limiting examples of modes of administration include bolus, buccal, infusion, intrarticular, intrabronchial, intraabdominal, intracapsular, intracartilaginous, intracavitary, intracelial, intracerebellar, intracerebroventricular, intracolic, intracervical, intragastric, intrahepatic, intralesional, intramuscular, intramyocardial, intranasal, intraocular, intraosseous, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intratumoral, intravenous, intravesical, oral, parenteral, rectal, sublingual, subcutaneous, transdermal or vaginal means.

A composition of the disclosure can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement;” Hsieh, D. S., Eds., pp. 59-90 (Marcel Dekker, Inc. New York 1994,), or with oxidizing agents that enable the application of formulations containing proteins and peptides onto the skin (WO 98/53847), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

For parenteral administration, any composition disclosed herein can be formulated as a solution, suspension, emulsion, particle, powder, or lyophilized powder in association, or separately provided, with a pharmaceutically acceptable parenteral vehicle. Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthtetic mono— or di— or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446.

Formulations for oral administration rely on the co-administration of adjuvants (e.g., resorcinols and nonionic surfactants, such as polyoxyethylene oleyl ether and n-hexadecylpolyethylene ether) to increase artificially the permeability of the intestinal walls, as well as the co-administration of enzymatic inhibitors (e.g., pancreatic trypsin inhibitors, diisopropylfluorophosphate (DFF) and trasylol) to inhibit enzymatic degradation. Formulations for delivery of hydrophilic agents including proteins and protein scaffolds and a combination of at least two surfactants intended for oral, buccal, mucosal, nasal, pulmonary, vaginal transmembrane, or rectal administration are described in U.S. Pat. No. 6,309,663. The active constituent compound of the solid-type dosage form for oral administration can be mixed with at least one additive, including sucrose, lactose, cellulose, mannitol, trehalose, raffinose, maltitol, dextran, starches, agar, arginates, chitins, chitosans, pectins, gum tragacanth, gum arabic, gelatin, collagen, casein, albumin, synthetic or semisynthetic polymer, and glyceride. These dosage forms can also contain other type(s) of additives, e.g., inactive diluting agent, lubricant, such as magnesium stearate, paraben, preserving agent, such as sorbic acid, ascorbic acid, .alpha.-tocopherol, antioxidant such as cysteine, disintegrator, binder, thickener, buffering agent, sweetening agent, flavoring agent, perfuming agent, etc.

Tablets and pills can be further processed into enteric-coated preparations. The liquid preparations for oral administration include emulsion, syrup, elixir, suspension and solution preparations allowable for medical use. These preparations can contain inactive diluting agents ordinarily used in said field, e.g., water. Liposomes have also been described as drug delivery systems for insulin and heparin (U.S. Pat. No. 4,239,754). More recently, microspheres of artificial polymers of mixed amino acids (proteinoids) have been used to deliver pharmaceuticals (U.S. Pat. No. 4,925,673). Furthermore, carrier compounds described in U.S. Pat. Nos. 5,879,681 and 5,871,753 and used to deliver biologically active agents orally are known in the art.

For pulmonary administration, preferably, a composition or pharmaceutical composition described herein is delivered in a particle size effective for reaching the lower airways of the lung or sinuses. The composition or pharmaceutical composition can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers (e.g., jet nebulizer, ultrasonic nebulizer), dry powder generators, sprayers, and the like. All such devices can use formulations suitable for the administration for the dispensing of a composition or pharmaceutical composition described herein in an aerosol. Such aerosols can be comprised of either solutions (both aqueous and non-aqueous) or solid particles. Additionally, a spray including a composition or pharmaceutical composition described herein can be produced by forcing a suspension or solution of at least one protein scaffold through a nozzle under pressure. In a metered dose inhaler (MDI), a propellant, a composition or pharmaceutical composition described herein, and any excipients or other additives are contained in a canister as a mixture including a liquefied compressed gas. Actuation of the metering valve releases the mixture as an aerosol, preferably containing particles in the size range of less than about 10 μm, preferably, about 1 μm to about 5 μm, and, most preferably, about 2 μm to about 3 μm. A more detailed description of pulmonary administration, formulations and related devices is disclosed in PCT Publication No. WO 2019/049816.

For absorption through mucosal surfaces, compositions include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. No. 5,514,670). Mucous surfaces suitable for application of the emulsions of the disclosure can include corneal, conjunctival, buccal, sublingual, nasal, vaginal, pulmonary, stomachic, intestinal, and rectal routes of administration. Formulations for vaginal or rectal administration, e.g., suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration, excipients include sugars, calcium stearate, magnesium stearate, pregelinatined starch, and the like (U.S. Pat. No. 5,849,695). A more detailed description of mucosal administration and formulations is disclosed in PCT Publication No. WO 2019/049816.

For transdermal administration, a composition or pharmaceutical composition disclosed herein is encapsulated in a delivery device, such as a liposome or polymeric nanoparticles, microparticle, microcapsule, or microspheres (referred to collectively as microparticles unless otherwise stated). A number of suitable devices are known, including microparticles made of synthetic polymers, such as polyhydroxy acids, such as polylactic acid, polyglycolic acid and copolymers thereof, polyorthoesters, polyanhydrides, and polyphosphazenes, and natural polymers, such as collagen, polyamino acids, albumin and other proteins, alginate and other polysaccharides, and combinations thereof (U.S. Pat. No. 5,814,599). A more detailed description of transdermal administration, formulations and suitable devices is disclosed in PCT Publication No. WO 2019/049816.

It can be desirable to deliver the disclosed compounds to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid, such as phosphoric acid, sulfuric acid, citric acid, tartaric acid, tannic acid, pamoic acid, alginic acid, polyglutamic acid, naphthalene mono— or di— sulfonic acids, polygalacturonic acid, and the like; (b) a salt with a polyvalent metal cation, such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b), e.g., a zinc tannate salt. Additionally, the disclosed compounds or, preferably, a relatively insoluble salt, such as those just described, can be formulated in a gel, for example, an aluminum monostearate gel with, e.g., sesame oil, suitable for injection. Particularly preferred salts are zinc salts, zinc tannate salts, pamoate salts, and the like. Another type of slow release depot formulation for injection would contain the compound or salt dispersed for encapsulation in a slow degrading, non-toxic, non-antigenic polymer, such as a polylactic acid/polyglycolic acid polymer for example as described in U.S. Pat. No. 3,773,919. The compounds or, preferably, relatively insoluble salts, such as those described above, can also be formulated in cholesterol matrix silastic pellets, particularly for use in animals. Additional slow release, depot or implant formulations, e.g., gas or liquid liposomes, are known in the literature (U.S. Pat. No. 5,770,222 and “Sustained and Controlled Release Drug Delivery Systems”, J. R. Robinson ed., Marcel Dekker, Inc., N.Y., 1978).

Suitable dosages are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif (2000); Nursing 2001 Handbook of Drugs, 21st edition, Springhouse Corp., Springhouse, Pa., 2001; Health Professional's Drug Guide 2001, ed., Shannon, Wilson, Stang, Prentice-Hall, Inc, Upper Saddle River, N.J. Preferred doses can optionally include about 0.1-99 and/or 100-500 mg/kg/administration, or any range, value or fraction thereof, or to achieve a serum concentration of about 0.1-5000 μg/ml serum concentration per single or multiple administration, or any range, value or fraction thereof. A preferred dosage range for the compositions or pharmaceutical compositions disclosed herein is from about 1 mg/kg, up to about 3, about 6 or about 12 mg/kg of body weight of the subject.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and preferably, 0.1 to 10 milligrams per kilogram per administration or in sustained release form is effective to obtain desired results.

As a non-limiting example, treatment of humans or animals can be provided as a one-time or periodic dosage of the compositions or pharmaceutical compositions disclosed herein about 0.1 to 100 mg/kg or any range, value or fraction thereof per day, on at least one of day 1-40, or, alternatively or additionally, at least one of week 1-52, or, alternatively or additionally, at least one of 1-20 years, or any combination thereof, using single, infusion or repeated doses.

Dosage forms suitable for internal administration generally contain from about 0.001 milligram to about 500 milligrams of active ingredient per unit or container. In these pharmaceutical compositions the active ingredient will ordinarily be present in an amount of about 0.5-99.999% by weight based on the total weight of the composition.

An effective amount can comprise an amount of about 0.001 to about 500 mg/kg per single (e.g., bolus), multiple or continuous administration, or to achieve a serum concentration of 0.01-5000 μg/ml serum concentration per single, multiple, or continuous administration, or any effective range or value therein, as done and determined using known methods, as described herein or known in the relevant arts.

In aspects where the compositions to be administered to a subject in need thereof are modified cells as disclosed herein, the cells can be administered between about 1×10³ and 1×10¹⁵ cells; about 1×10⁴ and 1×10¹² cells; about 1×10⁵ and 1×10¹⁰ cells; about 1×10⁶ and 1×10⁹ cells; about 1×10⁶ and 1×10⁸ cells; about 1×10⁶ and 1×10⁷ cells; or about 1×10⁶ and 25×10⁶ cells. In one aspect the cells are administered between about 5×10⁶ and 25×10⁶ cells.

A more detailed description of pharmaceutically acceptable excipients, formulations, dosages and methods of administration of the disclosed compositions and pharmaceutical compositions is disclosed in PCT Publication No. WO 2019/049816.

Methods of Using the Compositions of the Disclosure

The disclosure provides the use of a disclosed composition or pharmaceutical composition for the treatment of a disease or disorder in a cell, tissue, organ, animal, or subject, as known in the art or as described herein, using the disclosed compositions and pharmaceutical compositions, e.g., administering or contacting the cell, tissue, organ, animal, or subject with a therapeutic effective amount of the composition or pharmaceutical composition. In one aspect, the subject is a mammal. Preferably, the subject is human. The terms “subject” and “patient” are used interchangeably herein.

The disclosure provides a method for modulating or treating at least one malignant disease or disorder in a cell, tissue, organ, animal or subject. Preferably, the malignant disease is cancer. Non-limiting examples of a malignant disease or disorder include leukemia, acute leukemia, acute lymphoblastic leukemia (ALL), acute lymphocytic leukemia, B-cell, T-cell or FAB ALL, acute myeloid leukemia (AML), acute myelogenous leukemia, chronic myelocytic leukemia (CML), chronic lymphocytic leukemia (CLL), hairy cell leukemia, myelodysplastic syndrome (MDS), a lymphoma, Hodgkin's disease, a malignant lymphoma, non-Hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumors, bladder cancer, breast cancer, colorectal cancer, endometrial cancer, head cancer, neck cancer, hereditary nonpolyposis cancer, Hodgkin's lymphoma, liver cancer, lung cancer, non-small cell lung cancer, ovarian cancer, pancreatic cancer, prostate cancer, renal cell carcinoma, testicular cancer, adenocarcinomas, sarcomas, malignant melanoma, hemangioma, metastatic disease, cancer related bone resorption, cancer related bone pain, and the like.

In preferred aspects, the treatment of a malignant disease or disorder comprises adoptive cell therapy. For example, in one aspect, the disclosure provides modified cells that express at least one disclosed antibody (e.g., scFv) and/or CAR comprising an antibody (e.g., scFv) that have been selected and/or expanded for administration to a subject in need thereof. Modified cells can be formulated for storage at any temperature including room temperature and body temperature. Modified cells can be formulated for cryopreservation and subsequent thawing. Modified cells can be formulated in a pharmaceutically acceptable carrier for direct administration to a subject from sterile packaging. Modified cells can be formulated in a pharmaceutically acceptable carrier with an indicator of cell viability and/or CAR expression level to ensure a minimal level of cell function and CAR expression. Modified cells can be formulated in a pharmaceutically acceptable carrier at a prescribed density with one or more reagents to inhibit further expansion and/or prevent cell death.

Any can comprise administering an effective amount of any composition or pharmaceutical composition disclosed herein to a cell, tissue, organ, animal or subject in need of such modulation, treatment or therapy. Such a method can optionally further comprise co-administration or combination therapy for treating such diseases or disorders, wherein the administering of any composition or pharmaceutical composition disclosed herein, further comprises administering, before concurrently, and/or after, at least one chemotherapeutic agent (e.g., an alkylating agent, an a mitotic inhibitor, a radiopharmaceutical).

In some aspects, the subject does not develop graft vs. host (GvH) and/or host vs. graft (HvG) following administration. In one aspect, the administration is systemic. Systemic administration can be any means known in the art and described in detail herein. Preferably, systemic administration is by an intravenous injection or an intravenous infusion. In one aspect, the administration is local. Local administration can be any means known in the art and described in detail herein. Preferably, local administration is by intra-tumoral injection or infusion, intraspinal injection or infusion, intracerebroventricular injection or infusion, intraocular injection or infusion, or intraosseous injection or infusion.

In some aspects, the therapeutically effective dose is a single dose. In some aspects, the single dose is one of at least 2, 5, 10, 15, 20, 25, 30, 35, 40, 45, 50, 55, 60, 65, 70, 75, 80, 85, 90, 95, 100 or any number of doses in between that are manufactured simultaneously. In some aspects, where the composition is autologous cells or allogeneic cells, the dose is an amount sufficient for the cells to engraft and/or persist for a sufficient time to treat the disease or disorder.

In one example, the disclosure provides a method of treating cancer in a subject in need thereof, comprising administering to the subject a composition comprising an antibody (e.g., scFv) or a CAR comprising an antibody (e.g., scFv) the antibody or CAR specifically binds to an antigen on a tumor cell. In aspects where the composition comprises a modified cell or cell population, the cell or cell population may be autologous or allogeneic.

In some aspects of the methods of treatment described herein, the treatment can be modified or terminated. Specifically, in aspects where the composition used for treatment comprises an inducible proapoptotic polypeptide, apoptosis may be selectively induced in the cell by contacting the cell with an induction agent. A treatment may be modified or terminated in response to, for example, a sign of recovery or a sign of decreasing disease severity/progression, a sign of disease remission/cessation, and/or the occurrence of an adverse event. In some aspects, the method comprises the step of administering an inhibitor of the induction agent to inhibit modification of the cell therapy, thereby restoring the function and/or efficacy of the cell therapy (for example, when a sign or symptom of the disease reappear or increase in severity and/or an adverse event is resolved).

Antibody/scFv Production, Screening and Purification

At least one antibody (e.g., monoclonal antibody, a chimeric antibody, a single domain antibody, a VHH, a VH, a single chain variable fragment (scFv), an antigen-binding fragment (Fab) or a Fab fragment) of the disclosure can be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2nd Edition, Cold Spring Harbor, N.Y. (1989); Harlow and Lane, Antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001).

Amino acids from an scFv can be altered, added and/or deleted to reduce immunogenicity or reduce, enhance or modify binding, affinity, on-rate, off-rate, avidity, specificity, half-life, stability, solubility or any other suitable characteristic, as known in the art.

Optionally, an scFv can be engineered with retention of high affinity for the antigen and other favorable biological properties. To achieve this goal, the scaffold proteins can be optionally prepared by a process of analysis of the parental sequences and various conceptual engineered products using three-dimensional models of the parental and engineered sequences. Three-dimensional models are commonly available and are familiar to those skilled in the art. Computer programs are available which illustrate and display probable three-dimensional conformational structures of selected candidate sequences and can measure possible immunogenicity (e.g., Immunofilter program of Xencor, Inc. of Monrovia, Calif). Inspection of these displays permits analysis of the likely role of the residues in the functioning of the candidate sequence, i.e., the analysis of residues that influence the ability of the candidate scFv to bind its antigen. In this way, residues can be selected and combined from the parent and reference sequences so that the desired characteristic, such as affinity for the target antigen(s), is achieved. Alternatively, or in addition to, the above procedures, other suitable methods of engineering can be used.

Screening of an scFv for specific binding to similar proteins or fragments can be conveniently achieved using nucleotide (DNA or RNA display) or peptide display libraries, for example, in vitro display. This method involves the screening of large collections of peptides for individual members having the desired function or structure. The displayed nucleotide or peptide sequences can be from 3 to 5000 or more nucleotides or amino acids in length, frequently from 5-100 amino acids long, and often from about 8 to 25 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT Patent Publication Nos. WO 91/185, WO 91/18980, WO 91/19818, and WO 93/08278.

Other systems for generating libraries of peptides have aspects of both in vitro chemical synthesis and recombinant methods. See, PCT Patent Publication Nos. WO 92/05258, WO 92/14843, and WO 96/19256. See also, U.S. Pat. Nos. 5,658,754; and 5,643,768. Peptide display libraries, vector, and screening kits are commercially available from such suppliers as Invitrogen (Carlsbad, Calif.), and Cambridge Antibody Technologies (Cambridgeshire, UK). See, e.g., U.S. Pat. Nos. 4,704,692, 4,939,666, 4,946,778, 5,260,203, 5,455,030, 5,518,889, 5,534,621, 5,656,730, 5,763,733, 5,767,260, 5,856,456, assigned to Enzon; U.S. Pat. Nos. 5,223,409, 5,403,484, 5,571,698, 5,837,500, assigned to Dyax, U.S. Pat. Nos. 5,427,908, 5,580,717, assigned to Affymax; 5,885,793, assigned to Cambridge Antibody Technologies; 5,750,373, assigned to Genentech, U.S. Pat. Nos. 5,618,920, 5,595,898, 5,576,195, 5,698,435, 5,693,493, 5,698,417, assigned to Xoma, Colligan, supra; Ausubel, supra; or Sambrook, supra.

An scFv of the disclosure can bind human or other mammalian proteins with a wide range of affinities (KD). In a preferred aspect, at least one scFv of the present disclosure can optionally bind to a target protein with high affinity, for example, with a KD equal to or less than about 10⁻⁷M, such as but not limited to, 0.1-9.9 (or any range or value therein)×10⁻⁸, 10⁻⁹, 10⁻¹⁰, 10⁻¹¹, 10⁻¹², 10⁻¹³, 10⁻¹⁴, 10⁻¹⁵ or any range or value therein, as determined by surface plasmon resonance or the Kinexa method, as practiced by those of skill in the art.

The affinity or avidity of a scFv for an antigen can be determined experimentally using any suitable method. (See, for example, Berzofsky, et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein). The measured affinity of a particular scFv-antigen interaction can vary if measured under different conditions (e.g., salt concentration, pH). Thus, measurements of affinity and other antigen-binding parameters (e.g., K D, Kon, Koff) are preferably made with standardized solutions of protein scaffold and antigen, and a standardized buffer, such as the buffer described herein.

Competitive assays can be performed with the scFv of the disclosure in order to determine what proteins, antibodies, and other antagonists compete for binding to a target protein with the scFv of the present disclosure and/or share the epitope region. These assays as readily known to those of ordinary skill in the art evaluate competition between antagonists or ligands for a limited number of binding sites on a protein. The protein and/or antibody is immobilized or insolubilized before or after the competition and the sample bound to the target protein is separated from the unbound sample, for example, by decanting (where the protein/antibody was pre-insolubilized) or by centrifuging (where the protein/antibody was precipitated after the competitive reaction). Also, the competitive binding may be determined by whether function is altered by the binding or lack of binding of the scFv to the target protein, e.g., whether the scFv molecule inhibits or potentiates the enzymatic activity of, for example, a label. ELISA and other functional assays may be used, as well known in the art.

Nucleic Acid Molecules

Nucleic acid molecules of the disclosure encoding an scFv can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules of the disclosure can include nucleic acid molecules comprising an open reading frame (ORF), optionally, with one or more introns, e.g., but not limited to, at least one specified portion of at least one scFv; nucleic acid molecules comprising the coding sequence for a protein scaffold or loop region that binds to the target protein; and nucleic acid molecules which comprise a nucleotide sequence substantially different from those described above but which, due to the degeneracy of the genetic code, still encode the protein scaffold as described herein and/or as known in the art. Of course, the genetic code is well known in the art. Thus, it would be routine for one skilled in the art to generate such degenerate nucleic acid variants that code for a specific scFv of the present disclosure. See, e.g., Ausubel, et al., supra, and such nucleic acid variants are included in the present disclosure.

As indicated herein, nucleic acid molecules of the disclosure which comprise a nucleic acid encoding a scFv can include, but are not limited to, those encoding the amino acid sequence of a scFv fragment, by itself; the coding sequence for the entire protein scaffold or a portion thereof; the coding sequence for a scFv, fragment or portion, as well as additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example, ribosome binding and stability of mRNA); an additional coding sequence that codes for additional amino acids, such as those that provide additional functionalities. Thus, the sequence encoding a protein scaffold can be fused to a marker sequence, such as a sequence encoding a peptide that facilitates purification of the fused protein scaffold comprising a protein scaffold fragment or portion.

Polynucleotides Selectively Hybridizing to Polynucleotide as Described Herein

The disclosure provides isolated nucleic acids that hybridize under selective hybridization conditions to a polynucleotide disclosed herein. Thus, the polynucleotides can be used for isolating, detecting, and/or quantifying nucleic acids comprising such polynucleotides. For example, polynucleotides of the present disclosure can be used to identify, isolate, or amplify partial or full-length clones in a deposited library. The polynucleotides can be genomic or cDNA sequences isolated, or otherwise complementary to, a cDNA from a human or mammalian nucleic acid library.

Preferably, the cDNA library comprises at least 80% full-length sequences, preferably, at least 85% or 90% full-length sequences, and, more preferably, at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences.

Optionally, polynucleotides will encode at least a portion of a protein scaffold encoded by the polynucleotides described herein. The polynucleotides embrace nucleic acid sequences that can be employed for selective hybridization to a polynucleotide encoding a protein scaffold of the present disclosure. See, e.g., Ausubel, supra; Colligan, supra, each entirely incorporated herein by reference.

Construction of Nucleic Acids

The isolated nucleic acids of the disclosure can be made using (a) recombinant methods, (b) synthetic techniques, (c) purification techniques, and/or (d) combinations thereof, as well-known in the art.

The nucleic acids can conveniently comprise nucleotide sequences in addition to a polynucleotide of the present disclosure. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the disclosure. For example, a hexa-histidine marker sequence provides a convenient means to purify the proteins of the disclosure. The nucleic acid of the disclosure, excluding the coding sequence, is optionally a vector, adapter, or linker for cloning and/or expression of a polynucleotide of the disclosure.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra).

Recombinant Methods for Constructing Nucleic Acids

The isolated nucleic acid compositions of this disclosure, such as RNA, cDNA, genomic DNA, or any combination thereof, can be obtained from biological sources using any number of cloning methodologies known to those of skill in the art. In some aspects, oligonucleotide probes that selectively hybridize, under stringent conditions, to the polynucleotides of the present disclosure are used to identify the desired sequence in a cDNA or genomic DNA library. The isolation of RNA, and construction of cDNA and genomic libraries are well known to those of ordinary skill in the art. (See, e.g., Ausubel, supra; or Sambrook, supra).

Nucleic Acid Screening and Isolation Methods

A cDNA or genomic library can be screened using a probe based upon the sequence of a polynucleotide of the disclosure. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent, such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the disclosure without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.)

For instance, polymerase chain reaction (PCR) technology can be used to amplify the sequences of polynucleotides of the disclosure and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

Synthetic Methods for Constructing Nucleic Acids

The isolated nucleic acids of the disclosure can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

Recombinant Expression Cassettes

The disclosure further provides recombinant expression cassettes comprising a nucleic acid of the disclosure. A nucleic acid sequence of the disclosure, for example, a cDNA or a genomic sequence encoding a protein scaffold of the disclosure, can be used to construct a recombinant expression cassette that can be introduced into at least one desired host cell. A recombinant expression cassette will typically comprise a polynucleotide of the disclosure operably linked to transcriptional initiation regulatory sequences that will direct the transcription of the polynucleotide in the intended host cell. Both heterologous and non-heterologous (i.e., endogenous) promoters can be employed to direct expression of the nucleic acids of the disclosure.

In some aspects, isolated nucleic acids that serve as promoter, enhancer, or other elements can be introduced in the appropriate position (upstream, downstream or in the intron) of a non-heterologous form of a polynucleotide of the disclosure so as to up or down regulate expression of a polynucleotide of the disclosure. For example, endogenous promoters can be altered in vivo or in vitro by mutation, deletion and/or substitution.

Expression Vectors and Host Cells

The disclosure also relates to vectors that include isolated nucleic acid molecules of the disclosure, host cells that are genetically engineered with the recombinant vectors, and the production of at least one protein scaffold by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, ampicillin, zeocin (Sh bla gene), puromycin (pac gene), hygromycin B (hygB gene), G418/Geneticin (neo gene), DHFR (encoding Dihydrofolate Reductase and conferring resistance to Methotrexate), mycophenolic acid, or glutamine synthetase (GS, U.S. Pat. Nos. 5,122,464; 5,770,359; 5,827,739), blasticidin (bsd gene), resistance genes for eukaryotic cell culture as well as ampicillin, zeocin (Sh bla gene), puromycin (pac gene), hygromycin B (hygB gene), G418/Geneticin (neo gene), kanamycin, spectinomycin, streptomycin, carbenicillin, bleomycin, erythromycin, polymyxin B, or tetracycline resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

Expression vectors will preferably but optionally include at least one selectable cell surface marker for isolation of cells modified by the compositions and methods of the disclosure. Selectable cell surface markers of the disclosure comprise surface proteins, glycoproteins, or group of proteins that distinguish a cell or subset of cells from another defined subset of cells. Preferably the selectable cell surface marker distinguishes those cells modified by a composition or method of the disclosure from those cells that are not modified by a composition or method of the disclosure. Such cell surface markers include, e.g., but are not limited to, “cluster of designation” or “classification determinant” proteins (often abbreviated as “CD”) such as a truncated or full length form of CD19, CD271, CD34, CD22, CD20, CD33, CD52, or any combination thereof. Cell surface markers further include the suicide gene marker RQR8 (Philip B et al. Blood. 2014 Aug 21; 124(8):1277-87).

Expression vectors will preferably but optionally include at least one selectable drug resistance marker for isolation of cells modified by the compositions and methods of the disclosure. Selectable drug resistance markers of the disclosure may comprise wild-type or mutant Neo, DHFR, TYMS, FRANCF, RAD51C, GCS, MDR1, ALDH1, NKX2.2, or any combination thereof.

At least one protein scaffold of the disclosure can be expressed in a modified form, such as a fusion protein, and can include not only secretion signals, but also additional heterologous functional regions. For instance, a region of additional amino acids, particularly charged amino acids, can be added to the N-terminus of a protein scaffold to improve stability and persistence in the host cell, during purification, or during subsequent handling and storage. Also, peptide moieties can be added to a protein scaffold of the disclosure to facilitate purification. Such regions can be removed prior to final preparation of a protein scaffold or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid encoding a protein of the disclosure. Alternatively, nucleic acids of the disclosure can be expressed in a host cell by turning on (by manipulation) in a host cell that contains endogenous DNA encoding a protein scaffold of the disclosure. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the protein scaffolds, specified portions or variants thereof, are bacterial, yeast, and mammalian cells as known in the art. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Ag14 cells (ATCC Accession Number CRL-1851). In a preferred aspect, the recombinant cell is a P3X63Ab8.653 or an SP2/0-Ag14 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present disclosure are known and/or available, for instance, from the American Type Culture Collection Catalogue of Cell Lines and Hybridomas (www.atcc.org) or other known or commercial sources.

When eukaryotic host cells are employed, polyadenylation or transcription terminator sequences are typically incorporated into the vector. An example of a terminator sequence is the polyadenylation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

scFv Purification

An scFv can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

An scFv of the disclosure include purified products, products of chemical synthetic procedures, and products produced by recombinant techniques from a prokaryotic or eukaryotic host, including, for example, E. coli, yeast, higher plant, insect and mammalian cells. Depending upon the host employed in a recombinant production procedure, the protein scaffold of the disclosure can be glycosylated or can be non-glycosylated. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

Amino Acid Codes

The amino acids that make up protein scaffolds of the disclosure are often abbreviated. The amino acid designations can be indicated by designating the amino acid by its single letter code, its three letter code, name, or three nucleotide codon(s) as is well understood in the art (see Alberts, B., et al., Molecular Biology of The Cell, Third Ed., Garland Publishing, Inc., New York, 1994). A protein scaffold of the disclosure can include one or more amino acid substitutions, deletions or additions, from spontaneous or mutations and/or human manipulation, as specified herein. Amino acids in a protein scaffold of the disclosure that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to, at least one neutralizing activity. Sites that are critical for protein scaffold binding can also be identified by structural analysis, such as crystallization, nuclear magnetic resonance or photoaffinity labeling (Smith, et al., J. Mol. Biol. 224:899-904 (1992) and de Vos, et al., Science 255:306-312 (1992)).

As those of skill will appreciate, the disclosure includes at least one biologically active protein scaffold of the disclosure. Biologically active protein scaffolds have a specific activity at least 20%, 30%, or 40%, and, preferably, at least 50%, 60%, or 70%, and, most preferably, at least 80%, 90%, or 95%-99% or more of the specific activity of the native (non-synthetic), endogenous or related and known protein scaffold. Methods of assaying and quantifying measures of enzymatic activity and substrate specificity are well known to those of skill in the art.

In another aspect, the disclosure relates to protein scaffolds and fragments, as described herein, which are modified by the covalent attachment of an organic moiety. Such modification can produce a protein scaffold fragment with improved pharmacokinetic properties (e.g., increased in vivo serum half-life). The organic moiety can be a linear or branched hydrophilic polymeric group, fatty acid group, or fatty acid ester group. In particular aspect, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified protein scaffolds and fragments of the disclosure can comprise one or more organic moieties that are covalently bonded, directly or indirectly, to the antibody. Each organic moiety that is bonded to a protein scaffold or fragment of the disclosure can independently be a hydrophilic polymeric group, a fatty acid group or a fatty acid ester group. As used herein, the term “fatty acid” encompasses mono-carboxylic acids and di— carboxylic acids. A “hydrophilic polymeric group,” as the term is used herein, refers to an organic polymer that is more soluble in water than in octane. For example, polylysine is more soluble in water than in octane. Thus, a protein scaffold modified by the covalent attachment of polylysine is encompassed by the disclosure. Hydrophilic polymers suitable for modifying protein scaffolds of the disclosure can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the protein scaffold of the disclosure has a molecular weight of about 800 to about 150,000 Daltons as a separate molecular entity. For example, PEG5000 and PEG20,000, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying protein scaffolds of the disclosure can be saturated or can contain one or more units of unsaturation. Fatty acids that are suitable for modifying protein scaffolds of the disclosure include, for example, n-dodecanoate (C12, laurate), n-tetradecanoate (C14, myristate), n-octadecanoate (C18, stearate), n-eicosanoate (C20, arachidate), n-docosanoate (C22, behenate), n-triacontanoate (C30), n-tetracontanoate (C40), cis-Δ9-octadecanoate (C18, oleate), all cis-Δ5,8,11,14-eicosatetraenoate (C20, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably, one to about six, carbon atoms.

The modified protein scaffolds and fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups, such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example, a divalent C1-C12 group wherein one or more carbon atoms can be replaced by a heteroatom, such as oxygen, nitrogen or sulfur. Suitable linker moieties include, for example, tetraethylene glycol, —(CH₂)₃—, —NH—(CH₂)₆—NH—, —(CH₂)₂—NH— and —CH2—O—CH₂—CH₂—O—CH₂—CH₂—O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate, as described, or can be reacted with maleic anhydride and the resulting product cyclized to produce an activated maleimide derivative of the fatty acid. (See, for example, Thompson, et al., WO 92/16221, the entire teachings of which are incorporated herein by reference.)

The modified protein scaffolds of the disclosure can be produced by reacting a protein scaffold or fragment with a modifying agent. For example, the organic moieties can be bonded to the protein scaffold in a non-site specific manner by employing an amine-reactive modifying agent, for example, an NHS ester of PEG. Modified protein scaffolds and fragments comprising an organic moiety that is bonded to specific sites of a protein scaffold of the disclosure can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6(10):2233-2241 (1997); Itoh et al., Bioorg. Chem., 24(1): 59-68 (1996); Capellas et al., Biotechnol. Bioeng., 56(4):456-463 (1997)), and the methods described in Hermanson, G. T., Bioconjugate Techniques, Academic Press: San Diego, Calif. (1996).

### Definitions

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more standard deviations. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The disclosure provides isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various aspects, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals.

The disclosure provides fragments and variants of the disclosed DNA sequences and proteins encoded by these DNA sequences. As used throughout the disclosure, the term “fragment” refers to a portion of the DNA sequence or a portion of the amino acid sequence and hence protein encoded thereby. Fragments of a DNA sequence comprising coding sequences may encode protein fragments that retain biological activity of the native protein and hence DNA recognition or binding activity to a target DNA sequence as herein described. Alternatively, fragments of a DNA sequence that are useful as hybridization probes generally do not encode proteins that retain biological activity or do not retain promoter activity. Thus, fragments of a DNA sequence may range from at least about 20 nucleotides, about 50 nucleotides, about 100 nucleotides, and up to the full-length polynucleotide of the disclosure.

Nucleic acids or proteins of the disclosure can be constructed by a modular approach including preassembling monomer units and/or repeat units in target vectors that can subsequently be assembled into a final destination vector. Polypeptides of the disclosure may comprise repeat monomers of the disclosure and can be constructed by a modular approach by preassembling repeat units in target vectors that can subsequently be assembled into a final destination vector. The disclosure provides polypeptide produced by this method as well nucleic acid sequences encoding these polypeptides. The disclosure provides host organisms and cells comprising nucleic acid sequences encoding polypeptides produced this modular approach.

The term “antibody” is used in the broadest sense and specifically covers single monoclonal antibodies (including agonist and antagonist antibodies) and antibody compositions with polyepitopic specificity. It is also within the scope hereof to use natural or synthetic analogs, mutants, variants, alleles, homologs and orthologs (herein collectively referred to as “analogs”) of the antibodies hereof as defined herein. Thus, according to an aspect hereof, the term “antibody hereof” in its broadest sense also covers such analogs. Generally, in such analogs, one or more amino acid residues may have been replaced, deleted and/or added, compared to the antibodies hereof as defined herein.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′—SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific or multivalent structures formed from antibody fragments. In an antibody fragment comprising one or more heavy chains, the heavy chain(s) can contain any constant domain sequence (e.g., CHI in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s). The term further includes single domain antibodies (“sdAB”) which generally refers to an antibody fragment having a single monomeric variable antibody domain, (for example, from camelids). Such antibody fragment types will be readily understood by a person having ordinary skill in the art.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of” shall mean excluding more than trace elements of other ingredients and substantial method steps. Aspects defined by each of these transition terms are within the scope of this disclosure.

The term “epitope” refers to an antigenic determinant of a polypeptide. An epitope could comprise three amino acids in a spatial conformation, which is unique to the epitope. Generally, an epitope consists of at least 4, 5, 6, or 7 such amino acids, and more usually, consists of at least 8, 9, or 10 such amino acids. Methods of determining the spatial conformation of amino acids are known in the art, and include, for example, x-ray crystallography and two-dimensional nuclear magnetic resonance.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristylation, and glycosylation.

“Modulation” or “regulation” of gene expression refers to a change in the activity of a gene. Modulation of expression can include, but is not limited to, gene activation and gene repression.

The term “operatively linked” or its equivalents (e.g., “linked operatively”) means two or more molecules are positioned with respect to each other such that they are capable of interacting to affect a function attributable to one or both molecules or a combination thereof.

Non-covalently linked components and methods of making and using non-covalently linked components, are disclosed. The various components may take a variety of different forms as described herein. For example, non-covalently linked (i.e., operatively linked) proteins may be used to allow temporary interactions that avoid one or more problems in the art. The ability of non-covalently linked components, such as proteins, to associate and dissociate enables a functional association only or primarily under circumstances where such association is needed for the desired activity. The linkage may be of duration sufficient to allow the desired effect.

A method for directing proteins to a specific locus in a genome of an organism is disclosed. The method may comprise the steps of providing a DNA localization component and providing an effector molecule, wherein the DNA localization component and the effector molecule are capable of operatively linking via a non-covalent linkage.

The term “scFv” refers to a single-chain variable fragment. scFv is a fusion protein of the variable regions of the heavy (VH) and light chains (VL) of immunoglobulins, connected with a linker peptide. The linker peptide may be from about 5 to 40 amino acids or from about 10 to 30 amino acids or about 5, 10, 15, 20, 25, 30, 35, or 40 amino acids in length. Single-chain variable fragments lack the constant Fc region found in complete antibody molecules, and, thus, the common binding sites (e.g., Protein G) used to purify antibodies. The term further includes a scFv that is an intrabody, an antibody that is stable in the cytoplasm of the cell, and which may bind to an intracellular protein.

The term “single domain antibody” means an antibody fragment having a single monomeric variable antibody domain which is able to bind selectively to a specific antigen. A single-domain antibody generally is a peptide chain of about 110 amino acids long, comprising one variable domain (VH) of a heavy-chain antibody, or of a common IgG, which generally have similar affinity to antigens as whole antibodies, but are more heat-resistant and stable towards detergents and high concentrations of urea. Examples are those derived from camelid or fish antibodies. Alternatively, single-domain antibodies can be made from common murine or human IgG with four chains.

The terms “specifically bind” and “specific binding” as used herein refer to the ability of an antibody, an antibody fragment or a nanobody to preferentially bind to a particular antigen that is present in a homogeneous mixture of different antigens. In some aspects, a specific binding interaction will discriminate between desirable and undesirable antigens in a sample. In some aspects, more than about ten- to 100-fold or more (e.g., more than about 1000- or 10,000-fold). “Specificity” refers to the ability of an immunoglobulin or an immunoglobulin fragment, such as a nanobody, to bind preferentially to one antigenic target versus a different antigenic target and does not necessarily imply high affinity.

A “target site” or “target sequence” is a nucleic acid sequence that defines a portion of a nucleic acid to which a binding molecule will bind, provided sufficient conditions for binding exist.

The terms “nucleic acid” or “oligonucleotide” or “polynucleotide” refer to at least two nucleotides covalently linked together. The depiction of a single strand also defines the sequence of the complementary strand. Thus, a nucleic acid may also encompass the complementary strand of a depicted single strand. A nucleic acid of the disclosure also encompasses substantially identical nucleic acids and complements thereof that retain the same structure or encode for the same protein.

Probes of the disclosure may comprise a single stranded nucleic acid that can hybridize to a target sequence under stringent hybridization conditions. Thus, nucleic acids of the disclosure may refer to a probe that hybridizes under stringent hybridization conditions.

Nucleic acids of the disclosure may be single- or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo- and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally-occur, rendering the entire nucleic acid sequence non-naturally occurring. Nucleic acids of the disclosure may contain one or more duplicated, inverted or repeated sequences, the resultant sequence of which does not naturally-occur, rendering the entire nucleic acid sequence non-naturally occurring. Nucleic acids of the disclosure may contain modified, artificial, or synthetic nucleotides that do not naturally-occur, rendering the entire nucleic acid sequence non-naturally occurring.

Given the redundancy in the genetic code, a plurality of nucleotide sequences may encode any particular protein. All such nucleotides sequences are contemplated herein.

As used throughout the disclosure, the term “operably linked” refers to the expression of a gene that is under the control of a promoter with which it is spatially connected. A promoter can be positioned 5′ (upstream) or 3′ (downstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

As used throughout the disclosure, the term “promoter” refers to a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, EF-1 Alpha promoter, CAG promoter, SV40 early promoter or SV40 late promoter and the CMV IE promoter.

As used throughout the disclosure, the term “substantially complementary” refers to a first sequence that is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540, or more nucleotides or amino acids, or that the two sequences hybridize under stringent hybridization conditions.

As used throughout the disclosure, the term “substantially identical” refers to a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540 or more nucleotides or amino acids, or with respect to nucleic acids, if the first sequence is substantially complementary to the complement of the second sequence.

As used throughout the disclosure, the term “variant” when used to describe a nucleic acid, refers to (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

As used throughout the disclosure, the term “vector” refers to a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. A vector may comprise a combination of an amino acid with a DNA sequence, an RNA sequence, or both a DNA and an RNA sequence.

As used throughout the disclosure, the term “variant” when used to describe a peptide or polypeptide, refers to a peptide or polypeptide that differs in amino acid sequence by the insertion, deletion, or conservative substitution of amino acids, but retain at least one biological activity. Variant can also mean a protein with an amino acid sequence that is substantially identical to a referenced protein with an amino acid sequence that retains at least one biological activity.

A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157: 105-132 (1982). The hydropathic index of an amino acid is based on a consideration of its hydrophobicity and charge. Amino acids of similar hydropathic indexes can be substituted and still retain protein function. In an aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example immunogenicity. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Tables A, B, or C below. In some aspects, fusion polypeptides and/or nucleic acids encoding such fusion polypeptides include conservative substitutions have been introduced by modification of polynucleotides encoding polypeptides of the disclosure. Amino acids can be classified according to physical properties and contribution to secondary and tertiary protein structure. A conservative substitution is a substitution of one amino acid for another amino acid that has similar properties. Exemplary conservative substitutions are set out in Table A.

TABLE A
Conservative Substitutions I

| Side chain characteristics | | Amino Acid |
|---|---|---|
| Aliphatic | Non-polar | G A P I L V F |
| | Polar-uncharged | C S T M N Q |
| | Polar-charged | D E K R |

| | |
|---|---|
| Aromatic | H F W Y |
| Other | N Q D E |

Alternately, conservative amino acids can be grouped as described in Lehninger, (Biochemistry, Second Edition; Worth Publishers, Inc. NY, N.Y. (1975), pp. 71-77) as set forth in Table B.

TABLE B
Conservative Substitutions II

| Side Chain Characteristic | | Amino Acid |
|---|---|---|
| Non-polar (hydrophobic) | Aliphatic: | A L I V P |
| | Aromatic: | F W Y |
| | Sulfur-containing: | M |
| | Borderline: | G Y |
| Uncharged-polar | Hydroxyl: | S T Y |
| | Amides: | N Q |
| | Sulfhydryl: | C |
| | Borderline: | G Y |

| | |
|---|---|
| Positively Charged (Basic): | K R H |
| Negatively Charged (Acidic): | D E |

Alternately, exemplary conservative substitutions are set out in Table C.

TABLE C
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the polypeptides of the disclosure are intended to include polypeptides bearing one or more insertions, deletions, or substitutions, or any combination thereof, of amino acid residues as well as modifications other than insertions, deletions, or substitutions of amino acid residues. Polypeptides or nucleic acids of the disclosure may contain one or more conservative substitution.

As used throughout the disclosure, the term “more than one” of the aforementioned amino acid substitutions refers to 2, 3, 4, 5, 6, 7, 8, 9, 10, 11, 12, 13, 14, 15, 16, 17, 18, 19, or 20 or more of the recited amino acid substitutions. The term “more than one” may refer to 2, 3, 4, or 5 of the recited amino acid substitutions.

Polypeptides and proteins of the disclosure, either their entire sequence, or any portion thereof, may be non-naturally occurring. Polypeptides and proteins of the disclosure may contain one or more mutations, substitutions, deletions, or insertions that do not naturally-occur, rendering the entire amino acid sequence non-naturally occurring. Polypeptides and proteins of the disclosure may contain one or more duplicated, inverted or repeated sequences, the resultant sequence of which does not naturally-occur, rendering the entire amino acid sequence non-naturally occurring. Polypeptides and proteins of the disclosure may contain modified, artificial, or synthetic amino acids that do not naturally-occur, rendering the entire amino acid sequence non-naturally occurring.

As used throughout the disclosure, “sequence identity” may be determined by using the stand-alone executable BLAST engine program for blasting two sequences (b12seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to a specified percentage of residues that are the same over a specified region of each of the sequences. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

As used throughout the disclosure, the term “endogenous” refers to nucleic acid or protein sequence naturally associated with a target gene or a host cell into which it is introduced.

As used throughout the disclosure, the term “exogenous” refers to nucleic acid or protein sequence not naturally associated with a target gene or a host cell into which it is introduced, including non-naturally occurring multiple copies of a naturally occurring nucleic acid, e.g., DNA sequence, or naturally occurring nucleic acid sequence located in a non-naturally occurring genome location.

The disclosure provides methods of introducing a polynucleotide construct comprising a DNA sequence into a host cell. By “introducing” is intended presenting to the cell the polynucleotide construct in such a manner that the construct gains access to the interior of the host cell. The methods of the disclosure do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

### EXAMPLES

### Example 1. Expression of CSRs on K562 Cells

Materials and Methods

K562 cells were thawed into RPMI with 10% FBS and culture at 37° C. In preparation for electroporation, K562 cells were centrifuged and resuspended in appropriate buffer (Lonza). Cells were electroporated with CSR encoded by an mRNA using Lonza Nucleofector. The reaction was supplemented with RPMI+10% FBS medum and cells were rested overnight at 32° C. 24 hrs after electroporation, flow cytometry was used to assess expression of CSR via CD2 expression with anti CD2 primary antibody (R&D Systems) and FITC secondary antibody (Jackson Immuno Research).

Results

K562 cells do not express endogenous CD2. Expression of the CSRs was checked 24 hours post delivery via flow cytometry using a CD2 polyclonal antibody. Expression of all CSRs of the disclosure (CD2z.V2; 2DH.TF2z; 2DH.2TF2z; 82DH.TF2z; 82DH.2TF2z) was successfully detected on K562 cells, relative to a mock control (cells were electroporated with no mRNA). Table 1 summarizes mean fluorescence intensity (MFI) of CD2 expression in K562 cells that received CSR mRNA.

TABLE 1
Expression of CSRs on K562 cells

| Condition | Mean CD2 Expression in K562s (MFI) ± SEM |
|---|---|
| Mock | 832 ± 289.6 |
| CD2z.V2 (control) | 4192 ± 521.5 |
| 2DH.TF2z | 5455 ± 386.5 |
| 2DH.2TF2z | 6475 ± 1474 |
| 82DH.TF2z | 5154 |
| 82DH.2TF2z | 5624 ± 784.5 |

### Example 2. Expression and Functional Characterization of CSRs on Pan-T Cells

Materials and Methods

Pan-T cells were isolated by CD4/CD8 positive selection (CliniMACS) from healthy donor apheresis material (OBI or StemExpress). Frozen Pan T cells were thawed into Immunocult medium (StemCell Technologies) and rested overnight at 37° C. In preparation for electroporation, T cells were centrifuged and resuspended in appropriate buffer (Lonza). Cells were electroporated with CSR mRNA using Lonza Nucleofector. The reaction was supplemented with Immunocult medium and cultured overnight at 32° C. 24 hrs post electroporation, flow cytometry was used to assess expression of CSR via CD2 expression using an anti CD2 primary antibody (R&D Systems) and FITC secondary antibody (Jackson Immuno Research). Cells were transferred to 37° C. and cells were activated with Immunocult Human CD2 T cell activator (StemCell Technologies). 4 and 24 hours after activation, flow cytometry was used to assess expression of CSR signaling using anti-pSTAT5-AF647 antibody (BD).

Results

Expression of the CSRs was checked 24 hours post delivery via flow cytometry using a CD2 polyclonal antibody. Both frequency and MFI of CD2 expression was greater than mock control (cells were electroporated with no mRNA) for all CSRs tested (CD2z.V2; 2DH.TF2z; 2DH.2TF2z; 82DH.TF2z; 82DH.2TF2z). Table 2 shows the frequency and MFI of CD2 expression in pan T cells in two different donors.

TABLE 2
Expression of CSRs on Pan-T cells.

| | Mean Frequency of | | | |
|---|---|---|---|---|
| | CD2 Expression | | Mean CD2 Expression | |
| | (% of live cells) ± SEM | | (MFI) ± SEM | |
| Condition | Donor 1 | Donor 2 | Donor 1 | Donor 2 |
| Mock | 1.99 ± 0.46 | 2.25 ± 0.07 | 3463 ± 5.00 | 2909 ± 4.50 |
| CD2z.V2 | 24.20 ± 0.70 | 13.75 ± 3.05 | 5356 ± 33.00 | 4459 ± 288.00 |
| (control) | | | | |
| 2DH.TF2z | 3.355 ± 0.45 | 2.35 ± 0.10 | 3946 ± 24.00 | 3121 ± 4.50 |
| 2DH.2TF2z | 6.56 ± 0.89 | 5.29 ± 0.44 | 4443 ± 48.00 | 3637 ± 17.00 |
| 82DH.TF2z | 2.815 ± 0.51 | 2.73 | 3810 ± 76.5 | 3213 |
| 82DH.2TF2z | 7.00 ± 0.46 | 5.56 ± 0.94 | 4365 ± 168.5 | 3615 ± 50 |

### Example 3. Expression and Functional Characterization of CSRs on Treg Cells

Materials and Methods

Treg cells were isolated from healthy donor apheresis material (OBI, StemExpress, or StemCell Technologies) using Easy Sep Human CD4+CD127^{low}CD25+ Regulatory T cell Isolation kit (StemCell Technologies) following kit protocol. Purity of isolated Treg cells was confirmed by flow cytometry. Isolated Treg cells were cultured in X-VIVO 15 medium (Lonza) supplemented with 5% human AB serum (Lonza). In preparation for electroporation, Tregs were centrifuged and resuspended in appropriate buffer (Lonza). Cells were electroporated with CSR mRNA using Lonza Nucleofector. The reaction was supplemented with medium and rested overnight at 32° C. 24 hrs after electroporation, flow cytometry was used to assess expression of CSR via CD2 expression with anti CD2 primary antibody (R&D Systems) and FITC secondary antibody (Jackson Immuno Research). Cells were transferred to 37° C. and activated with Immunocult Human CD2 T cell activator (StemCell Technologies). 16 and 24 hours after activation, flow cytometry was used to assess the expression of CSR signaling using anti-pSTAT5-AF647 antibody (BD).

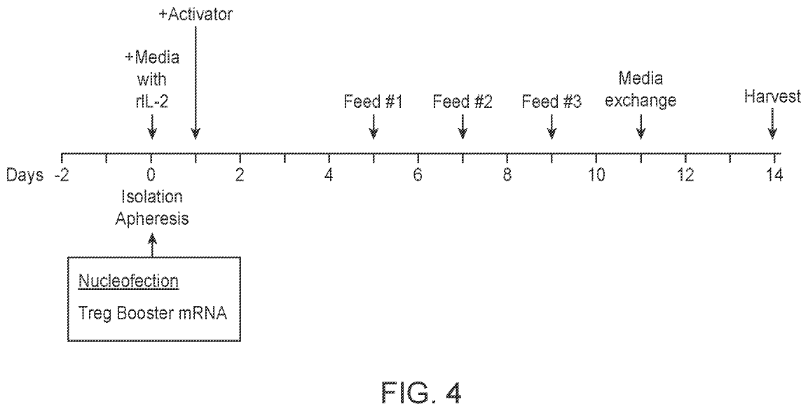

To track cellular expansion and viability, Treg cells were cultured in X-VIVO 15 medium (Lonza) supplemented with 5% human AB serum (Lonza) and 500U/mL IL-2 (Peprotech) and activated with Immunocult Human CD3/CD28/CD2 T cell activator (StemCell Technologies), cell counts were performed on days 0, 1, 5, 7, 9, 11, and 14 post electroporation using Luna Automated cell counter. Medium is supplemented at day 5, 7, and 9. Medium was exchanged on day 11. A schematic of the protocol is shown in FIG. 4.

Results

CSRs were delivered to pan T cells from two donors by electroporation. 24 hours later pan T cells were activated with Immunocult CD2 activator. Expression of pSTAT5, a readout of downstream CSR signaling, was tested via flow cytometry at baseline, 4 hours and 24 hours post activation of the cells with CD2 activator. Table 3 shows the time course of pSTAT5 expression triggered by anti-CD2 activation, in pan T cells that express the CSR, in two donors.

TABLE 3
pSTAT5 expression in Pan-Tcells expressing CSRs

| | Time course of pSTAT5 Expression post | | | | | |
|---|---|---|---|---|---|---|
| | activation in pan T cells (% of live cells) | | | | | |
| | Baseline | | +4 hours | | +24 hours | |
| Condition | Donor 1 | Donor 2 | Donor 1 | Donor 2 | Donor 1 | Donor 2 |
| Mock | 1.34 | 5.77 | 1.58 | 6.6 | 2.36 | 4.54 |
| CD2z.V2 | 2.09 | 29.4 | 3.52 | 41.1 | 17.7 | 47.9 |
| (control) | | | | | | |
| 2DH.TF2z | 3.27 | 2.85 | 5.43 | 4.2 | 5.67 | 5.17 |
| 2DH.2TF2z | 3.94 | 2.35 | 4.73 | 2.78 | 8.34 | 15.8 |
| 82DH.TF2z | 3.9 | 7.64 | 5.49 | 5.09 | 6.16 | 10.1 |
| 82DH.2TF2z | 2.03 | 6.84 | 3.24 | 11 | 5.5 | 6.36 |

Treg cells (CD4+CD25+CD127−) were purified using a StemCell Technologies Isolation kit. CSR mRNA was delivered by electroporation. Expression of the CSRs was interrogated 24 hours post-delivery by flow cytometry using a CD2 polyclonal antibody. Both frequency and MFI of CD2 expression was greater than mock control (cells were electroporated with no mRNA) for all CSRs tested (CD2z.V2; 2DH.TF2z; 2DH.2TF2z; 82DH.TF2z; 82DH.2TF2z). Table 4 shows the frequency and MFI of CD2 expression in Treg cells in two different donors.

TABLE 4
CSR expression in Treg Cells from two donors.

| | Frequency of CD2 Expression | | CD2 Expression | |
|---|---|---|---|---|
| | (% of live cells) | | (MFI) | |
| Condition | Donor 3 | Donor 4 | Donor 3 | Donor 4 |
| Mock | 0.28 | 1.15 | 536 | 4711 |
| CD2z.V2 | 12 | 14.4 | 1852 | 8016 |
| (control) | | | | |
| 2DH.TF2z | 3.24 | 7.5 | 1656 | 6953 |
| 2DH.2TF2z | 12.8 | 19.1 | 2318 | 11166 |
| 82DH.TF2z | 7.43 | 9.63 | 2180 | 8983 |
| 82DH.2TF2z | 25.5 | 16 | 3205 | 9510 |

CSRs were delivered to Treg cells from two donors by electroporation. 24 hours later, Treg cells were activated with Immunocult CD2-activator. Expression of pSTAT5, a readout of CSR signaling, was tested via flow cytometry at baseline, 16 hours and 24 hours after activation of the cells with CD2-activator. Table 5 shows the time course of pSTAT5 expression in pan T cells delivered CSRs after CD2 activation in two donors. 2DH.2TF2z shows the highest pSTAT5 activation in Treg cells.

TABLE 5
pSTAT5 expression in Treg cells from two donors

| | Time course of pSTAT5 Expression post | | | | | |
|---|---|---|---|---|---|---|
| | activation in Treg cells (% of live cells) | | | | | |
| | Baseline | | +16 hours | | +24 hours | |
| Condition | Donor 4 | Donor 5 | Donor 4 | Donor 5 | Donor 4 | Donor 5 |
| Mock | 1.3 | 0.87 | 3.6 | 1.92 | 5.3 | 2.72 |
| CD2z.V2 | 2.95 | 0.58 | 7.1 | 10.3 | 10.5 | 11.1 |
| (control) | | | | | | |
| 2DH.TF2z | 2.04 | — | 9.24 | — | 8.64 | — |
| 2DH.2TF2z | 5.775 | 5.29 | 13.86 | 10.3 | 15.75 | 13.5 |
| 82DH.TF2z | 4.05 | — | 10.5 | — | 11.55 | — |
| 82DH.2TF2z | 5.5425 | 6.94 | 13.32 | 7.45 | 13.635 | 9.16 |

Fold expansion and viability of Treg cells were assessed. A Treg cellular expansion of greater than 10% is evidence of successful expansion. For Donor 3, fold expansion and % cell viability was assessed on days 0, 5, 7, 11 and 14. On day 14, Treg cells expressing CSRs (CD2z.V2; 2DH.TF2z; 2DH.2TF2z; 82DH.TF2z; 82DH.2TF2z) resulted in at least 8-fold to 23-fold expansion. The viability of cells were comparable to the mock control (cells electroporated with no mRNA). For Donor 6, fold expansion and % cell viability was assessed on days 0, 1, 3, 9, 11 and 14. On day 14, Treg cells expressing CSRs (CD2z.V2; 2DH.TF2z; 2DH.2TF2z; 82DH.TF2z; 82DH.2TF2z) resulted in at least 13-fold to 16-fold expansion. The viability of cells were comparable to the mock control (cells electroporated with no mRNA).

### Example 4. Expression of CSR on T Cells Enhances Ex Vivo Expansion of Regulatory T Cells and Effector T Cells

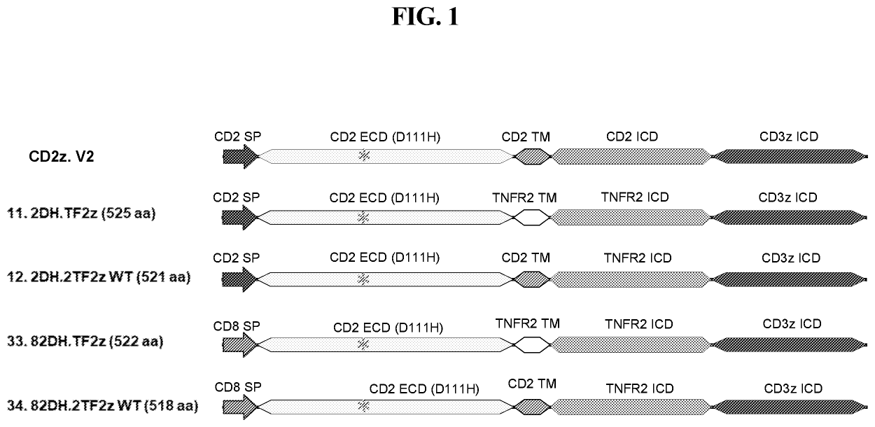

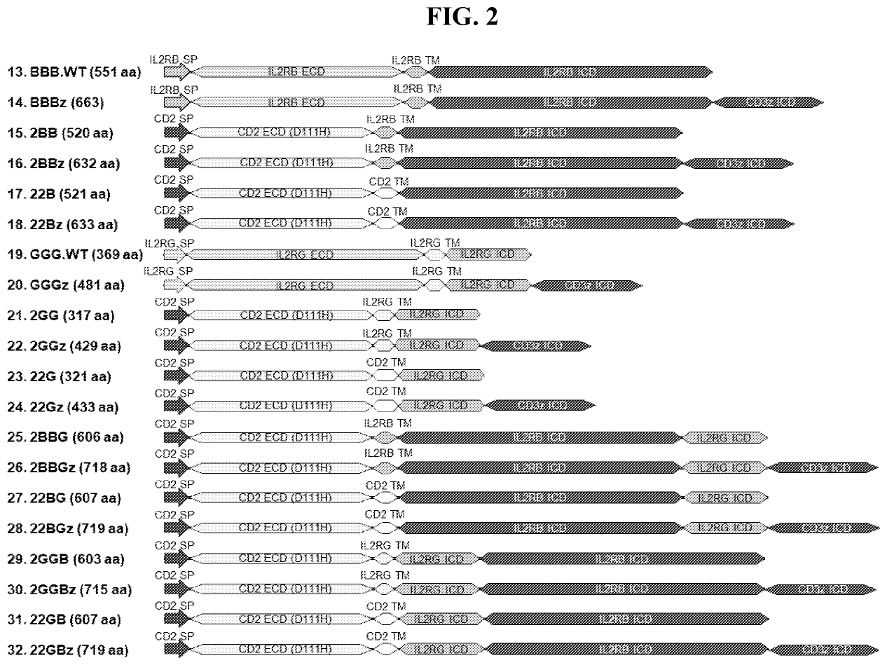

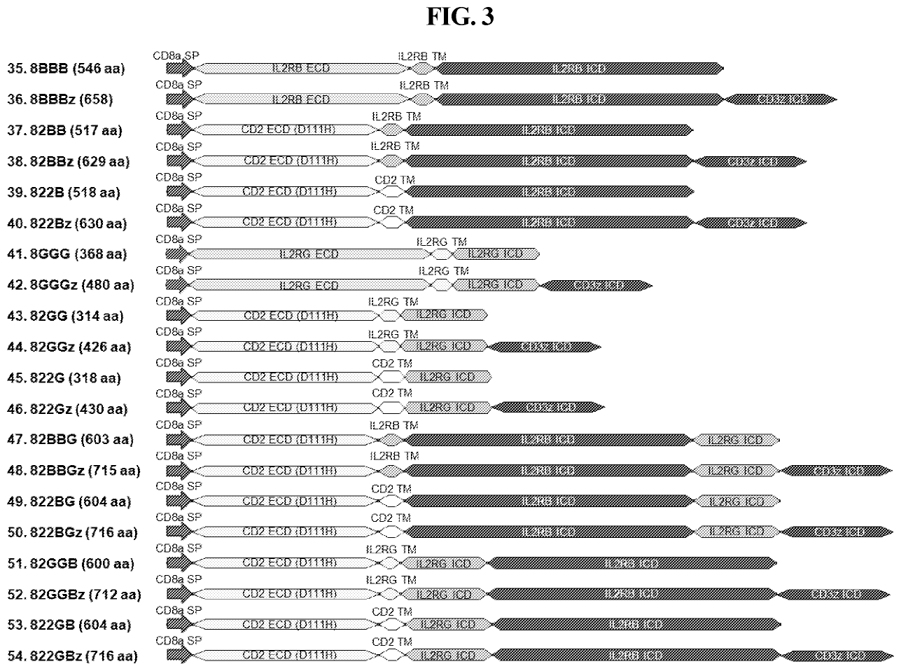

The CSRs delivering TNFR2 signals (FIG. 1) are designed to specifically promote expansion of Treg or Treg-like cells, alone or in combination with one or more of the other CSR constructs listed herein encoding the IL2RB or IL2RG intracellular signaling domains that are designed to deliver IL-2 signals (FIGS. 2 and 3).

Pan T cells, CD4+ or CD8+ T cells, isolated from normal donor blood were genetically modified to express one or more of the CSRs described herein, either with or without additional modification using the piggyBac® (PB) DNA modification system, as well as possible further editing using the Cas-CLOVER™ gene-editing system. Cells were electroporated in a single reaction with one or more CSRs, possibly also a transposon encoding a selection gene and an mRNA encoding the super piggyBac™ transposase enzyme, and possibly an mRNA encoding Cas-CLOVER™ with the appropriate guide RNA (gRNA) for site-specific gene editing. If a PB transposon was delivered, it may encode one or more of the following: a promoter, a CAR, an inducible proapoptotic polypeptide, a selection gene, FoxP3, and/or an activation-induced gene expression system. If Cas-CLOVER was delivered to cells, it may have been co-delivered with gRNA targeting genes associated with immune rejection like any of those that comprise the TCR, like TCRα, TCRβ or CD3, and/or a structural component of HLA, such as β2-microglobulin. The cells were subsequently stimulated, once or more, with agonist mAbs anti-CD2, anti-CD3 and/or anti-CD28, and were later selected for genetic modification over the course of an ex vivo culture period. Ex vivo culture may or may not be supplemented with exogenous cytokines such as IL-2. At the end of the initial culture period, T cells were phenotypically characterized by flow cytometry, indicating successful engineering. The relative expansion is determined by comparing the expression of cell surface markers on engineered cells with matched cells that are not engineered, thereby indicating Treg or Teff expansion.

Tregs will be detected using the following markers: CD4+CD25+CD127^{low} or CD4+CD25+ FoxP3+ or CD4+CD25+ FoxP3+CD127^{low}. Optionally, CD45RA surface expression may be used to characterize naïve Tregs. The function of Tregs will be assessed by testing their ability to suppress T effector cells in vitro and/or testing their ability to secrete cytokines such as TGF beta and IL-10 when stimulated using ELISA-like or FACS assays.

Teffs will be detected using the following markers: CD3+CD4+ or CD3+CD8+. A typical effector T cell also is CD62L−, CCR7−, CD45RA+ and CD45RO−. Effector T cell function can be assessed by a variety of standard methods. For example, the production of IFN gamma upon stimulation can be measured by ELISA assay or by flow cytometry. Stimulation may include TCR stimulation or CD3 bead stimulation.

### Example 5. Effects of CSR on JAK1/3, STAT5 and AKT Phosphorylation in Modified Regulatory T Cells and Effector T Cells

The CSRs delivering TNFR2 signals (FIG. 1) are designed to specifically promote expansion of Treg or Treg-like cells, in combination with one or more of the other CSR constructs listed herein encoding the IL2RB or IL2RG intracellular signaling domains that are designed to deliver IL−2 signals (FIGS. 2 and 3). A combination of one or more of the CSR constructs listed herein encoding the IL2RB or IL2RG intracellular signaling domains that are designed to deliver IL−2 signals can be used to specifically promote expansion of effector or effector-like T cells.

Treg cells that have a CD4+, CD25+, FOXP3+ and/or CSR+ expression profile were isolated and assessed for the phosphorylation of downstream signaling protein by western blot or FACS following stimulation of the CSR or other Treg-specific receptor. CSRs with TNFR2 intracellular domains were assessed for AKT and STAT5 activation. CSRs with IL2RB and IL2RG intracellular domains were assessed for JAK1/3 and STAT5 activation. Phosphorylation of these proteins indicates successful downstream signaling of the CSR. For example, protocols for determining JAK1/3 phosphorylation are described in Cheng et. al. Mol Cell Biol. 2008 April; 28(7): 2271-2282. Effector T cells that have a CD3+CD4+ or CD3+CD8+ and/or CSR+ expression profile were isolated and assessed for the phosphorylation of downstream signaling protein by western blot or FACS following stimulation of the CSR or other effector T cell receptor. CSRs with IL2RB and IL2RG intracellular domains were assess for JAK1/3 and STATS activation.

### Example 6. Therapeutic Function of CSR Modified Regulatory T Cells and Effector

T cells

An in vitro method to assess the therapeutic function of the CSR modified Tregs is to determine the ability of the stimulated Treg to produce suppressive cytokines, such as TGF beta and/or IL−10. Another method to assess therapeutic function is to determine the ability of stimulated Tregs to suppress effector T cell function in a co-culture assay.

An in vitro methods to assess the therapeutic function a CSR modified effector T cell is to determine the ability of stimulated effector T cell to product inflammatory cytokines, such as IFN gamma, IL−2, TNF alpha. Another method to assess therapeutic function of the effector T cell is to determine the ability of stimulated effector T cells to kill a specific target cell.

For in vivo assessment of therapeutic efficacy, standard animal models can be used for either Tregs or effector T cells. For example, exemplary models to assess therapeutic Tregs in vivo, include: 1) Tregs to prevent allo transplant rejection, tested in mice (Noyan et. al. Am J Transplant. 2017 April; 17(4):917-930. doi: 10.1111/ajt.14175); 2) Tregs to prevent allo transplant rejection, tested in mice (MacDonald et. al. J Clin Invest. 2016 Apr 1; 126(4): 1413-1424); 3) Tregs tested in a murine model of rheumatoid arthritis (Sun et. al. Inflammation. 2018 March;41(2):485-495); 4) Tregs tested in a murine model of Crohn's disease (Clough et. al Gut. 2020 Jan 24. pii: gutjn1-2019-319850); and 5) Tregs tested in a murine model of multiple sclerosis (Keeler et. al. Mol Ther. 2018 Jan 3;26(1):173-183). There are many exemplary models to assess therapeutic effector T cells in vivo, some of which are reviewed (Stromnes et. A1. Immunol Rev. 2014 January; 257(1): 145-164).

### Other Embodiments

While particular embodiments of the disclosure have been illustrated and described, various other changes and modifications can be made without departing from the spirit and scope of the disclosure. The scope of the appended claims includes all such changes and modifications that are within the scope of this disclosure.

What is claimed is:

1. A non-naturally occurring chimeric stimulatory receptor (CSR) comprising:
(a) an ectodomain comprising a activation component, wherein the activation component is isolated or derived from a first protein;
(b) a transmembrane domain; and
(c) at least one intracellular domain, wherein the intracellular domain is isolated or derived from a second protein; and
wherein the second protein is Tumor Necrosis Factor Receptor 2 (TNFR2).

2. The CSR of claim 1, wherein the activation component comprises an Interleukin-2 Receptor subunit Beta (IL2RB) extracellular domain, an Interleukin-2 Receptor subunit Gamma (IL2RG) extracellular domain or a CD2 extracellular domain.

3. The CSR of claim 1, wherein the activation component comprises a modification.

4. The CSR of claim 3, wherein the modification comprises a mutation or a truncation of the amino acid sequence of the activation component of the first protein when compared to a wild type sequence of the activation component of the first protein.

5. The CSR of claim 4, wherein the mutation or a truncation of the amino acid sequence of the activation component comprises a mutation or truncation of a CD2 extracellular domain or a portion thereof to which an agonist binds.

6. The CSR of claim 5, wherein the CSR comprising a mutation or truncation of a CD2 extracellular domain or a portion thereof to which an agonist binds does not bind CD58.

7. The CSR of claim 5, wherein the CD2 extracellular domain comprising the mutation or truncation comprises an amino acid sequence of SEQ ID NO: 4.

8. The CSR of claim 1, wherein the intracellular domain further a CD3 protein.

9. The CSR of claim 1, wherein the TNFR2 intracellular domain comprises the amino acid sequence of SEQ ID NO: 6.

10. The CSR of claim 8, wherein the CD3λ intracellular domain comprises the amino acid sequence of SEQ ID NO: 5.

11. The CSR of claim 1, wherein the ectodomain further comprises a signal peptide.

12. The CSR of claim 11, wherein the signal peptide is derived from a third protein.

13. The CSR of claim 12, wherein the first protein and the third protein are identical.

14. The CSR of claim 11, wherein the signal peptide comprises a CD2 signal peptide, an IL2RB signal peptide, an IL2RG signal peptide or a CD8 signal peptide.

15. The CSR of claim 14, wherein the CD2 signal peptide comprises an amino acid sequence of SEQ ID NO: 9.

16. The CSR of claim 14, wherein the CD8 signal peptide comprises an amino acid sequence of SEQ ID NO: 12.

17. The CSR of claim 1, wherein the transmembrane domain is isolated or derived from a fourth protein.

18. The CSR of claim 17, wherein the first protein and the fourth protein are identical.

19. The CSR of claim 17, wherein the transmembrane domain comprises a TNFR2 transmembrane domain, CD2 transmembrane domain, an IL2RB transmembrane domain or an IL2RG transmembrane domain.

20. The CSR of claim 19, wherein the TNFR2 transmembrane domain comprises an amino acid sequence of SEQ ID NO: 13.

21. The CSR of claim 19, wherein the CD2 transmembrane domain comprises an amino acid sequence of SEQ ID NO: 14.

22. The CSR of claim 1 comprising an amino acid sequence at least 95% identical to any one of SEQ ID NO: 17, 18, 37 or 38.

23. The CSR of claim 1 comprising an amino acid sequence at least 99% identical to any one of SEQ ID NO: 17, 18, 37 or 38.

24. The CSR of claim 1 comprising an amino acid sequence of any one of SEQ ID NO:
17, 18, 37 or 38.

25. A non-naturally occurring chimeric stimulatory receptor (CSR) comprising:
(a) an ectodomain comprising a signal peptide and an activation component, wherein the signal peptide comprises a CD2 signal peptide or a portion thereof or comprises a CD8 signal peptide or a portion thereof; and
wherein the activation component comprises a CD2 extracellular domain or a portion thereof and wherein the CD2 extracellular domain or a portion thereof comprises a mutation or truncation when compared to a wild type sequence of CD2;
(b) a transmembrane domain, wherein the transmembrane domain comprises a TNFR2 transmembrane domain or a portion thereof or comprises a CD2 transmembrane domain or portion thereof; and
(c) an intracellular domain, wherein the intracellular domain comprises a TNFR2 intracellular domain or a portion thereof and a CD3ζ protein or a portion thereof.

26. A nucleic acid sequence encoding the CSR of any one of claims 1-25.

27. A cell comprising the CSR of any one of claims 1-25.

28. A cell comprising the nucleic acid of claim 26.

29. The cell of claim 27 or 28, wherein the cell is an allogeneic cell.

30. The cell of claim 27 or 28, wherein the cell is an autologous cell.

31. The cell of claim 29 or 30, wherein the cell is a T-lymphocyte (T cell).

32. The cell of claim 31, wherein the T cell is a regulatory T cell.

33. The modified T cell of claim 61, wherein the CSR is transiently expressed in the modified T cell.

34. The modified T cell of claim 61, wherein the CSR is stably expressed in the modified T cell.

35. A composition comprising a population of modified T cells, wherein a plurality of the modified T cells of the population comprise the CSR according to any one of claims 1-25.

36. The composition according claim 35 for use in the treatment of a disease or disorder.

37. A method of treating a disease or disorder comprising administering to a subject in need thereof a therapeutically-effective amount of the composition according to claim 35.

38. A method of producing a population of modified T cells comprising introducing into a plurality of primary human T cells a composition comprising the CSR of claims 1-25 or a sequence encoding the same to produce a plurality of modified T cells under conditions that stably express the CSR within the plurality of modified T cells.

39. A method of producing a population of modified T cells comprising introducing into a plurality of primary human T cells a composition comprising the CSR of claims 1-25 or a sequence encoding the same to produce a plurality of modified T cells under conditions that transiently express the CSR within the plurality of modified T cells.

40. A method of expanding modified Treg cells within the population of modified T cells produced by the method of claim 38 or 39 comprising culturing the modified T cells with an activator composition under conditions to expand a plurality of activated modified Treg cells, wherein expansion of the plurality of modified Treg cells is at least 5% higher than the expansion of a plurality of Treg cells not stably expressing the CSR under the same conditions.

41. The method of claim 40, wherein the expansion of the plurality of modified T cells is at least 10% higher than the expansion of a plurality of wild-type T cells not stably expressing the CSR under the same conditions.

42. A composition comprising a population of modified T cells expanded by the method of claim 40 or 41.

43. The composition of claim 42 for use in the treatment of a disease or disorder.
